(19) United States
(12) Patent Application Publication
(10) Pub. No.: US 2010/0029003 A1
(43) Pub. Date: Feb. 4, 2010

(54) System and methods for identifying miRNA targets and for altering miRNA and target expression

(75) Inventors: David Bartel, Brookline, MA (US); Benjamin P. Lewis, Cambridge, MA (US); Matthew W. Jones-Rhoades, Somerville, MA (US); Christopher B. Burge, Belmont, MA (US)

Correspondence Address: WOLF GREENFIELD & SACKS, P.C., 600 ATLANTIC AVENUE, BOSTON, MA, 02210-2206, US

(73) Assignees: Whitehead Institute for Biomedical Research, Cambridge, MA (US); Massachusetts Institute of Technology, Cambridge, MA (US)

(21) Appl. No.: 12/321,489

(22) Filed: Jan. 21, 2009

Related U.S. Application Data
(63) Continuation of application No. 11/317,660, filed on Dec. 23, 2005, now abandoned.
(60) Provisional application No. 60/639,231, filed on Dec. 23, 2004.

(51) Int. Cl. C12N 15/85

(52) U.S. Cl. 435/455

(57) ABSTRACT

The present invention generally relates to microRNAs such as vertebrate microRNA (miRNA), for example, mammalian miRNA. Various aspects of the invention are directed to the detection, production, or expression of miRNA. In one aspect, the invention provides systems and methods for identifying targets of miRNA sequences. For instance, in one embodiment, gene sequences comprising UTRs are compared with miRNA sequences to determine the degree of interaction, for example, by determining a free energy measurement between the miRNA sequence and the UTR, and/or by determining complementarity between at least a portion of the miRNA sequence and the UTR. In another aspect, the invention is directed to the regulation of gene expression using miRNA. For example, gene expression within a cell may be altered by exposing the cell to an oligonucleotide comprising a sequence that is substantially antisense to at least a portion of an miRNA region of the gene, for example, antisense to a 6-mer or 7-mer portion of the miRNA. In still another aspect, the invention is directed to the treatment of cancer. For instance, in one set of embodiments, an isolated oligonucleotide comprising a sequence that is substantially antisense to an miRNA, or a portion of an miRNA, is administered to a subject having or being at risk of cancer. Yet other aspects of the invention are directed to compositions or kits including oligonucleotides comprising a sequence that is substantially antisense to an miRNA (or a portion of an miRNA), methods of promoting any of the above aspects, or the like.

### RELATED APPLICATIONS

This application is a continuation of U.S. patent application Ser. No. 11/317,660, filed Dec. 23, 2005, entitled “Systems and Methods for Identifying miRNA Targets and for Altering miRNA and Target Expression,” by Bartel, et al., which application claims the benefit of U.S. Provisional Patent Application Ser. No. 60/639,231, filed Dec. 23, 2004, entitled “Vertebrate miRNA and Systems and Methods of Detection Thereof,” by Lewis, et al. Each of these is incorporated herein by reference.

### FEDERALLY SPONSORED RESEARCH

Research leading to various aspects of the present invention were sponsored, at least in part, by the National Institutes of Health and the U.S. Department of Energy. The U.S. Government may have certain rights in the invention.

### FIELD OF INVENTION

The present invention generally relates to miRNA production and expression, including its use in the treatment of cancer through the regulation of gene expression.

### BACKGROUND

MicroRNAs are endogenous ˜22-nt (nucleotide) RNAs that play important gene regulatory roles by pairing to the messages of protein-coding genes to specify mRNA cleavage or repression of productive translation. The first to be discovered were the lin-4 and let-7 miRNAs, which are components of the gene regulatory network that controls the timing of C. elegans larval development. Other miRNA functions include the control of cell proliferation, cell death, and fat metabolism in flies, and the control of leaf and flower development in plants.

MicroRNA genes are one of the more abundant classes of regulatory genes in animals, estimated to comprise between 0.5 and 1 percent of the predicted genes in worms, flies, and humans. The possibility that many mammalian miRNAs play important roles during development and other processes is supported by their tissue-specific or developmental stage-specific expression patterns as well as their evolutionary conservation, which is very strong within mammals and often extends to invertebrate homologs. Indeed, miR-181, one of the many miRNAs conserved among vertebrates, can be expressed in the B-lymphocytes of mouse bone marrow, and the ectopic expression of this miRNA in hematopoietic stem/progenitor cells modulates blood cell development such that the proportion of B-lymphocytes increases.

Finding regulatory targets for miRNAs is relatively easy in plants. In a systematic search for the targets of 13 Arabidopsis miRNA families, 49 unique targets were found with a signal-to-noise ratio exceeding 10:1, by looking for Arabidopsis messages with near-perfect complementarity to the miRNAs (see, e.g., U.S. patent application Ser. No. 10/884,374, filed Jul. 1, 2004, entitled “MicroRNAs in Plants,” by Reinhart, et al., incorporated herein by reference). Confidence in many of these miRNAs was bolstered by the observation that complementarity is conserved among rice orthologs of the miRNAs and messages. These targets were greatly enriched in transcription factors involved in developmental patterning or stem cell maintenance and identity, suggesting that many plant miRNAs function during cellular differentiation to clear regulatory gene transcripts from daughter cell lineages, perhaps enabling more rapid differentiation without having to depend on regulatory genes having constitutively unstable messages.

### SUMMARY OF THE INVENTION

The present invention generally relates to miRNA production and expression, including its use in the treatment of cancer through the regulation of gene expression. The subject matter of the present invention involves, in some cases, interrelated products, alternative solutions to a particular problem, and/or a plurality of different uses of one or more systems and/or articles.

One aspect of the invention is a method of cancer treatment. According to one set of embodiments, the method includes administering, to a subject having or being at risk of cancer, a composition comprising an isolated oligonucleotide comprising a sequence that is substantially antisense to an miRNA. The sequence may be selected from the group consisting of SEQ ID NO: 682 to SEQ ID NO: 761, and/or the sequences of SEQ ID NO: 762 to SEQ ID NO: 1227 that are antisense to an miRNA. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. In some cases, the miRNA human miRNA. In one embodiment, the composition further comprises a pharmaceutically acceptable carrier.

In another set of embodiments, the method includes acts of operating a computer to: receive input of a conserved miRNA sequence and an mRNA of a gene comprising a UTR; define at least 6 nucleotides of the conserved miRNA sequence as an miRNA seed; identify, within the UTR, one or more segments of perfect complementarity with the miRNA seed; define an extended portion within the UTR that includes an identified segment of perfect complementarity, each base of the extended portion within the UTR being matched with one base of the miRNA as one of a A:U pair, a U:A pair, a C:G pair, a G:C pair, a G:U pair, or a U:G pair; define an extended portion within the miRNA corresponding to the extended portion within the UTR; determine base-pairing of at least 35 bases of the miRNA that is 3′ of the extended portion within the miRNA with at least 35 bases of the UTR that is 5′ of the extended portion within the UTR; and calculate a free energy measurement of the association of the extended portion within the miRNA and the at least 35 bases of the miRNA that is 3′ of the extended portion within the miRNA with the extended portion within the UTR and the at least 35 bases of the UTR that is 5′ of the extended portion within the UTR. The method also includes, in some cases, determining whether the miRNA adequately binds to the gene using the free energy measurement; and if adequate binding is determined, synthesizing an oligonucleotide comprising a sequence that is substantially antisense to the conserved miRNA sequence; and introducing the synthesized oligonucleotide into a tumor cell.

In still another set of embodiments, the method includes operating a computer to: provide a conserved miRNA sequence; provide a genome of an organism; define at least 6 nucleotides of the conserved miRNA sequence as an miRNA seed; identify a conserved UTR of a gene within the genome of the organism; and identify the gene as a target of the miRNA by determining whether the conserved UTR comprises a segment having perfect complementarity with the miRNA seed. The method also includes, in some cases, e.g., if the gene is a target of the miRNA, synthesizing an oligonucleotide comprising a sequence that is substantially antisense to the conserved miRNA sequence, and introducing the synthesized oligonucleotide into a tumor cell.

Another aspect of the invention is generally directed to a method of identifying a target to an miRNA in an organism. In one set of embodiments, the method includes acts of providing a conserved miRNA sequence and a genome of an organism, defining at least 6 nucleotides of the conserved miRNA sequence as an miRNA seed, identifying a conserved UTR of a gene within the genome of the organism, and identifying the gene as a target of the miRNA by determining whether the conserved UTR comprises a segment having perfect complementarity with the miRNA seed. In some embodiments, the conserved miRNA sequence is selected from the group consisting of the miRNA sequences, but not the miRNA-like control sequences, of SEQ ID NO: 3 to SEQ ID NO: 468, and in certain embodiments, the conserved miRNA sequence arises from a vertebrate, a mammal, or a human. The miRNA seed, in some cases, is selected from the group consisting of SEQ ID NO: 469 to SEQ ID NO: 535. In one embodiment, the method includes defining exactly 6 nucleotides or exactly 7 nucleotides of the conserved miRNA sequence as an miRNA seed. In some embodiments, the method further includes, if the gene is a target of the miRNA, synthesizing an oligonucleotide comprising a sequence that is substantially antisense to the conserved miRNA sequence. In addition, in some cases, the method further comprises administering the synthesized oligonucleotide to a cell or to a subject, for example, a human. In another embodiment, the method further comprises if the gene is a target of the miRNA, synthesizing an oligonucleotide comprising a sequence that is substantially antisense to the conserved miRNA sequence, and introducing the synthesized oligonucleotide into a cell, e.g., a tumor cell.

In another set of embodiments, the method includes providing a conserved miRNA sequence and an mRNA of a gene comprising a UTR; defining at least 6 nucleotides of the conserved miRNA sequence as an miRNA seed; identifying, within the UTR, one or more segments of perfect complementarity with the miRNA seed; defining an extended portion within the UTR that includes an identified segment of perfect complementarity, each base of the extended portion within the UTR being matched with one base of the miRNA as one of a A:U pair, a U:A pair, a C:G pair, a G:C pair, a G:U pair, or a U:G pair; defining an extended portion within the miRNA corresponding to the extended portion within the UTR; determining base-pairing of at least 35 bases of the miRNA that is 3′ of the extended portion within the miRNA with at least 35 bases of the UTR that is 5′ of the extended portion within the UTR; calculating a free energy measurement of the association of the extended portion within the miRNA and the at least 35 bases of the miRNA that is 3′ of the extended portion within the miRNA with the extended portion within the UTR and the at least 35 bases of the UTR that is 5′ of the extended portion within the UTR; and determining whether the miRNA adequately binds to the gene using the free energy measurement. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. The conserved miRNA sequence may arise from a vertebrate, a mammal, or a human. In one embodiment, the method includes defining exactly 6 nucleotides or exactly 7 nucleotides of the conserved miRNA sequence as an miRNA seed. The method, in some instances, also comprises determining base-pairing of remaining bases of the miRNA that are 3′ of the extended portion within the miRNA with the remaining bases of the UTR that are 5′ of the extended portion within the UTR. In certain instances, the method also includes, if adequate binding is determined, synthesizing an oligonucleotide comprising a sequence that is substantially antisense to the miRNA, and in some cases, administering the synthesized oligonucleotide to a cell or a subject, such as a human. In some embodiments, if the gene is a target of the miRNA, the method may also include synthesizing an oligonucleotide comprising a sequence that is substantially antisense to the conserved miRNA sequence, and introducing the synthesized oligonucleotide into a cell, such a tumor cell.

In one set of embodiments, the method includes altering, in a cell such as a vertebrate cell, expression of a gene regulated by binding of miRNA to an miRNA binding region of the gene by exposing the cell to an oligonucleotide comprising a sequence that is substantially antisense to at least a portion of the miRNA binding region of the gene. The sequence may be selected from the group consisting of SEQ ID NO: 682 to SEQ ID NO: 761, and/or the sequences of SEQ ID NO: 762 to SEQ ID NO: 1227 that are antisense to an miRNA. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. The cell may a mammalian cell or a human cell in some cases. In certain embodiments, the cell may be part of an in vitro culture, or part of a living organism.

In another set of embodiments, the method includes transfecting a cell such as a vertebrate cell with a sequence encoding an miRNA that, when expressed by the cell, causes the cell to overexpress the miRNA. The sequence may be selected from the group consisting of SEQ ID NO: 682 to SEQ ID NO: 761, and/or the sequences of SEQ ID NO: 762 to SEQ ID NO: 1227 that are antisense to an miRNA. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. The cell may a mammalian cell or a human cell in some cases. In certain embodiments, the cell may be part of an in vitro culture, or part of a living organism.

In still another set of embodiments, the method is a method of increasing expression of a gene in a cell. In some cases, the method includes introducing, into a cell, an isolated oligonucleotide comprising an miRNA sequence. The isolated oligonucleotide, in some embodiments, may have a stem-loop structure an/or be able to from an miRNA duplex. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. The cell may a mammalian cell or a human cell in some cases. In certain embodiments, the cell may be part of an in vitro culture, or part of a living organism.

Yet another aspect of the invention relates to an article including a machine-readable medium having a program stored thereon. According to one set of embodiments, the program has instructions for, when executed, performing analysis of a conserved miRNA sequence and a genome of an organism, defining at least 6 nucleotides of the conserved miRNA sequence as an miRNA seed, identifying a conserved UTR of a gene within the genome of the organism, and identifying the gene as a target of the miRNA by determining whether the conserved UTR comprises a segment having perfect complementarity with the miRNA seed.

In another set of embodiments, the article includes a machine-readable medium having a program stored thereon, which program has instructions for, when executed, performing analysis of a conserved miRNA sequence and an mRNA of a gene comprising a UTR; defining at least 6 nucleotides of the conserved miRNA sequence as an miRNA seed; identifying, within the UTR, one or more segments of perfect complementarity with the miRNA seed; defining an extended portion within the UTR that includes an identified segment of perfect complementarity, each base of the extended portion within the UTR being matched with one base of the miRNA as one of a A:U pair, a U:A pair, a C:G pair, a G:C pair, a G:U pair, or a U:G pair; defining an extended portion within the miRNA corresponding to the extended portion within the UTR; determining base-pairing of at least 35 bases of the miRNA that is 3′ of the extended portion within the miRNA with at least 35 bases of the UTR that is 5′ of the extended portion within the UTR; calculating a free energy measurement of the association of the extended portion within the miRNA and the at least 35 bases of the miRNA that is 3′ of the extended portion within the miRNA with the extended portion within the UTR and the at least 35 bases of the UTR that is 5′ of the extended portion within the UTR; and determining whether the miRNA adequately binds to the gene using the free energy measurement.

In another aspect, the article includes a cell, such as a vertebrate cell, transfected with a genetic sequence that causes the cell to overexpress an miRNA. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. The cell may a mammalian cell or a human cell in some cases. In certain embodiments, the cell may be part of an in vitro culture, or part of a living organism.

In yet another set of embodiments, the article includes a cell, such as a vertebrate cell, transfected with a genetic sequence that causes the cell to overexpress an antisense miRNA inhibitor. The sequence may be selected from the group consisting of SEQ ID NO: 682 to SEQ ID NO: 761, and/or the sequences of SEQ ID NO: 762 to SEQ ID NO: 1227 that are antisense to an miRNA. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. The cell may a mammalian cell or a human cell in some cases. In certain embodiments, the cell may be part of an in vitro culture, or part of a living organism.

Still another aspect of the invention contemplates a composition. According to one set of embodiments, the composition comprises an isolated oligonucleotide (for example, RNA) comprising a sequence that is substantially antisense to an miRNA. The sequence may be selected from the group consisting of SEQ ID NO: 682 to SEQ ID NO: 761, and/or the sequences of SEQ ID NO: 762 to SEQ ID NO: 1227 that are antisense to an miRNA. The miRNA may be selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. In certain embodiments, the miRNA is vertebrate, mammal, or human miRNA. The sequence, or the isolated oligonucleotide comprising the sequence, may have from 18 to 26 nucleotides, or from 20 to 24 nucleotides. In one embodiment, the composition further comprises a pharmaceutically acceptable carrier.

According to another set of embodiments, the composition includes an isolated oligonucleotide comprising a sequence that is an miRNA selected from the group consisting of the miRNA sequences, but not the miRNA-like control sequences, of SEQ ID NO: 3 to SEQ ID NO: 468, and/or SEQ ID NO: 469 to SEQ ID NO: 537 and SEQ ID NO: 542 to 551. In certain embodiments, the miRNA is vertebrate, mammal, or human miRNA. The sequence, or the isolated oligonucleotide comprising the sequence, may have from 18 to 26 nucleotides, or from 20 to 24 nucleotides.

In some embodiments the composition includes a pharmaceutically acceptable carrier. The pharmaceutically acceptable carrier may include components specific for the therapeutic indication. For instance, for the treatment of a chronic disease the composition may be formulated in a depo preparation or a controlled release formulation. Some therapeutic indications may call for pulmonary delivery. In such instances the compositions may be formulated in pulmonary delivery device such as a nebulizer.

In other embodiments, the compositions may be formulated in therapeutic cocktails including the oligonucleotide and an additional therapeutic agent, such as an anti-cancer agent.

In yet other embodiments the compositions are therapeutic mixtures of different oligonucleotides. For instance the composition may include more than one oligonucleotide that is an miRNA or is antisense to an miRNA.

In another aspect, the invention provides a method. In one set of embodiments, the method includes providing an miRNA, and an UTR of a gene sequence; determining, within the UTR, a first sequence perfectly complementary to a first portion of the miRNA; defining an extended portion within the miRNA that comprises the first portion of the miRNA, where the extended portion is complementary to an extended sequence of the UTR, the extended sequence comprising the first sequence of the UTR; optionally, determining a second portion of the miRNA able to bind to a second sequence of the UTR, where the second sequence is 5′ of the extended sequence of the UTR; and calculating a free energy measurement of a configuration in which the UTR and the miRNA are bound via binding of the extended portion with the extended sequence and optionally, the second portion with the second sequence. The method, according to another set of embodiments, includes regulating expression of a gene comprising a UTR and a coding region in a mammalian cell by binding miRNA to the UTR.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention is directed to a method of making one or more of the embodiments described herein. In yet another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D are schematic diagrams illustrating the prediction of miRNA targets, according to certain embodiments of the invention;

FIGS. 2A-2C are graphs illustrating target conservation in multiple genomes, in accordance with some embodiments of the invention;

FIG. 3 is a block diagram illustrating an example of a storage system that may be used as part of a computer system to implement some embodiments of the invention.

FIG. 4 is a table illustrating targets of certain mammalian miRNAs, in another embodiment of the invention;

FIG. 5 is a table illustrating the molecular function of certain miRNA targets of the invention;

FIG. 6 illustrates certain miRNA sequences and control sequences used in various examples of the invention;

FIG. 7 illustrates certain miRNA targets, in accordance with an embodiment of the invention;

FIG. 8 illustrates certain miRNA targets, in accordance with another embodiment of the invention;

FIG. 9 illustrates the functional classes of certain miRNA targets, in accordance with yet another embodiment of the invention;

FIG. 10 illustrates certain predicted targets of control sequences, used to estimate the specificity of miRNA target prediction in one example of the invention;

FIGS. 11A-11H illustrates the identification of certain miRNA targets, in one embodiment of the invention;

FIGS. 12A-12D illustrates the importance of Watson-Crick matching to the miRNA seed region for achieving high specificity of target prediction, in one embodiment of the invention;

FIG. 13 illustrates the classification of functions of certain miRNA targets, in another embodiment of the invention;

FIG. 14 illustrates various matches for a miR-125 seed heptamer and its shuffled derivatives, in yet another embodiment of the invention;

FIG. 15 illustrates certain miRNAs, used in certain embodiments of the invention;

FIG. 16 is a block diagram illustrating an example of a computer system on which some embodiments of the invention may be implemented; and

### BRIEF DESCRIPTION OF THE SEQUENCES

SEQ ID NO: 1 is GGGCCCGGGULLLLLLACCCGGGCCC, an artificial stem-loop RNA sequence;

SEQ ID NO: 2 is AGCTCTATACGCGTCTCAAGCTTACTGCTAGCGT, a sequence containing multiple cloning sites;

SEQ ID NO: 3 is UGAGGUAGUAGGUUGUAUAGUU, an miRNA sequence;

SEQ ID NO: 4 is UUGAUGGUAGUGAUGUGGUAUA, an miRNA-like control sequence for the SEQ ID NO:3 miRNA;

SEQ ID NO: 5 is UGGUUUAUGGAGUAUAGGGUAU, an miRNA-like control sequence for the SEQ ID NO:3 miRNA;

SEQ ID NO: 6 is UAUGGGUGUAUAUGGAGUUAGU, an miRNA-like control sequence for the SEQ ID NO:3 miRNA;

SEQ ID NO: 7 is UUAUGUUGAGUAGGUAGGUAUG, an miRNA-like control sequence for the SEQ ID NO:3 miRNA;

SEQ ID NO: 8 is UGAGGUAGUAGGUUGUGUGGUU, an miRNA sequence;

SEQ ID NO: 9 is UGAGGUAGUAGGUUGUAUGGUU, an miRNA sequence;

SEQ ID NO: 10 is AGAGGUAGUAGGUUGCAUAGU, an miRNA sequence;

SEQ ID NO: 11 is UGAGGUAGGAGGUUGUAUAGU, an miRNA sequence;

SEQ ID NO: 12 is UGAGGUAGUAGAUUGUAUAGUU, an miRNA sequence;

SEQ ID NO: 13 is UGAGGUAGUAGUUUGUACAGUU, an miRNA sequence;

SEQ ID NO: 14 is UGAGGUAGUAGUUUGUGCUGUU, an miRNA sequence;

SEQ ID NO: 15 is UGGAAUGUAAAGAAGUAUGUAU, an miRNA sequence;

SEQ ID NO: 16 is UGUAAAUUUAAUAGGGGAGAUA, an miRNA-like control sequence;

SEQ ID NO: 17 is UAAUUUAGGGAGUAUAAGGAAU, an miRNA-like control sequence;

SEQ ID NO: 18 is UGAAGAGUUAGAUUAGAAUGUA, an miRNA-like control sequence;

SEQ ID NO: 19 is UGAAGUAAGAGUAUUUGAGAUA, an miRNA-like control sequence;

SEQ ID NO: 20 is UGGAAGACUAGUGAUUUUGUU, an miRNA sequence;

SEQ ID NO: 21 is UCAAUGUUUGAAUAUGUGGUG, an miRNA-like control sequence;

SEQ ID NO: 22 is UAUUUUGGUUCAAGGAGAUGU, an miRNA-like control sequence;

SEQ ID NO: 23 is UGAUUUCUGGAUUGUAUGAAG, an miRNA-like control sequence;

SEQ ID NO: 24 is UUGUGGGAUGAGAUAAUCUUU, an miRNA-like control sequence;

SEQ ID NO: 25 is UCUUUGGUUAUCUAGCUGUAUGA, an miRNA sequence;

SEQ ID NO: 26 is UGCUCUUUUUUAGCGAUUGAGA, an miRNA-like control sequence;

SEQ ID NO: 27 is UUCUGAAUUUUUGGCGAGUUCAU, an miRNA-like control sequence;

SEQ ID NO: 28 is UAACUCUGUAUGGGUAUCUUUGU, an miRNA-like control sequence;

SEQ ID NO: 29 is UUGCUCUUGUGUGUACAUGAUA, an miRNA-like control sequence;

SEQ ID NO: 30 is UACCCUGUAGAUCCGAAUUUGUG, an miRNA sequence;

SEQ ID NO: 31 is UACCCUGUAGAACCGAAUUUGU, an miRNA sequence;

SEQ ID NO: 32 is UAUGCCAUUUACCGAAUGCUGA, an miRNA-like control sequence;

SEQ ID NO: 33 is UAUGCUGCGAAAUACAUUUGCC, an miRNA-like control sequence;

SEQ ID NO: 34 is UAUGAUCAACUGACACGUUGCU, an miRNA-like control sequence;

SEQ ID NO: 35 is UACCUCACGGCUGAUGUUUAAA, an miRNA-like control sequence;

SEQ ID NO: 36 is UAGCAGCACAUAAUGGUUUGUG, an miRNA sequence;

SEQ ID NO: 37 is UAGCAGCACAUCAUGGUUUACA, an miRNA sequence;

SEQ ID NO: 38 is UAGCAGCACGUAAAUAUUGGCG, an miRNA sequence;

SEQ ID NO: 39 is UAGAAAUAGUCACGCAGGUCGU, an miRNA-like control sequence;

SEQ ID NO: 40 is UACAGAGAAGCGCUAAUCGUGU, an miRNA-like control sequence;

SEQ ID NO: 41 is UAGAGGAAAUCGCUUAGCCGAU, an miRNA-like control sequence;

SEQ ID NO: 42 is UAGAAAUACGUGACGUGUCCAG, an miRNA-like control sequence;

SEQ ID NO: 43 is ACUGCAGUGAAGGCACUUGU, an miRNA sequence;

SEQ ID NO: 44 is UAAGGUGCAUCUAGUGCAGAUA, an miRNA sequence;

SEQ ID NO: 45 is UCUUCUAUGAAGAACGAAUGGG, an miRNA-like control sequence;

SEQ ID NO: 46 is UUGGGUAAAAAGUCUCAUCGAG, an miRNA-like control sequence;

SEQ ID NO: 47 is UCAGGGUAAAACGAUUGGACUU, an miRNA-like control sequence;

SEQ ID NO: 48 is UGAAUAUGCAGUGAGUGAUACC, an miRNA-like control sequence;

SEQ ID NO: 49 is UGUGCAAAUCUAUGCAAAACUGA, an miRNA sequence;

SEQ ID NO: 50 is UGCCUUCCUGAAAAAUAUAGGAA, an miRNA-like control sequence;

SEQ ID NO: 51 is UGGAACCAAAUAUGACUUCUGAA, an miRNA-like control sequence;

SEQ ID NO: 52 is UGACUGAGCAUGAAUUUACAACA, an miRNA-like control sequence;

SEQ ID NO: 53 is UUCAAUAAGGACCUUGAUACAAG, an miRNA-like control sequence;

SEQ ID NO: 54 is UGUGCAAAUCCAUGCAAAACUGA, an miRNA sequence;

SEQ ID NO: 55 is UAAAGUGCUUAUAGUGCAGGUAG, an miRNA sequence;

SEQ ID NO: 56 is UGGUGCUGGAACAUGAUAAUAGU, an miRNA-like control sequence;

SEQ ID NO: 57 is UGAGGAUGUACAUAUUGUCAAGG, an miRNA-like control sequence;

SEQ ID NO: 58 is UGUAAAUGCAGUGUGACAGUAUG, an miRNA-like control sequence;

SEQ ID NO: 59 is UAAUGAGGAUCUGGGAUCAUGUA, an miRNA-like control sequence;

SEQ ID NO: 60 is UAGCUUAUCAGACUGAUGUUGA, an miRNA sequence;

SEQ ID NO: 61 is UUAGUUAUGCAGGGUUAUCACA, an miRNA-like control sequence;

SEQ ID NO: 62 is UAUUAGUGACUUCAGGGACUUA, an miRNA-like control sequence;

SEQ ID NO: 63 is UCUAUAAUGUCAAGUAGUUGGC, an miRNA-like control sequence;

SEQ ID NO: 64 is UUAGUUAUUUACAUGGACAGGC, an miRNA-like control sequence;

SEQ ID NO: 65 is AAGCUGCCAGUUGAAGAACUGU, an miRNA sequence;

SEQ ID NO: 66 is AGAGGUGGACUUUGAAACUCCA, an miRNA-like control sequence;

SEQ ID NO: 67 is AAGAUGUGUCAACACCAGUUGG, an miRNA-like control sequence;

SEQ ID NO: 68 is AAGGGGCACUCUUUAAGAAGUC, an miRNA-like control sequence;

SEQ ID NO: 69 is AACAUGAAAGCCUGUGUUGGCA, an miRNA-like control sequence;

SEQ ID NO: 70 is AUCACAUUGCCAGGGAUUUCC, an miRNA sequence;

SEQ ID NO: 71 is AUCAUUCUGCAAGCCUCUAGG, an miRNA-like control sequence;

SEQ ID NO: 72 is AUCCUCCAAGCUGUCUGAAUG, an miRNA-like control sequence;

SEQ ID NO: 73 is AUCCUGGCUAAAUCUGACCUG, an miRNA-like control sequence;

SEQ ID NO: 74 is AUCUCCCAUUTUGAGAGGCCA, an miRNA-like control sequence;

SEQ ID NO: 75 is AUCACAUUGCCAGGGAUUACCAC, an miRNA sequence;

SEQ ID NO: 76 is UGGCUCAGUUCAGCAGGAACAG, an miRNA sequence;

SEQ ID NO: 77 is UGGCCAGGAAGGCAAUGCAUUC, an miRNA-like control sequence;

SEQ ID NO: 78 is UGAGGGAAAUCCUCCCUGAGAG, an miRNA-like control sequence;

SEQ ID NO: 79 is UGCCAGGGGCAAGAAAUUGCCU, an miRNA-like control sequence;

SEQ ID NO: 80 is UGCUCUGGAAAGCCCAAUAGGG, an miRNA-like control sequence;

SEQ ID NO: 81 is CAUUGCACUUGUCUCGGUCUGA, an miRNA sequence;

SEQ ID NO: 82 is CCCCCAAUUGAUCGUGUUGGUU, an miRNA-like control sequence;

SEQ ID NO: 83 is CUUGAGACCCGUUGGUCUCAUU, an miRNA-like control sequence;

SEQ ID NO: 84 is CAUUGGCUCGUCCUCUAAGUUG, an miRNA-like control sequence;

SEQ ID NO: 85 is CCAUUGGUAUUCGGUUCACCUG, an miRNA-like control sequence;

SEQ ID NO: 86 is UUCAAGUAAUCCAGGAUAGGCU, an miRNA sequence;

SEQ ID NO: 87 is UUACUUCAGAAGGGUACUGAAC, an miRNA-like control sequence;

SEQ ID NO: 88 is UUACUGCAGGUAAGCUUAAGAC, an miRNA-like control sequence;

SEQ ID NO: 89 is UCAAGUUAUGGGACCUGACAAU, an miRNA-like control sequence;

SEQ ID NO: 90 is UAACCCUCUGGAGGGUAAAUUA, an miRNA-like control sequence;

SEQ ID NO: 91 is UUCAAGUAAUUCAGGAUAGGUU, an miRNA sequence;

SEQ ID NO: 92 is UUCACAGUGGCUAAGUUCCGCC, an miRNA sequence;

SEQ ID NO: 93 is UGACAGCAUCGCUCAGCCUUGU, an miRNA-like control sequence;

SEQ ID NO: 94 is ACUGCAUGGGACCAUUCGU, an miRNA-like control sequence;

SEQ ID NO: 95 is UAAAAUCCUGUCUGGCCCCGUG, an miRNA-like control sequence;

SEQ ID NO: 96 is UAUGAAAGCCCCGGUUUGCCUC, an miRNA-like control sequence;

SEQ ID NO: 97 is UUCACAGUGGCUAAGUUCUG, an miRNA sequence;

SEQ ID NO: 98 is AAGGAGCUCACAGUCUAUUGAG, an miRNA sequence;

SEQ ID NO: 99 is CUAGCACCAUCUGAAAUCGGUU, an miRNA sequence;

SEQ ID NO: 100 is CCUCACUACGAAUUAAGGGCUU, an miRNA-like control sequence;

SEQ ID NO: 101 is CUGAUAGACGAAUGCACCCUUU, an miRNA-like control sequence;

SEQ ID NO: 102 is CACUAAGUCGGCAAUUGUCUCA, an miRNA-like control sequence;

SEQ ID NO: 103 is CUCUAGAUCAAGACUUCGCAGU, an miRNA-like control sequence;

SEQ ID NO: 104 is UAGCACCAUUUGAAAUCAGUGUU, an miRNA sequence;

SEQ ID NO: 105 is UAUUAGAAACCUGCUCUUGUAAG, an miRNA-like control sequence;

SEQ ID NO: 106 is UAUGCAGAACUCUCAAUUAGUUG, an miRNA-like control sequence;

SEQ ID NO: 107 is UUACUUUAAGGACAGGAAUUCCU, an miRNA-like control sequence;

SEQ ID NO: 108 is UAUGUUCUCCCAUUGGAUAAAAG, an miRNA-like control sequence;

SEQ ID NO: 109 is UAGCACCAUUUGAAAUCGGUUA, an miRNA sequence;

SEQ ID NO: 110 is UGUAAACAUCCUCGACUGGAAGC, an miRNA sequence;

SEQ ID NO: 111 is UGUAAACAUCCUACACUCAGC, an miRNA sequence;

SEQ ID NO: 112 is UUGUCACACACAUCCACAUAG, an miRNA-like control sequence;

SEQ ID NO: 113 is UCCAGAGCAACACCUAUUCUA, an miRNA-like control sequence;

SEQ ID NO: 114 is UAAGCCCAUGUCCAUUACACA, an miRNA-like control sequence;

SEQ ID NO: 115 is UCUGUCCACACAUGACCAUAA, an miRNA-like control sequence;

SEQ ID NO: 116 is UGUAAACAUCCUACACUCUCAGC, an miRNA sequence;

SEQ ID NO: 117 is UGUAAACAUCCCCGACUGGAAG, an miRNA sequence;

SEQ ID NO: 118 is UGUAAACAUCCUUGACUGG, an miRNA sequence;

SEQ ID NO: 119 is GGCAAGAUGCUGGCAUAGCUG, an miRNA sequence;

SEQ ID NO: 120 is GGGCAACUGAGUCCUUAGAGG, an miRNA-like control sequence;

SEQ ID NO: 121 is GUUGAGGCUAGUCAGGCACAG, an miRNA-like control sequence;

SEQ ID NO: 122 is GAAUGGGCAUGGAUUGGCCCA, an miRNA-like control sequence;

SEQ ID NO: 123 is GGUACAAGGCAAGGUCUGGUC, an miRNA-like control sequence;

SEQ ID NO: 124 is UAUUGCACAUUACUAAGUUGC, an miRNA sequence;

SEQ ID NO: 125 is GUGCAUUGUAGUUGCAUUG, an miRNA sequence;

SEQ ID NO: 126 is GGUUUGAUCAUCUAGGGUU, an miRNA-like control sequence;

SEQ ID NO: 127 is GGGGUUCUUGAUUAGCUUA, an miRNA-like control sequence;

SEQ ID NO: 128 is GAUCUUGGCUAAGGUGUUU, an miRNA-like control sequence;

SEQ ID NO: 129 is GAUUGUUGUAGGUCACUUG, an miRNA-like control sequence;

SEQ ID NO: 130 is GUGCAUUGCUGUUGCAUUG, an miRNA sequence;

SEQ ID NO: 131 is UGGCAGUGUCUUAGCUGGUUGU, an miRNA sequence;

SEQ ID NO: 132 is UGUGGCUGAUUCUCUAUGGGGU, an miRNA-like control sequence;

SEQ ID NO: 133 is UGGGCCUGUGUUUGUGUGUAAC, an miRNA-like control sequence;

SEQ ID NO: 134 is UUUGUGUGUCAGUGGGAGUCUC, an miRNA-like control sequence;

SEQ ID NO: 135 is UUCCUGGAGGGUCUGGUAUGUU, an miRNA-like control sequence;

SEQ ID NO: 136 is UAUUGCACUUGUCCCGGCCUGU, an miRNA sequence;

SEQ ID NO: 137 is AAAGUGCUGUUCGUGCAGGUAG, an miRNA sequence;

SEQ ID NO: 138 is AACAGGUUGCCGGAGAUGUGUU, an miRNA-like control sequence;

SEQ ID NO: 139 is AAGUGUGGCGUAAAGUGCUUGC, an miRNA-like control sequence;

SEQ ID NO: 140 is AUUUUGGAGCGGUCAGCUAGAG, an miRNA-like control sequence;

SEQ ID NO: 141 is AGCACUGGCGGUUUAAUUGGGA, an miRNA-like control sequence;

SEQ ID NO: 142 is AAAGUGCUGACAGUGCAGAU, an miRNA sequence;

SEQ ID NO: 143 is UUCAACGGGUAUUUAUUGAGCA, an miRNA sequence;

SEQ ID NO: 144 is UUUGGCACUAGCACAUUUUUGC, an miRNA sequence;

SEQ ID NO: 145 is UGAAAUCCUUGGAUGUUCUCUC, an miRNA-like control sequence;

SEQ ID NO: 146 is UGCUUCACUGGUUUAACCAGUU, an miRNA-like control sequence;

SEQ ID NO: 147 is UGAAAGUCCUUCUGUCAUUUGC, an miRNA-like control sequence;

SEQ ID NO: 148 is UUGUUGCAGGCACAUCCUUUUA, an miRNA-like control sequence;

SEQ ID NO: 149 is UGAGGUAGUAAGUUGUAUUGUU, an miRNA sequence;

SEQ ID NO: 150 is ACCCGUAGAUCCGAUCUUGU, an miRNA sequence;

SEQ ID NO: 151 is AUUGUACGAUCCCGCUGUCA, an miRNA-like control sequence;

SEQ ID NO: 152 is AUAUUCCGUGAGACCGCUCU, an miRNA-like control sequence;

SEQ ID NO: 153 is AUCCGAUCGGCACUUGUAUC, an miRNA-like control sequence;

SEQ ID NO: 154 is AUUACCGACGUACUGGCUCU, an miRNA-like control sequence;

SEQ ID NO: 155 is CACCCGUAGAACCGACCUUGCG, an miRNA sequence;

SEQ ID NO: 156 is AACCCGUAGAUCCGAACUUGUG, an miRNA sequence;

SEQ ID NO: 157 is ACGCAUAACGUGGGUUUCAACC, an miRNA-like control sequence;

SEQ ID NO: 158 is AGCGAUUACUCAACUCGUCAGG, an miRNA-like control sequence;

SEQ ID NO: 159 is AUAAGCGCAUUCCCGGGAUUCA, an miRNA-like control sequence;

SEQ ID NO: 160 is AGUACGGCUAUAUGGCUACACC, an miRNA-like control sequence;

SEQ ID NO: 161 is UACAGUACUGUGAUAACUGA, an miRNA sequence;

SEQ ID NO: 162 is UUAUACACUUAGUAAGAGGC, an miRNA-like control sequence;

SEQ ID NO: 163 is UUAUAUAGAAGCUAGACUGC, an miRNA-like control sequence;

SEQ ID NO: 164 is UAUGCUAUGUAGCCAAUAGA, an miRNA-like control sequence;

SEQ ID NO: 165 is UAAUAUAGGGUGCUAUCAAC, an miRNA-like control sequence;

SEQ ID NO: 166 is AGCAGCAUUGUACAGGGCUAUGA, an miRNA sequence;

SEQ ID NO: 167 is AUCAGGGCAGUAUUGAGAUGACC, an miRNA-like control sequence;

SEQ ID NO: 168 is AAGUGAGAGUAGGCUCUAGACUC, an miRNA-like control sequence;

SEQ ID NO: 169 is AGGGCUCUAAUGGACAGGAUAUC, an miRNA-like control sequence;

SEQ ID NO: 170 is AUGGAAGUUUCCUCGGAGCAAAG, an miRNA-like control sequence;

SEQ ID NO: 171 is AGCAACAUUGUACAGGGCUAUGA, an miRNA sequence;

SEQ ID NO: 172 is UCAACAUCAGUCUGAUAAGCUA, an miRNA sequence;

SEQ ID NO: 173 is UUUCAUAGAAGAAAACCCUUCG, an miRNA-like control sequence;

SEQ ID NO: 174 is UCUAAAGUCAGGAUACAUUACC, an miRNA-like control sequence;

SEQ ID NO: 175 is UUGAAUCUCACACAGUAGAUCA, an miRNA-like control sequence;

SEQ ID NO: 176 is UGCAGAAUUACCCUUAAGACUA, an miRNA-like control sequence;

SEQ ID NO: 177 is UCAAAUGCUCAGACUCCUGU, an mRNA sequence;

SEQ ID NO: 178 is AAAAGUGCUUACAGUGCAGGUAGC, an miRNA sequence;

SEQ ID NO: 179 is AGCAGCAUUGUACAGGGCUAUCA, an miRNA sequence;

SEQ ID NO: 180 is AUAAGGAUUUUUAGGGGCAUU, an miRNA sequence;

SEQ ID NO: 181 is AUGUUAUGAGGCAAUGAUUUG, an miRNA-like control sequence;

SEQ ID NO: 182 is AUAAGGGAAUUGUGGAUUCUU, an miRNA-like control sequence;

SEQ ID NO: 183 is AGUUGUGUUGUUAGAUCAAAG, an miRNA-like control sequence;

SEQ ID NO: 184 is AGUAGAUGAAGAGUUUGUUUC, an miRNA-like control sequence;

SEQ ID NO: 185 is UGGAGUGUGACAAUGGUGUUUGU, an miRNA sequence;

SEQ ID NO: 186 is UGGUACAGGUUGUGGGAAUGUUU, an miRNA-like control sequence;

SEQ ID NO: 187 is UGGGUAGGAUUUGUUCUUGGAGA, an miRNA-like control sequence;

SEQ ID NO: 188 is UUGUAGCUGUAAGUGAUUUGGGG, an miRNA-like control sequence;

SEQ ID NO: 189 is UUAAGUUAUUGUGGGGUGCAGUG, an miRNA-like control sequence;

SEQ ID NO: 190 is CAUUAUUACUUUUGGUACGCG, an miRNA sequence;

SEQ ID NO: 191 is CUAAUAUUUUGCGCGUGCUUA, an miRNA-like control sequence;

SEQ ID NO: 192 is CUUAUAUUUUCCGCGAUGUGA, an miRNA-like control sequence;

SEQ ID NO: 193 is CUAUAAUUUUCGUUACGCUGG, an miRNA-like control sequence;

SEQ ID NO: 194 is CAUGUAUUUCGGAUUCUUACG, an miRNA-like control sequence;

SEQ ID NO: 195 is UUAAGGCACGCGGUGAAUGCCA, an miRNA sequence;

SEQ ID NO: 196 is UGCAGAACGGAGGCGACAUCUU, an miRNA-like control sequence;

SEQ ID NO: 197 is UUAGAGAGCGUCGAAGGACUCC, an miRNA-like control sequence;

SEQ ID NO: 198 is UUGAUGGCCGAUAACCGCAGAG, an miRNA-like control sequence;

SEQ ID NO: 199 is UGCAGGACGUCAUCCGAAGGAU, an miRNA-like control sequence;

SEQ ID NO: 200 is UCCCUGAGACCCUUUAACCUGUG, an miRNA sequence;

SEQ ID NO: 201 is UCCCUGAGACCCUAACUUGUGA, an mRNA sequence;

SEQ ID NO: 202 is UCUGGGCCAAUAUGCAUCCACU, an miRNA-like control sequence;

SEQ ID NO: 203 is UCCACCUGCAGACAUUGUAGCU, an miRNA-like control sequence;

SEQ ID NO: 204 is UCAGCCCAUCUGCAGUACAGUU, an miRNA-like control sequence;

SEQ ID NO: 205 is UAACCCAGCUCUCCUGGGUAAU, an miRNA-like control sequence;

SEQ ID NO: 206 is UCGUACCGUGAGUAAUAAUGC, an miRNA sequence;

SEQ ID NO: 207 is UAUCGCGACUUAGUACAGUGA, an miRNA-like control sequence;

SEQ ID NO: 208 is UCGUAUCGUAAGAUAGUGACC, an miRNA-like control sequence;

SEQ ID NO: 209 is UACGAUCGCUAAUCAUGGGUA, an miRNA-like control sequence;

SEQ ID NO: 210 is UCCGUACGGAAGACUUAUGUA, an miRNA-like control sequence;

SEQ ID NO: 211 is UCGGAUCCGUCUGAGCUUGGCU, an miRNA sequence;

SEQ ID NO: 212 is UCACAGUGAACCGGUCUCUUUU, an miRNA sequence;

SEQ ID NO: 213 is UCUGCUCAAGUUCGCUCAAUGU, an miRNA-like control sequence;

SEQ ID NO: 214 is UAACUGAACUGCGUUUCCUCUG, an miRNA-like control sequence;

SEQ ID NO: 215 is UGGCCCUGCAUUACCUAUGUAU, an miRNA-like control sequence;

SEQ ID NO: 216 is UGACAGAACCUGUUUCGCUCUU, an miRNA-like control sequence;

SEQ ID NO: 217 is UCACAGUGAACCGGUCUCUUUC, an miRNA sequence;

SEQ ID NO: 218 is CUUUUUCGGUCUGGGCUUGC, an miRNA sequence;

SEQ ID NO: 219 is CUUUUUGCGGUCUGGGCUUGC, an miRNA sequence;

SEQ ID NO: 220 is CUCCUUUGUGUUUGGGUCCGG, an miRNA-like control sequence;

SEQ ID NO: 221 is CUUGGGGGUUUCCGGUUCUUC, an miRNA-like control sequence;

SEQ ID NO: 222 is CCUUUUGGCGUUUGGCUUGGC, an miRNA-like control sequence;

SEQ ID NO: 223 is CUCCUUUUGUUCUGGUUGGGCG, an miRNA-like control sequence;

SEQ ID NO: 224 is CAGUGCAAUGUUAAAAGGGC, an miRNA sequence;

SEQ ID NO: 225 is CAUGAGAGGUGGACUUCAAA, an mRNA-like control sequence;

SEQ ID NO: 226 is CUGAAUGCAGACUUGGAAGA, an miRNA-like control sequence;

SEQ ID NO: 227 is CUGUUGGAGGGAAACAUAAC, an miRNA-like control sequence;

SEQ ID NO: 228 is CAGCUCAAAUUGAGGAUGGA, an miRNA-like control sequence;

SEQ ID NO: 229 is CAGUGCAAUGAUGAAAGGGC, an miRNA sequence;

SEQ ID NO: 230 is UAAAGCUAGAUAACCGAAAGU, an miRNA sequence;

SEQ ID NO: 231 is UAAGAUAAACGUGAAUGCACA, an miRNA-like control sequence;

SEQ ID NO: 232 is UAGAAGAUCCGAUGUAAAACA, an miRNA-like control sequence;

SEQ ID NO: 233 is UAUGAAACGAGCCUAAAAGUA, an miRNA-like control sequence;

SEQ ID NO: 234 is UAUGACAACAAAGUCGAGAUA, an miRNA-like control sequence;

SEQ ID NO: 235 is UAACAGUCUACAGCCAUGGUCGC, an miRNA sequence;

SEQ ID NO: 236 is UAGCAUCCCAUAGUCGGAGAUCC, an miRNA-like control sequence;

SEQ ID NO: 237 is UCACAUCCUGGUACGGAAGACCU, an miRNA-like control sequence;

SEQ ID NO: 238 is UAAUGCCAUACUGCCUACCGGAG, an miRNA-like control sequence;

SEQ ID NO: 239 is UACUGGCAUUCCGACAGUACAGC, an miRNA-like control sequence;

SEQ ID NO: 240 is UUGGUCCCCUUCAACCAGCUGU, an miRNA sequence;

SEQ ID NO: 241 is UGCCCACCUCAUUGCUUGUCAG, an miRNA-like control sequence;

SEQ ID NO: 242 is UCUGGUUCUCAUGAAGCCUCCC, an miRNA-like control sequence;

SEQ ID NO: 243 is UCAAGUCCCCCUUGCCUUUAGG, an miRNA-like control sequence;

SEQ ID NO: 244 is UGCAUCUCUUUGGCCCACAGCU, an miRNA-like control sequence;

SEQ ID NO: 245 is UUGGUCCCCUUCAACCAGCUA, an miRNA sequence;

SEQ ID NO: 246 is UGUGACUGGUUGACCAGAGGGG, an miRNA sequence;

SEQ ID NO: 247 is UAUGGCUUUUUAUUCCUAUGUGAU, an miRNA sequence;

SEQ ID NO: 248 is UACUCUGGUUUUUUGUGUACUAAU, an miRNA-like control sequence;

SEQ ID NO: 249 is UAUGCAUGUUGUGCUAUUUUUAUC, an miRNA-like control sequence;

SEQ ID NO: 250 is UAGUUCUUGGCUAUUUAUAUUUGC, an miRNA-like control sequence;

SEQ ID NO: 251 is UAUUUAUGUUAGGUUUUCUGCUAC, an miRNA-like control sequence;

SEQ ID NO: 252 is ACUCCAUUUGUUUUGAUGAUGGA, an miRNA sequence;

SEQ ID NO: 253 is UAUUGCUUAAGAAUACGCGUAG, an miRNA sequence;

SEQ ID NO: 254 is UAGAAGUCUUACGAUUAACGGU, an miRNA-like control sequence;

SEQ ID NO: 255 is UACAAGUGACGAAUGUUACGUU, an miRNA-like control sequence;

SEQ ID NO: 256 is UAGAGAUUAAUACGCGUACUUG, an miRNA-like control sequence;

SEQ ID NO: 257 is UUUACUAAUAGACGUGAGAUCG, an mRNA-like control sequence;

SEQ ID NO: 258 is AGCUGGUGUUGUGAAUC, an miRNA sequence;

SEQ ID NO: 259 is AGCCUGUGUAUUUGGAG, an miRNA-like control sequence;

SEQ ID NO: 260 is AUCAGUGGUUACUUGGG, an miRNA-like control sequence;

SEQ ID NO: 261 is AUGGAGGGUGAUUUCCU, an miRNA-like control sequence;

SEQ ID NO: 262 is AUGGAUUUGUAGCCUGG, an miRNA-like control sequence;

SEQ ID NO: 263 is UCUACAGUGCACGUGUCUCCAGU, an miRNA sequence;

SEQ ID NO: 264 is AGUGGUUUUACCCUAUGGUAG, an miRNA sequence;

SEQ ID NO: 265 is AGCAUGUGAUGGUAUCCUGUU, an miRNA-like control sequence;

SEQ ID NO: 266 is ACAUUGUUGGCUGGGUAUACU, an miRNA-like control sequence;

SEQ ID NO: 267 is AGUGGGCUUUUCUUGACGAAU, an miRNA-like control sequence;

SEQ ID NO: 268 is AAGAGUCCUUUUUCGGGUUAG, an miRNA-like control sequence;

SEQ ID NO: 269 is AACACUGUCUGGUAAAGAUGG, an miRNA sequence;

SEQ ID NO: 270 is AUGGCAGAAAUGUGCUCAGAU, an miRNA-like control sequence;

SEQ ID NO: 271 is ACUGAUUUGCAAGUGAGCAGA, an miRNA-like control sequence;

SEQ ID NO: 272 is AGUGAGGAGCCAGUUAACAUU, an miRNA-like control sequence;

SEQ ID NO: 273 is AGGUGGGAUCAAGCUCAUUAA, an miRNA-like control sequence;

SEQ ID NO: 274 is UGUAGUGUUUCCUACUUUAUGG, an miRNA sequence;

SEQ ID NO: 275 is UGUGGUAUCUUGACUUCUAUUG, an miRNA-like control sequence;

SEQ ID NO: 276 is UAUAGCCUUCUUGUAGGUGUUU, an miRNA-like control sequence;

SEQ ID NO: 277 is UUGUAGUACUUGUUUGCUACUG, an miRNA-like control sequence;

SEQ ID NO: 278 is UACUAGCUUUGGCUUGUUGUAU, an miRNA-like control sequence;

SEQ ID NO: 279 is CCCAUAAAGUAGAAAGCACUAC, an miRNA sequence;

SEQ ID NO: 280 is CAGAGUCAUAAGCCAUAAACAC, an miRNA-like control sequence;

SEQ ID NO: 281 is CAGAAGAUAAUAAACCAUGCCC, an miRNA-like control sequence;

SEQ ID NO: 282 is CCACUAAAAGAGCAGACAUACU, an miRNA-like control sequence;

SEQ ID NO: 283 is CUACCAAAAAAUCGAAAGCCUG, an miRNA-like control sequence; SEQ ID NO: 284 is UGAGAUGAAGCACUGUAGCUCA, an miRNA sequence; SEQ ID NO: 285 is UAAUGUGGAGCUCACACAGUGA, an miRNA-like control sequence;

SEQ ID NO: 286 is UCAGAAUAGAUGGCUCAGUGCA, an miRNA-like control sequence;

SEQ ID NO: 287 is UCAGUGGAAGGAAUACCUGACU, an miRNA-like control sequence;

SEQ ID NO: 288 is UGUCCCCAUAAGAAGUGAGAUG, an miRNA-like control sequence;

SEQ ID NO: 289 is UACAGUAUAGAUGAUGUACUAG, an miRNA sequence;

SEQ ID NO: 290 is UUAUAUUAUUGCGAAAGAGAGC, an miRNA-like control sequence;

SEQ ID NO: 291 is UUAUGUAUAAGGGUUCAACGAA, an miRNA-like control sequence;

SEQ ID NO: 292 is UAUAAUGUCGUCUAAAGGAAUG, an miRNA-like control sequence;

SEQ ID NO: 293 is UUAAUAUAGGCAUUGUGCGAAA, an miRNA-like control sequence;

SEQ ID NO: 294 is GUCCAGUUUUCCCAGGAAUCCCUU, an miRNA sequence;

SEQ ID NO: 295 is GUCCCCCUGCAAGAGUUUUUCAUC, an miRNA-like control sequence;

SEQ ID NO: 296 is GUUCCAGCUCUUGCCCUUGCAAAU, an miRNA-like control sequence;

SEQ ID NO: 297 is GCACCCCUUGCUGUUCAAGACUUU, an miRNA-like control sequence;

SEQ ID NO: 298 is GAGGCCACUCCAGCUUCAUCUUUU, an miRNA-like control sequence;

SEQ ID NO: 299 is UGAGAACUGAAUUCCAUGGGUU, an miRNA sequence;

SEQ ID NO: 300 is UUCUGGAUGGCUUACAAAUGAG, an miRNA-like control sequence;

SEQ ID NO: 301 is UGAAUGGAUUCAGUUGCACAGU, an miRNA-like control sequence;

SEQ ID NO: 302 is UGGAGUUUCUAACAGUUGAAGC, an miRNA-like control sequence;

SEQ ID NO: 303 is UUGAGGACUGAGCUUGUUAACA, an miRNA-like control sequence;

SEQ ID NO: 304 is GUGUGUGGAAAUGCUUCUGCC, an miRNA sequence;

SEQ ID NO: 305 is UCAGUGCACUACAGAACUUUGU, an miRNA sequence;

SEQ ID NO: 306 is UGCCCUGCUUGAUAUCAAGAAU, an miRNA-like control sequence;

SEQ ID NO: 307 is UCUGUGAGUAAAAUGCCACUUC, an miRNA-like control sequence;

SEQ ID NO: 308 is UGCAUUUACCUGAAGUUACCAG, an miRNA-like control sequence;

SEQ ID NO: 309 is UCAGUGAACUGCUAUUCUGCAA, an miRNA-like control sequence;

SEQ ID NO: 310 is UCAGUGCAUCACAGAACUUUGU, an miRNA sequence;

SEQ ID NO: 311 is UCUGGCUCCGUGUCUUCACUCC, an miRNA sequence;

SEQ ID NO: 312 is UCUCCCAACCCUUGUACCAGUGU, an miRNA sequence;

SEQ ID NO: 313 is CUAGACUGAAGCUCCUUGAGG, an miRNA sequence;

SEQ ID NO: 314 is UCAGUGCAUGACAGAACUUGG, an miRNA sequence;

SEQ ID NO: 315 is UUGCAUAGUCACAAAAGUGA, an miRNA sequence;

SEQ ID NO: 316 is UUAAGCCUAAGAUGAACAUG, an mRNA-like control sequence;

SEQ ID NO: 317 is UGAGUUGUAAAGCCCAAUAA, an miRNA-like control sequence;

SEQ ID NO: 318 is UCCAAUGUCUAAGAAUAAGG, an miRNA-like control sequence;

SEQ ID NO: 319 is UUAGAGUGACAACACUUAAG, an miRNA-like control sequence;

SEQ ID NO: 320 is UAGGUUAUCCGUGUUGCCUUCG, an miRNA sequence;

SEQ ID NO: 321 is UUAAUGCUAAUUGUGAUAGGGG, an miRNA sequence;

SEQ ID NO: 322 is UAGUUGAAUGUUUAGGGUCAGA, an miRNA-like control sequence;

SEQ ID NO: 323 is UGAGUGAAUGGUUCAAGUGUAU, an miRNA-like control sequence;

SEQ ID NO: 324 is UAUUUAGGAGGGAACAUGUUGU, an miRNA-like control sequence;

SEQ ID NO: 325 is UUGUAGAGUAUUGGUCAAUGAG, an miRNA-like control sequence;

SEQ ID NO: 326 is AACAUUCAACGCUGUCGGUGAGU, an miRNA sequence;

SEQ ID NO: 327 is AUUCUGUGAACAUCGGACGUCAG, an miRNA-like control sequence;

SEQ ID NO: 328 is AAGUGUUUCCGAGAACUAUCGGC, an miRNA-like control sequence;

SEQ ID NO: 329 is AAGUUUCUGAUCGUCAGACGGCA, an miRNA-like control sequence;

SEQ ID NO: 330 is ACUGAGAAGGCCGCGUUUCAUAU, an miRNA-like control sequence;

SEQ ID NO: 331 is AACAUUCAUUGCUGUCGGUGGGUU, an miRNA sequence;

SEQ ID NO: 332 is AACAUUCAACCUGUCGGUGAGU, an miRNA sequence;

SEQ ID NO: 333 is UUUGGCAAUGGUAGAACUCACA, an miRNA sequence;

SEQ ID NO: 334 is UCUGCAAGAGCAGAAUAGUUCU, an miRNA-like control sequence;

SEQ ID NO: 335 is UUGCCAAAUUGGAGAACUGUAC, an miRNA-like control sequence;

SEQ ID NO: 336 is UGAAUUUGAGUCAUGACCAGAC, an miRNA-like control sequence;

SEQ ID NO: 337 is UUGUCAAGGAUAGCCCAAUUAG, an miRNA-like control sequence;

SEQ ID NO: 338 is UAUGGCACUGGUAGAAUUCACUG, an miRNA sequence;

SEQ ID NO: 339 is UAACUAUGGAGCAGCUGGUUUCA, an miRNA-like control sequence;

SEQ ID NO: 340 is UAUGCACUUGUGGUGAGCAUCAA, an miRNA-like control sequence;

SEQ ID NO: 341 is UCUGGUUACACAUCAGUUAAGGG, an miRNA-like control sequence;

SEQ ID NO: 342 is UAUACAGGCCAUGACUGUUUGAG, an miRNA-like control sequence;

SEQ ID NO: 343 is UGGACGGAGAACUGAUAAGGGU, an miRNA sequence;

SEQ ID NO: 344 is UGACGUGGGACAGGAGAUAAUG, an miRNA-like control sequence;

SEQ ID NO: 345 is UAGGAACGGAGGAGCAUUAGUG, an miRNA-like control sequence;

SEQ ID NO: 346 is UCCGGAGAGGAAAGUGUGGAUA, an miRNA-like control sequence;

SEQ ID NO: 347 is UAGGAACGGAGAGUAAGCUGUG, an miRNA-like control sequence;

SEQ ID NO: 348 is UGGAGAGAAAGGCAGUUC, an miRNA sequence;

SEQ ID NO: 349 is CAAAGAAUUCUCCUUUUGGGCUU, an miRNA sequence;

SEQ ID NO: 350 is UCGUGUCUUGUGUUGCAGCCGG, an miRNA sequence;

SEQ ID NO: 351 is UCCUCCGUUUUGCGGGUUAGGG, an miRNA-like control sequence;

SEQ ID NO: 352 is UCCGUGUUUCGGCAUCUGGGUG, an miRNA-like control sequence;

SEQ ID NO: 353 is UCCGUGGCGGGGAUGUUUUCCU, an miRNA-like control sequence;

SEQ ID NO: 354 is UCCGUGUUGCUUGCGGCUUGGA, an miRNA-like control sequence;

SEQ ID NO: 355 is CAUCCCUUGCAUGGUGGAGGGU, an miRNA sequence;

SEQ ID NO: 356 is GUGCCUACUGAGCUGACAUCAGU, an miRNA sequence;

SEQ ID NO: 357 is UGAUAUGUUUGAUAUAUUAGGU, an miRNA sequence;

SEQ ID NO: 358 is UGUGGUAUUAGAUUAUAUUGAU, an miRNA-like control sequence;

SEQ ID NO: 359 is UGUAGUUAGUUUGUAAUAUUGUA, an miRNA-like control sequence;

SEQ ID NO: 360 is UGUGAGUAGAUGUUAUUAUUAU, an miRNA-like control sequence;

SEQ ID NO: 361 is UGUAUAAUGUUAUAGGUUUAGU, an mRNA-like control sequence;

SEQ ID NO: 362 is CAACGGAAUCCCAAAAGCAGCU, an miRNA sequence;

SEQ ID NO: 363 is CUGACCUAUGAAUUGACAGCC, an miRNA sequence;

SEQ ID NO: 364 is CCCUAAUAGUCAGCAAGGUCU, an miRNA-like control sequence;

SEQ ID NO: 365 is CAGGCUAUCCUCAAUCUGAGA, an miRNA-like control sequence;

SEQ ID NO: 366 is CUACCUUACAGGGGCCAAUUA, an miRNA-like control sequence;

SEQ ID NO: 367 is CCAUGGUACCCUCAAUUAGAG, an miRNA-like control sequence;

SEQ ID NO: 368 is AACUGGCCUACAAAGUCCCAG, an miRNA sequence;

SEQ ID NO: 369 is UGUAACAGCAACUCCAUGUGGA, an miRNA sequence;

SEQ ID NO: 370 is UGGUCCUUACCCAGAAGGAAUA, an miRNA-like control sequence;

SEQ ID NO: 371 is UUCCAUGCAGUAGAGAUGCCAA, an miRNA-like control sequence;

SEQ ID NO: 372 is UGGGACAUAGAACCAUCAUGCU, an miRNA-like control sequence;

SEQ ID NO: 373 is UCUAAAGUGAGCUAAUCCAGGC, an miRNA-like control sequence;

SEQ ID NO: 374 is UAGCAGCACAGAAAUAUUGGC, an miRNA sequence;

SEQ ID NO: 375 is UAGGUAGUUUCAUGUUGUUGGG, an miRNA sequence;

SEQ ID NO: 376 is UGUAGAUAGUUUGGUUUCUGGG, an miRNA-like control sequence;

SEQ ID NO: 377 is UAUUGGUAGGGGUCAUUUUGUG, an miRNA-like control sequence;

SEQ ID NO: 378 is UGGUUAUAGUUUUGAUGGCUGG, an miRNA-like control sequence;

SEQ ID NO: 379 is UGUGUAGUUUGGACAGGUGUUU, an miRNA-like control sequence;

SEQ ID NO: 380 is UUCACCACCUUCUCCACCCAGC, an miRNA sequence;

SEQ ID NO: 381 is GGUCCAGAGGGGAGAUAGG, an miRNA sequence;

SEQ ID NO: 382 is CCCAGUGUUCAGACUACCUGUUC, an miRNA sequence;

SEQ ID NO: 383 is CCUCAUCUACCAUUGAGCCUGUG, an miRNA-like control sequence;

SEQ ID NO: 384 is CUGUCUGCUCCAGUUCCAGAUAC, an miRNA-like control sequence;

SEQ ID NO: 385 is CUGUCACUCUGGGCAUCCACUUA, an miRNA-like control sequence;

SEQ ID NO: 386 is CCUGGGGUUUACAACCUAUCCUC, an miRNA-like control sequence;

SEQ ID NO: 387 is CCCAGUGUUUAGACUAUCUGUUC, an miRNA sequence;

SEQ ID NO: 388 is UAACACUGUCUGGUAACGAUG, an miRNA sequence;

SEQ ID NO: 389 is UAAUACUGCCUGGUAAUGAUGAC, an miRNA sequence;

SEQ ID NO: 390 is UACUGAGAAUGGUAUCCAGUACU, an miRNA-like control sequence;

SEQ ID NO: 391 is UAGUGGCUAACUAUUGGACACUA, an miRNA-like control sequence;

SEQ ID NO: 392 is UAUGAGGACAGUGUACUUAACUC, an miRNA-like control sequence;

SEQ ID NO: 393 is UACAUGGACUAUUAGUGGAUCCA, an miRNA-like control sequence;

SEQ ID NO: 394 is UACUCAGUAAGGCAUUGUUCU, an miRNA sequence;

SEQ ID NO: 395 is AGAGGUAUAGCGCAUGGGAAGA, an miRNA sequence;

SEQ ID NO: 396 is AGAGAUAUGGACGUAGGGGCAA, an miRNA-like control sequence;

SEQ ID NO: 397 is AUAAGUAGGGAACGGGCUGAGA, an miRNA-like control sequence;

SEQ ID NO: 398 is AGGGAGUAAGACAGGACGAUGU, an miRNA-like control sequence;

SEQ ID NO: 399 is AUGAGUACGGUAGGAAGGGACA, an miRNA-like control sequence;

SEQ ID NO: 400 is UGAAAUGUUUAGGACCACUAGA, an miRNA sequence;

SEQ ID NO: 401 is UACAUUUGGGACACAAUGAUGA, an miRNA-like control sequence;

SEQ ID NO: 402 is UAAAGUCUAGUAAAUGAUGGCC, an miRNA-like control sequence;

SEQ ID NO: 403 is UAGAACAACAAUCUGUGUGUGA, an miRNA-like control sequence;

SEQ ID NO: 404 is UAAUGGAAUGAUGAUUAGCACC, an miRNA-like control sequence;

SEQ ID NO: 405 is UUCCCUUUGUCAUCCUAUGCCUG, an miRNA sequence;

SEQ ID NO: 406 is UCACUUUUGUUGUCCCCCCUAUG, an miRNA-like control sequence;

SEQ ID NO: 407 is UUCUCCUUGCCUGUACUUGCUCA, an miRNA-like control sequence;

SEQ ID NO: 408 is UUCCUUCUAGGUCUCUCCUGACU, an miRNA-like control sequence;

SEQ ID NO: 409 is UUUCUCCCCCCUGUACAGUUGUU, an miRNA-like control sequence;

SEQ ID NO: 410 is UCCUUCAUUCCACCGGAGUCUG, an miRNA sequence;

SEQ ID NO: 411 is UAGGAAUUCCUUCGGCCUUCCC, an miRNA-like control sequence;

SEQ ID NO: 412 is UCUACUUUCCCACAGUGCGCUG, an miRNA-like control sequence;

SEQ ID NO: 413 is UUGCCCCCAAUCGGGCUUUCUA, an miRNA-like control sequence;

SEQ ID NO: 414 is UUGUUCCAUCGGGCCUUCCAAC, an miRNA-like control sequence;

SEQ ID NO: 415 is UGGAAUGUAAGGAAGUGUGUGG, an miRNA sequence;

SEQ ID NO: 416 is GCUUCUCCUGGCUCUCCUCCCUC, an miRNA sequence;

SEQ ID NO: 417 is AUAAGACGAGCAAAAAGCUUGU, an miRNA sequence;

SEQ ID NO: 418 is AUGCGACAAAGAAAUGAUCAUG, an miRNA-like control sequence;

SEQ ID NO: 419 is ACGAGCAUGGUUAAAAAUGAAC, an miRNA-like control sequence;

SEQ ID NO: 420 is AGCGUUAAAACAAGAAGUUGAC, an miRNA-like control sequence;

SEQ ID NO: 421 is AGACGACUUGAUGCUAAGAAAA, an miRNA-like control sequence;

SEQ ID NO: 422 is CUGUGCGUGUGACAGCGGCUG, an miRNA sequence;

SEQ ID NO: 423 is CGUAGGCCUGUCGGGCUUGGA, an miRNA-like control sequence;

SEQ ID NO: 424 is CGUUAGCCACGUGGGGGGCUU, an miRNA-like control sequence;

SEQ ID NO: 425 is CGUUAUCCUCCGGGGGGUGAG, an miRNA-like control sequence;

SEQ ID NO: 426 is CCCGUGGAGAUGCUGGUUGCG, an miRNA-like control sequence;

SEQ ID NO: 427 is UUCCCUUUGUCAUCCUUCGCCU, an miRNA sequence;

SEQ ID NO: 428 is UAACAGUCUCCAGUCACGGCC, an miRNA sequence;

SEQ ID NO: 429 is ACCAUCGACCGUUGAUUGUACC, an miRNA sequence;

SEQ ID NO: 430 is ACCGAGAUCUCCUUCGCUAGUA, an miRNA-like control sequence;

SEQ ID NO: 431 is AUCGUGACCACGUAGCCUUUAC, an miRNA-like control sequence;

SEQ ID NO: 432 is AAUCGUCUAUGCCAGCGUCUCA, an miRNA-like control sequence;

SEQ ID NO: 433 is AUCGUCACCACGAUGGUAUUCC, an miRNA-like control sequence;

SEQ ID NO: 434 is ACAGCAGGCACAGACAGGCAG, an miRNA sequence;

SEQ ID NO: 435 is AGGCAGAGACCAAGACCAGGC, an miRNA-like control sequence;

SEQ ID NO: 436 is ACAGGCAGCACCACAGAGGAG, an miRNA-like control sequence;

SEQ ID NO: 437 is ACAGGAAGGGAAGCAGCCCAC, an miRNA-like control sequence;

SEQ ID NO: 438 is ACAGGAGGGAAGCCCCCAAGA, an miRNA-like control sequence;

SEQ ID NO: 439 is AUGACCUAUGAAUUGACAGAC, an miRNA sequence;

SEQ ID NO: 440 is UAAUCUCAGCUGGCAACUGUG, an miRNA sequence;

SEQ ID NO: 441 is UGCUGUCAAGAAUGUCUCCAG, an miRNA-like control sequence;

SEQ ID NO: 442 is UAACCUCAAGGGUGCUUUGAC, an miRNA-like control sequence;

SEQ ID NO: 443 is UACCAUUUGCAGGCAUGCAUG, an miRNA-like control sequence;

SEQ ID NO: 444 is UCUAAAUAGCAGCCCUUGGUG, an miRNA-like control sequence;

SEQ ID NO: 445 is UACUGCAUCAGGAACUGAUUGGAU, an miRNA sequence;

SEQ ID NO: 446 is UUGUGCUUGAUCUAACCAUGU, an miRNA sequence;

SEQ ID NO: 447 is UCACACUUGUAGUCUGUGAUU, an miRNA-like control sequence;

SEQ ID NO: 448 is UUUGUGUGUUCUCCACAAGUA, an miRNA-like control sequence;

SEQ ID NO: 449 is UGCUUCCUCAGUGUUIJAUAGA, an miRNA-like control sequence;

SEQ ID NO: 450 is UUCAUUAAGUAGCUUGUGUCC, an miRNA-like control sequence;

SEQ ID NO: 451 is UGAUUGUCCAAACGCAAUUCU, an miRNA sequence;

SEQ ID NO: 452 is UUAGGCUCUACCCUAUGAUAA, an miRNA-like control sequence;

SEQ ID NO: 453 is UUAGCUAGACCUAUGCUAACU, an miRNA-like control sequence;

SEQ ID NO: 454 is UGGGCUAACUUACCUAUAACU, an miRNA-like control sequence;

SEQ ID NO: 455 is UUUACCCCUAGUGGACAUAAU, an miRNA-like control sequence;

SEQ ID NO: 456 is CCACACCGUAUCUGACACUUU, an miRNA sequence;

SEQ ID NO: 457 is AGCUACAUUGUCUGCUGGGUUUC, an miRNA sequence;

SEQ ID NO: 458 is AUAUGUGGGUGCCUUUCUCCAUG, an miRNA-like control sequence;

SEQ ID NO: 459 is ACAUCCUAGUUCUGCUUUGGGGU, an miRNA-like control sequence;

SEQ ID NO: 460 is AUACCUCUUCAGUUGGGUGGCUU, an miRNA-like control sequence;

SEQ ID NO: 461 is AUAUGUUCUUGCUGGUUGGCCAC, an miRNA-like control sequence;

SEQ ID NO: 462 is AGCUACAUCUGGCUACUGGGUCUC, an miRNA sequence;

SEQ ID NO: 463 is UGUCAGUUUGUCAAAUACCCCAA, an miRNA sequence;

SEQ ID NO: 464 is UAACUUGUGAGCAUCCAAAUCUC, an miRNA-like control sequence;

SEQ ID NO: 465 is UGCUCACUGUAAUCAGAAAUUCC, an miRNA-like control sequence;

SEQ ID NO: 466 is UACAACUCCUGAUUCAUUAGCAG, an miRNA-like control sequence;

SEQ ID NO: 467 is UAUUCAACAGCUGUUCCAACUGA, an miRNA-like control sequence;

SEQ ID NO: 468 is CAAGUCACUAGUGGUUCCGUUUA, an miRNA sequence;

SEQ ID NO: 469 is GAGGUAG, an miRNA seed sequence;

SEQ ID NO: 470 is ACAGUAC, an miRNA seed sequence;

SEQ ID NO: 471 is GCAGCAU, an mRNA seed sequence;

SEQ ID NO: 472 is ACCCUGU, an miRNA seed sequence;

SEQ ID NO: 473 is GGAGUGU, an miRNA seed sequence;

SEQ ID NO: 475 is CCCUGAG, an miRNA seed sequence;

SEQ ID NO: 476 is CACAGUG, an miRNA seed sequence;

SEQ ID NO: 477 is AGUGCAA, an miRNA seed sequence;

SEQ ID NO: 478 is AACAGUC, an miRNA seed sequence;

SEQ ID NO: 479 is UGGUCCC, an miRNA seed sequence;

SEQ ID NO: 480 is GCUGGUG, an miRNA seed sequence;

SEQ ID NO: 481 is ACAGUAU, an miRNA seed sequence;

SEQ ID NO: 482 is UCCAGUU, an miRNA seed sequence;

SEQ ID NO: 483 is GAGAACU, an mRNA seed sequence;

SEQ ID NO: 484 is CAGUGCA, an miRNA seed sequence;

SEQ ID NO: 485 is ACAUUCA, an miRNA seed sequence;

SEQ ID NO: 486 is UUGGCAA, an miRNA seed sequence;

SEQ ID NO: 487 is GGACGGA, an miRNA seed sequence;

SEQ ID NO: 488 is GUAACAG, an miRNA seed sequence;

SEQ ID NO: 489 is AGGUAGU, an miRNA seed sequence;

SEQ ID NO: 490 is CCAGUGU, an miRNA seed sequence;

SEQ ID NO: 491 is GUGCAAA, an miRNA seed sequence;

SEQ ID NO: 492 is GGAAUGU, an miRNA seed sequence;

SEQ ID NO: 493 is AAAGUGC, an miRNA seed sequence;

SEQ ID NO: 494 is AAUACUG, an miRNA seed sequence;

SEQ ID NO: 495 is GAGGUAU, an miRNA seed sequence;

SEQ ID NO: 496 is AAUCUCA, an miRNA seed sequence;

SEQ ID NO: 497 is UCACAUU, an miRNA seed sequence;

SEQ ID NO: 498 is GGCUCAG, an miRNA seed sequence;

SEQ ID NO: 499 is AUUGCAC, an miRNA seed sequence;

SEQ ID NO: 500 is UCAAGUA, an miRNA seed sequence;

SEQ ID NO: 501 is AGCACCA, an miRNA seed sequence;

SEQ ID NO: 502 is GUAAACA, an miRNA seed sequence;

SEQ ID NO: 503 is CUUUGGU, an miRNA seed sequence;

SEQ ID NO: 504 is AGCAGCA, an miRNA seed sequence;

SEQ ID NO: 505 is UCACAGU, an miRNA seed sequence;

SEQ ID NO: 506 is AAGUGCU, an miRNA seed sequence;

SEQ ID NO: 507 is UUUUUGC, an miRNA seed sequence;

SEQ ID NO: 508 is AUGGCUU, an miRNA seed sequence;

SEQ ID NO: 509 is ACACUGU, an miRNA seed sequence;

SEQ ID NO: 510 is AAGGUGC, an miRNA seed sequence;

SEQ ID NO: 511 is CAGCAGG, an miRNA seed sequence;

SEQ ID NO: 512 is AGCUGCC, an miRNA seed sequence;

SEQ ID NO: 513 is GGCAGUG, an miRNA seed sequence;

SEQ ID NO: 514 is UUGGCAC, an miRNA seed sequence;

SEQ ID NO: 515 is GUGGUUU, an miRNA seed sequence;

SEQ ID NO: 516 is CCAUAAA, an miRNA seed sequence;

SEQ ID NO: 517 is GAGAUGA, an miRNA seed sequence;

SEQ ID NO: 518 is AUGGCAC, an miRNA seed sequence;

SEQ ID NO: 519 is GAAAUGU, an miRNA seed sequence;

SEQ ID NO: 520 is UGUGCGU, an miRNA seed sequence;

SEQ ID NO: 521 is UGUGCUU, an miRNA seed sequence;

SEQ ID NO: 522 is GCUACAU, an miRNA seed sequence;

SEQ ID NO: 523 is GCAAGAU, an miRNA seed sequence;

SEQ ID NO: 524 is UGCAUUG, an miRNA seed sequence;

SEQ ID NO: 525 is GGAAGAC, an miRNA seed sequence;

SEQ ID NO: 526 is AUUGCUU, an miRNA seed sequence;

SEQ ID NO: 527 is UGCAUAG, an miRNA seed sequence;

SEQ ID NO: 528 is UAAUGCU, an miRNA seed sequence;

SEQ ID NO: 529 is UCCCUUU, an miRNA seed sequence;

SEQ ID NO: 530 is GUCAGUU, an miRNA seed sequence;

SEQ ID NO: 531 is UAGCACC, an miRNA seed sequence;

SEQ ID NO: 532 is GUAGUGU, an miRNA seed sequence;

SEQ ID NO: 533 is AAAGCUA, an miRNA seed sequence;

SEQ ID NO: 534 is GAUAUGU, an miRNA seed sequence;

SEQ ID NO: 535 is CCUUCAU, an miRNA seed sequence;

SEQ ID NO: 536 is ACCCGUA, an miRNA seed sequence;

SEQ ID NO: 537 is GAUUGUC, an miRNA seed sequence;

SEQ ID NO: 538 is UGCCUCUGGAAAACUAUUGAGCCUUGCAUGUACUUGAAG, a portion of the human SMAD-1 gene;

SEQ ID NO: 539 is GAGCCUUGAUAAUACUUGAC, a portion of the human SMAD-1 gene;

SEQ ID NO: 540 is 6 nt-UGCCUCUGGAA-18 nt-GUACUUGAAG-36 nt-GAGCCUUGAUAAUACUUGAC-5-nt, a portion of the 3′ UTR of the WT human SMAD-1 gene;

SEQ ID NO: 541 is 6 nt-UGCCUCUGGAA-18 nt-GUUCGUUAAG-36 nt-GAGCCUUGAUAAUUCGUUAC-5nt, a portion of a mutated portion of the 3′ UTR of the WT human SMAD-1 gene;

SEQ ID NO: 542 is AAGGCAC, an miRNA seed sequence;

SEQ ID NO: 543 is UGACCUA, an miRNA seed sequence;

SEQ ID NO: 545 is AACACUG, an miRNA seed sequence;

SEQ ID NO: 546 is AGCUUAU, an miRNA seed sequence;

SEQ ID NO: 547 is CGUACCG, an miRNA seed sequence;

SEQ ID NO: 548 is CGUGUCU, an miRNA seed sequence;

SEQ ID NO: 549 is UGAAAUG, an miRNA seed sequence;

SEQ ID NO: 550 is ACUGCAU, an miRNA seed sequence;

SEQ ID NO: 551 is UUGUUCG, an miRNA seed sequence;

SEQ ID NO: 552 is AAGAAGUAUGUA, a portion of the 3′ end of an miRNA sequence;

SEQ ID NO: 553 is AGGAAGUGUGUGG, a portion of the 3′ end of an miRNA sequence;

SEQ ID NO: 554 is AGGUUGUAUAGUU, a portion of the 3′ end of an miRNA sequence;

SEQ ID NO: 555 is AGGUUGUGUGGUU, a portion of the 3′ end of an miRNA sequence;

SEQ ID NO: 556 is AGGUUGUAUGGUU, a portion of the 3′ end of an miRNA sequence;

SEQ ID NO: 557 is AGGUUGCAUAGU, a portion of the 3′ end of an miRNA sequence;

SEQ ID NO: 558 is AGGUUGUAUAGU, a portion of the 3′ end of an miRNA sequence;

SEQ ID NO: 559 is AGAUUGUAUAGUU, a portion of the 3′ end of an miRNA sequence;

SEQ ID NO: 560 is AGUUUGUACAGU, a portion of the 3′ end of an miRNA sequence;

SEQ ID NO: 561 is AGUUUGUGCU, a portion of the 3′ end of an miRNA sequence;

SEQ ID NO: 562 is AAGUUGUAUUGUU, a portion of the 3′ end of an miRNA sequence;

SEQ ID NO: 563 is UCAUGUUGUUGG, a portion of the 3′ end of an miRNA sequence;

SEQ ID NO: 564 is UCCUGUUGUUGG, a portion of the 3′ end of an miRNA sequence;

SEQ ID NO: 565 is GAUCCGAAUUUGUG, a portion of the 3′ end of an miRNA sequence;

SEQ ID NO: 566 is GAACCGAAUUUGU, a portion of the 3′ end of an miRNA sequence;

SEQ ID NO: 567 is AUAAUGGUUUGUG, a portion of the 3′ end of an miRNA sequence;

SEQ ID NO: 568 is AUCAUGGUUUACA, a portion of the 3′ end of an miRNA sequence;

SEQ ID NO: 569 is GUAAAUAUUGGCG, a portion of the 3′ end of an miRNA sequence;

SEQ ID NO: 570 is AGAAAUAUUGGC, a portion of the 3′ end of an mRNA sequence;

SEQ ID NO: 571 is GUACAGGGCUAUGA, a portion of the 3′ end of an miRNA sequence;

SEQ ID NO: 572 is GUACAGGGCUAUCA, a portion of the 3′ end of an miRNA sequence;

SEQ ID NO: 573 is CUAUGCAAAACUGA, a portion of the 3′ end of an miRNA sequence;

SEQ ID NO: 574 is CCAUGCAAAACUGA, a portion of the 3′ end of an miRNA sequence;

SEQ ID NO: 575 is GUUAAAAGGGC, a portion of the 3′ end of an miRNA sequence;

SEQ ID NO: 576 is GAUGAAAGGGCAU, a portion of the 3′ end of an miRNA sequence;

SEQ ID NO: 577 is AGUAUUGUCAAAGC, a portion of the 3′ end of an miRNA sequence;

SEQ ID NO: 578 is UACAGAACUUUGU, a portion of the 3′ end of an miRNA sequence;

SEQ ID NO: 579 is CACAGAACUUUGU, a portion of the 3′ end of an miRNA sequence;

SEQ ID NO: 580 is GACAGAACUUGG, a portion of the 3′ end of an miRNA sequence;

SEQ ID NO: 581 is UUCGUGCAGGUAG, a portion of the 3′ end of an miRNA sequence;

SEQ ID NO: 582 is CCAUGUUUGGUGA, a portion of the 3′ end of an miRNA sequence;

SEQ ID NO: 583 is CCAUGUUUUAGUAG, a portion of the 3′ end of an miRNA sequence;

SEQ ID NO: 584 is CCAUGUUUCAGUGG, a portion of the 3′ end of an miRNA sequence;

SEQ ID NO: 585 is CCAUGUUUGAGUGU, a portion of the 3′ end of an miRNA sequence;

SEQ ID NO: 586 is CGACAUUUGAGCGU, a portion of the 3′ end of an miRNA sequence;

SEQ ID NO: 587 is CGAUUUUGGGGUGU, a portion of the 3′ end of an miRNA sequence;

SEQ ID NO: 588 is UAUAGUGCAGGUA, a portion of the 3′ end of an miRNA sequence;

SEQ ID NO: 589 is UACAGUGCAGGUAGU, a portion of the 3′ end of an miRNA sequence;

SEQ ID NO: 590 is UACAGUGCAGGUAGC, a portion of the 3′ end of an miRNA sequence;

SEQ ID NO: 591 is GACAGUGCAGAU, a portion of the 3′ end of an miRNA sequence;

SEQ ID NO: 592 is CCAGGGAUUUCC, a portion of the 3′ end of an miRNA sequence;

SEQ ID NO: 593 is CCAGGGAUUACCAC, a portion of the 3′ end of an miRNA sequence;

SEQ ID NO: 594 is UGUCUCGGUCUGA, a portion of the 3′ end of an miRNA sequence;

SEQ ID NO: 595 is UUACUAAGUUGC, a portion of the 3′ end of an miRNA sequence;

SEQ ID NO: 596 is UGUCCCGGCCUGU, a portion of the 3′ end of an miRNA sequence;

SEQ ID NO: 597 is UUAGCAAUGGUGA, a portion of the 3′ end of an miRNA sequence;

SEQ ID NO: 598 is UCCAGGAUAGGCU, a portion of the 3′ end of an miRNA sequence;

SEQ ID NO: 599 is UUCAGGAUAGGU, a portion of the 3′ end of an miRNA sequence;

SEQ ID NO: 600 is GCUAAGUUCCGCC, a portion of the 3′ end of an miRNA sequence;

SEQ ID NO: 601 is GCUAAGUUCUG, a portion of the 3′ end of an miRNA sequence;

SEQ ID NO: 602 is ACCGGUCUCUUUU, a portion of the 3′ end of an miRNA sequence;

SEQ ID NO: 603 is ACCGGUCUCUUUC, a portion of the 3′ end of an miRNA sequence;

SEQ ID NO: 604 is UUGAAAUCAGU, a portion of the 3′ end of an miRNA sequence;

SEQ ID NO: 605 is UUGAAAUCGGUUA, a portion of the 3′ end of an miRNA sequence;

SEQ ID NO: 606 is CCUCGACUGGAAGC, a portion of the 3′ end of an miRNA sequence;

SEQ ID NO: 607 is CCUACACUCAGC, a portion of the 3′ end of an miRNA sequence;

SEQ ID NO: 608 is CCUACACUCUCAGC, a portion of the 3′ end of an miRNA sequence;

SEQ ID NO: 609 is CCCCGACUGGAAG, a portion of the 3′ end of an miRNA sequence;

SEQ ID NO: 610 is CCUUGACUGGA, a portion of the 3′ end of an miRNA sequence;

SEQ ID NO: 611 is CUUAGCUGGUUGU, a portion of the 3′ end of an miRNA sequence;

SEQ ID NO: 612 is CAUUAGCUGAUUG, a portion of the 3′ end of an miRNA sequence;

SEQ ID NO: 613 is AGUUAGCUGAUUG, a portion of the 3′ end of an miRNA sequence;

SEQ ID NO: 614 is AUCCGAACUUGUG, a portion of the 3′ end of an miRNA sequence;

SEQ ID NO: 615 is AUCCGAUCUUGUG, a portion of the 3′ end of an miRNA sequence;

SEQ ID NO: 616 is AACCGACCUUGCG, a portion of the 3′ end of an miRNA sequence;

SEQ ID NO: 617 is GCGGUGAAUGCC, a portion of the 3′ end of an miRNA sequence;

SEQ ID NO: 618 is CGGUGAAUGCCA, a portion of the 3′ end of an miRNA sequence;

SEQ ID NO: 619 is CCCUAACUUGUGA, a portion of the 3′ end of an miRNA sequence;

SEQ ID NO: 620 is CCCUUUAACCUGUG, a portion of the 3′ end of an miRNA sequence;

SEQ ID NO: 621 is UUCAACCAGCUGU, a portion of the 3′ end of an miRNA sequence;

SEQ ID NO: 622 is UUCAACCAGCUA, a portion of the 3′ end of an miRNA sequence;

SEQ ID NO: 623 is UUAUUCCUAUGUGA, a portion of the 3′ end of an miRNA sequence;

SEQ ID NO: 624 is UCAUUCCUAUGUG, a portion of the 3′ end of an miRNA sequence;

SEQ ID NO: 625 is CGCUGUCGGUGAGU, a portion of the 3′ end of an miRNA sequence;

SEQ ID NO: 626 is UGCUGUCGGUGGGUU, a portion of the 3′ end of an miRNA sequence;

SEQ ID NO: 627 is CCUGUCGGUGAGU, a portion of the 3′ end of an miRNA sequence;

SEQ ID NO: 628 is GAAUUGACAGCC, a portion of the 3′ end of an miRNA sequence;

SEQ ID NO: 629 is GAAUUGACAGAC, a portion of the 3′ end of an miRNA sequence;

SEQ ID NO: 630 is CAGACUACCUGUUC, a portion of the 3′ end of an miRNA sequence;

SEQ ID NO: 631 is UAGACUAUCUGUUC, a portion of the 3′ end of an miRNA sequence;

SEQ ID NO: 632 is CUGGUAACGAUGU, a portion of the 3′ end of an miRNA sequence;

SEQ ID NO: 633 is CUGGUAAAGAUGG, a portion of the 3′ end of an miRNA sequence;

SEQ ID NO: 634 is CUGGUAAUGAUG, a portion of the 3′ end of an miRNA sequence;

SEQ ID NO: 635 is CGGGUAAUGAUGGA, a portion of the 3′ end of an miRNA sequence;

SEQ ID NO: 636 is UCAUCCUAUGCCU, a portion of the 3′ end of an miRNA sequence;

SEQ ID NO: 637 is UCAUCCUUCGCCU, a portion of the 3′ end of an miRNA sequence;

SEQ ID NO: 638 is GUCUGCUGGGUUUC, a portion of the 3′ end of an miRNA sequence;

SEQ ID NO: 639 is UGGCUACUGGGUCUC, a portion of the 3′ end of an miRNA sequence;

SEQ ID NO: 640 is AGUGAUUUUGUU, a portion of the 3′ end of an miRNA sequence;

SEQ ID NO: 641 is AUCUAGCUGUAUGA, a portion of the 3′ end of an miRNA sequence;

SEQ ID NO: 642 is UCUAGUGCAGAUA, a portion of the 3′ end of an miRNA sequence;

SEQ ID NO: 643 is AGACUGAUGUUGA, a portion of the 3′ end of an miRNA sequence;

SEQ ID NO: 644 is GUUGAAGAACUGU, a portion of the 3′ end of an miRNA sequence;

SEQ ID NO: 645 is UCAGCAGGAACAG, a portion of the 3′ end of an miRNA sequence;

SEQ ID NO: 646 is AGUUGCAUUG, a portion of the 3′ end of an miRNA sequence;

SEQ ID NO: 647 is GUGAUAACUGAAG, a portion of the 3′ end of an miRNA sequence;

SEQ ID NO: 648 is ACAAUGGUGUUUGU, a portion of the 3′ end of an miRNA sequence;

SEQ ID NO: 649 is GAGUAAUAAUGC, a portion of the 3′ end of an miRNA sequence;

SEQ ID NO: 650 is AGAAUACGCGUAG, a portion of the 3′ end of an miRNA sequence;

SEQ ID NO: 651 is UGUGAAUC, a portion of the 3′ end of an miRNA sequence;

SEQ ID NO: 652 is ACCCUAUGGUAG, a portion of the 3′ end of an miRNA sequence;

SEQ ID NO: 653 is UCCUACUUUAUGGA, a portion of the 3′ end of an miRNA sequence;

SEQ ID NO: 654 is GAUGAUGUACUAG, a portion of the 3′ end of an miRNA sequence;

SEQ ID NO: 655 is AAUUCCAUGGGUU, a portion of the 3′ end of an miRNA sequence;

SEQ ID NO: 656 is CACAAAAGUGA, a portion of the 3′ end of an miRNA sequence;

SEQ ID NO: 657 is AUCGUGAUAGGGG, a portion of the 3′ end of an miRNA sequence;

SEQ ID NO: 658 is GGUAGAAUUCACUG, a portion of the 3′ end of an miRNA sequence;

SEQ ID NO: 659 is AACUGAUAAGGGU, a portion of the 3′ end of an miRNA sequence;

SEQ ID NO: 660 is GUGUUGCAGCCG, a portion of the 3′ end of an miRNA sequence;

SEQ ID NO: 661 is UGAUAUAUUAGGU, a portion of the 3′ end of an miRNA sequence;

SEQ ID NO: 662 is AACUCCAUGUGGA, a portion of the 3′ end of an miRNA sequence;

SEQ ID NO: 663 is UUAGGACCACUAG, a portion of the 3′ end of an miRNA sequence;

SEQ ID NO: 664 is CCACCGGAGUCUG, a portion of the 3′ end of an miRNA sequence;

SEQ ID NO: 665 is CUGGCAACUGUG, a portion of the 3′ end of an miRNA sequence;

SEQ ID NO: 666 is AGGAACUGAUUGGAU, a portion of the 3′ end of an miRNA sequence;

SEQ ID NO: 667 is AUCUAACCAUGU, a portion of the 3′ end of an miRNA sequence;

SEQ ID NO: 668 is AAACGCAAUUCU, a portion of the 3′ end of an miRNA sequence;

SEQ ID NO: 669 is GUCAAAUACCCC, a portion of the 3′ end of an miRNA sequence;

SEQ ID NO: 670 is UCGGCUCGCGUGA, a portion of the 3′ end of an miRNA sequence;

SEQ ID NO: 671 is AAUAAA, a polyadenylation signal;

SEQ ID NO: 672 is AUUAAA, a polyadenylation signal;

SEQ ID NO: 673 is UGUA, a conserved element of the PUM2 binding site consensus;

SEQ ID NO: 674 is CAGUGCC, a suitable control sequence for the miR-125 heptamer;

SEQ ID NO: 675 is CGGACCU, an inappropriate control sequence for the miR-125 heptamer;

SEQ ID NO: 676 is CGCGUAC, an inappropriate control sequence for the miR-125 heptamer;

SEQ ID NO: 677 is AAAAAAGGAAAAGUAGGCAAAUGUGAAAAUAGUUUCAAUAUAUC, a segment of the UTR of human HIC;

SEQ ID NO: 678 is CAAAAGAAAAAUAGGCAAAUGUGAAAACAGUUUUAGCAUAUU, a segment of the UTR of mouse HIC;

SEQ ID NO: 679 is CAAAAGAAAAAUAGGCAAAUGUGAAAACAGUUUUAGCAUAUU, a segment of the UTR of rat HIC;

SEQ ID NO: 680 is AAGAACCAAAGUAGGAAAAUGUGAAAAUAGUUUCAGUGUAUG, a segment of the UTR of dog HIC;

SEQ ID NO: 681 is AGAAUUAGAAGGAGACAAAUGUGAAAAUAGUUUAAGUAAAG, a segment of the UTR of chicken HIC;

SEQ ID NO: 682 is AUCACAUUGCCGAGGGAUUUCC, which is the miRNA sequence miR-23a;

SEQ ID NO: 683 is CUACCUC, a sequence that is antisense to an miRNA seed;

SEQ ID NO: 684 is GUACUGU, a sequence that is antisense to an miRNA seed;

SEQ ID NO: 685 is AUGCUGC, a sequence that is antisense to an miRNA seed;

SEQ ID NO: 686 is ACAGGGU, a sequence that is antisense to an miRNA seed;

SEQ ID NO: 687 is ACACUCC, a sequence that is antisense to an miRNA seed;

SEQ ID NO: 688 is CUCAGGG, a sequence that is antisense to an miRNA seed;

SEQ ID NO: 689 is CACUGUG, a sequence that is antisense to an miRNA seed;

SEQ ID NO: 690 is UUGCACU, a sequence that is antisense to an miRNA seed;

SEQ ID NO: 691 is GACUGUU, a sequence that is antisense to an miRNA seed;

SEQ ID NO: 692 is GGGACCA, a sequence that is antisense to an miRNA seed;

SEQ ID NO: 693 is CACCAGC, a sequence that is antisense to an miRNA seed;

SEQ ID NO: 694 is AUACUGU, a sequence that is antisense to an miRNA seed;

SEQ ID NO: 695 is AACUGGA, a sequence that is antisense to an miRNA seed;

SEQ ID NO: 696 is AGUUCUC, a sequence that is antisense to an miRNA seed;

SEQ ID NO: 697 is UGCACUG, a sequence that is antisense to an miRNA seed;

SEQ ID NO: 698 is UGAAUGU, a sequence that is antisense to an miRNA seed;

SEQ ID NO: 699 is UUGCCAA, a sequence that is antisense to an miRNA seed;

SEQ ID NO: 700 is UCCGUCC, a sequence that is antisense to an miRNA seed;

SEQ ID NO: 701 is CUGUUAC, a sequence that is antisense to an miRNA seed;

SEQ ID NO: 702 is ACUACCU, a sequence that is antisense to an miRNA seed;

SEQ ID NO: 703 is ACACUGG, a sequence that is antisense to an miRNA seed;

SEQ ID NO: 704 is UUUGCAC, a sequence that is antisense to an miRNA seed;

SEQ ID NO: 705 is ACAUUCC, a sequence that is antisense to an miRNA seed;

SEQ ID NO: 706 is GCACUUU, a sequence that is antisense to an miRNA seed;

SEQ ID NO: 707 is CAGUAUU, a sequence that is antisense to an miRNA seed;

SEQ ID NO: 708 is AUACCUC, a sequence that is antisense to an miRNA seed;

SEQ ID NO: 709 is UGAGAUU, a sequence that is antisense to an miRNA seed;

SEQ ID NO: 710 is AAUGUGA, a sequence that is antisense to an miRNA seed;

SEQ ID NO: 711 is CUGAGCC, a sequence that is antisense to an miRNA seed;

SEQ ID NO: 712 is GUGCAAU, a sequence that is antisense to an miRNA seed;

SEQ ID NO: 713 is UACUUGA, a sequence that is antisense to an miRNA seed;

SEQ ID NO: 714 is UGGUGCU, a sequence that is antisense to an miRNA seed;

SEQ ID NO: 715 is UGUUUAC, a sequence that is antisense to an miRNA seed;

SEQ ID NO: 716 is ACCAAAG, a sequence that is antisense to an miRNA seed;

SEQ ID NO: 717 is UGCUGCU, a sequence that is antisense to an miRNA seed;

SEQ ID NO: 718 is ACUGUGA, a sequence that is antisense to an miRNA seed;

SEQ ID NO: 719 is AGCACUU, a sequence that is antisense to an miRNA seed;

SEQ ID NO: 720 is GCAAAAA, a sequence that is antisense to an miRNA seed;

SEQ ID NO: 721 is AAGCCAU, a sequence that is antisense to an miRNA seed;

SEQ ID NO: 722 is ACAGUGU, a sequence that is antisense to an miRNA seed;

SEQ ID NO: 723 is GCACCUU, a sequence that is antisense to an miRNA seed;

SEQ ID NO: 724 is CCUGCUG, a sequence that is antisense to an miRNA seed;

SEQ ID NO: 725 is GGCAGCU, a sequence that is antisense to an miRNA seed;

SEQ ID NO: 726 is CACUGCC, a sequence that is antisense to an miRNA seed;

SEQ ID NO: 727 is GUGCCAA, a sequence that is antisense to an miRNA seed;

SEQ ID NO: 728 is AAACCAC, a sequence that is antisense to an miRNA seed;

SEQ ID NO: 729 is UUUAUGG, a sequence that is antisense to an miRNA seed;

SEQ ID NO: 730 is UCAUCUC, a sequence that is antisense to an miRNA seed;

SEQ ID NO: 731 is GUGCCAU, a sequence that is antisense to an miRNA seed;

SEQ ID NO: 732 is ACAUUUC, a sequence that is antisense to an miRNA seed;

SEQ ID NO: 733 is ACGCACA, a sequence that is antisense to an miRNA seed;

SEQ ID NO: 734 is AAGCACA, a sequence that is antisense to an miRNA seed;

SEQ ID NO: 735 is AUGUAGC, a sequence that is antisense to an miRNA seed;

SEQ ID NO: 736 is AUCUUGC, a sequence that is antisense to an miRNA seed;

SEQ ID NO: 737 is CAAUGCA, a sequence that is antisense to an miRNA seed;

SEQ ID NO: 738 is GUCUUCC, a sequence that is antisense to an miRNA seed;

SEQ ID NO: 739 is AAGCAAU, a sequence that is antisense to an miRNA seed;

SEQ ID NO: 740 is CUAUGCA, a sequence that is antisense to an miRNA seed;

SEQ ID NO: 741 is AGCAUUA, a sequence that is antisense to an miRNA seed;

SEQ ID NO: 742 is AAAGGGA, a sequence that is antisense to an miRNA seed;

SEQ ID NO: 743 is AACUGAC, a sequence that is antisense to an miRNA seed;

SEQ ID NO: 744 is GGUGCUA, a sequence that is antisense to an miRNA seed;

SEQ ID NO: 745 is ACACUAC, a sequence that is antisense to an miRNA seed;

SEQ ID NO: 746 is UAGCUUU, a sequence that is antisense to an miRNA seed;

SEQ ID NO: 747 is ACAUAUC, a sequence that is antisense to an miRNA seed;

SEQ ID NO: 748 is AUGAAGG, a sequence that is antisense to an miRNA seed;

SEQ ID NO: 749 is UACGGGU, a sequence that is antisense to an miRNA seed;

SEQ ID NO: 750 is GACAAUC, a sequence that is antisense to an miRNA seed;

SEQ ID NO: 751 is GUGCCUU, a sequence that is antisense to an miRNA seed;

SEQ ID NO: 752 is UAGGUCA, a sequence that is antisense to an miRNA seed;

SEQ ID NO: 753 is CAGUGUU, a sequence that is antisense to an miRNA seed;

SEQ ID NO: 754 is AUAAGCU, a sequence that is antisense to an miRNA seed;

SEQ ID NO: 755 is CGGUACG, a sequence that is antisense to an miRNA seed;

SEQ ID NO: 756 is AGACACG, a sequence that is antisense to an miRNA seed;

SEQ ID NO: 757 is CAUUUCA, a sequence that is antisense to an miRNA seed;

SEQ ID NO: 758 is AUGCAGU, a sequence that is antisense to an miRNA seed;

SEQ ID NO: 759 is CGAACAA, a sequence that is antisense to an miRNA seed;

SEQ ID NO: 760 is AGGUCCG, a sequence that is antisense to an miRNA seed;

SEQ ID NO: 761 is GUACGCG, a sequence that is antisense to an miRNA seed;

SEQ ID NO: 762 is AACUAUACAACCUACUACCUCA, a sequence that is antisense to an miRNA;

SEQ ID NO: 763 is UAUACCACAUCACUACCAUCAA, a sequence that is antisense to an miRNA-like control sequence;

SEQ ID NO: 764 is AUACCCUAUACUCCAUAAACCA, a sequence that is antisense to an miRNA-like control sequence;

SEQ ID NO: 765 is ACUAACUCCAUAUACACCCAUA, a sequence that is antisense to an miRNA-like control sequence;

SEQ ID NO: 766 is CAUACCUACCUACUCAACAUAA, a sequence that is antisense to an miRNA-like control sequence;

SEQ ID NO: 767 is AACCACACAACCUACUACCUCA, a sequence that is antisense to an miRNA;

SEQ ID NO: 768 is AACCAUACAACCUACUACCUCA, a sequence that is antisense to an miRNA;

SEQ ID NO: 769 is ACUAUGCAACCUACUACCUCU, a sequence that is antisense to an miRNA;

SEQ ID NO: 770 is ACUAUACAACCUCCUACCUCA, a sequence that is antisense to an miRNA;

SEQ ID NO: 771 is AACUAUACAAUCUACUACCUCA, a sequence that is antisense to an miRNA;

SEQ ID NO: 772 is AACUGUACAAACUACUACCUCA, a sequence that is antisense to an miRNA;

SEQ ID NO: 773 is AACAGCACAAACUACUACCUCA, a sequence that is antisense to an miRNA;

SEQ ID NO: 774 is AUACAUACUUCUUUACAUUCCA, a sequence that is antisense to an miRNA;

SEQ ID NO: 775 is UAUCUCCCCUAUUAAAUUUACA, a sequence that is antisense to an miRNA-like control sequence;

SEQ ID NO: 776 is AUUCCUUAUACUCCCUAAAUUA, a sequence that is antisense to an miRNA-like control sequence;

SEQ ID NO: 777 is UACAUUCUAAUCUAACUCUUCA, a sequence that is antisense to an mRNA-like control sequence;

SEQ ID NO: 778 is UAUCUCAAAUACUCUUACUUCA, a sequence that is antisense to an miRNA-like control sequence;

SEQ ID NO: 779 is AACAAAAUCACUAGUCUUCCA, a sequence that is antisense to an miRNA;

SEQ ID NO: 780 is CACCACAUAUUCAAACAUUGA, a sequence that is antisense to an miRNA-like control sequence;

SEQ ID NO: 781 is ACAUCUCCUUGAACCAAAAUA, a sequence that is antisense to an miRNA-like control sequence;

SEQ ID NO: 782 is CUUCAUACAAUCCAGAAAUCA, a sequence that is antisense to an miRNA-like control sequence;

SEQ ID NO: 783 is AAAGAUUAUCUCAUCCCACAA, a sequence that is antisense to an miRNA-like control sequence;

SEQ ID NO: 784 is UCAUACAGCUAGAUAACCAAAGA, a sequence that is antisense to an miRNA;

SEQ ID NO: 785 is UCUCAAUCGCUAAAAAAAGAGCA, a sequence that is antisense to an miRNA-like control sequence;

SEQ ID NO: 786 is AUGAACUCGCCAAAAAUUCAGAA, a sequence that is antisense to an miRNA-like control sequence;

SEQ ID NO: 787 is ACAAAGAUACCCAUACAGAGUUA, a sequence that is antisense to an miRNA-like control sequence;

SEQ ID NO: 788 is UAUCAUGUACACAACAAGAGCAA, a sequence that is antisense to an mRNA-like control sequence;

SEQ ID NO: 789 is CACAAAUUCGGAUCUACAGGGUA, a sequence that is antisense to an miRNA;

SEQ ID NO: 790 is ACAAAUUCGGUUCUACAGGGUA, a sequence that is antisense to an miRNA;

SEQ ID NO: 791 is UCAGCAUUCGGUAAAUGGCAUA, a sequence that is antisense to an miRNA-like control sequence;

SEQ ID NO: 792 is GGCAAAUGUAUUUCGCAGCAUA, a sequence that is antisense to an miRNA-like control sequence;

SEQ ID NO: 793 is AGCAACGUGUCAGUUGAUCAUA, a sequence that is antisense to an miRNA-like control sequence;

SEQ ID NO: 794 is UUUAAACAUCAGCCGUGAGGUA, a sequence that is antisense to an miRNA-like control sequence;

SEQ ID NO: 795 is CACAAACCAUUAUGUGCUGCUA, a sequence that is antisense to an miRNA;

SEQ ID NO: 796 is UGUAAACCAUGAUGUGCUGCUA, a sequence that is antisense to an miRNA;

SEQ ID NO: 797 is CGCCAAUAUUUACGUGCUGCUA, a sequence that is antisense to an miRNA;

SEQ ID NO: 798 is ACGACCUGCGUGACUAUUUCUA, a sequence that is antisense to an miRNA-like control sequence;

SEQ ID NO: 799 is ACACGAUUAGCGCUUCUCUGUA, a sequence that is antisense to an miRNA-like control sequence;

SEQ ID NO: 800 is AUCGGCUAAGCGAUUUCCUCUA, a sequence that is antisense to an miRNA-like control sequence;

SEQ ID NO: 801 is CUGGACACGUCACGUAUUUCUA, a sequence that is antisense to an miRNA-like control sequence;

SEQ ID NO: 802 is ACAAGUGCCUUCACUGCAGU, a sequence that is antisense to an miRNA;

SEQ ID NO: 803 is UAUCUGCACUAGAUGCACCUUA, a sequence that is antisense to an miRNA;

SEQ ID NO: 804 is CCCAUUCGUUCUUCAUAGAAGA, a sequence that is antisense to an miRNA-like control sequence;

SEQ ID NO: 805 is CUCGAUGAGACUUUUUACCCAA, a sequence that is antisense to an miRNA-like control sequence;

SEQ ID NO: 806 is AAGUCCAAUCGUUUUACCCUGA, a sequence that is antisense to an miRNA-like control sequence;

SEQ ID NO: 807 is GGUAUCACUCACUGCAUAUUCA, a sequence that is antisense to an miRNA-like control sequence;

SEQ ID NO: 808 is UCAGUUUUGCAUAGAUUUGCACA, a sequence that is antisense to an miRNA;

SEQ ID NO: 809 is UUCCUAUAUUUUUCAGGAAGGCA, a sequence that is antisense to an mRNA-like control sequence;

SEQ ID NO: 810 is UUCAGAAGUCAUAUUUGGUUCCA, a sequence that is antisense to an miRNA-like control sequence;

SEQ ID NO: 811 is UGUUGUAAAUUCAUGCUCAGUCA, a sequence that is antisense to an miRNA-like control sequence;

SEQ ID NO: 812 is CUUGUAUCAAGGUCCUUAUUGAA, a sequence that is antisense to an miRNA-like control sequence;

SEQ ID NO: 813 is UCAGUUUUGCAUGGAUUUGCACA, a sequence that is antisense to an miRNA;

SEQ ID NO: 814 is CUACCUGCACUAUAAGCACUUUA, a sequence that is antisense to an miRNA;

SEQ ID NO: 815 is ACUAUUAUCAUGUUCCAGCACCA, a sequence that is antisense to an miRNA-like control sequence;

SEQ ID NO: 816 is CCUUGACAAUAUGUACAUCCUCA, a sequence that is antisense to an miRNA-like control sequence;

SEQ ID NO: 817 is CAUACUGUCACACUGCAUUUACA, a sequence that is antisense to an miRNA-like control sequence;

SEQ ID NO: 818 is UACAUGAUCCCAGAUCCUCAUUA, a sequence that is antisense to an miRNA-like control sequence;

SEQ ID NO: 819 is UCAACAUCAGUCUGAUAAGCUA, a sequence that is antisense to an miRNA;

SEQ ID NO: 820 is UGUGAUAACCCUGCAUAACUAA, a sequence that is antisense to an miRNA-like control sequence;

SEQ ID NO: 821 is UAAGUCCCUGAAGUCACUAAUA, a sequence that is antisense to an miRNA-like control sequence;

SEQ ID NO: 822 is GCCAACUACUUGACAUUAUAGA, a sequence that is antisense to an miRNA-like control sequence;

SEQ ID NO: 823 is GCCUGUCCAUGUAAAUAACUAA, a sequence that is antisense to an miRNA-like control sequence;

SEQ ID NO: 824 is ACAGUUCUUCAACUGGCAGCUU, a sequence that is antisense to an miRNA;

SEQ ID NO: 825 is UGGAGUUUCAAAGUCCACCUCU, a sequence that is antisense to an miRNA-like control sequence;

SEQ ID NO: 826 is CCAACUGGUGUUGACACAUCUU, a sequence that is antisense to an miRNA-like control sequence;

SEQ ID NO: 827 is GACUUCUUAAAGAGUGCCCCUU, a sequence that is antisense to an miRNA-like control sequence;

SEQ ID NO: 828 is UGCCAACACAGGCUUUCAUGUU, a sequence that is antisense to an miRNA-like control sequence;

SEQ ID NO: 829 is GGAAAUCCCUGGCAAUGUGAU, a sequence that is antisense to an miRNA;

SEQ ID NO: 830 is CCUAGAGGCUUGCAGAAUGAU, a sequence that is antisense to an miRNA-like control sequence;

SEQ ID NO: 831 is CAUUCAGACAGCUUGGAGGAU, a sequence that is antisense to an miRNA-like control sequence;

SEQ ID NO: 832 is CAGGUCAGAUUUAGCCAGGAU, a sequence that is antisense to an miRNA-like control sequence;

SEQ ID NO: 833 is UGGCCUCUCAAAAUGGGAGAU, a sequence that is antisense to an miRNA-like control sequence;

SEQ ID NO: 834 is GUGGUAAUCCCUGGCAAUGUGAU, a sequence that is antisense to an miRNA;

SEQ ID NO: 835 is CUGUUCCUGCUGAACUGAGCCA, a sequence that is antisense to an miRNA;

SEQ ID NO: 836 is GAAUGCAUUGCCUUCCUGGCCA, a sequence that is antisense to an miRNA-like control sequence;

SEQ ID NO: 837 is CUCUCAGGGAGGAUUUCCCUCA, a sequence that is antisense to an miRNA-like control sequence;

SEQ ID NO: 838 is AGGCAAUUUCUUGCCCCUGGCA, a sequence that is antisense to an miRNA-like control sequence;

SEQ ID NO: 839 is CCCUAUUGGGCUUUCCAGAGCA, a sequence that is antisense to an miRNA-like control sequence;

SEQ ID NO: 840 is UCAGACCGAGACAAGUGCAAUG, a sequence that is antisense to an miRNA;

SEQ ID NO: 841 is AACCAACACGAUCAAUUGGGGG, a sequence that is antisense to an miRNA-like control sequence;

SEQ ID NO: 842 is AAUGAGACCAACGGGUCUCAAG, a sequence that is antisense to an miRNA-like control sequence;

SEQ ID NO: 843 is CAACUUAGAGGACGAGCCAAUG, a sequence that is antisense to an miRNA-like control sequence;

SEQ ID NO: 844 is CAGGUGAACCGAAUACCAAUGG, a sequence that is antisense to an miRNA-like control sequence;

SEQ ID NO: 845 is AGCCUAUCCUGGAUUACUUGAA, a sequence that is antisense to an miRNA;

SEQ ID NO: 846 is GUUCAGUACCCUUCUGAAGUAA, a sequence that is antisense to an miRNA-like control sequence;

SEQ ID NO: 847 is GUCUUAAGCUUACCUGCAGUAA, a sequence that is antisense to an miRNA-like control sequence;

SEQ ID NO: 848 is AUUGUCAGGUCCCAUAACUUGA, a sequence that is antisense to an miRNA-like control sequence;

SEQ ID NO: 849 is UAAUUUACCCUCCAGAGGGUUA, a sequence that is antisense to an miRNA-like control sequence;

SEQ ID NO: 850 is AACCUAUCCUGAAUUACUUGAA, a sequence that is antisense to an miRNA;

SEQ ID NO: 851 is GGCGGAACUUAGCCACUGUGAA, a sequence that is antisense to an miRNA;

SEQ ID NO: 852 is ACAAGGCUGAGCGAUGCUGUCA, a sequence that is antisense to an miRNA-like control sequence;

SEQ ID NO: 853 is ACGAAUGGUCCCAUGCAGU, a sequence that is antisense to an miRNA-like control sequence;

SEQ ID NO: 854 is CACGGGGCCAGACAGGAUUUUA, a sequence that is antisense to an miRNA-like control sequence;

SEQ ID NO: 855 is GAGGCAAACCGGGGCUUUCAUA, a sequence that is antisense to an miRNA-like control sequence;

SEQ ID NO: 856 is CAGAACUUAGCCACUGUGAA, a sequence that is antisense to an miRNA;

SEQ ID NO: 857 is CUCAAUAGACUGUGAGCUCCUU, a sequence that is antisense to an miRNA;

SEQ ID NO: 858 is AACCGAUUUCAGAUGGUGCUAG, a sequence that is antisense to an miRNA;

SEQ ID NO: 859 is AAGCCCUUAAUUCGUAGUGAGG, a sequence that is antisense to an miRNA-like control sequence;

SEQ ID NO: 860 is AAAGGGUGCAUUCGUCUAUCAG, a sequence that is antisense to an miRNA-like control sequence;

SEQ ID NO: 861 is UGAGACAAUUGCCGACUUAGUG, a sequence that is antisense to an miRNA-like control sequence;

SEQ ID NO: 862 is ACUGCGAAGUCUUGAUCUAGAG, a sequence that is antisense to an miRNA-like control sequence;

SEQ ID NO: 863 is AACACUGAUUUCAAAUGGUGCUA, a sequence that is antisense to an miRNA;

SEQ ID NO: 864 is CUUACAAGAGCAGGUUUCUAAUA, a sequence that is antisense to an miRNA-like control sequence;

SEQ ID NO: 865 is CAACUAAUUGAGAGUUCUGCAUA, a sequence that is antisense to an miRNA-like control sequence;

SEQ ID NO: 866 is AGGAAUUCCUGUCCUUAAAGUAA, a sequence that is antisense to an miRNA-like control sequence; SEQ ID NO: 867 is CUUUUAUCCAAUGGGAGAACAUA, a sequence that is antisense to an miRNA-like control sequence;

SEQ ID NO: 868 is UAACCGAUUUCAAAUGGUGCUA, a sequence that is antisense to an miRNA;

SEQ ID NO: 869 is GCUUCCAGUCGAGGAUGUUUACA, a sequence that is antisense to an miRNA;

SEQ ID NO: 870 is GCUGAGUGUAGGAUGUUUACA, a sequence that is antisense to an miRNA;

SEQ ID NO: 871 is CUAUGUGGAUGUGUGUGACAA, a sequence that is antisense to an miRNA-like control sequence;

SEQ ID NO: 872 is UAGAAUAGGUGUUGCUCUGGA, a sequence that is antisense to an miRNA-like control sequence;

SEQ ID NO: 873 is UGUGUAAUGGACAUGGGCUUA, a sequence that is antisense to an miRNA-like control sequence;

SEQ ID NO: 874 is UUAUGGUCAUGUGUGGACAGA, a sequence that is antisense to an miRNA-like control sequence;

SEQ ID NO: 875 is GCUGAGAGUGUAGGAUGUUUACA, a sequence that is antisense to an miRNA;

SEQ ID NO: 876 is CUUCCAGUCGGGGAUGUUUACA, a sequence that is antisense to an miRNA;

SEQ ID NO: 877 is CCAGUCAAGGAUGUUUACA, a sequence that is antisense to an miRNA;

SEQ ID NO: 878 is CAGCUAUGCCAGCAUCUUGCC, a sequence that is antisense to an miRNA;

SEQ ID NO: 879 is CCUCUAAGGACUCAGUUGCCC, a sequence that is antisense to an miRNA-like control sequence;

SEQ ID NO: 880 is CUGUGCCUGACUAGCCUCAAC, a sequence that is antisense to an miRNA-like control sequence;

SEQ ID NO: 881 is UGGGCCAAUCCAUGCCCAUUC, a sequence that is antisense to an miRNA-like control sequence;

SEQ ID NO: 882 is GACCAGACCUUGCCUUGUACC, a sequence that is antisense to an miRNA-like control sequence;

SEQ ID NO: 883 is GCAACUUAGUAAUGUGCAAUA, a sequence that is antisense to an miRNA;

SEQ ID NO: 884 is CAAUGCAACUACAAUGCAC, a sequence that is antisense to an miRNA;

SEQ ID NO: 885 is AACCCUAGAUGAUCAAACC, a sequence that is antisense to an miRNA-like control sequence;

SEQ ID NO: 886 is UAAGCUAAUCAAGAACCCC, a sequence that is antisense to an miRNA-like control sequence;

SEQ ID NO: 887 is AAACACCUUAGCCAAGAUC, a sequence that is antisense to an miRNA-like control sequence;

SEQ ID NO: 888 is CAAGUGACCUACAACAAUC, a sequence that is antisense to an miRNA-like control sequence;

SEQ ID NO: 889 is CAAUGCAACAGCAAUGCAC, a sequence that is antisense to an miRNA;

SEQ ID NO: 890 is ACAACCAGCUAAGACACUGCCA, a sequence that is antisense to an miRNA;

SEQ ID NO: 891 is ACCCCAUAGAGAAUCAGCCACA, a sequence that is antisense to an miRNA-like control sequence;

SEQ ID NO: 892 is GUUACACACAAACACAGGCCCA, a sequence that is antisense to an miRNA-like control sequence;

SEQ ID NO: 893 is GAGACUCCCACUGACACACAAA, a sequence that is antisense to an miRNA-like control sequence;

SEQ ID NO: 894 is AACAUACCAGACCCUCCAGGAA, a sequence that is antisense to an miRNA-like control sequence;

SEQ ID NO: 895 is ACAGGCCGGGACAAGUGCAAUA, a sequence that is antisense to an miRNA;

SEQ ID NO: 896 is CUACCUGCACGAACAGCACUUU, a sequence that is antisense to an miRNA;

SEQ ID NO: 897 is AACACAUCUCCGGCAACCUGUU, a sequence that is antisense to an miRNA-like control sequence;

SEQ ID NO: 898 is GCAAGCACUUUACGCCACACUU, a sequence that is antisense to an miRNA-like control sequence;

SEQ ID NO: 899 is CUCUAGCUGACCGCUCCAAAAU, a sequence that is antisense to an miRNA-like control sequence;

SEQ ID NO: 900 is UCCCAAUUAAACCGCCAGUGCU, a sequence that is antisense to an miRNA-like control sequence;

SEQ ID NO: 901 is AUCUGCACUGUCAGCACUUU, a sequence that is antisense to an miRNA;

SEQ ID NO: 902 is UGCUCAAUAAAUACCCGUUGAA, a sequence that is antisense to an miRNA;

SEQ ID NO: 903 is GCAAAAAUGUGCUAGUGCCAAA, a sequence that is antisense to an miRNA;

SEQ ID NO: 904 is GAGAGAACAUCCAAGGAUUUCA, a sequence that is antisense to an miRNA-like control sequence;

SEQ ID NO: 905 is AACUGGUUAAACCAGUGAAGCA, a sequence that is antisense to an miRNA-like control sequence;

SEQ ID NO: 906 is GCAAAUGACAGAAGGACUUUCA, a sequence that is antisense to an miRNA-like control sequence;

SEQ ID NO: 907 is UAAAAGGAUGUGCCUGCAACAA, a sequence that is antisense to an miRNA-like control sequence;

SEQ ID NO: 908 is AACAAUACAACUUACUACCUCA, a sequence that is antisense to an miRNA;

SEQ ID NO: 909 is ACAAGAUCGGAUCUACGGGU, a sequence that is antisense to an miRNA;

SEQ ID NO: 910 is UGACAGCGGGAUCGUACAAU, a sequence that is antisense to an miRNA-like control sequence;

SEQ ID NO: 911 is AGAGCGGUCUCACGGAAUAU, a sequence that is antisense to an miRNA-like control sequence; SEQ ID NO: 912 is GAUACAAGUGCCGAUCGGAU, a sequence that is antisense to an miRNA-like control sequence;

SEQ ID NO: 913 is AGAGCCAGUACGUCGGUAAU, a sequence that is antisense to an miRNA-like control sequence;

SEQ ID NO: 914 is CGCAAGGUCGGUUCUACGGGUG, a sequence that is antisense to an miRNA;

SEQ ID NO: 915 is CACAAGUUCGGAUCUACGGGUU, a sequence that is antisense to an miRNA;

SEQ ID NO: 916 is GGUUGAAACCCACGUUAUGCGU, a sequence that is antisense to an miRNA-like control sequence;

SEQ ID NO: 917 is CCUGACGAGUUGAGUAAUCGCU, a sequence that is antisense to an miRNA-like control sequence;

SEQ ID NO: 918 is UGAAUCCCGGGAAUGCGCUUAU, a sequence that is antisense to an miRNA-like control sequence;

SEQ ID NO: 919 is GGUGUAGCCAUAUAGCCGUACU, a sequence that is antisense to an miRNA-like control sequence;

SEQ ID NO: 920 is UCAGUUAUCACAGUACUGUA, a sequence that is antisense to an miRNA;

SEQ ID NO: 921 is GCCUCUUACUAAGUGUAUAA, a sequence that is antisense to an miRNA-like control sequence;

SEQ ID NO: 922 is GCAGUCUAGCUUCUAUAUAA, a sequence that is antisense to an miRNA-like control sequence;

SEQ ID NO: 923 is UCUAUUGGCUACAUAGCAUA, a sequence that is antisense to an miRNA-like control sequence;

SEQ ID NO: 924 is GUUGAUAGCACCCUAUAUUA, a sequence that is antisense to an miRNA-like control sequence;

SEQ ID NO: 925 is UCAUAGCCCUGUACAAUGCUGCU, a sequence that is antisense to an miRNA;

SEQ ID NO: 926 is GGUCAUCUCAAUACUGCCCUGAU, a sequence that is antisense to an miRNA-like control sequence;

SEQ ID NO: 927 is GAGUCUAGAGCCUACUCUCACUU, a sequence that is antisense to an miRNA-like control sequence;

SEQ ID NO: 928 is GAUAUCCUGUCCAUUAGAGCCCU, a sequence that is antisense to an miRNA-like control sequence;

SEQ ID NO: 929 is CUUUGCUCCGAGGAAACUUCCAU, a sequence that is antisense to an miRNA-like control sequence;

SEQ ID NO: 930 is UCAUAGCCCUGUACAAUGUUGCU, a sequence that is antisense to an miRNA;

SEQ ID NO: 931 is UAGCUUAUCAGACUGAUGUUGA, a sequence that is antisense to an miRNA;

SEQ ID NO: 932 is CGAAGGGUUUUCUUCUAUGAAA, a sequence that is antisense to an miRNA-like control sequence;

SEQ ID NO: 933 is GGUAAUGUAUCCUGACUUUAGA, a sequence that is antisense to an miRNA-like control sequence;

SEQ ID NO: 934 is UGAUCUACUGUGUGAGAUUCAA, a sequence that is antisense to an miRNA-like control sequence;

SEQ ID NO: 935 is UAGUCUUAAGGGUAAUUCUGCA, a sequence that is antisense to an miRNA-like control sequence;

SEQ ID NO: 936 is ACAGGAGUCUGAGCAUUUGA, a sequence that is antisense to an miRNA;

SEQ ID NO: 937 is GCUACCUGCACUGUAAGCACUUUU, a sequence that is antisense to an miRNA;

SEQ ID NO: 938 is UGAUAGCCCUGUACAAUGCUGCU, a sequence that is antisense to an miRNA;

SEQ ID NO: 939 is AAUGCCCCUAAAAAUCCUUAU, a sequence that is antisense to an miRNA;

SEQ ID NO: 940 is CAAAUCAUUGCCUCAUAACAU, a sequence that is antisense to an miRNA-like control sequence;

SEQ ID NO: 941 is AAGAAUCCACAAUUCCCUUAU, a sequence that is antisense to an miRNA-like control sequence;

SEQ ID NO: 942 is CUUUGAUCUAACAACACAACU, a sequence that is antisense to an miRNA-like control sequence;

SEQ ID NO: 943 is GAAACAAACUCUUCAUCUACU, a sequence that is antisense to an miRNA-like control sequence;

SEQ ID NO: 944 is ACAAACACCAUUGUCACACUCCA, a sequence that is antisense to an miRNA;

SEQ ID NO: 945 is AAACAUUCCCACAACCUGUACCA, a sequence that is antisense to an miRNA-like control sequence;

SEQ ID NO: 946 is UCUCCAAGAACAAAUCCUACCCA, a sequence that is antisense to an miRNA-like control sequence;

SEQ ID NO: 947 is CCCCAAAUCACUUACAGCUACAA, a sequence that is antisense to an miRNA-like control sequence;

SEQ ID NO: 948 is CACUGCACCCCACAAUAACUUAA, a sequence that is antisense to an miRNA-like control sequence;

SEQ ID NO: 949 is CGCGUACCAAAAGUAAUAAUG, a sequence that is antisense to an miRNA;

SEQ ID NO: 950 is UAAGCACGCGCAAAAUAUUAG, a sequence that is antisense to an miRNA-like control sequence;

SEQ ID NO: 951 is UCACAUCGCGGAAAAUAUAAG, a sequence that is antisense to an miRNA-like control sequence;

SEQ ID NO: 952 is CCAGCGUAACGAAAAUUAUAG, a sequence that is antisense to an miRNA-like control sequence;

SEQ ID NO: 953 is CGUAAGAAUCCGAAAUACAUG, a sequence that is antisense to an miRNA-like control sequence;

SEQ ID NO: 954 is UGGCAUUCACCGCGUGCCUUAA, a sequence that is antisense to an miRNA;

SEQ ID NO: 955 is AAGAUGUCGCCUCCGUUCUGCA, a sequence that is antisense to an miRNA-like control sequence;

SEQ ID NO: 956 is GGAGUCCUUCGACGCUCUCUAA, a sequence that is antisense to an miRNA-like control sequence;

SEQ ID NO: 957 is CUCUGCGGUUAUCGGCCAUCAA, a sequence that is antisense to an miRNA-like control sequence;

SEQ ID NO: 958 is AUCCUUCGGAUGACGUCCUGCA, a sequence that is antisense to an miRNA-like control sequence;

SEQ ID NO: 959 is CACAGGUUAAAGGGUCUCAGGGA, a sequence that is antisense to an miRNA;

SEQ ID NO: 960 is UCACAAGUUAGGGUCUCAGGGA, a sequence that is antisense to an miRNA;

SEQ ID NO: 961 is AGUGGAUGCAUAUUGGCCCAGA, a sequence that is antisense to an miRNA-like control sequence;

SEQ ID NO: 962 is AGCUACAAUGUCUGCAGGUGGA, a sequence that is antisense to an mRNA-like control sequence;

SEQ ID NO: 963 is AACUGUACUGCAGAUGGGCUGA, a sequence that is antisense to an miRNA-like control sequence;

SEQ ID NO: 964 is AUUACCCAGGAGAGCUGGGUUA, a sequence that is antisense to an miRNA-like control sequence;

SEQ ID NO: 965 is GCAUUAUUACUCACGGUACGA, a sequence that is antisense to an miRNA;

SEQ ID NO: 966 is UCACUGUACUAAGUCGCGAUA, a sequence that is antisense to an miRNA-like control sequence;

SEQ ID NO: 967 is GGUCACUAUCUUACGAUACGA, a sequence that is antisense to an miRNA-like control sequence;

SEQ ID NO: 968 is UACCCAUGAUUAGCGAUCGUA, a sequence that is antisense to an miRNA-like control sequence;

SEQ ID NO: 969 is UACAUAAGUCUUCCGUACGGA, a sequence that is antisense to an miRNA-like control sequence;

SEQ ID NO: 970 is AGCCAAGCUCAGACGGAUCCGA, a sequence that is antisense to an miRNA;

SEQ ID NO: 971 is AAAAGAGACCGGUUCACUGUGA, a sequence that is antisense to an miRNA;

SEQ ID NO: 972 is ACAUUGAGCGAACUUGAGCAGA, a sequence that is antisense to an miRNA-like control sequence;

SEQ ID NO: 973 is CAGAGGAAACGCAGUUCAGUUA, a sequence that is antisense to an miRNA-like control sequence;

SEQ ID NO: 974 is AUACAUAGGUAAUGCAGGGCCA, a sequence that is antisense to an miRNA-like control sequence;

SEQ ID NO: 975 is AAGAGCGAAACAGGUUCUGUCA, a sequence that is antisense to an miRNA-like control sequence;

SEQ ID NO: 976 is GAAAGAGACCGGUUCACUGUGA, a sequence that is antisense to an miRNA;

SEQ ID NO: 977 is GCAAGCCCAGACCGAAAAAG, a sequence that is antisense to an miRNA;

SEQ ID NO: 978 is GCAAGCCCAGACCGCAAAAAG, a sequence that is antisense to an miRNA;

SEQ ID NO: 979 is CCGGACCCAAACACAAAGGAG, a sequence that is antisense to an miRNA-like control sequence;

SEQ ID NO: 980 is GAAGAACCGGAAACCCCCAAG, a sequence that is antisense to an miRNA-like control sequence;

SEQ ID NO: 981 is GCCAAGCCAAACGCCAAAAGG, a sequence that is antisense to an miRNA-like control sequence;

SEQ ID NO: 982 is CGCCCAACCAGAACAAAGGAG, a sequence that is antisense to an miRNA-like control sequence;

SEQ ID NO: 983 is GCCCUUUUAACAUUGCACUG, a sequence that is antisense to an miRNA;

SEQ ID NO: 984 is UUUGAAGUCCACCUCUCAUG, a sequence that is antisense to an miRNA-like control sequence;

SEQ ID NO: 985 is UCUUCCAAGUCUGCAUUCAG, a sequence that is antisense to an mRNA-like control sequence;

SEQ ID NO: 986 is GUUAUGUUUCCCUCCAACAG, a sequence that is antisense to an miRNA-like control sequence;

SEQ ID NO: 987 is UCCAUCCUCAAUUUGAGCUG, a sequence that is antisense to an miRNA-like control sequence;

SEQ ID NO: 988 is GCCCUUUCAUCAUUGCACUG, a sequence that is antisense to an miRNA;

SEQ ID NO: 989 is ACUUUCGGUUAUCUAGCUUUA, a sequence that is antisense to an miRNA;

SEQ ID NO: 990 is UGUGCAUUCACGUUUAUCUUA, a sequence that is antisense to an miRNA-like control sequence;

SEQ ID NO: 991 is UGUUUUACAUCGGAUCUUCUA, a sequence that is antisense to an miRNA-like control sequence;

SEQ ID NO: 992 is UACUUUUAGGCUCGUUUCAUA, a sequence that is antisense to an miRNA-like control sequence;

SEQ ID NO: 993 is UAUCUCGACUUUGUUGUCAUA, a sequence that is antisense to an miRNA-like control sequence;

SEQ ID NO: 994 is GCGACCAUGGCUGUAGACUGUUA, a sequence that is antisense to an miRNA;

SEQ ID NO: 995 is GGAUCUCCGACUAUGGGAUGCUA, a sequence that is antisense to an miRNA-like control sequence;

SEQ ID NO: 996 is AGGUCUUCCGUACCAGGAUGUGA, a sequence that is antisense to an miRNA-like control sequence;

SEQ ID NO: 997 is CUCCGGUAGGCAGUAUGGCAUUA, a sequence that is antisense to an miRNA-like control sequence;

SEQ ID NO: 998 is GCUGUACUGUCGGAAUGCCAGUA, a sequence that is antisense to an miRNA-like control sequence;

SEQ ID NO: 999 is ACAGCUGGUUGAAGGGGACCAA, a sequence that is antisense to an miRNA;

SEQ ID NO: 1000 is CUGACAAGCAAUGAGGUGGGCA, a sequence that is antisense to an miRNA-like control sequence;

SEQ ID NO: 1001 is GGGAGGCUUCAUGAGAACCAGA, a sequence that is antisense to an miRNA-like control sequence;

SEQ ID NO: 1002 is CCUAAAGGCAAGGGGGACUUGA, a sequence that is antisense to an miRNA-like control sequence;

SEQ ID NO: 1003 is AGCUGUGGGCCAAAGAGAUGCA, a sequence that is antisense to an miRNA-like control sequence;

SEQ ID NO: 1004 is UAGCUGGUUGAAGGGGACCAA, a sequence that is antisense to an miRNA;

SEQ ID NO: 1005 is CCCCUCUGGUCAACCAGUCACA, a sequence that is antisense to an miRNA;

SEQ ID NO: 1006 is AUCACAUAGGAAUAAAAAGCCAUA, a sequence that is antisense to an miRNA;

SEQ ID NO: 1007 is AUUAGUACACAAAAAACCAGAGUA, a sequence that is antisense to an miRNA-like control sequence;

SEQ ID NO: 1008 is GAUAAAAAUAGCACAACAUGCAUA, a sequence that is antisense to an miRNA-like control sequence;

SEQ ID NO: 1009 is GCAAAUAUAAAUAGCCAAGAACUA, a sequence that is antisense to an miRNA-like control sequence;

SEQ ID NO: 1010 is GUAGCAGAAAACCUAACAUAAAUA, a sequence that is antisense to an miRNA-like control sequence;

SEQ ID NO: 1011 is UCCAUCAUCAAAACAAAUGGAGU, a sequence that is antisense to an miRNA;

SEQ ID NO: 1012 is CUACGCGUAUUCUUAAGCAAUA, a sequence that is antisense to an miRNA;

SEQ ID NO: 1013 is ACCGUUAAUCGUAAGACUUCUA, a sequence that is antisense to an miRNA-like control sequence;

SEQ ID NO: 1014 is AACGUAACAUUCGUCACUUGUA, a sequence that is antisense to an mRNA-like control sequence;

SEQ ID NO: 1015 is CAAGUACGCGUAUUAAUCUCUA, a sequence that is antisense to an miRNA-like control sequence;

SEQ ID NO: 1016 is CGAUCUCACGUCUAUUAGUAAA, a sequence that is antisense to an miRNA-like control sequence;

SEQ ID NO: 1017 is GAUUCACAACACCAGCU, a sequence that is antisense to an miRNA;

SEQ ID NO: 1018 is CUCCAAAUACACAGGCU, a sequence that is antisense to an miRNA-like control sequence;

SEQ ID NO: 1019 is CCCAAGUAACCACUGAU, a sequence that is antisense to an miRNA-like control sequence;

SEQ ID NO: 1020 is AGGAAAUCACCCUCCAU, a sequence that is antisense to an miRNA-like control sequence;

SEQ ID NO: 1021 is CCAGGCUACAAAUCCAU, a sequence that is antisense to an miRNA-like control sequence;

SEQ ID NO: 1022 is ACUGGAGACACGUGCACUGUAGA, a sequence that is antisense to an miRNA;

SEQ ID NO: 1023 is CUACCAUAGGGUAAAACCACU, a sequence that is antisense to an miRNA;

SEQ ID NO: 1024 is AACAGGAUACCAUCACAUGCU, a sequence that is antisense to an miRNA-like control sequence;

SEQ ID NO: 1025 is AGUAUACCCAGCCAACAAUGU, a sequence that is antisense to an miRNA-like control sequence;

SEQ ID NO: 1026 is AUUCGUCAAGAAAAGCCCACU, a sequence that is antisense to an miRNA-like control sequence;

SEQ ID NO: 1027 is CUAACCCGAAAAAGGACUCUU, a sequence that is antisense to an miRNA-like control sequence;

SEQ ID NO: 1028 is CCAUCUUUACCAGACAGUGUU, a sequence that is antisense to an miRNA;

SEQ ID NO: 1029 is AUCUGAGCACAUUUCUGCCAU, a sequence that is antisense to an miRNA-like control sequence;

SEQ ID NO: 1030 is UCUGCUCACUUGCAAAUCAGU, a sequence that is antisense to an miRNA-like control sequence;

SEQ ID NO: 1031 is AAUGUUAACUGGCUCCUCACU, a sequence that is antisense to an miRNA-like control sequence;

SEQ ID NO: 1032 is UUAAUGAGCUUGAUCCCACCU, a sequence that is antisense to an miRNA-like control sequence;

SEQ ID NO: 1033 is CCAUAAAGUAGGAAACACUACA, a sequence that is antisense to an miRNA;

SEQ ID NO: 1034 is CAAUAGAAGUCAAGAUACCACA, a sequence that is antisense to an miRNA-like control sequence;

SEQ ID NO: 1035 is AAACACCUACAAGAAGGCUAUA, a sequence that is antisense to an miRNA-like control sequence;

SEQ ID NO: 1036 is CAGUAGCAAACAAGUACUACAA, a sequence that is antisense to an miRNA-like control sequence;

SEQ ID NO: 1037 is AUACAACAAGCCAAAGCUAGUA, a sequence that is antisense to an miRNA-like control sequence;

SEQ ID NO: 1038 is GUAGUGCUUUCUACUUUAUGGG, a sequence that is antisense to an miRNA;

SEQ ID NO: 1039 is GUGUUUAUGGCUUAUGACUCUG, a sequence that is antisense to an miRNA-like control sequence;

SEQ ID NO: 1040 is GGGCAUGGUUUAUUAUCUUCUG, a sequence that is antisense to an miRNA-like control sequence;

SEQ ID NO: 1041 is AGUAUGUCUGCUCUUUUAGUGG, a sequence that is antisense to an miRNA-like control sequence;

SEQ ID NO: 1042 is CAGGCUUUCGAUUUUUUGGUAG, a sequence that is antisense to an miRNA-like control sequence;

SEQ ID NO: 1043 is UGAGCUACAGUGCUUCAUCUCA, a sequence that is antisense to an miRNA;

SEQ ID NO: 1044 is UCACUGUGUGAGCUCCACAUUA, a sequence that is antisense to an miRNA-like control sequence;

SEQ ID NO: 1045 is UGCACUGAGCCAUCUAUUCUGA, a sequence that is antisense to an miRNA-like control sequence;

SEQ ID NO: 1046 is AGUCAGGUAUUCCUUCCACUGA, a sequence that is antisense to an miRNA-like control sequence;

SEQ ID NO: 1047 is CAUCUCACUUCUUAUGGGGACA, a sequence that is antisense to an miRNA-like control sequence;

SEQ ID NO: 1048 is CUAGUACAUCAUCUAUACUGUA, a sequence that is antisense to an miRNA;

SEQ ID NO: 1049 is GCUCUCUUUCGCAAUAAUAUAA, a sequence that is antisense to an miRNA-like control sequence;

SEQ ID NO: 1050 is UUCGUUGAACCCUUAUACAUAA, a sequence that is antisense to an miRNA-like control sequence;

SEQ ID NO: 1051 is CAUUCCUUUAGACGACAUUAUA, a sequence that is antisense to an miRNA-like control sequence;

SEQ ID NO: 1052 is UUUCGCACAAUGCCUAUAUUAA, a sequence that is antisense to an miRNA-like control sequence;

SEQ ID NO: 1053 is AAGGGAUUCCUGGGAAAACUGGAC, a sequence that is antisense to an miRNA;

SEQ ID NO: 1054 is GAUGAAAAACUCUUGCAGGGGGAC, a sequence that is antisense to an miRNA-like control sequence;

SEQ ID NO: 1055 is AUUUGCAAGGGCAAGAGCUGGAAC, a sequence that is antisense to an miRNA-like control sequence;

SEQ ID NO: 1056 is AAAGUCUUGAACAGCAAGGGGUGC, a sequence that is antisense to an miRNA-like control sequence;

SEQ ID NO: 1057 is AAAAGAUGAAGCUGGAGUGGCCUC, a sequence that is antisense to an miRNA-like control sequence;

SEQ ID NO: 1058 is AACCCAUGGAAUUCAGUUCUCA, a sequence that is antisense to an miRNA;

SEQ ID NO: 1059 is CUCAUUUGUAAGCCAUCCAGAA, a sequence that is antisense to an miRNA-like control sequence;

SEQ ID NO: 1060 is ACUGUGCAACUGAAUCCAUUCA, a sequence that is antisense to an miRNA-like control sequence;

SEQ ID NO: 1061 is GCUUCAACUGUUAGAAACUCCA, a sequence that is antisense to an miRNA-like control sequence;

SEQ ID NO: 1062 is UGUUAACAAGCUCAGUCCUCAA, a sequence that is antisense to an miRNA-like control sequence;

SEQ ID NO: 1063 is GGCAGAAGCAUUUCCACACAC, a sequence that is antisense to an miRNA;

SEQ ID NO: 1064 is ACAAAGUUCUGUAGUGCACUGA, a sequence that is antisense to an miRNA;

SEQ ID NO: 1065 is AUUCUUGAUAUCAAGCAGGGCA, a sequence that is antisense to an miRNA-like control sequence;

SEQ ID NO: 1066 is GAAGUGGCAUUUUACUCACAGA, a sequence that is antisense to an miRNA-like control sequence;

SEQ ID NO: 1067 is CUGGUAACUUCAGGUAAAUGCA, a sequence that is antisense to an miRNA-like control sequence;

SEQ ID NO: 1068 is UUGCAGAAUAGCAGUUCACUGA, a sequence that is antisense to an miRNA-like control sequence;

SEQ ID NO: 1069 is ACAAAGUUCUGUGAUGCACUGA, a sequence that is antisense to an miRNA;

SEQ ID NO: 1070 is GGAGUGAAGACACGGAGCCAGA, a sequence that is antisense to an miRNA;

SEQ ID NO: 1071 is ACACUGGUACAAGGGUUGGGAGA, a sequence that is antisense to an miRNA;

SEQ ID NO: 1072 is CCUCAAGGAGCUUCAGUCUAG, a sequence that is antisense to an miRNA;

SEQ ID NO: 1073 is CCAAGUUCUGUCAUGCACUGA, a sequence that is antisense to an miRNA;

SEQ ID NO: 1074 is UCACUUUUGUGACUAUGCAA, a sequence that is antisense to an miRNA;

SEQ ID NO: 1075 is CAUGUUCAUCUUAGGCUUAA, a sequence that is antisense to an miRNA-like control sequence;

SEQ ID NO: 1076 is UUAUUGGGCUUUACAACUCA, a sequence that is antisense to an miRNA-like control sequence;

SEQ ID NO: 1077 is CCUUAUUCUUAGACAUUGGA, a sequence that is antisense to an miRNA-like control sequence;

SEQ ID NO: 1078 is CUUAAGUGUUGUCACUCUAA, a sequence that is antisense to an miRNA-like control sequence;

SEQ ID NO: 1079 is CGAAGGCAACACGGAUAACCUA, a sequence that is antisense to an miRNA;

SEQ ID NO: 1080 is CCCCUAUCACAAUUAGCAUUAA, a sequence that is antisense to an miRNA;

SEQ ID NO: 1081 is UCUGACCCUAAACAUUCAACUA, a sequence that is antisense to an miRNA-like control sequence;

SEQ ID NO: 1082 is AUACACUUGAACCAUUCACUCA, a sequence that is antisense to an miRNA-like control sequence;

SEQ ID NO: 1083 is ACAACAUGUUCCCUCCUAAAUA, a sequence that is antisense to an miRNA-like control sequence;

SEQ ID NO: 1084 is CUCAUUGACCAAUACUCUACAA, a sequence that is antisense to an miRNA-like control sequence;

SEQ ID NO: 1085 is ACUCACCGACAGCGUUGAAUGUU, a sequence that is antisense to an miRNA;

SEQ ID NO: 1086 is CUGACGUCCGAUGUUCACAGAAU, a sequence that is antisense to an miRNA-like control sequence;

SEQ ID NO: 1087 is GCCGAUAGUUCUCGGAAACACUU, a sequence that is antisense to an miRNA-like control sequence;

SEQ ID NO: 1088 is UGCCGUCUGACGAUCAGAAACUU, a sequence that is antisense to an miRNA-like control sequence;

SEQ ID NO: 1089 is AUAUGAAACGCGGCCUUCUCAGU, a sequence that is antisense to an miRNA-like control sequence;

SEQ ID NO: 1090 is AACCCACCGACAGCAAUGAAUGUU, a sequence that is antisense to an miRNA;

SEQ ID NO: 1091 is ACUCACCGACAGGUUGAAUGUU, a sequence that is antisense to an miRNA;

SEQ ID NO: 1092 is UGUGAGUUCUACCAUUGCCAAA, a sequence that is antisense to an miRNA;

SEQ ID NO: 1093 is AGAACUAUUCUGCUCUUGCAGA, a sequence that is antisense to an mRNA-like control sequence;

SEQ ID NO: 1094 is GUACAGUUCUCCAAUUUGGCAA, a sequence that is antisense to an miRNA-like control sequence;

SEQ ID NO: 1095 is GUCUGGUCAUGACUCAAAUUCA, a sequence that is antisense to an miRNA-like control sequence;

SEQ ID NO: 1096 is CUAAUUGGGCUAUCCUUGACAA, a sequence that is antisense to an miRNA-like control sequence;

SEQ ID NO: 1097 is CAGUGAAUUCUACCAGUGCCAUA, a sequence that is antisense to an miRNA;

SEQ ID NO: 1098 is UGAAACCAGCUGCUCCAUAGUUA, a sequence that is antisense to an miRNA-like control sequence;

SEQ ID NO: 1099 is UUGAUGCUCACCACAAGUGCAUA, a sequence that is antisense to an miRNA-like control sequence;

SEQ ID NO: 1100 is CCCUUAACUGAUGUGUAACCAGA, a sequence that is antisense to an miRNA-like control sequence;

SEQ ID NO: 1101 is CUCAAACAGUCAUGGCCUGUAUA, a sequence that is antisense to an miRNA-like control sequence;

SEQ ID NO: 1102 is ACCCUUAUCAGUUCUCCGUCCA, a sequence that is antisense to an miRNA;

SEQ ID NO: 1103 is CAUUAUCUCCUGUCCCACGUCA, a sequence that is antisense to an miRNA-like control sequence;

SEQ ID NO: 1104 is CACUAAUGCUCCUCCGUUCCUA, a sequence that is antisense to an miRNA-like control sequence;

SEQ ID NO: 1105 is UAUCCACACUUUCCUCUCCGGA, a sequence that is antisense to an miRNA-like control sequence;

SEQ ID NO: 1106 is CACAGCUUACUCUCCGUUCCUA, a sequence that is antisense to an miRNA-like control sequence;

SEQ ID NO: 1107 is GAACUGCCUUUCUCUCCA, a sequence that is antisense to an miRNA;

SEQ ID NO: 1108 is AAGCCCAAAAGGAGAAUUCUUUG, a sequence that is antisense to an miRNA;

SEQ ID NO: 1109 is CCGGCUGCAACACAAGACACGA, a sequence that is antisense to an miRNA;

SEQ ID NO: 1110 is CCCUAACCCGCAAAACGGAGGA, a sequence that is antisense to an miRNA-like control sequence;

SEQ ID NO: 1111 is CACCCAGAUGCCGAAACACGGA, a sequence that is antisense to an miRNA-like control sequence;

SEQ ID NO: 1112 is AGGAAAACAUCCCCGCCACGGA, a sequence that is antisense to an miRNA-like control sequence;

SEQ ID NO: 1113 is UCCAAGCCGCAAGCAACACGGA, a sequence that is antisense to an miRNA-like control sequence;

SEQ ID NO: 1114 is ACCCUCCACCAUGCAAGGGAUG, a sequence that is antisense to an miRNA;

SEQ ID NO: 1115 is ACUGAUGUCAGCUCAGUAGGCAC, a sequence that is antisense to an mRNA;

SEQ ID NO: 1116 is ACCUAAUAUAUCAAACAUAUCA, a sequence that is antisense to an miRNA;

SEQ ID NO: 1117 is AUCAAUAUAAUCUAAUACCACA, a sequence that is antisense to an mRNA-like control sequence;

SEQ ID NO: 1118 is UACAAUAUUACAACUAACUACA, a sequence that is antisense to an miRNA-like control sequence;

SEQ ID NO: 1119 is AUAAUAAUAACAUCUACUCACA, a sequence that is antisense to an miRNA-like control sequence;

SEQ ID NO: 1120 is ACUAAACCUAUAACAUUAUACA, a sequence that is antisense to an miRNA-like control sequence;

SEQ ID NO: 1121 is AGCUGCUUUUGGGAUUCCGUUG, a sequence that is antisense to an miRNA;

SEQ ID NO: 1122 is GGCUGUCAAUUCAUAGGUCAG, a sequence that is antisense to an miRNA;

SEQ ID NO: 1123 is AGACCUUGCUGACUAUUAGGG, a sequence that is antisense to an miRNA-like control sequence;

SEQ ID NO: 1124 is UCUCAGAUUGAGGAUAGCCUG, a sequence that is antisense to an miRNA-like control sequence;

SEQ ID NO: 1125 is UAAUUGGCCCCUGUAAGGUAG, a sequence that is antisense to an miRNA-like control sequence;

SEQ ID NO: 1126 is CUCUAAUUGAGGGUACCAUGG, a sequence that is antisense to an miRNA-like control sequence;

SEQ ID NO: 1127 is CUGGGACUUUGUAGGCCAGUU, a sequence that is antisense to an miRNA;

SEQ ID NO: 1128 is UCCACAUGGAGUUGCUGUUACA, a sequence that is antisense to an miRNA;

SEQ ID NO: 1129 is UAUUCCUUCUGGGUAAGGACCA, a sequence that is antisense to an miRNA-like control sequence;

SEQ ID NO: 1130 is UUGGCAUCUCUACUGCAUGGAA, a sequence that is antisense to an miRNA-like control sequence;

SEQ ID NO: 1131 is AGCAUGAUGGUUCUAUGUCCCA, a sequence that is antisense to an miRNA-like control sequence;

SEQ ID NO: 1132 is GCCUGGAUUAGCUCACUUUAGA, a sequence that is antisense to an miRNA-like control sequence;

SEQ ID NO: 1133 is GCCAAUAUUUCUGUGCUGCUA, a sequence that is antisense to an miRNA;

SEQ ID NO: 1134 is CCCAACAACAUGAAACUACCUA, a sequence that is antisense to an miRNA;

SEQ ID NO: 1135 is CCCAGAAACCAAACUAUCUACA, a sequence that is antisense to an miRNA-like control sequence;

SEQ ID NO: 1136 is CACAAAAUGACCCCUACCAAUA, a sequence that is antisense to an miRNA-like control sequence;

SEQ ID NO: 1137 is CCAGCCAUCAAAACUAUAACCA, a sequence that is antisense to an miRNA-like control sequence;

SEQ ID NO: 1138 is AAACACCUGUCCAAACUACACA, a sequence that is antisense to an mRNA-like control sequence;

SEQ ID NO: 1139 is GCUGGGUGGAGAAGGUGGUGAA, a sequence that is antisense to an miRNA;

SEQ ID NO: 1140 is CCUAUCUCCCCUCUGGACC, a sequence that is antisense to an miRNA;

SEQ ID NO: 1141 is GAACAGGUAGUCUGAACACUGGG, a sequence that is antisense to an miRNA;

SEQ ID NO: 1142 is CACAGGCUCAAUGGUAGAUGAGG, a sequence that is antisense to an miRNA-like control sequence;

SEQ ID NO: 1143 is GUAUCUGGAACUGGAGCAGACAG, a sequence that is antisense to an miRNA-like control sequence;

SEQ ID NO: 1144 is UAAGUGGAUGCCCAGAGUGACAG, a sequence that is antisense to an miRNA-like control sequence;

SEQ ID NO: 1145 is GAGGAUAGGUUGUAAACCCCAGG, a sequence that is antisense to an miRNA-like control sequence;

SEQ ID NO: 1146 is GAACAGAUAGUCUAAACACUGGG, a sequence that is antisense to an miRNA;

SEQ ID NO: 1147 is CAUCGUUACCAGACAGUGUUA, a sequence that is antisense to an miRNA;

SEQ ID NO: 1148 is GUCAUCAUUACCAGGCAGUAUUA, a sequence that is antisense to an miRNA;

SEQ ID NO: 1149 is AGUACUGGAUACCAUUCUCAGUA, a sequence that is antisense to an miRNA-like control sequence;

SEQ ID NO: 1150 is UAGUGUCCAAUAGUUAGCCACUA, a sequence that is antisense to an miRNA-like control sequence;

SEQ ID NO: 1151 is GAGUUAAGUACACUGUCCUCAUA, a sequence that is antisense to an miRNA-like control sequence;

SEQ ID NO: 1152 is UGGAUCCACUAAUAGUCCAUGUA, a sequence that is antisense to an miRNA-like control sequence;

SEQ ID NO: 1153 is AGAACAAUGCCUUACUGAGUA, a sequence that is antisense to an miRNA;

SEQ ID NO: 1154 is UCUUCCCAUGCGCUAUACCUCU, a sequence that is antisense to an miRNA;

SEQ ID NO: 1155 is UUGCCCCUACGUCCAUAUCUCU, a sequence that is antisense to an miRNA-like control sequence;

SEQ ID NO: 1156 is UCUCAGCCCGUUCCCUACUUAU, a sequence that is antisense to an miRNA-like control sequence;

SEQ ID NO: 1157 is ACAUCGUCCUGUCUUACUCCCU, a sequence that is antisense to an miRNA-like control sequence;

SEQ ID NO: 1158 is UGUCCCUUCCUACCGUACUCAU, a sequence that is antisense to an miRNA-like control sequence;

SEQ ID NO: 1159 is UCUAGUGGUCCUAAACAUUUCA, a sequence that is antisense to an miRNA;

SEQ ID NO: 1160 is UCAUCAUUGUGUCCCAAAUGUA, a sequence that is antisense to an miRNA-like control sequence;

SEQ ID NO: 1161 is GGCCAUCAUUUACUAGACUUUA, a sequence that is antisense to an miRNA-like control sequence;

SEQ ID NO: 1162 is UCACACACAGAUUGUUGUUCUA, a sequence that is antisense to an mRNA-like control sequence;

SEQ ID NO: 1163 is GGUGCUAAUCAUCAUUCCAUUA, a sequence that is antisense to an miRNA-like control sequence;

SEQ ID NO: 1164 is CAGGCAUAGGAUGACAAAGGGAA, a sequence that is antisense to an miRNA;

SEQ ID NO: 1165 is CAUAGGGGGGACAACAAAAGUGA, a sequence that is antisense to an miRNA-like control sequence;

SEQ ID NO: 1166 is UGAGCAAGUACAGGCAAGGAGAA, a sequence that is antisense to an miRNA-like control sequence;

SEQ ID NO: 1167 is AGUCAGGAGAGACCUAGAAGGAA, a sequence that is antisense to an miRNA-like control sequence;

SEQ ID NO: 1168 is AACAACUGUACAGGGGGGAGAAA, a sequence that is antisense to an miRNA-like control sequence;

SEQ ID NO: 1169 is CAGACUCCGGUGGAAUGAAGGA, a sequence that is antisense to an miRNA;

SEQ ID NO: 1170 is GGGAAGGCCGAAGGAAUUCCUA, a sequence that is antisense to an miRNA-like control sequence;

SEQ ID NO: 1171 is CAGCGCACUGUGGGAAAGUAGA, a sequence that is antisense to an miRNA-like control sequence;

SEQ ID NO: 1172 is UAGAAAGCCCGAUUGGGGGCAA, a sequence that is antisense to an miRNA-like control sequence;

SEQ ID NO: 1173 is GUUGGAAGGCCCGAUGGAACAA, a sequence that is antisense to an miRNA-like control sequence;

SEQ ID NO: 1174 is CCACACACUUCCUUACAUUCCA, a sequence that is antisense to an miRNA;

SEQ ID NO: 1175 is GAGGGAGGAGAGCCAGGAGAAGC, a sequence that is antisense to an miRNA;

SEQ ID NO: 1176 is ACAAGCUUUUUGCUCGUCUUAU, a sequence that is antisense to an miRNA;

SEQ ID NO: 1177 is CAUGAUCAUUUCUUUGUCGCAU, a sequence that is antisense to an miRNA-like control sequence;

SEQ ID NO: 1178 is GUUCAUUUUUAACCAUGCUCGU, a sequence that is antisense to an miRNA-like control sequence;

SEQ ID NO: 1179 is GUCAACUUCUUGUUUUAACGCU, a sequence that is antisense to an miRNA-like control sequence;

SEQ ID NO: 1180 is UUUUCUUAGCAUCAAGUCGUCU, a sequence that is antisense to an miRNA-like control sequence;

SEQ ID NO: 1181 is CAGCCGCUGUCACACGCACAG, a sequence that is antisense to an miRNA;

SEQ ID NO: 1182 is UCCAAGCCCGACAGGCCUACG, a sequence that is antisense to an miRNA-like control sequence;

SEQ ID NO: 1183 is AAGCCCCCCACGUGGCUAACG, a sequence that is antisense to an mRNA-like control sequence;

SEQ ID NO: 1184 is CUCACCCCCCGGAGGAUAACG, a sequence that is antisense to an miRNA-like control sequence;

SEQ ID NO: 1185 is CGCAACCAGCAUCUCCACGGG, a sequence that is antisense to an miRNA-like control sequence;

SEQ ID NO: 1186 is AGGCGAAGGAUGACAAAGGGAA, a sequence that is antisense to an miRNA;

SEQ ID NO: 1187 is GGCCGUGACUGGAGACUGUUA, a sequence that is antisense to an miRNA;

SEQ ID NO: 1188 is GGUACAAUCAACGGUCGAUGGU, a sequence that is antisense to an miRNA;

SEQ ID NO: 1189 is UACUAGCGAAGGAGAUCUCGGU, a sequence that is antisense to an miRNA-like control sequence;

SEQ ID NO: 1190 is GUAAAGGCUACGUGGUCACGAU, a sequence that is antisense to an miRNA-like control sequence;

SEQ ID NO: 1191 is UGAGACGCUGGCAUAGACGAUU, a sequence that is antisense to an miRNA-like control sequence;

SEQ ID NO: 1192 is GGAAUACCAUCGUGGUGACGAU, a sequence that is antisense to an miRNA-like control sequence;

SEQ ID NO: 1193 is CUGCCUGUCUGUGCCUGCUGU, a sequence that is antisense to an miRNA;

SEQ ID NO: 1194 is GCCUGGUCUUGGUCUCUGCCU, a sequence that is antisense to an miRNA-like control sequence;

SEQ ID NO: 1195 is CUCCUCUGUGGUGCUGCCUGU, a sequence that is antisense to an miRNA-like control sequence;

SEQ ID NO: 1196 is GUGGGCUGCUUCCCUUCCUGU, a sequence that is antisense to an miRNA-like control sequence;

SEQ ID NO: 1197 is UCUUGGGGGCUUCCCUCCUGU, a sequence that is antisense to an miRNA-like control sequence;

SEQ ID NO: 1198 is GUCUGUCAAUUCAUAGGUCAU, a sequence that is antisense to an miRNA;

SEQ ID NO: 1199 is CACAGUUGCCAGCUGAGAUUA, a sequence that is antisense to an miRNA;

SEQ ID NO: 1200 is CUGGAGACAUUCUUGACAGCA, a sequence that is antisense to an miRNA-like control sequence;

SEQ ID NO: 1201 is GUCAAAGCACCCUUGAGGUUA, a sequence that is antisense to an miRNA-like control sequence;

SEQ ID NO: 1202 is CAUGCAUGCCUGCAAAUGGUA, a sequence that is antisense to an mRNA-like control sequence;

SEQ ID NO: 1203 is CACCAAGGGCUGCUAUUUAGA, a sequence that is antisense to an miRNA-like control sequence;

SEQ ID NO: 1204 is AUCCAAUCAGUUCCUGAUGCAGUA, a sequence that is antisense to an miRNA;

SEQ ID NO: 1205 is ACAUGGUUAGAUCAAGCACAA, a sequence that is antisense to an miRNA;

SEQ ID NO: 1206 is AAUCACAGACUACAAGUGUGA, a sequence that is antisense to an miRNA-like control sequence;

SEQ ID NO: 1207 is UACUUGUGGAGAACACACAAA, a sequence that is antisense to an miRNA-like control sequence;

SEQ ID NO: 1208 is UCUAUAAACACUGAGGAAGCA, a sequence that is antisense to an miRNA-like control sequence;

SEQ ID NO: 1209 is GGACACAAGCUACUUAAUGAA, a sequence that is antisense to an miRNA-like control sequence;

SEQ ID NO: 1210 is AGAAUUGCGUUUGGACAAUCA, a sequence that is antisense to an miRNA;

SEQ ID NO: 1211 is UUAUCAUAGGGUAGAGCCUAA, a sequence that is antisense to an miRNA-like control sequence;

SEQ ID NO: 1212 is AGUUAGCAUAGGUCUAGCUAA, a sequence that is antisense to an miRNA-like control sequence;

SEQ ID NO: 1213 is AGUUAUAGGUAAGUUAGCCCA, a sequence that is antisense to an miRNA-like control sequence;

SEQ ID NO: 1214 is AUUAUGUCCACUAGGGGUAAA, a sequence that is antisense to an miRNA-like control sequence;

SEQ ID NO: 1215 is AAAGUGUCAGAUACGGUGUGG, a sequence that is antisense to an miRNA;

SEQ ID NO: 1216 is GAAACCCAGCAGACAAUGUAGCU, a sequence that is antisense to an miRNA;

SEQ ID NO: 1217 is CAUGGAGAAAGGCACCCACAUAU, a sequence that is antisense to an miRNA-like control sequence;

SEQ ID NO: 1218 is ACCCCAAAGCAGAACUAGGAUGU, a sequence that is antisense to an miRNA-like control sequence;

SEQ ID NO: 1219 is AAGCCACCCAACUGAAGAGGUAU, a sequence that is antisense to an miRNA-like control sequence;

SEQ ID NO: 1220 is GUGGCCAACCAGCAAGAACAUAU, a sequence that is antisense to an miRNA-like control sequence;

SEQ ID NO: 1221 is GAGACCCAGUAGCCAGAUGUAGCU, a sequence that is antisense to an miRNA;

SEQ ID NO: 1222 is UUGGGGUAUUUGACAAACUGACA, a sequence that is antisense to an miRNA;

SEQ ID NO: 1223 is GAGAUUUGGAUGCUCACAAGUUA, a sequence that is antisense to an miRNA-like control sequence;

SEQ ID NO: 1224 is GGAAUUUCUGAUUACAGUGAGCA, a sequence that is antisense to an miRNA-like control sequence;

SEQ ID NO: 1225 is CUGCUAAUGAAUCAGGAGUUGUA, a sequence that is antisense to an miRNA-like control sequence;

SEQ ID NO: 1226 is UCAGUUGGAACAGCUGUUGAAUA, a sequence that is antisense to an miRNA-like control sequence; and SEQ ID NO: 1227 is UAAACGGAACCACUAGUGACUUG, a sequence that is antisense to an miRNA.

### BRIEF DESCRIPTION OF THE APPENDICES

Appendix A is a computer program listing appendix of a program entitled “Appendix A.txt,” file created on Dec. 20, 2005 and having a filesize of 98,879 bytes, used in one embodiment of the invention, which program is incorporated herein by reference; and

Appendix B is a computer program listing appendix of a program used in Example 1 entitled “Appendix B.txt,” file created on Dec. 20, 2005 and having a filesize of 1,240,283 bytes, which program is incorporated herein by reference.

### DETAILED DESCRIPTION

The present invention generally relates to microRNAs such as vertebrate microRNA (miRNA), for example, mammalian miRNA. Various aspects of the invention are directed to the detection, production, or expression of miRNA. In one aspect, the invention provides systems and methods for identifying targets of miRNA sequences. For instance, in one embodiment, gene sequences comprising UTRs are compared with miRNA sequences to determine the degree of interaction, for example, by determining a free energy measurement between the miRNA sequence and the UTR, and/or by determining complementarity between at least a portion of the miRNA sequence and the UTR. In another aspect, the invention is directed to the regulation of gene expression using miRNA. For example, gene expression within a cell may be altered by exposing the cell to an oligonucleotide comprising a sequence that is substantially antisense to at least a portion of an miRNA region of the gene, for example, antisense to a 6-mer or 7-mer portion of the miRNA or it may be altered by increasing the level of miRNA available in the cell. In still another aspect, the invention is directed to the treatment of diseases such as cancer, autoimmune disease, arthritis, inflammatory disorders, osteogenesis, neurodegenerative disorders such as Alzheimer's, cardiovascular disease, kidney disease, hematopoiesis, hypercholesterolemia, and diabetes. For instance, in one set of embodiments, an isolated oligonucleotide comprising a sequence that is substantially antisense to an miRNA, or a portion of an miRNA, is administered to a subject having or being at risk of one or more of these diseases. Yet other aspects of the invention are directed to compositions or kits including oligonucleotides comprising a sequence that is substantially antisense to an miRNA (or a portion of an miRNA), methods of promoting any of the above aspects, or the like.

The following definitions will aid in the understanding of the invention. The term “nucleic acid,” as used herein, is given its ordinary meaning as used in the art, e.g., RNA (ribonucleic acid) or DNA (deoxyribonucleic acid). Typically, a nucleic acid includes multiple nucleotides, for example, adenosine (“A”), guanosine (“G”), uridine (“U”), or cytidine (“C”). Nucleotides typically are formed from molecules comprising a sugar (e.g. ribose or deoxyribose) linked to a phosphate group and an exchangeable organic base. A sugar and a base (without the phosphate) together form a nucleoside. Examples of organic bases include, but are not limited to, various pyrimidines or purines.

As used herein, terms such as “polynucleotide” or “oligonucleotide” generally refer to a polymer of at least two nucleotides. For example, the oligonucleotide may have 5, 6, 7, 8, 9, 10, 11, 12, 13, 14, 15, 16, 17, 18, 19, 20, 21, 22, 23, 24, 25, 26, 27, etc. bases or nucleotides. Those of ordinary skill in the art will recognize that these terms are not always precisely defined in terms of the number of bases present within the polymer. Polynucleotides where the sugars are predominantly deoxyribose are referred to as DNA or deoxyribonucleic acid, while polynucleotides where the sugars are predominantly ribose are referred to as RNA or ribonucleic acid.

As used herein, the term “sample” is used in its broadest sense. In one sense, it can refer to a vertebrate cell or tissue, for example, a fish cell (e.g., a zebrafish cell, or a pufferfish cell, etc.), an amphibian cell (e.g., a frog cell), an avian cell, a reptilian cell, a mammalian cell, etc. Examples of mammals include humans or non-human mammals, such as a monkey, ape, cow, sheep, goat, buffalo, antelope, oxen, horse, donkey, mule, deer, elk, caribou, water buffalo, camel, llama, alpaca, rabbit, pig, mouse, rat, guinea pig, hamster, dog, cat, etc. In another sense, the term “sample” is meant to include a specimen or culture obtained from any source (including those described above), as well as biological and environmental samples. Biological samples may be obtained from any vertebrate and encompass fluids, solids, tissues, and gases. Environmental samples include environmental material such as surface matter, soil, water, industrial samples, etc. These examples are not to be construed as limiting the sample types applicable to the present invention.

As used herein, “antisense” is given its ordinary meaning as used in the art, i.e., a first sequence (or portion of a sequence) that is antisense to a second sequence (or portion of the sequence) exhibits perfectly complementary Watson-Crick pairing (e.g., A:T, A:U and C:G pairing) with the second sequence when the sequences are properly aligned (i.e., in an antiparallel orientation). Similarly, sequences that are “substantially antisense” can, but do not necessarily exhibit perfectly complementary Watson-Crick pairing, but have enough complementarity that the sequences are able to specifically bind together in a defined, predictable orientation. For example, a first sequence (or portion) may be substantially antisense to the second sequence (or portion) if the sequences are perfectly complementary except for 1 nucleotide mismatch (for example, a G:U pairing), or a 2 nucleotide mismatch. As a non-limiting example, in some embodiments of the invention, an oligonucleotide may be prepared that is substantially antisense to a given miRNA sequence (or portion thereof), i.e., the oligonucleotide has either perfectly complementary Watson-Crick pairing with the given miRNA sequence, or includes 1 or 2 mismatches with the given miRNA sequence. In one embodiment; the oligonucleotide that is prepared has 18, 19, 20, 21, 22, 23, 24, 25, or 26 nucleotides, and at least a portion of the oligonucleotide is substantially antisense to the given miRNA sequence (or portion thereof). In another embodiment, the oligonucleotide that is prepared has 6, 7, or 8 nucleotides, and at least a portion of the oligonucleotide is substantially antisense to the given miRNA sequence, or portion thereof, for example, to a “seed” region within the given miRNA sequence. Non-limiting examples of sequences that are antisense to miRNA or miRNA-like sequences include SEQ ID NO: 762 to SEQ ID NO: 1227, and non-limiting examples of sequences that are antisense to miRNA seed regions include SEQ ID NO: 683 to SEQ ID NO: SEQ ID NO: 761.

Various aspects of the invention are directed to microRNAs such as vertebrate microRNA (miRNA), for example, mammalian miRNA. As used herein, “miRNA” or “microRNA” is given its ordinary meaning in the art. Typically, the miRNA is a RNA molecule derived from genomic loci processed from transcripts that can form local RNA precursor miRNA structures, and can be recognized by those of ordinary skill in the art. The mature miRNA usually has 20, 21, 22, 23, or 24 nucleotides, although in some cases, other numbers of nucleotides may be present, for example, between 18 and 26 nucleotides. miRNAs are often detectable on Northern blots. The miRNA has the potential to pair to flanking genomic sequences, placing the mature miRNA within an imperfect RNA duplex which may be needed for its processing from a longer precursor transcript. In animals, this processing typically occurs through the action of Drosha and Dicer endonucleases, which excise a miRNA duplex from the hairpin portion of the longer primary transcript. The miRNA duplex comprises the miRNA and a similar-sized segment, known as the miRNA* (miRNA star), from the other arm of the stem-loop. The miRNA is the strand that enters the silencing complex, whereas the miRNA* degrades. In addition, miRNAs are typically derived from a segment of the genome that is distinct from predicted protein-coding regions.

Thus, in various aspects of the present invention, the miRNAs can be processed from a portion of an miRNA transcript (i.e., a precursor miRNA) that, in some embodiments, can fold into a stable hairpin (i.e., a duplex) or a stem-loop structure. Typically, a portion of the precursor miRNA is cleaved to produce the final miRNA molecule. The hairpin structures may range from, for example, about 50 to about 80 nucleotides, or about 60 nucleotides to about 70 nucleotides (counting the miRNA residues, those pairing to the miRNA, and any intervening segment(s), but excluding more distal base pairs).

Those of ordinary skill in the art will be able to determine whether a given RNA sequence is an miRNA (or a portion thereof). Examples of considerations that those of ordinary skill in the art may look to in identifying miRNA include, but are not limited to, the following. (1) miRNAs derive from genomic loci distinct from other recognized genes. (2) miRNAs are processed from transcripts that can form local RNA hairpin precursor structures. (3) A single miRNA molecule predominately accumulates from one arm of each miRNA hairpin precursor molecule. (4) miRNA sequences are typically conserved in related organisms.

Non-limiting examples of miRNA sequences include the miRNA sequences of SEQ ID NO: 3 to SEQ ID NO: 468. Other non-limiting examples of miRNA include let-7a, let-7b, let-7c, let-7d, let-7e, let-7f, let-7g, let-71, miR-1b, miR-7, miR-9, miR-10b, miR-10a, miR-15a, miR-15b, miR-16, miR-18, miR-19a, miR-19b, miR-20, miR-21, miR-22, miR-23a, miR-23b, miR-24, miR-25, miR-26a, miR-26b, miR-27a, miR-27b, miR-29a, miR-29b, miR-29c, miR-30a, miR-30b, miR-30c, miR-30d, miR-30e, miR-31, miR-32, miR-33, miR-33b, miR-34, miR-92, miR-93, miR-94, miR-96, miR-98, miR-99a, miR-99b, miR-100, miR-101, miR-103, miR-104, miR-106, miR-107, miR-108, miR-122a, miR-123, miR-124a, miR-125a, miR-125b, miR-126, miR-128, miR-128b, miR-129b, miR-130, miR-130b, miR-131, miR-132, miR-133, miR-133b, miR-135b, miR-137, miR-138, miR-140, miR-141, miR-142s, miR-142 as, miR-143, miR-144, miR-145, miR-146, miR-148, miR-148b, miR-152, miR-153, miR-155, miR-181a, miR-181b, miR-181c, miR-182, miR-183, miR-184, miR-187, miR-190, miR-192, miR-194, miR-195, miR-196, miR-199a, miR-199b, miR-200b, miR-202, miR-203, miR-204, miR-205, miR-206, miR-208, miR-210, miR-211, miR-212, miR-213, miR-214, miR-215, miR-216, miR-218, miR-219, miR-221, miR-222, or miR-223.

In some embodiments, the miRNA may be isolated, e.g., from vertebrate cells such as mammalian cells. An “isolated” molecule, as used herein, is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing. Because an isolated molecular species of the invention may be admixed with a pharmaceutically-acceptable carrier in a pharmaceutical preparation, the molecular species may comprise only a small percentage by weight of the preparation. The molecular species is nonetheless isolated in that it has been substantially separated from the substances with which it may be associated in living systems.

One aspect of the invention provides systems and methods of identifying a target gene sequence of an miRNA sequence (i.e., a gene sequence, typically encoding a protein, to which the miRNA is associated with). As discussed in more detail below, binding of an isolated oligonucleotide comprising a sequence that is substantially antisense to the miRNA may alter expression of the target gene, for example, by interacting with the mRNA produced from the target gene sequence, which can thus prevent or at least inhibit expression of the gene.

In one set of embodiments, target gene sequences for an miRNA sequence can be determined by comparing the sequence of potential target gene sequences with the miRNA sequence for complementary matches (e.g., for Watson-Crick complementarity pairing and/or G:U pairing). For example, the UTR of potential target gene sequences can be compared with the miRNA sequence for complementary matches, and used to identify those gene sequences with higher degrees of complementarity as being target gene sequences. The determination may be performed manually, or with the aid of a machine such as a computer system, e.g., as further described below. The potential target gene sequences to be searched may be from one, or several species (for example, for comparative studies, i.e., human and mouse, human and rat, mouse and rat, human and pufferfish (Fugu), human and dog, human and chicken, etc.).

In some embodiments, an miRNA “seed” region for comparison may be designated in the miRNA sequence, and the UTR of potential target gene sequences may be selected on the basis of complementarity or perfect complementarity with the seed region of the miRNA. In some cases, the seed region nucleates binding between an miRNA and its complement, for example, a sequence that is substantially antisense to the miRNA sequence. The “seed” region is also referred to herein as a first portion of the miRNA sequence. The seed region of the miRNA may be any suitable portion of the miRNA, for example, 3, 4, 5, 6, 7, 8, 9, or 10 consecutive nucleotides within the miRNA sequence. Preferably, the seed region of the miRNA is 6, 7, or 8 consecutive nucleotides within the miRNA sequence. For instance, the seed region of the miRNA sequence may advantageously be inclusively defined as nucleotides 1 through 7, 1 through 8, 2 through 7, or 2 through 8 from the 5′ end of the oligonucleotide. Other examples include 1 through 9, 1 through 10, 2 through 7, 2 through 8, 2 through 9, 2 through 10, 3 through 10, 4 through 12, etc. from the 5′ end of the oligonucleotide. Non-limiting examples of miRNA seed sequences include SEQ ID NO: 469 to SEQ ID NO: 537 or SEQ ID NO: 542 to SEQ ID NO: 551. The portion of the UTR complementary to the seed region may be referred to as a “seed match” region or a first sequence of the UTR. After determining a match between the seed region of the miRNA and the seed match region of the UTR of the potential target gene sequence, an “extended” portion may be defined within the miRNA, where the extended portion includes nucleotides within the miRNA that are at least partially complementary (i.e., including G:U pairing), if not perfectly complementary, to the UTR of the potential target gene sequence. The sequence within the UTR that the extended portion of the miRNA binds to may also be referred to as an extended sequence within the UTR. In some cases, the extended portion of the miRNA may be defined by proceeding in the 3′ and/or 5′ directions from the seed region of the miRNA as far as possible, until a mismatch is found. In other cases, the extended portion may be defined as a portion of this. In some instances, the extended portion may have 1, 2, 3, 4, or more nucleotides, in addition to the seed region. In other instances, however, the extended portion may be determined to be the same as the seed region.

In some embodiments, after determining the seed region and/or the extended portion, the remaining portions of the miRNA and the UTR of the potential target gene sequence may also be compared to determine if the other regions are also complementary; and, in some cases, such portions may be optimized to determine the degree of complementarity between these regions.

The degree of interaction between the miRNA and the UTR of the target gene sequence may also be determined in some cases, for instance, to determine the degree of specificity, the binding affinity of such a match, etc. The degree of interaction can be determined, for instance, by determining a measure of the free energy of the interaction between the miRNA and the UTR, for example, when the miRNA and the UTR are bound or otherwise associated via the seed region and/or the extended portion, and/or via other portions of the miRNA and the UTR that may be associated (e.g., in an optimized configuration, as previously discussed). For instance, a free energy measurement may be given to each base-pair interaction between the miRNA and the UTR of the target gene sequence, and the sum of the free energy measurements may be determined in some fashion.

By determining such free energy measurements, miRNA binding to the UTRs of different target gene sequences (or of the same target gene sequences that arise from different organisms or species) may be assessed in various embodiments of the invention. For example, miRNA binding to a UTR of a first gene sequence from a first organism or species may be compared to the binding of miRNA to a UTR of a second gene sequence, a second UTR of the first gene sequence, a UTR of a gene sequence in a second organism or species, etc. In some cases, the free energy measurement may be compared to a reference free energy measurement, for example, a free energy measurement indicative of substantial binding between the miRNA and the UTR of the target gene sequence. In some cases, the target encoded by the target gene sequence may then be designated as a target requiring further study or experiments, or the target may be designated as a target in a subject that can be treated by applying miRNA or other agent able to alter expression of the target in some fashion. Non-limiting examples of such programs are provided in Appendices A and B, each of which is a computer program listing appendix, and each of which is incorporated herein by reference.

In another set of embodiments, a target gene sequence for an miRNA sequence may be identified or determined by defining at least 6 nucleotides of a conserved miRNA sequence as an miRNA seed, identifying a conserved UTR of a gene within the genome of the organism, and identifying the gene as a target of the miRNA by determining whether the conserved UTR comprises a segment having perfect complementarity with the miRNA seed.

Various embodiments of the invention can be implemented, e.g., as described above, in one or more computer systems. These computer systems, may be, for example, general-purpose computers such as those based on Intel PENTIUM-type and XScale-type processors, Motorola PowerPC, Motorola DragonBall, IBM HPC, Sun UltraSPARC, Hewlett-Packard PA-RISC processors, any of a variety of processors available from Advanced Micro Devices (AMD) or any other type of processor. It should be appreciated that one or more of any type of computer system may be used to implement various embodiments of the invention. A general-purpose computer system according to one embodiment of the invention is configured to perform any of the functions described above. It should be appreciated that the system may perform other functions and the invention is not limited to having any particular function or set of functions.

For example, various embodiments of the invention may be implemented as specialized software executing in a general-purpose computer system 1000 such as that shown in FIG. 16. The computer system 1000 may include a processor 1003 connected to one or more memory devices 1004, such as a disk drive, memory, or other device for storing data. Memory 1004 is typically used for storing programs and data during operation of the computer system 1000. Components of computer system 1000 may be coupled by an interconnection mechanism 1005, which may include one or more buses (e.g., between components that are integrated within a same machine) and/or a network (e.g., between components that reside on separate discrete machines). The interconnection mechanism 1005 enables communications (e.g., data, instructions) to be exchanged between system components of system 1000. Computer system 1000 also includes one or more input devices 1002, for example, a keyboard, mouse, trackball, microphone, touch screen, and one or more output devices 1001, for example, a printing device, display screen, or speaker. In addition, computer system 1000 may contain one or more interfaces (not shown) that connect computer system 1000 to a communication network (in addition or as an alternative to the interconnection mechanism 1005.

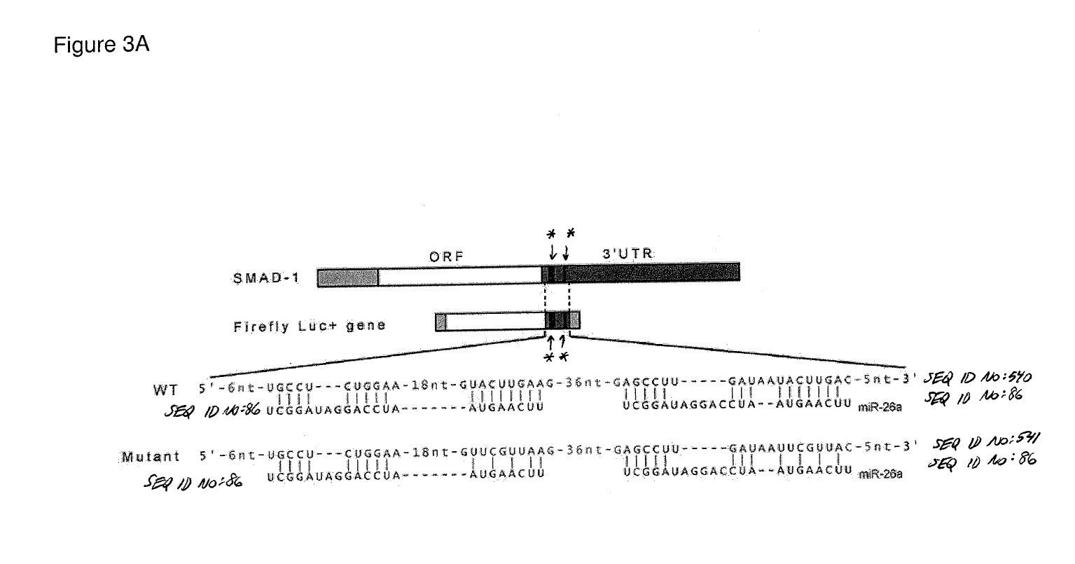

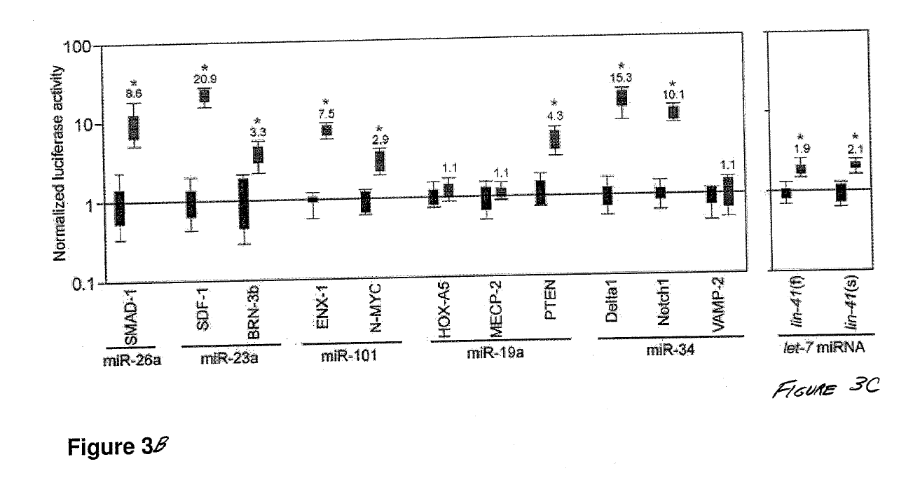

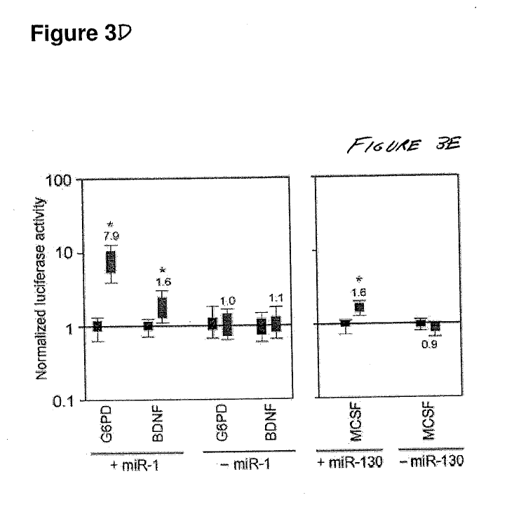

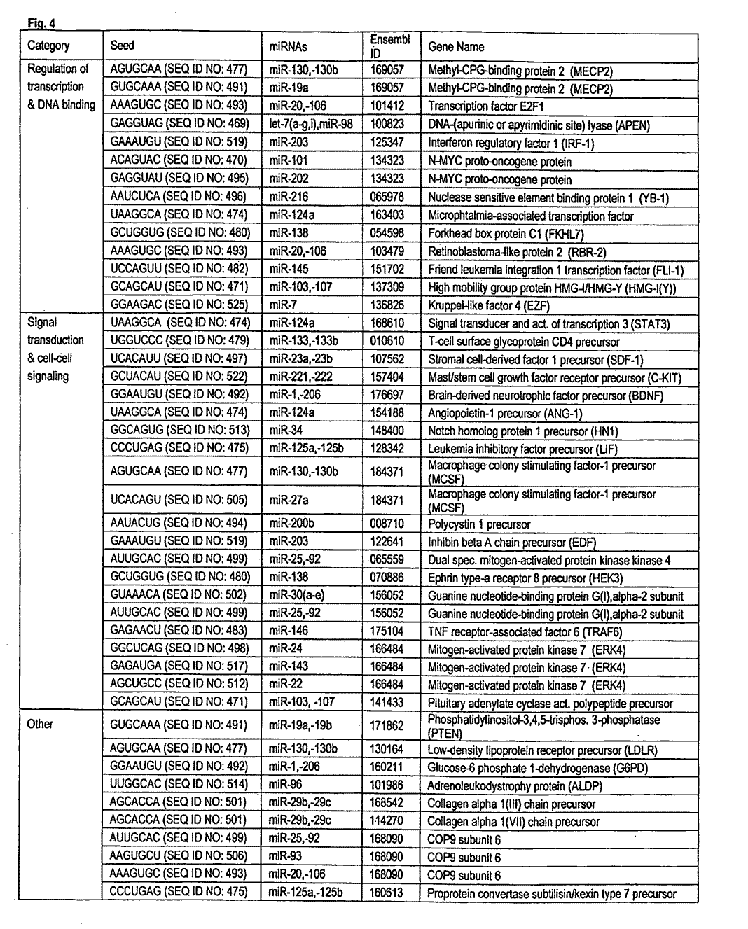

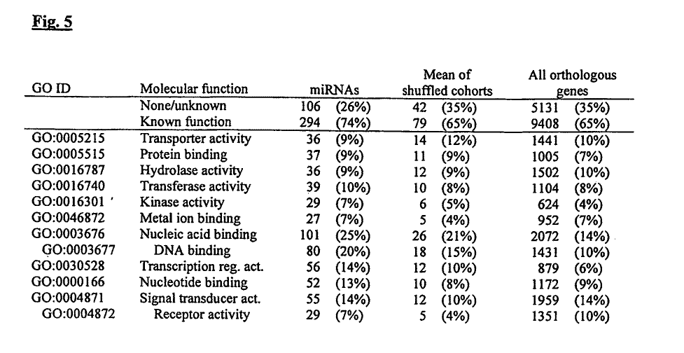

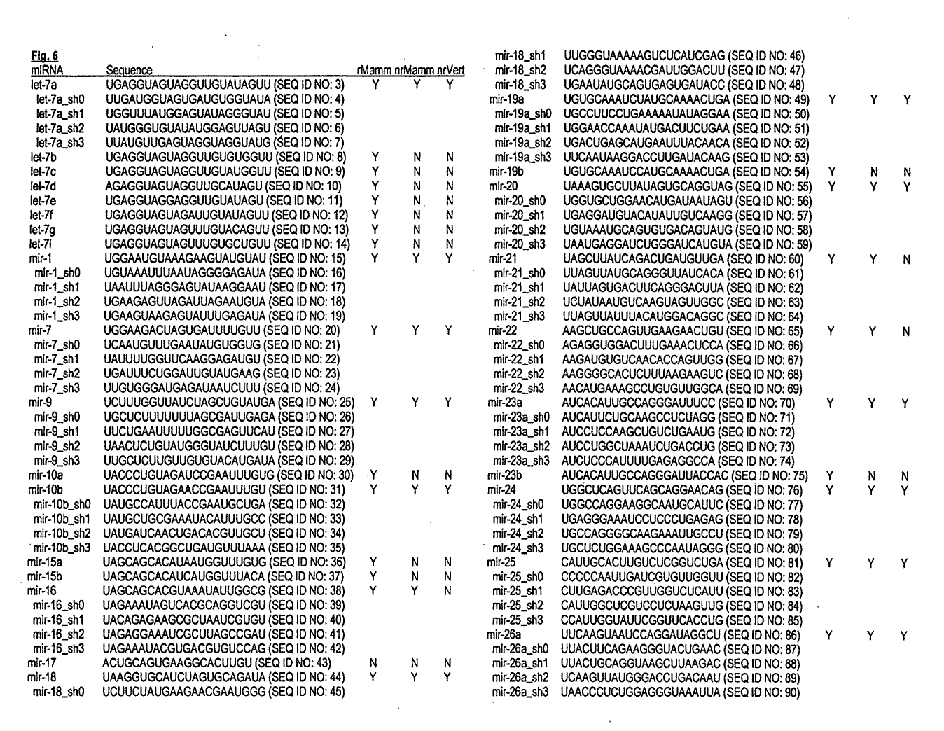

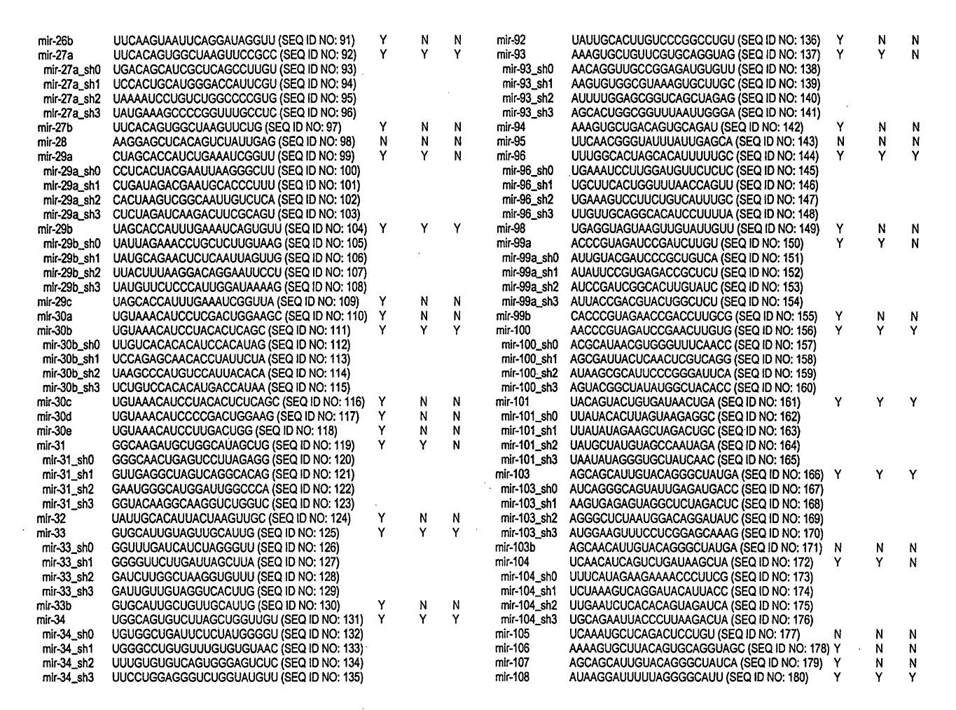

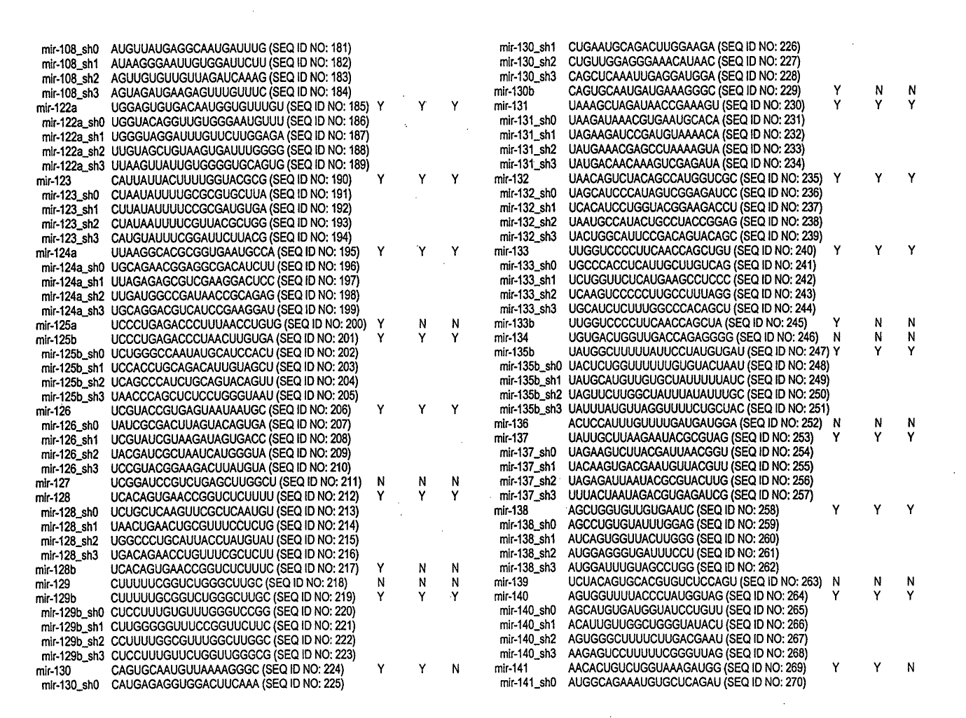

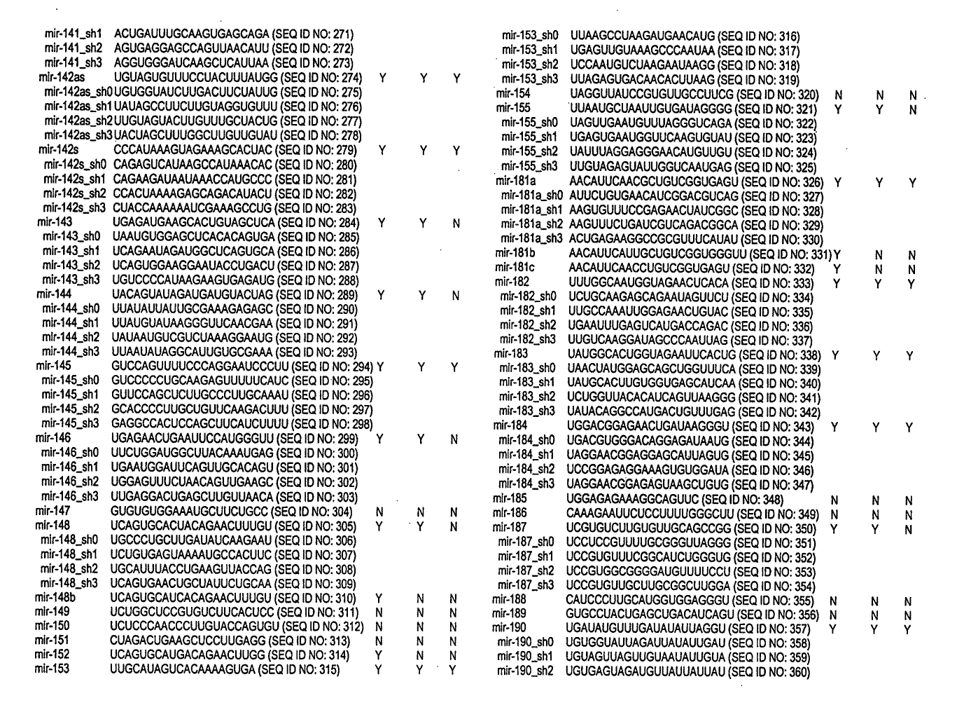

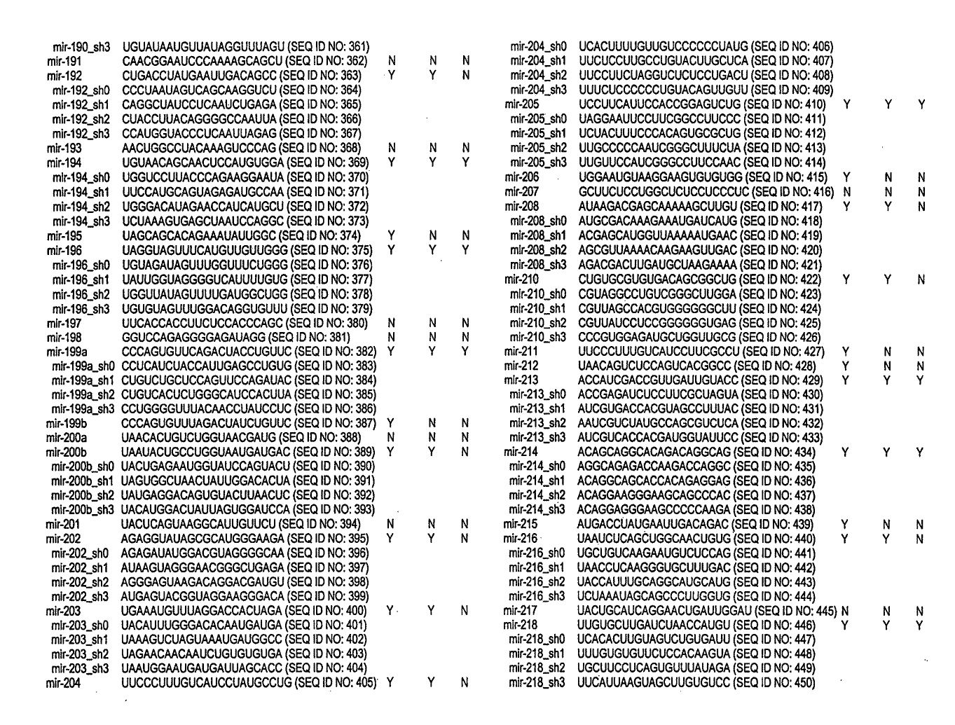

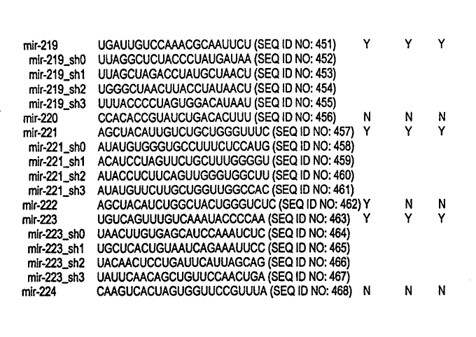

The storage system 1006, shown in greater detail in FIG. 3, typically includes a computer readable and writeable nonvolatile recording medium 1101 in which signals are stored that define a program to be executed by the processor or information stored on or in the medium 1101 to be processed by the program. The medium may, for example, be a disk or flash memory. Other non-limiting examples of computer-readable media include, but are not limited to, silicon and other semiconductor microchips or integrated circuits, bar codes, radio frequency tags or circuits, CDs, DVDs, insertable memory devices (e.g., memory cards, memory chips, memory sticks, memory plugs, etc.), “flash” memory, magnetic media (e.g., magnetic strips, magnetic tape, DATs, tape cartridges, etc.), floppy disks (e.g., 5.25 inch or 90 mm (3.5 inch) disks), optical disks, or the like. Typically, in operation, the processor causes data to be read from the nonvolatile recording medium 1101 into another memory 1102 that allows for faster access to the information by the processor than does the medium 1101. This memory 1102 is typically a volatile, random access memory such as a dynamic random access memory (DRAM) or static memory (SRAM). It may be located in storage system 1006, as shown, or in memory system 1004 (not shown). The processor 1003 generally manipulates the data within the integrated circuit memory 1004, 1102 and then copies the data to the medium 1101 after processing is completed. A variety of mechanisms are known for managing data movement between the medium 1101 and the integrated circuit memory element 1004, 1102, and the invention is not limited thereto. The invention is not limited to a particular memory system 1004 or storage system 1006.

In some embodiments, the computer system may include specially-programmed, special-purpose hardware, for example, an application-specific integrated circuit (ASIC). Various embodiments of the invention may be implemented in software, hardware or firmware, or any combination thereof. Further, such methods, acts, systems, system elements and components thereof may be implemented as part of the computer system described above or as an independent component.

Although computer system 1000 is shown by way of example as one type of computer system upon which various embodiments of the invention may be practiced, it should be appreciated that embodiments of the invention are not limited to being implemented on the computer system as shown in FIG. 16. For instance, various embodiments of the invention may be practiced on one or more computers having a different architecture or components that that shown in FIG. 16.

Computer system 1000 may be a general-purpose computer system that is programmable using a high-level computer programming language. Computer system 1000 may be also implemented using specially programmed, special purpose hardware. In computer system 1000, processor 1003 is typically a commercially available processor such as the well-known Pentium class processor available from the Intel Corporation. Many other processors are available. Such a processor usually executes an operating system which may be, for example, the Windows® 95, Windows® 98, Windows NT®, Windows® 2000 (Windows® ME), Windows® XP, Windows CE® or Pocket PC® operating systems available from the Microsoft Corporation, MAC OS System X available from Apple Computer, the Solaris Operating System available from Sun Microsystems, Linux available from various sources, UNIX available from various sources or Palm OS® available from Palmsource, Inc. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

One or more portions of the computer system may be distributed across one or more computer systems (not shown) coupled to a communications network. These computer systems also may be general-purpose computer systems. For example, various embodiments of the invention may be distributed among one or more computer systems configured to provide a service (e.g., servers) to one or more client computers, or to perform an overall task as part of a distributed system. For example, various embodiments of the invention may be performed on a client-server system that includes components distributed among one or more server systems that perform various functions according to various embodiments of the invention. These components may be executable, intermediate (e.g., IL) or interpreted (e.g., Java) code which communicate over a communication network (e.g., the Internet) using a communication protocol (e.g., TCP/IP).

It should be appreciated that the invention is not limited to executing on any particular system or group of systems. Also, it should be appreciated that the invention is not limited to any particular distributed architecture, network, or communication protocol. Various embodiments of the present invention may be programmed using an object-oriented programming language, such as SmallTalk, Java, C++, Ada, or C# (C-Sharp). Other object-oriented programming languages may also be used. Alternatively, functional, scripting, and/or logical programming languages may be used. Various embodiments of the invention may be implemented in a non-programmed environment (e.g., documents created in HTML, XML or other format that, when viewed in a window of a browser program, render embodiments of a graphical-user interface (GUI) or perform other functions). Various embodiments of the invention may be implemented as programmed or non-programmed elements, or any combination thereof. Further, various embodiments of the invention may be implemented using Microsoft.NET technology available from Microsoft Corporation.

In some embodiments, if the gene identified above is a target of the miRNA, an oligonucleotide may then subsequently be synthesized that comprises a sequence that is substantially antisense to the conserved miRNA sequence, using techniques known to those of ordinary skill in the art, and the synthesized oligonucleotide may be introduced into a cell. Examples of such techniques are described in more detail herein.

In another aspect, the present invention provides methods and compositions for regulating the expression of a gene, for example, in vertebrate cells, such as mammalian cells. Gene expression may be inhibited such that production of functional proteins is reduced. This may be accomplished by increasing the amount or stability of specific miRNAs in a cell. The amount of miRNA in a cell may be increased by adding exogenous miRNA. This may be accomplished by administering an miRNA oligonucleotide, for instance, in the form of a duplex or a stem-loop structure to the cell. The duplex can be the miRNA duplex, comprised of the miRNA and mRNA* produced after cleavage of the miRNA stem-loop. Alternatively, the miRNA duplex can be more or less extensively paired, with 2-nucleotide 3′ overhangs characteristic of some silencing RNA duplexes, including miRNA duplexes. More or less paired refers to the perfect complement it or including one or more mismatches, e.g. 7 or less. The exogenously added miRNA will cause translational repression of one or more genes resulting in the specific downregulation of protein production. Alternatively or additionally, a cell may be transfected with a sequence encoding an miRNA that, when expressed by the cell, causes the cell to overexpress the miRNA. For example, a vector comprising an miRNA sequence under the control of regulatory elements may be transfected into a cell using techniques known to those of ordinary skill in the art, which sequence may be expressed by the cell (in addition to any normal miRNA), thereby resulting in overexpression of the miRNA, e.g., such that the levels of miRNA within the cell are substantially higher than normal expression levels of miRNA.

As used herein gene expression is considered to be “decreased” or “inhibited” when any decrease in corresponding protein production is observed after the amount or stability of an miRNA function is increased compared to a control cell in which the amount or stability of miRNA function is not increased. Although applicant is not bound by a mechanism, it is believed that increasing miRNA function reduces the amount of protein made from the mRNA, for example by destabilizing the mRNA and/or by causing the mRNA to be less efficiently used in protein production. Thus, binding of an exogenously added miRNA to the UTR thus may prevent or at least partially inhibit the cell from expressing the gene, similar to an endogenously produced miRNA. Non-limiting examples of genes that are regulated by miRNAs which can bind to a UTR of the corresponding mRNA are shown in FIG. 4, 7, or 8.

Gene expression may also be increased in a cell by reducing the function of miRNA in a cell. miRNA function may be reduced by administering a composition, such as an isolated oligonucleotide that interferes with miRNA activity. An oligonucleotide that interferes with miRNA activity may be, for instance, an oligonucleotide that is substantially antisense to an miRNA and/or a portion thereof, such as an miRNA seed. Other types of oligonucleotides that interfere with the miRNA activity include oligonucleotides that are antisense to the miRNA binding region of the mRNA. For example, a cell may contain a gene sequence which produces an mRNA having a UTR and a coding region (i.e., a region that can be expressed to produce a protein), and an oligonucleotide may be delivered into the cell such that the oligonucleotide binds to a portion of the UTR of the mRNA or the miRNA sequence, thus blocking the ability of the miRNA to interact with the mRNA. In some cases, the oligonucleotide may pair to a complementary site within the UTR portion of an mRNA, which can trigger interference in mRNA function. As used herein, an “UTR” is an untranslated region of an mRNA sequence, i.e., a portion of an mRNA which is not expressed as a protein, but is expressed as mRNA. As used herein gene expression is considered to be “increased” when any increase in corresponding protein production is observed after miRNA function is reduced compared to a control cell in which miRNA function is not reduced.

Binding or other association of the miRNA to the target mRNA sequence may occur through limited base-pairing interactions with a complementary site within the UTR of the target mRNA sequence, for example, through Watson-Crick (“W—C”) complementarity pairs (A:U and C:G pairing) (i.e., “perfect” complementarity) and/or G:U pairing. The pairing may also be to a coding portion of an mRNA sequence in some cases, i.e., to a portion of an mRNA which is expressed (e.g., as a protein), i.e. that portion that encodes one or more amino acids that are expressed as a protein or a peptide, etc. Thus, it should be understood that the discussions herein with respect to binding of miRNAs to UTRs of mRNAs is by way of example only, and in other embodiments of the present invention, certain miRNAs may bind to coding portions of the mRNA, and/or both the coding portions and the UTR portions of the mRNA.

Some methods of the invention involve binding of an oligonucleotide to an miRNA or mRNA. In some cases, a portion of the oligonucleotide binds to the complementary site within the UTR of the target mRNA or within the miRNA. The portion may have perfect complementarity with the mRNA or miRNA sequence, i.e., through Watson-Crick complementarity pairing, and the portion may be 5, 6, 7, 8, or 9 nucleotides long. Longer portions are also possible in some instances. In other cases, however, the complementary region between the miRNA or UTR of the mRNA and the oligonucleotide portions may also include G:U pairings in addition to Watson-Crick complementarity pairing.

Thus, the invention involves delivery to cells of isolated nucleic acids, including but not limited to oligonucleotides that are substantially antisense to at least a portion of an mRNA, oligonucleotides that comprise an miRNA sequence (e.g., oligonucleotides having stem-loop structures or miRNA duplexes) and expression vectors that encode mRNA sequences. Any method or delivery system may be used for the delivery and/or transfection of the nucleic acids, and such delivery and/or transfection may occur in vitro or in vivo. If in vivo, the cell may be in a subject, for example, a human or non-human mammal, such as a monkey, ape, cow, sheep, goat, buffalo, antelope, oxen, horse, donkey, mule, deer, elk, caribou, water buffalo, camel, llama, alpaca, rabbit, pig, mouse, rat, guinea pig, hamster, dog, cat, etc. The oligonucleotide, or the nucleotide sequence able to be transcribed to produce the oligonucleotide, may be delivered to the cell alone, or in combination with other agents. Examples of delivery systems include, but are not limited to, particle gun technology, colloidal dispersion systems, electroporation, vectors, and the like. In its broadest sense, a “delivery system,” as used herein, is any vehicle capable of facilitating delivery of a nucleic acid (or nucleic acid complex) to a cell and/or uptake of the nucleic acid by the cell. Other non-limiting example delivery systems that can be used to facilitate uptake by a cell of the nucleic acid include calcium phosphate or other chemical mediators of intracellular transport, microinjection compositions, or homologous recombination compositions (e.g., for integrating a gene into a predetermined location within the chromosome of the cell).

The term “transfection,” as used herein, refers to the introduction of a nucleic acid into a cell, for example, miRNA, or a nucleotide sequence able to be transcribed to produce miRNA. Transfection may be accomplished by a wide variety of means, as is known to those of ordinary skill in the art. Such methods include, but are not limited to, Agrobacterium-mediated transformation (e.g., Komari, et al., Curr. Opin. Plant Biol., 1:161 (1998)), particle bombardment mediated transformation (e.g., Finer, et al., Curr. Top. Microbiol. Immunol., 240:59 (1999)), protoplast electroporation (e.g., Bates, Methods Mol. Biol., 111:359 (1999)), viral infection (e.g., Porta and Lomonossoff, Mol. Biotechnol. 5:209 (1996)), microinjection, and liposome injection. Standard molecular biology techniques are common in the art (e.g., Sambrook, et al., Molecular Cloning: A Laboratory Manual, 2^{nd} ed., Cold Spring Harbor Laboratory Press, New York (1989)). For example, in one embodiment of the present invention, a mammalian cell or other vertebrate cell is transformed with a gene encoding an oligonucleotide comprising a sequence that is substantially antisense to an miRNA, or a sequence able to be transcribed to produce an oligonucleotide comprising a sequence that is substantially antisense to an miRNA.

In one set of embodiments, genetic material may be introduced into a cell using particle gun technology, also called microprojectile or microparticle bombardment, which involves the use of high velocity accelerated particles. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry oligonucleotides into the interiors of bombarded cells. It has been demonstrated that such microprojectiles can enter cells without causing death of the cells, and that they can effectively deliver foreign genetic material into intact tissue.

In another set of embodiments, a colloidal dispersion system may be used to facilitate delivery of a nucleic acid (or nucleic acid complex) into the cell, for example, an isolated oligonucleotide that is substantially antisense to an miRNA, a sequence able to be transcribed to produce an oligonucleotide comprising a sequence that is substantially antisense to an miRNA, a sequence that, when expressed by the cell, causes the cell to overexpress the miRNA, etc. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which can range in size from 0.2 to 4.0 micrometers, can encapsulate large macromolecules within the aqueous interior and these macromolecules can be delivered to cells in a biologically active form (e.g., Fraley, et al., Trends Biochem. Sci., 6:77 (1981)).

Lipid formulations for the transfection and/or intracellular delivery of nucleic acids are commercially available, for instance, from QIAGEN, for example as EFFECTENE® (a non-liposomal lipid with a special DNA condensing enhancer) and SUPER-FECT® (a novel acting dendrimeric technology) as well as Gibco BRL, for example, as LIPOFECTIN® and LIPOFECTACE®, which are formed of cationic lipids such as N-[1-(2,3-dioleyloxy)-propyl]-N,N,N-trimethylammonium chloride (“DOTMA”) and dimethyl dioctadecylammonium bromide (“DDAB”). Liposomes are well known in the art and have been widely described in the literature, for example, in Gregoriadis, G., Trends in Biotechnology 3:235-241 (1985).

Electroporation may be used, in another set of embodiments, to deliver a nucleic acid (or nucleic acid complex) to the cell, e.g., an isolated oligonucleotide that is substantially antisense to an miRNA, a sequence able to be transcribed to produce an oligonucleotide comprising a sequence that is substantially antisense to an miRNA, a sequence that, when expressed by the cell, causes the cell to overexpress the miRNA, etc. “Electroporation,” as used herein, is the application of electricity to a cell in such a way as to cause delivery of a nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by those of ordinary skill in the art.

In yet another set of embodiments, a nucleic acid (e.g., an isolated oligonucleotide that is substantially antisense to an miRNA or a mRNA UTR, a sequence able to be transcribed to produce an oligonucleotide comprising a sequence that is substantially antisense to an miRNA, a sequence that, when expressed by the cell, causes the cell to overexpress the miRNA, etc.) may be delivered to the cell in a vector. In its broadest sense, a “vector” is any vehicle capable of facilitating the transfer of the nucleic acid to the cell such that the nucleic acid can be processed and/or expressed in the cell. The vector may transport the nucleic acid to the cells with reduced degradation, relative to the extent of degradation that would result in the absence of the vector. The vector optionally includes gene expression sequences or other components able to enhance expression of the nucleic acid within the cell. The invention also encompasses the cells transfected with these vectors, including cells such as those previously described.

In general, vectors useful in the invention include, but are not limited to, plasmids, phagemids, viruses, other vehicles derived from viral or bacterial sources that have been manipulated by the insertion or incorporation of the nucleotide sequences (or precursor nucleotide sequences) of the invention. Viral vectors useful in certain embodiments include, but are not limited to, nucleic acid sequences from the following viruses: retroviruses such as Moloney murine leukemia viruses, Harvey murine sarcoma viruses, murine mammary tumor viruses, and Rouse sarcoma viruses; adenovirus, or other adeno-associated viruses; SV40-type viruses; polyoma viruses; Epstein-Barr viruses; papilloma viruses; herpes virus; vaccinia virus; polio viruses; or RNA viruses such as retroviruses. One can readily employ other vectors not named but known to the art. Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors may have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) are well known to those of ordinary skill in the art. Examples of standard protocols can be found in Kriegler, M., Gene Transfer and Expression, A Laboratory Manual, W.H. Freeman Co., New York (1990), or Murry, E. J. Ed., Methods in Molecular Biology, Vol. 7, Humana Press, Inc., Clifton, N.J. (1991).

Another example of a virus for certain applications is the adeno-associated virus, which is a double-stranded DNA virus. The adeno-associated virus can be engineered to be replication-deficient and is capable of infecting a wide range of cell types and species. The adeno-associated virus further has advantages, such as heat and lipid solvent stability; high transduction frequencies in cells of diverse lineages; and/or lack of superinfection inhibition, which may allow multiple series of transductions.

Another vector suitable for use with the invention is a plasmid vector. Plasmid vectors have been extensively described in the art and are well-known to those of skill in the art. See, e.g., Sambrook, et al., Molecular Cloning: A Laboratory Manual, Second Edition, Cold Spring Harbor Laboratory Press, 1989. These plasmids may have a promoter compatible with the host cell, and the plasmids can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence (which can, for instance, then be cleaved or otherwise processed within the cell to produce a precursor miRNA). These vectors may include a sequence encoding a nucleic acid and an in vivo expression element, as further described below. In some cases, the in vivo expression element includes at least one promoter.

The nucleic acid, in one embodiment, may be operably linked to a gene expression sequence which directs the expression of the nucleic acid within the cell (e.g., to produce an oligonucleotide that is substantially antisense to an miRNA, or a sequence able to be transcribed to produce an oligonucleotide comprising a sequence that is substantially antisense to an miRNA). The nucleic acid sequence and the gene expression sequence are said to be “operably linked” when they are covalently linked in such a way as to place the transcription of the nucleic acid sequence under the influence or control of the gene expression sequence. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis, et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). In some embodiments, the nucleic acid is linked to a gene expression sequence which permits expression of the nucleic acid in a vertebrate cell. A sequence which permits expression of the nucleic acid in a cell is one which is selectively active in the particular cell and thereby causes the expression of the nucleic acid in those cells. Those of ordinary skill in the art will be able to easily identify promoters that are capable of expressing a nucleic acid in a cell based on the type of cell.

The selection of a particular promoter and enhancer depends on what cell type is to be used and the mode of delivery. For example, a wide variety of promoters have been isolated from plants and animals, which are functional not only in the cellular source of the promoter, but also in numerous other species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc.

Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Thus, a variety of promoters and regulatory elements may be used in the expression vectors of the present invention. For example, in some embodiments, an inducible promoter is used to allow control of nucleic acid expression through the presentation of external stimuli (e.g., environmentally inducible promoters). The timing and amount of nucleic acid expression can be controlled in some cases. Non-limiting examples of expression systems, promoters, inducible promoters, environmentally inducible promoters, and enhancers are well known to those of ordinary skill in the art. Non-limiting examples include those described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472.

As used herein, an “expression element” can be any regulatory nucleotide sequence, such as a promoter sequence or promoter-enhancer combination, which facilitates the efficient expression of a nucleic acid, for example, an isolated oligonucleotide that is substantially antisense to an miRNA, a sequence able to be transcribed to produce an oligonucleotide comprising a sequence that is substantially antisense to an miRNA, a sequence that, when expressed by the cell, causes the cell to overexpress the miRNA, etc. The expression element may, for example, be a mammalian or viral promoter, such as a constitutive or inducible promoter. Constitutive mammalian promoters include, but are not limited to, polymerase promoters as well as the promoters for the following genes: hypoxanthine phosphoribosyl transferase (“HPTR”), adenosine deaminase, pyruvate kinase, and alpha-actin. Exemplary viral promoters which function constitutively in eukaryotic cells include, for example, promoters from the simian virus, papilloma virus, adenovirus, human immunodeficiency virus, Rous sarcoma virus, cytomegalovirus, the long terminal repeats of Moloney leukemia virus and other retroviruses, and the thymidine kinase promoter of herpes simplex virus. Other constitutive promoters are known to those of ordinary skill in the art. Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

Using any gene transfer technique, such as the above-listed techniques, an expression vector harboring a nucleic acid may be transformed into a cell to achieve temporary or prolonged expression. Any suitable expression system may be used, so long as it is capable of undergoing transformation and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (“GFP”) is used to allow simple selection of transfected cells and to monitor expression levels. Non-limiting examples of such vectors include Clontech's “Living Colors Vectors” pEYFP and pEYFP-C1.

In some cases, a selectable marker may be included with the nucleic acid being delivered to the cell. As used herein, the term “selectable marker” refers to the use of a gene that encodes an enzymatic or other detectable activity (e.g., luminescence or fluorescence) that confers the ability to grow in medium lacking what would otherwise be an essential nutrient. A selectable marker may also confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

Optionally, germ line cells may be used in the methods described herein rather than, or in addition to, somatic cells. The term “germ line cells” refers to cells in the organism which can trace their eventual cell lineage to either the male or female reproductive cells of the organism. Other cells, referred to as “somatic cells” are cells which do not directly give rise to gamete or germ line cells. Somatic cells, however, also may be used in some embodiments.

Thus, the alteration of the expression of a gene can be used, according to one set of embodiments, to systematically inhibit or express a gene within a cell in vitro, in vivo, or ex vivo, for example, by administering a composition such as an isolated oligonucleotide comprising a sequence that is substantially antisense to an miRNA of the cell. Thus, as an example, a normal cell may be rendered cancerous through the addition of an isolated oligonucleotide comprising a sequence that is substantially antisense to an miRNA of the cell, then rendered non-cancerous by not adding the oligonucleotide, i.e., stopping administration of the oligonucleotide. Tight control of the cancerous/non-cancerous behavior of a cell is a highly useful model of disease function and behavior.

According to yet another aspect of the invention, a plurality of genes in a subject may be modulated by administering, to the subject, a composition such as an isolated oligonucleotide comprising a sequence that is substantially antisense to an miRNA, or a sequence that, when expressed by the cell, causes the cell to overexpress the miRNA, etc. In many cases, multiple genes share common miRNA binding sites, for example, multiple genes within a particular pathway or network, such that the administration of an isolated oligonucleotide will affect some or all of those genes. As an example, application of isolated oligonucleotides that are antisense to the miR-15/16/195 family may repress numerous genes involved in stimulating cell proliferation and tumor growth. For instance, targets of miR-15/16/195 with roles in stimulating cell growth include FGF2 (mitogenic, angiogenic, neurotrophic factor), CCND2 (expressed highly in ovarian and testicular tumors), CCND1 (numerous cancers), CCNE1 (numerous cancers), or TGIF2 (ovarian cancers). As another example, the miR-17/20/106, miR-19, and miR-25/32/95 families, based on predicted targets as described herein and as listed in FIG. 4, 7, or 8, may be involved in promoting growth and proliferation. By administering isolated oligonucleotides that are antisense to the miRNA sequences in some or all of these families, pathways or networks involved in cell growth or control may be modulated. Thus, for instance, the isolated oligonucleotides can be administered to a subject to treat cancers or immune diseases characterized by improper or altered gene expression.

The alteration of the expression of a gene can also be used, according to still another aspect, to treat diseases that are characterized by altered gene expression, for example, cancer or other diseases in which cells reproduce uncontrollably. By administering, to a subject, a composition comprising an isolated oligonucleotide comprising a sequence that is substantially antisense to an miRNA, the expression of a gene involved in cell reproduction can thus be controlled to control cell growth. For instance, in some cases, such a method can be used to treat a cancer or a tumor. For example, the synthesized oligonucleotide may be introduced into a cancer cell. The oligonucleotide may then interact with the UTR of a gene sequence within the cancer cell to at least partially inhibit expression of the gene, thereby controlling, or killing, the cancer cell. Thus, one set of embodiments provides systems and methods for treating various forms of disease in a subject by manipulating gene expression through the regulation mRNA activity using the miRNA technology described herein. mRNA activity may be regulated by increasing the presence of miRNAs in a cell, chemically or through exogenous expression to decrease protein expression from the mRNA or by reducing miRNA levels using miRNA antisense technology to increase protein expression. The methods of the invention are useful, for instance, in treating diseases such as cancer, autoimmune disease, arthritis, inflammatory disorders, osteogenesis, neurodegenerative disorders such as Alzheimer's, cardiovascular disease, kidney disease, hematopoiesis, hypercholesterolemia, and diabetes.

As a particular example of a therapeutic protocol for cancer, transcription factor E2F1 may be modulated using the systems and methods described herein. E2F1 is targeted by miR-20 and miR-106 (see FIG. 4). Overexpression of E2 μl may lead to apoptosis. Thus it is desirable, according to the invention to, to reduce miR-20 expression in order to increase E2F1 expression. Alternatively, decreased expression of E2F4 may induce apoptosis. Thus, decreasing E2F4 levels by increasing miR-106 levels may also be useful in inducing apoptosis.

In another example, IRF-1 can be modulated by targeting miR-203. Suppression of IRF-1 by increasing levels of miR-203 may prevent cell growth, e.g., in cancer cells.

N-MYC, in yet another example, may be modulated by targeting miR-101 or miR-202. N-MYC is a proto-oncogene which may be modulated using the systems and methods of the invention. For instance, amplification of N-MYC by reducing levels of miR-101 or miR-202 may lead to growth inhibition or apoptosis, for example, in neuroblastoma or other solid tumors.

As still another example, YB-1 may be modulated by targeting miR-216. In tumor cells, suppression of YB-1 may result in a lowering of androgen, and increased cell survival. Thus, overexpression of YB-1 by reducing levels of miR-216 may be used to treat certain types of cancer, for example, prostate cancer.

As yet another example, FKHL7 can be modulated by targeting miR-138. In normal cells, FKHL7 may act as a tumor suppressor, e.g., by arresting the cell cycle. In certain cancer cells, FKHL7 is suppressed. Thus, overexpression of FKHL7 by reducing levels of miR-138 may be used to treat certain types of cancer, for example, endometrial or ovarian cancer.

RBR-2, according to another example, may be modulated by targeting miR-20 or miR-106. Down regulation of RBR-2 may play an important role in certain types of cancer, such as cervical cancer. Thus, increasing RBR-2 by targeting miR-20 or miR-106 may be useful as a cancer therapy.

FLI-1 can be modulated, in yet another example, to inhibit tumor growth, for example, in a cancer such as Ewing's sarcoma. Increasing FLI-1 expression to treat such cancers may be modulated by targeting miR-145.

In still another example, HMG-I or HMG-Y may be modulated by targeting miR-103 or miR-107. In cancer cells, these genes may be upregulated. Thus, by increasing levels of miR-103 or miR-107 to target HMG-I or HMG-Y, for example, to inhibit gene expression using the systems and methods described herein, certain types of tumors may be suppressed, for example, adenocarcinomas or pancreatic tumors.

EZF (Kruppel-like factor 4) may be modulated by targeting miR-7, according to yet another example. In cancer cells, EZF may be decreased. EZF is believed to be involved in suppressing cell growth. Thus, by increasing EZF, cancers such as gastric cancer may be treated using the systems and methods described herein.

In one example, STAT3 can be modulated by targeting miR-124a. STAT3 is involved in the regulation of many pathways important in oncogenesis, such as apoptosis, tumor angiogenesis, cell-cycle progression, tumor-cell invasion, or metastasis. By modulating STAT3, some or all of these oncogenesis pathways may be suppressed or at least inhibited.

Cell migration may be inhibited, in another example, by modulating SDF-1, for example, using miR-23a or miR-23b. The inhibition of cell migration may result in decreased metastatic events in cancer patients.

In yet another example, C-KIT can be modulated by targeting miR-221 or miR-222. Targeting of C-KIT, for example, to decrease expression levels, may be useful in treating cancers such as gastrointestinal stromal tumors.

ANG-1, in another example, may be modulated by targeting miR-124a. ANG-1 has been correlated with cancers, and is believed to be involved in differentiation. Thus, by targeting ANG-1, for example, to decrease expression levels, certain types of cancer, such as gastric cancer, may be treated.

In still another example, HN1 can be modulated by targeting miR-34. HN1 is often overexpressed in tumor cells, and is involved in signaling and transcription. Thus, by decreasing expression levels using the systems and methods described herein, certain types of cancer, such as gastrointestinal carcinoid tumors, may be treated.

In yet another example, ERK (e.g., ERK4) may be modulated by targeting miR-25, miR-92, miR-24, miR-143, or miR-22. ERK is a MAP kinase that is involved with mitogenesis and differentiation. Thus, by targeting an ERK, for example, to decrease expression levels, certain types of cancer, such as lymphomas, can be treated.

PTEN can be modulated by targeting miR-19a or miR-19b, according to another example. Loss of PTEN expression has been linked to shortened survival in patients having melanoma and other types of cancer. Thus, by increasing PTEN expression levels using the systems and methods described herein, melanoma survival rates may be increased.

As yet another example, proprotein convertase subtilisin-kexin type 7 precursor may be modulated by targeting miR-125a or miR-125b. This precursor is involved in many biological functions, including the generation of active peptides, proteins, hormones, and growth factors, and has been linked to tumorigenesis. High expression levels have been observed in various types of tumors. Thus, by decreasing expression levels using the systems and methods described herein, certain types of tumors may be treated.

Sema can be modulated, as another example, by targeting let-7a. Sema may regulate ligand mediated receptor activation, and overexpression of Sema has been observed in certain forms of cancer. Thus, by inhibiting Sema, certain types of cancers may be treated using the systems and methods described herein.

In another example, Naked Cuticle Homolog 1 can be modulated by targeting let-7a. Upregulation of this gene has been observed in certain forms of cancer, for example, gastric cancer, pancreatic cancer, or esophageal cancer. Thus, by inhibiting this gene, cancers such as these may be treated.

In one example, microphthalmia associated transcription factor may be modulated by targeting miR-124a. Amplification of this gene has been connected to metastasis of cancerous cells. Thus, by inhibiting this gene, certain types of cancers (e.g., highly metastatic cancers) may be treated.

Homeodomain Interacting Protein Kinase 3 (HIPK3), according to another example, may be modulated by targeting miR-124a. HIPK3 is believed to confer multidrug resistance in certain types of cancer cells. Thus, by inhibiting HIPK3 using the systems and methods described herein, various types of cancers, especially drug-resistant cancers, can be treated.

In yet another example, Mnt can be modulated by targeting miR-128. Mnt has been linked to cell proliferation and cell differentiation. Thus, certain forms of cancer, for example, carcinomas, may be treated by decreasing Mnt expression levels.

Checkpoint Suppressor 1 (CHES1) may be modulated, in still another example, by targeting miR-135b. An increased level of CHES1 may repress certain genes believed involved in cancer, for example, in tumorigenesis. Thus, by increasing CHES1 levels, certain forms of cancer may be treated.

As another example, CIS-6 can be modulated by targeting miR-19a. CIS-6 may be decreased in certain forms of cancer, such as breast cancer. By increasing CIS-6 expression levels, such cancers may be treated.

SOCS-5, in another example, may be modulated by targeting miR-19a. SOCS-5 is part of the cytokine signaling pathway. In certain cancers, SOCS-5 may be overexpressed. Thus, by inhibiting SOCS-5 using the systems and methods described herein, such cancers may be treated.

In yet another example, Dead-box Protein p68 can be modulated by targeting miR-1. p68 may be upregulated in cancer cells. Thus, by treating p68, such cancers can be treated.

As another example, Dead Ringer-Like 2 may be modulated by targeting miR-219. This protein may contribute to the transcription regulation of genes involved in differentiation. Thus, in certain types of cancer, an inhibition of Dead Ringer-Like 2 may be used to treat the cancer.

POU Domain Class 4, as yet another example, can be modulated by targeting miR-23a. POU Domain Class 4 may be overexpressed in certain types of cancer cells, for example, breast cancer. By inhibiting this gene, these types of cancers can be treated.

In another example, SMAD1 may be modulated by targeting miR-26a. SMAD1 binds certain factors, such as Ebfaz or Evi3, involved in B-cell disease and hematopoietic cancers. Thus, by increasing SMAD-1 expression, such diseases and cancers may be treated.

Pim-1 can be modulated, in yet another example, by targeting miR-26a. Pim-1 is often overexpressed in certain types of tumors, such as prostate tumors. Thus, by inhibiting Pim-1 expression, such tumors may be treated.

In still another example, nPKC delta may be modulated by targeting miR-26a. nPKC delta is often over expressed in certain types of cancer cells, such as melanoma cells. Thus, these cancers may be treated by inhibition of nPKC delta.

In yet another example, DAP-5 can be modulated by targeting miR-26a. DAP-5 has been linked to viability in certain types of cancer cells, such as neuroblastoma cells. By decreasing DAP-5 expression levels, such cancers may be treated.

ETS Factor 3 may be modulated by targeting miR-27a according to another example. ETS Factor 3 is involved in differentiation of epithelial cells and the like, and certain types of cancers may be treated by decreasing ETS Factor 3 expression.

In yet another example, DNMT3A may be modulated by targeting miR-29b. DNMT3A, a DNA methyltransferase, has been implicated in certain types of cancer, e.g., by promoting expression of other genes. By decreasing DNMT3A expression, such cancers may be treated.

Rhotekin can be modulated, in still another example, by targeting miR-138. In certain types of cancer, such as gastric cancer, Rhotekin is overexpressed. Thus, by inhibiting Rhotekin, such cancers may be treated.

In one example, NOTCH 1 can be modulated by targeting miR-34. NOTCH 1 is believed to facilitate tumor cell proliferation in vitro. By decreasing NOTCH 1 expression, tumor cell proliferation may be reduced.

Additional examples of cancers that can be treated using the compositions of the invention include, but are not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer. In general, an effective amount of the one or more compositions of the invention for treating cancer will be that amount necessary to inhibit mammalian cancer cell proliferation in situ. Those of ordinary skill in the art are well-schooled in the art of evaluating effective amounts of anti-cancer agents.

In some cases, the above-described treatment methods may be combined with known cancer treatment methods. The term “cancer treatment” as used herein, may include, but is not limited to, chemotherapy, radiotherapy, adjuvant therapy, surgery, or any combination of these and/or other methods. Particular forms of cancer treatment may vary, for instance, depending on the subject being treated. Examples include, but are not limited to, dosages, timing of administration, duration of treatment, etc. One of ordinary skill in the medical arts can determine an appropriate cancer treatment for a subject.

Manipulation of gene expression can also be used, according to another set of embodiments, to treat diseases that are characterized by alterations in immune system function. Many such immune diseases are characterized by improper gene expression. By administering, to a subject, a composition as described herein, such as an isolated oligonucleotide comprising a sequence that is substantially antisense to an miRNA, the expression of such genes may be controlled, thereby controlling the disease.

For instance, a gene such as T-cell surface glycoprotein CD4 precursor may be modulated by targeting miR-133 or miR-133b. Modulation of the CD4 precursor may be used to modulate the immune system. For example, increasing CD4 precursor may be used to stimulate production of dendritic cells.

As another example, a gene such as TPR Repeat Protein 7 may be modulated by targeting miR-125b. TPR Repeat Protein 7 is believed to control development of immune system cells. Thus, by overexpressing TPR Repeat Protein 7, the immune system may be stimulated, e.g., in patients having depressed immune systems.

In another aspect, the invention relates to a method for treating autoimmune disease by administering to a subject having or at risk of having an autoimmune disease an effective amount for treating or preventing the autoimmune disease of any of the compositions of the invention. Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

Megalin, for instance, can be modulated by targeting miR-19a. Megalin is implicated in certain autoimmune diseases, such as rheumatoid arthritis, systemic lupus erythematosus, Bechcet's disease, systemic sclerosis, and osteoarthritis. In some of these diseases, antibodies are produced to Megalin. Thus, overexpression of Megalin may be used to treat some of these diseases.

In another example, Tribbles Homolog 2 can be modulated by targeting miR-29b. Overexpression of Tribbles Homolog 2 has been observed in some types of autoimmune disease, such as autoimmune uveitis. By decreasing expression levels of Tribbles Homolog 2, such diseases may be treated.

As another example, a gene such as LIF may be modulated by targeting miR-125a or miR-125b. LIF has been linked to certain forms of arthritis. Thus, in some cases, increasing LIF expression levels may be used to treat, or reduce the severity of, conditions such as arthritis and other immune-mediated joint inflammatory diseases.

In yet another example, collagen alpha 1(I) chain precursor may be modulated by targeting let-7a. Reduced collagen expression has been observed in arthritis, osteogenesis imperfecta, and similar indications. Overexpression of collagen alpha 1(1) chain precursor may be used to treat such conditions.

As another example, a gene such as LIF may be modulated by targeting miR-125a or miR-125b. LIF has been linked to certain forms of arthritis. Thus, in some cases, increasing LIF expression levels may be used to treat, or reduce the severity of, conditions such as arthritis and other immune-mediated joint inflammatory diseases.

In yet another example, collagen alpha 1(I) chain precursor may be modulated by targeting let-7a. Reduced collagen expression has been observed in arthritis, osteogenesis imperfecta, and similar indications. Overexpression of collagen alpha 1(I) chain precursor may be used to treat such conditions.

As yet another example, VAMP-2 may be modulated by targeting miR-34. VAMP-2 is downregulated in states insulin deficiency, i.e., diabetes mellitus. Overexpressing VAMP-2 may thus be used to treat some forms of diabetes.

Thus the invention is useful for the treatment of diabetics. A diabetic is a patient that is affected by, or at risk of developing, diabetes and/or any of a group of related disorders in which there is a defect in the regulation of circulatory and/or intracellular glucose (sugar) levels. Diabetic patients include subjects with abnormally high levels of blood sugar (hyperglycemia) or abnormally low levels of blood sugar (hypoglycemia).

Diabetes is a highly debilitating and increasingly common disorder that is typically associated with impaired insulin signaling. Type 1 diabetes results from the body's impairment of insulin production due to loss of pancreatic beta cells. Conditions associated with type 1 diabetes include hyperglycemia, hypoglycemia, ketoacidosis and celiac disease. Some complications of type 1 diabetes include: heart disease (cardiovascular disease), blindness (retinopathy), nerve damage (neuropathy), and kidney damage (nephropathy).

Type 2 diabetes results from insulin resistance (a condition in which the body fails to properly use insulin—cellular sensitivity to circulating insulin is impaired), combined with relative insulin deficiency. Type 2 diabetes increases the risk for many serious complications including heart disease (cardiovascular disease), blindness (retinopathy), nerve damage (neuropathy), and kidney damage (nephropathy).

Pre-diabetes is a condition that occurs when a subject's blood glucose levels are higher than normal but not high enough for a diagnosis of type 2 diabetes. It is estimated that before subjects develop type 2 diabetes, they almost always have “pre-diabetes”—blood glucose levels that are higher than normal but not yet high enough to be diagnosed as diabetes. Recent research has shown that some long-term damage to the body, especially the heart and circulatory system, may already be occurring during pre-diabetes.

There are tests routinely used by those of ordinary skill in the art to establish if a subject is a “diabetic subject”. Two different tests that can be used to determine whether a subject is a “diabetic subject” are: the fasting plasma glucose test (FPG) or the oral glucose tolerance test (OGTT). The blood glucose levels measured after these tests can be used to determine whether a subject has a normal metabolism, or whether a subject is a “diabetic subject,” in other words whether a subject has pre-diabetes or diabetes. If the blood glucose level is abnormal following the FPG, the subject has impaired fasting glucose (IFG); if the blood glucose level is abnormal following the OGTT, the subject has impaired glucose tolerance (IGT). In the FPG test, the subject's blood glucose is measured first thing in the morning before eating. In the OGTT, the subject's blood glucose is tested after fasting and again 2 hours after drinking a glucose-rich drink.

Normal fasting blood glucose is below 100 mg/dl. A subject with pre-diabetes has a fasting blood glucose level between 100 and 125 mg/dl. If the blood glucose level rises to 126 mg/dl or above, the subject has diabetes. In the OGTT, the subject's blood glucose is measured after a fast and 2 hours after drinking a glucose-rich beverage. Normal blood glucose is below 140 mg/dl 2 hours after the drink. In pre-diabetes, the 2-hour blood glucose is 140 to 199 mg/dl. If the 2-hour blood glucose rises to 200 mg/dl or above, the subject has diabetes.

According to the invention, a subject at risk of developing diabetes or a related disorder is a subject that is predisposed to the disease or disorder due to genetic or other risk factors.

The invention is also useful for the treatment of neurodegenerative diseases such as Alzheimer's, for example, by altering expression of a gene involved in neural regulation pathways. The method may involve administering, to a subject, a composition such as an isolated oligonucleotide to regulate an miRNA involved in the expression of genes involved in neurodegenerative disease. For instance, BDNF may be modulated by targeting miR-1 or miR-206. BDNF decreases have been linked to late-stage Alzheimer's disease. Thus, using the systems and methods described herein, overexpression of BDNF may be used to treat Alzheimer's disease, or prevent its further progress.

Ataxin-1, as another example, can be modulated by targeting miR-101. Overexpression of Ataxin-1 may lead to neuronal degeneration and various neurodegenerative diseases. Thus, by inhibiting Ataxin-1, such neuronal degeneration may be prevented or at least inhibited, and such neurodegenerative diseases can thus be treated.

In still another example, Ras-related protein RAP-1B may be modulated by targeting miR-101. Expression of RAP-1B may cause neurite growth. Thus, certain types of neurodegenerative diseases, such as Alzheimer's disease, may be treated by overexpressing RAP-1B.

SHANK2 can be modulated by targeting miR-218, according to another example. SHANK2 is a scaffolding molecule involved in development. Thus, certain types of SHANK2 haploinsufficiency may be treated by overexpression of SHANK2 using the systems and methods described herein.

As another example, Neurofilament Triplet L Protein may be modulated by targeting miR-23a. This protein may be overexpressed in subjects having various neurological diseases, such as Alzheimer's disease. Thus, inhibition of this gene may be used to treat Alzheimer's disease and other neurological disorders.

“Neurodegenerative disease” is defined herein as a disorder in which progressive loss of neurons occurs either in the peripheral nervous system or in the central nervous system. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor) and Wernicke-Korsakoff's related dementia. Neurodegenerative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

Most of the chronic neurodegenerative diseases are typified by onset during the middle adult years and lead to rapid degeneration of specific subsets of neurons within the neural system, ultimately resulting in premature death. The compositions of the invention may be administered to a subject to treat or prevent neurodegenerative disease or to promote tissue generation alone or in combination with the administration of other therapeutic compounds for the treatment or prevention of these disorders or promotion of tissue generation. Many of these drugs are known in the art.

The invention also embraces methods of treatment for neuritic pain. Methods of treatment according to the present invention comprise the administration of nucleic acids that influence gene expression through the miRNA pathways in subjects experiencing neuritic pain. For instance, synapsin II can be modulated by targeting miR-25. Synapsin II is involved in pain sensation, e.g., in nociceptive behavior. By inhibiting Synapsis II, pain sensations may be reduced. Thus, certain types of acute or chronic pain may be treated by controlling Synapsin II. Subjects in need of treatment for neuritic pain include subjects with neurotransmitter-dysregulation pain syndromes and neuropathies.

“Neurotransmitter-dysregulation pain syndromes” generally involve normal nerves, but possess subtle alterations in quantity and quality of the various neurotransmitter molecules like serotonin, norepinephrine, and substance P which are released by the sending terminal of one neuron and interact with receptors on the receiving terminal of another neuron. These subtle alterations lead to modulation of a nerve signal such that it is interpreted as pain or as more painful.

More specifically, sensory neuropeptides are released from the afferent nerve ending of one nerve cell and received by receptors at the afferent end of another nerve cell. They are chemical messengers which transmit signal. There are numerous neuropeptides, including serotonin, dopamine, norepinephrine, somatostatin, substance P, and calcitonin gene-related peptide. Alterations in the quantity of neuropeptide release, changes in the afferent receptor, changes of re-uptake of the neuropeptides can all yield qualitative change of the neural signaling process, including an increase or decrease of pain modulation. Most pain states, including at a nerve receptor level in some of the peripheral neuropathies, and many “idiopathic” chronic pain conditions, have neuropeptide dysregulation as a feature of the nociceptive state. Other examples include reflex sympathetic dystrophy and myofascial pain syndrome. A

“Neuropathies” generally involve abnormalities in the nerve itself, such as degeneration of the axon or sheath. This derangement of nerve cell is experienced as pain. For example, there are neuropathies in which the cells of the myelin sheath, the Schwann cells, may be dysfunctional, degenerative, and/or may die off, while the axon remains unaffected. Alternatively, there are neuropathies where just the axon is disturbed, as well as combinations of both conditions. Neuropathies may also be distinguished by the process by which they occur and their location (e.g. arising in the spinal cord and extending outward or vice versa). Diphtheria polyneuropathy is an example of a myelin sheath disorder—although an infectious disease, there is no evidence of inflammatory cell infiltration. Arsenic poisoning neuropathy is an example of a more pure axonal neuropathy. Diabetes induces a mixed myelin-axonal neuropathy.

Neuropathies treatable by the methods of this invention include: (I) syndromes of acute ascending motor paralysis with variable disturbance of sensory function, (II) syndromes of subacute sensorimotor paralysis, (III) syndromes of acquired forms of chronic sensorimotor polyneuropathy, (IV) syndromes of determined forms of chronic polyneuropathy, genetically, (V) syndromes of recurrent or relapsing polyneuropathy, and (VI) syndromes of mononeuropathy or multiple neuropathies (Adams and Victor, Principles of Neurology, 4th ed., McGraw-Hill Information Services Company, p. 1036, 1989).

The methods of the invention are also useful for treating cardiovascular disease by regulating expression of one or more genes involved in maintaining cardiovascular function. Thus, in yet another set of embodiments, cardiovascular disease may be treated using the systems and methods of the invention. Many such diseases are characterized by improper gene expression. By administering, to a subject, a composition such as a nucleic acid influences the activity of one or more miRNAs, the expression of such genes may be controlled. For instance, BCNG-2 may be modulated by targeting miR-25. BCNG-2 is involved in ionic channel gaiting in both the brain or the heart. Inhibition of BCNG-2, using the systems and methods described herein, may thus slow activation of the ionic channels of the heart or the brain.

Pituitary adenylate cylcase-activating peptide, for instance, may be modulated by targeting miR-103 or miR-107. Pituitary adenylate cylcase-activating peptide may inhibit certain types of cell proliferation, for example, proliferation of cardiac fibroblasts. Thus, in certain types of diseases, for instance cardiovascular diseases such as myocardial fibrosis, heart failure, cardiomyopathy, or pulmonary hypertension, or certain types of kidney diseases such as chronic tubulointerstitial nephropathy, cell proliferation may be decreased by increasing pituitary adenylate cylcase-activating peptide expression levels.

As another example, SERCA2 may be modulated by targeting let-7a. Reduced SERCA2 function has been linked to systolic heart failure and systolic dysfunction. By increasing SERCA2 expression levels using the systems and methods described herein, heart performance may be improved.

In another example, LDLR can be modulated by targeting miR-130 or miR-130b. LDLR is involved in certain cell signaling pathways linked to hypercholesterolemia. Increasing LDR expression levels may be used to treat hypercholesterolemia.

Thus, the method of the invention includes therapies to treat or prevent cardiovascular disorders. The cardiovascular disorder may be a myocardial infarction, myocardial ischemia, angina (stable or unstable), stroke, and peripheral artery disease (e.g., peripheral ischemic cardiovascular disease), transient ischemic attack, claudication(s), vascular occlusion(s), heart failure, arrhythmia, cardiomyopathy, myocarditis, or valvular heart disease. The cardiovascular disorder can be any cardiovascular disorder associated with an atherosclerotic disease.

As used herein, a subject “at risk of developing a cardiovascular disorder” is a subject determined to be at risk according to conventional medical practice. (See, e.g., Harrison's Principles of Experimental Medicine, 15th Edition, McGraw-Hill, Inc., New York). Typically, an individual at risk of developing a cardiovascular disorder has one or more risk factors associated with cardiovascular disease. Such risk factors include family history of a cardiovascular disorder, hypertension, pre-hypertension, hyperlipidemia, elevated level(s) of a marker of systemic inflammation, diabetes, smoking, atherosclerosis, age, etc. In addition, atrial fibrillation, or recent stroke and/or myocardial infarction are important risk factors.

Hyperlipidemia is hypercholesterolemia and/or hypertriglyceridemia. Hypercholesterolemic human subjects and hypertriglyceridemic human subjects are associated with increased incidence of cardiovascular events. A hypercholesterolemic human subject is one who fits the current criteria established for a hypercholesterolemic human subject. A hypertriglyceridemic human subject is one who fits the current criteria established for a hypertriglyceridemic subject. A hypercholesterolemic subject has an LDL level of >160 mg/dL, or >130 mg/dL and at least two risk factors selected from the group consisting of: male gender, family history of premature coronary heart disease, cigarette smoking, hypertension, low HDL (<35 mg/dL), diabetes mellitus, hyperinsulinemia, abdominal obesity, high lipoprotein, and personal history of a cardiovascular event. A hypertriglyceridemic human subject has a triglyceride (TG) level of >250 mg/dL.

Hypertension is defined as a systolic blood pressure >140 mm Hg, and/or a diastolic pressure >90 mm Hg or both. Pre-hypertension is defined as systolic blood pressure between 115 and 140 mm Hg, and/or a diastolic pressure between 80 and 90 mm Hg.

Obesity is a state of excess adipose tissue mass. Although not a direct measure of adiposity, the most widely used method to gauge obesity is the body mass index (BMI), which is equal to weight/height² (in kg/m²) (See, e.g., Harrison's Principles of Experimental Medicine, 15th Edition, McGraw-Hill, Inc., N.Y.—hereinafter “Harrison's”). Based on data of substantial morbidity, a BMI of 30 is most commonly used as a threshold for obesity in both men and women. A BMI between 25 and 30 should be viewed as medically significant and worthy of therapeutic intervention, especially in the presence of risk factors that are influenced by adiposity, such as hypertension and glucose intolerance. Although often viewed as equivalent to increased body weight, this need not be the case. Lean but very muscular individuals may be overweight by arbitrary standards without having increased adiposity. Other approaches to quantifying obesity include anthropometry (skin-fold thickness), densitometry (underwater weighing), computed tomography (CT) or magnetic resonance imaging (MRI), and electrical impedance.

An elevated level(s) of a marker of systemic inflammation is a level that is above the average for a healthy human subject population (i.e., human subjects who have no signs and symptoms of disease). When the marker of systemic inflammation is CRP, a CRP level of ≧1 is considered an elevated level.

Other diseases that can be treated according to the methods of the invention include preclampsia, psoriasis and diseases associated with hematopoiesis.

In yet another example, MCSF can be modulated by targeting miR-130, miR-130b, or mi-27a. MCSF expression often is higher in preeclamptic women, and thus, the inhibition of MCSF using the systems and methods described herein may be used to treat preeclampsia.

CAT-1, as another example, may be modulated by targeting miR-122a. The expression of CAT-1 may facilitate erythroid hematopoiesis, for example, by transporting L-arginine intracellularly. Thus, expression of CAT-1 is linked to the differentiation of red blood cells. Accordingly, for certain conditions such as anemia, upregulation of CAT-1 may be used as a method of treatment, while for other conditions such as leukemia, CAT-1 may be inhibited using the systems and methods described herein to treat the disease. Additionally, overexpression of CAT-1 has been linked to skin conditions such as psoriasis. Accordingly, by downregulating or inhibiting CAT-1, psoriasis may be treated.

ORP-3 can be modulated by targeting miR-124a, according to another example. ORP-3 is believed to facilitate hematopoiesis. Thus, for conditions such as anemia, upregulation of ORP-3 may be used as a method of treatment, while for other conditions such as leukemia, ORP-3 may be inhibited to treat the disease.

In one example, EDF may be modulated by targeting miR-203. In patients with anemia, e.g., renal anemia, increasing EDF expression levels, in conjunction with the administration of erythropoietin, may be used as a form of therapeutic treatment. EDF expression may be increased using the systems and methods described herein.

Estrogen Receptor-Like 1, in still another example, may be modulated by targeting miR-135b. Inhibition of Estrogen Receptor-Like 1 may inhibit osteogenesis, or increase adipocyte formation. Overexpression of Estrogen Receptor-Like 1, on the other hand, may promote osteogenesis.

As mentioned, certain aspects of the invention include a method of administering a composition as described herein to a subject, for instance, an isolated oligonucleotide comprising a sequence that is substantially antisense to an miRNA, e.g., to control gene expression or cancer. When administered, the compositions of the invention are applied in a therapeutically effective, pharmaceutically acceptable amount as a pharmaceutically acceptable formulation. As used herein, the term “pharmaceutically acceptable” is given its ordinary meaning as used in the art. Pharmaceutically acceptable compounds are generally compatible with other materials of the formulation and are not generally deleterious to the subject. A composition of the invention may be administered to the subject in any therapeutically effective dose or treatment. A “therapeutically effective” dose or amount is capable of at least partially preventing or treating cancer or at least partially inhibiting gene expression, as previously described. A therapeutically effective amount may be determined by those of ordinary skill in the art, for instance, employing factors such as those further described below and using no more than routine experimentation.

In administering the compositions of the invention to a subject, dosing amounts, dosing schedules, routes of administration, and the like may be selected so as to affect known activities of the compositions of the invention. Dosages may be estimated based on the results of experimental models, optionally in combination with the results of assays of compositions of the present invention. Dosage may be adjusted appropriately to achieve desired drug levels, local or systemic, depending upon the mode of administration. The doses may be given in one or several administrations per day. In some cases, parenteral administration of the composition may be from one to several orders of magnitude lower dose per day, as compared to oral doses. In the event that the response of a particular subject is insufficient at such doses, even higher doses (or effectively higher doses by a different, more localized delivery route) may be employed to the extent that subject tolerance permits. Multiple doses per day are also contemplated, in certain cases, to achieve appropriate levels of the composition within the subject or within the active site of the subject, such as within the brain.

The dose of the composition to the subject may be such that a therapeutically effective amount of the composition (or a portion thereof) reaches or enters an active site. The dosage may be given in some cases at the maximum amount while avoiding or minimizing any potentially detrimental side effects to the subject. The dosage of the composition that is actually administered is dependent upon factors such as the final concentration desired at the active site, the method of administration to the subject, the efficacy of the composition, the longevity (i.e., half-life) within the subject of the composition, the frequency of treatment, the effect of concurrent treatments, etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition (or portion thereof) at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject. The specific dosage(s) given to the subject can thus be determined by those of ordinary skill in the art, using no more than routine experimentation.

Administration of the compositions of the invention may be accomplished by any medically acceptable method which allows the composition (or portion thereof) to reach its target. The particular mode selected will depend, of course, upon factors such as the particular composition, the severity of the state of the subject being treated, or the dosage required for therapeutic efficacy. As used herein, a “medically acceptable” mode of treatment is a mode able to produce effective levels of the composition (or portion thereof) within the subject, without causing clinically unacceptable adverse effects. A “target” or “active site” is the location where a composition (or portion thereof) of the invention is able to bind to at least partially prevent or treat cancer or at least partially inhibit gene expression, as previously described.

Any medically acceptable method may be used to administer the composition to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally through parenteral injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system.

Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound of the composition. Other oral compositions suitable for use with the invention include solutions or suspensions in aqueous or non-aqueous liquids such as a syrup, an elixir, or an emulsion. In another set of embodiments, the composition may be used to fortify a food or a beverage.

In certain embodiments of the invention, the administration of the composition of the invention may be designed so as to result in sequential exposures to the composition over a certain time period, for example, hours, days, weeks, months, or years. This may be accomplished by repeated administrations of the composition by one of the methods described above, or by a sustained or controlled release delivery system in which the composition is delivered over a prolonged period without repeated administrations. Administration of the composition using such a delivery system may be, for example, by oral dosage forms, bolus injections, transdermal patches, or subcutaneous implants.

Other delivery systems suitable for use with the present invention (e.g., where alteration and/or control of the release kinetics is desired) include time-release, delayed release, sustained release, or controlled release delivery systems. Such systems may avoid repeated administrations of the composition in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix (for example, as described in U.S. Pat. Nos. 4,452,775, 4,675,189, and 5,736,152), or diffusional systems in which an active component controls the release rate (for example, as described in U.S. Pat. Nos. 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Use of a long-term release implant may be particularly suitable in some embodiments of the invention. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Administration of the compositions of the invention (e.g., an isolated oligonucleotide comprising a sequence that is substantially antisense to an miRNA, a sequence that, when expressed by the cell, causes the cell to overexpress the miRNA, etc.) can be alone, or in combination with other therapeutic agents and/or compositions (e.g., other agents or compositions that can be used to treat cancer, such as those described below). In certain embodiments, the compositions of the invention can be combined with a suitable pharmaceutically acceptable carrier, for example, as incorporated into a liposome, incorporated into a polymer release system, or suspended in a liquid, e.g., in a dissolved form or a colloidal form. The carrier may be either soluble or insoluble, depending on the application. Compositions of the invention that may be pharmaceutically acceptable include not only the active compound, but also formulation ingredients such as salts, carriers, buffering agents, emulsifiers, diluents, excipients, chelating agents, drying agents, antioxidants, antimicrobials, preservatives, binding agents, bulking agents, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or alginic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid, or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation. In some cases, the pharmaceutically acceptable carrier(s) may be formulated such that the pH of the carrier(s) is at a desired value, e.g., through the use of buffering agents as described above. In some embodiments of the invention, generally high pH values are desired, e.g., a pH of at least about 9, at least about 10, at least about 11, at least about 12, or at least about 13. In other embodiments, however, generally low pH values may be desired, e.g., a pH of less than about 5, less than about 4, less than about 3, less than about 2, or less than about 1. A neutral pH may also be desired in some cases, e.g., a pH of between about 5 and 9, or a pH of between about 6 and 8.

In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the invention are combined to facilitate the application of the composition. The carrier may be co-mingled or otherwise mixed with one or more active compounds of the present invention, and with each other, in a manner such that there is no interaction which would substantially impair the desired pharmaceutical efficacy. Pharmaceutically acceptable carriers include, for example, diluents, emulsifiers, fillers, salts, buffers, excipients, drying agents, antioxidants, preservatives, binding agents, bulking agents, chelating agents, stabilizers, solubilizers, silicas, and other materials well-known in the art.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, peanut oil, injectable organic esters such as ethyl oleate, or fixed oils including synthetic mono or di-glycerides. Aqueous carriers include water, alcoholic/aqueous solutions, emulsions, or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and/or other additives may also be present such as, for example, antimicrobials, antioxidants, chelating agents and inert gases and the like. Those of skill in the art can readily determine the various parameters for preparing and formulating the compositions of the invention without resort to undue experimentation.

In some embodiments, the present invention includes a step of bringing a composition or compound of the invention into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product.

In some embodiments, a compound of the present invention may be present as a pharmaceutically acceptable salt. The term “pharmaceutically acceptable salts” includes salts of the compound, prepared in combination with, for example, acids or bases, depending on the particular compounds found within the composition and the treatment modality desired. Pharmaceutically acceptable salts can be prepared as alkaline metal salts, such as lithium, sodium, or potassium salts; or as alkaline earth salts, such as beryllium, magnesium, or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

The compositions may be administered alone, with carriers, or with other therapeutics to treat or prevent the diseases described herein. When the compounds are administered with other therapeutics, they may be administered together in the same composition, at the same time but in separate compositions, by the same or different routes of administration, or at different times, such as times that are separated by hours, days, weeks or months.

For instance, therapies for treating or preventing cardiovascular disorders include but are not limited to diet and/or exercise and/or therapies with: anti-lipemic agents, anti-inflammatory agents, anti-thrombotic agents, fibrinolytic agents, anti-platelet agents, direct thrombin inhibitors, glycoprotein II b/IIIa receptor inhibitors, agents that bind to cellular adhesion molecules and inhibit the ability of white blood cells to attach to such molecules (e.g. anti-cellular adhesion molecule antibodies), alpha-adrenergic blockers, beta-adrenergic blockers, cyclooxygenase-2 inhibitors, angiotensin system inhibitor, anti-arrhythmics, calcium channel blockers, diuretics, inotropic agents, vasodilators, vasopressors, and/or any combinations thereof.

Anti-lipemic agents are agents that reduce total cholesterol, reduce LDLC, reduce triglycerides, or increase HDLC. Anti-lipemic agents include statins and non-statin anti-lipemic agents, and/or combinations thereof. Statins are a class of medications that have been shown to be effective in lowering human total cholesterol, LDLC and triglyceride levels. Statins act at the step of cholesterol synthesis. By reducing the amount of cholesterol synthesized by the cell, through inhibition of the HMG-CoA reductase gene, statins initiate a cycle of events that culminates in the increase of LDLC uptake by liver cells. As LDLC uptake is increased, total cholesterol and LDLC levels in the blood decrease. Lower blood levels of both factors are associated with lower risk of atherosclerosis and heart disease, and the statins are widely used to reduce atherosclerotic morbidity and mortality.

Examples of statins include, but are not limited to, simvastatin (Zocor) (U.S. Pat. No. 4,444,784), lovastatin (Mevacor) (U.S. Pat. No. 4,231,938), pravastatin (Pravachol) (U.S. Pat. No. 4,346,227), fluvastatin (Lescol) (U.S. Pat. No. 4,739,073), atorvastatin (Lipitor) (U.S. Pat. No. 5,273,995), cerivastatin (Baycol), rosuvastatin (Crestor), pitivastatin and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402.

Examples of statins already approved for use in humans include atorvastatin, cerivastatin, fluvastatin, pravastatin, simvastatin and rosuvastatin. The reader is referred to the following references for further information on HMG-CoA reductase inhibitors: Drugs and Therapy Perspectives (May 12, 1997), 9: 1-6; Chong (1997) Pharmacotherapy 17:1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368. Another statin drug of note is compound 3a (S-4522) in Watanabe (1997) Bioorganic and Medicinal Chemistry 5: 437-444.

Non-statin anti-lipemic agents include but are not limited to fibric acid derivatives (fibrates), bile acid sequestrants or resins, nicotinic acid agents, cholesterol absorption inhibitors, acyl-coenzyme A: cholesterol acyl transferase (ACAT) inhibitors, cholesteryl ester transfer protein (CETP) inhibitors, LDL receptor antagonists, farnesoid X receptor (FXR) antagonists, sterol regulatory binding protein cleavage activating protein (SCAP) activators, microsomal triglyceride transfer protein (MTP) inhibitors, squalene synthase inhibitors, and peroxisome proliferation activated receptor (PPAR) agonists.

Examples of fibric acid derivatives include but are not limited to gemfibrozil (Lopid), fenofibrate (Tricor), clofibrate (Atromid) and bezafibrate.

Examples of bile acid sequestrants or resins include but are not limited to colesevelam (WelChol), cholestyramine (Questran or Prevalite) and colestipol (Colestid), DMD-504, GT-102279, HBS-107 and S-8921.

Examples of nicotinic acid agents include but are not limited to niacin and probucol.

Examples of cholesterol absorption inhibitors include but are not limited to ezetimibe (Zetia).

Examples of ACAT inhibitors include but are not limited to Avasimibe, CI-976 (Parke Davis), CP-113818 (Pfizer), PD-138142-15 (Parke Davis), F1394, and numerous others described in U.S. Pat. Nos. 6,204,278, 6,165,984, 6,127,403, 6,063,806, 6,040,339, 5,880,147, 5,621,010, 5,597,835, 5,576,335, 5,321,031, 5,238,935, 5,180,717, 5,149,709, and 5,124,337.

Examples of CETP inhibitors include but are not limited to Torcetrapib, CP-529414, CETi-1, JTT-705, and numerous others described in U.S. Pat. Nos. 6,727,277, 6,723,753, 6,723,752, 6,710,089, 6,699,898, 6,696,472, 6,696,435, 6,683,099, 6,677,382, 6,677,380, 6,677,379, 6,677,375, 6,677,353, 6,677,341, 6,605,624, 6,586,448, 6,521,607, 6,482,862, 6,479,552, 6,476,075, 6,476,057, 6,462,092, 6,458,852, 6,458,851, 6,458,850, 6,458,849, 6,458,803, 6,455,519, 6,451,830, 6,451,823, 6,448,295, 5,512,548.

One example of an FXR antagonist is Guggulsterone. One example of a SCAP activator is GW532 (GlaxoSmithKline).

Examples of MTP inhibitors include but are not limited to Implitapide and R-103757.

Examples of squalene synthase inhibitors include but are not limited to zaragozic acids.

Examples of PPAR agonists include but are not limited to GW-409544, GW-501516, and LY-510929.

Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic agents and/or fibrinolytic agents include Plasminogen (to plasmin via interactions of prekallikrein, kininogens, Factors XII, XIIIa, plasminogen proactivator, and tissue plasminogen activator TPA]) Streptokinase; Urokinase: Anisoylated Plasminogen-Streptokinase Activator Complex; Pro-Urokinase; (Pro-UK); rTPA (alteplase or activase; r denotes recombinant); rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

Anti-platelet agents include Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide.

Direct thrombin inhibitors include hirudin, hirugen, hirulog, agatroban, PPACK, thrombin aptamers.

Glycoprotein IIb/IIIa receptor Inhibitors are both antibodies and non-antibodies, and include but are not limited to ReoPro (abcixamab), lamifiban, tirofiban.

Agents that bind to cellular adhesion molecules and inhibit the ability of white blood cells to attach to such molecules include polypeptide agents. Such polypeptides include polyclonal and monoclonal antibodies, prepared according to conventional methodology. Such antibodies already are known in the art and include anti-ICAM 1 antibodies as well as other such antibodies.

Examples of alpha-adrenergic blockers include: doxazocin, prazocin, tamsulosin, and tarazosin.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

Cyclooxygenase-2 (COX-2) is a recently identified new form of a cyclooxygenase. Cyclooxygenase is an enzyme complex present in most tissues that produces various prostaglandins and thromboxanes from arachidonic acid. A number of selective COX-2 inhibitors are known in the art. These include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors”; U.S. Pat. No. 5,677,318 Diphenyl-1,2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-dihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated-styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.” nd therefore part of the present invention.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin I and angiotensin II are synthesized by the enzymatic renin-angiotensin pathway. The synthetic process is initiated when the enzyme renin acts on angiotensinogen, a pseudoglobulin in blood plasma, to produce the decapeptide angiotensin I. Angiotensin I is converted by angiotensin converting enzyme (ACE) to angiotensin II (angiotensin-[1-8]octapeptide). The latter is an active pressor substance which has been implicated as a causative agent in several forms of hypertension in various mammalian species, e.g., humans.

Angiotensin (renin-angiotensin) system inhibitors are compounds that act to interfere with the production of angiotensin II from angiotensinogen or angiotensin I or interfere with the activity of angiotensin II. Such inhibitors are well known to those of ordinary skill in the art and include compounds that act to inhibit the enzymes involved in the ultimate production of angiotensin II, including renin and ACE. They also include compounds that interfere with the activity of angiotensin II, once produced. Examples of classes of such compounds include antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists. In a preferred embodiment of the invention, the renin-angiotensin system inhibitors are renin inhibitors, ACE inhibitors, and angiotensin II antagonists.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include: peptidic compounds (e.g., saralasin, [(San¹)(Val⁵)(Ala⁸)] angiotensin -(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(−1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl]1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G.D.Searle and Company).

Angiotensin converting enzyme (ACE), is an enzyme which catalyzes the conversion of angiotensin I to angiotensin II. ACE inhibitors include amino acids and derivatives thereof, peptides, including di and tri peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides, and analogs thereof (U.S. Pat. Nos. 5,071,837, 5,064,965, 5,063,207, 5,036,054, 5,036,053, 5,034,512, and 4,894,437).

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogenous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11-2933), phenytoin, barbiturates, and the peptides dynorphin, omega-conotoxin, and omega-agatoxin, and the like and/or pharmaceutically acceptable salts thereof.

Diuretics include but are not limited to: carbonic anhydrase inhibitors, loop diuretics, potassium-sparing diuretics, thiazides and related diuretics.

Vasodilators include but are not limited to coronary vasodilators and peripheral vasodilators.

Inotropic agents include but are not limited to glycosides such as digitalis, digoxin, amrinone and milrinone.

Anti-arrhythmics include but are not limited to quinidien, procainamide, disopyramide, moricizine, lidocaine, mexiletine, phenytoin, tocainide, encainide, flecainide, propafenone, indecainide, propranolol, acebutolol, esmolol, amiodarone, bretylium, verapamil, and diltiazem.

Examples of anti-cancer agents and drugs that can be used in combination with one or more compositions of the invention (e.g., an isolated oligonucleotide comprising a sequence that is substantially antisense to an miRNA) include, but are not limited to, any one or more of 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, carn 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, polymorphs, derivatives, enantiomers, and/or functionally equivalent compositions thereof.

In still another aspect, the present invention provides any of the above-mentioned systems or methods in kits, optionally including instructions for use of the composition, e.g., for the inhibition of a gene. In one set of embodiments, the “kit” may include a computer system and/or computer-readable media, optionally in conjunction with instructions. In some cases, the computer system and/or computer-readable media may contain a program able to perform any of the above-mentioned methods, for example, methods of identifying a target of an miRNA sequence. In another set of embodiments, the “kit” defines a package including one or more of the above-described compositions of the invention and the instructions, and/or analogs, derivatives, or functionally equivalent compositions thereof. Thus, for example, the kit can include a description of use of a composition for participation in any technique associated with the inhibition of genes. The kit can also include a description of use of the compositions as discussed herein. Instructions also may be provided for use of the composition in any suitable technique as previously described. The instructions may be of any form provided in connection with the composition.

The kits described herein may also contain one or more containers, which may contain the inventive composition and other ingredients as previously described. The kits also may contain instructions for mixing, diluting, and/or administrating the compositions in some cases. The kits also can include other containers with one or more solvents, surfactants, preservative and/or diluents (e.g., normal saline (0.9% NaCl), or 5% dextrose) as well as containers for mixing, diluting and/or administrating the compositions.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the active compound(s) within the composition. Suitable solvents are well known, for example as previously described, and are available in the literature.

The kit, in one set of embodiments, may comprise a carrier that is compartmentalized to receive in close confinement one or more container means such as vials, tubes, and the like, each of the compartments comprising one of the separate elements to be used in the method. For example, one of the compartments may comprise a positive control for an assay. Additionally, the kit may include containers for other components of the compositions, for example, buffers useful in the assay.

The invention also involves, in yet another aspect, promotion of any of the systems, methods, or compositions described herein. As used herein, “promotion” includes all methods of doing business including, but not limited to, methods of selling, advertising, assigning, licensing, contracting, instructing, educating, researching, importing, exporting, negotiating, financing, loaning, trading, vending, reselling, distributing, replacing, or the like that can be associated with the systems, methods, or compositions of the invention, e.g., as discussed herein. Promoting may also include, in some cases, seeking approval from a government agency to sell a composition of the invention for medicinal purposes. Methods of promotion can be performed by any party including, but not limited to, businesses (public or private), contractual or sub-contractual agencies, educational institutions such as colleges and universities, research institutions, hospitals or other clinical institutions, governmental agencies, etc. Promotional activities may include instructions or communications of any form (e.g., written, oral, and/or electronic communications, such as, but not limited to, e-mail, telephonic, facsimile, Internet, Web-based, etc.) that are clearly associated with the invention. As used herein, “instructions” can define a component of instructional utility (e.g., directions, guides, warnings, labels, notes, FAQs (“frequently asked questions”), etc., and typically involve written instructions on or associated with the composition and/or with the packaging of the composition, for example, use or administration of the composition. Instructions can also include instructional communications in any form (e.g., oral, electronic, digital, optical, visual, etc.), provided in any manner such that a user will clearly recognize that the instructions are to be associated with the composition, e.g., as discussed herein.

These documents are each incorporated herein by reference: Lewis, et al., “Prediction of Mammalian MicroRNA Targets,” Cell, 115:787-798 (2003); Lewis, et al., “Conserved Seed Pairing, Often Flanked by Adenosines, Indicates that Thousands of Human Genes are MicroRNA Targets,” Cell, 120:15-20 (2005); Lim, et al., “Vertebrate MicroRNA Genes,” Science, 299:1540 (2003); U.S. Provisional Patent Application Ser. No. 60/493,239, filed on Aug. 7, 2003, entitled “Methods and Products for Expression of MicroRNAs,” by Chen, et al.; U.S. patent application Ser. No. 10/913,288, filed on Aug. 6, 2004, entitled “Methods and Products for Expression of MicroRNAs,” by Chen, et al., published as U.S. Patent Application Publication 2005/0075492 on Apr. 7, 2005; International Patent Application No. PCT/US2004/025572, filed on Aug. 6, 2004, entitled “Methods and Products for Expression of MicroRNAs,” by Chen, et al., published as WO 2005/047505 on May 26, 2005; and U.S. Provisional Patent Application Ser. No. 60/639,231, filed Dec. 23, 2004, entitled “Vertebrate miRNA and Systems and Methods of Detection Thereof,” by Lewis, et al.

The following examples are intended to illustrate certain embodiments of the present invention, but do not exemplify the full scope of the invention.

### Example 1

This example illustrates one method of identifying targets of miRNA sequences, in accordance with an embodiment of the invention. This method combines thermodynamics-based modeling of RNA:RNA duplex interactions with comparative sequence analysis to predict miRNA targets conserved across multiple genomes.

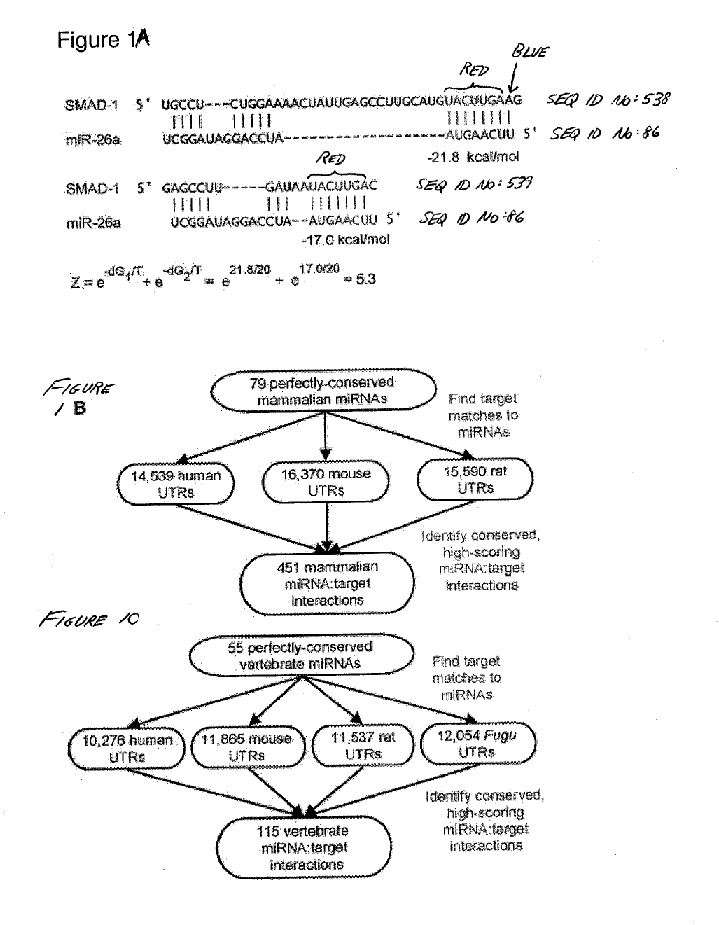

This example method is briefly outlined in FIG. 1 for an example system. A more detailed description can be seen in Example 9. FIG. 1A illustrates the structures, energies and scoring for RNA duplexes involving human miR-26a and two target sites in the 3′ UTR of the human SMAD-1 gene, with seeds and seed matches in red, and seed extensions in blue. FIGS. 1B-1C is a general schematic for the identification of targets conserved across mammals (FIG. 1B) and targets conserved in mammals and fish (FIG. 1C). The number of genes from each organism with identified orthologs in every other organism is indicated. In FIG. 1D, the positions of two target sites for miR-26a (lower line segments) in orthologous SMAD-1 3′ UTR sequences from human (Hs), mouse (Mm), rat (Rn), and Fugu (Fr), are shown, with the Z-score and rank of each miRNA:UTR pair, with T=20.

Given a miRNA that is conserved in multiple organisms and a set of orthologous 3′ UTR sequences from these organisms, the method in this example 1) searches the UTRs in the first organism for segments of perfect Watson-Crick complementarity to bases 2 to 8 of the miRNA (numbered from the 5′ end) (the 7-nucleotide segment of the miRNA is referred to in this example as the “miRNA seed” and the UTR heptamers with perfect Watson-Crick complementarity to the seed is referred to as “seed matches”; 2) extends each seed match with additional base pairs to the miRNA as far as possible in each direction, allowing G:U pairs, but stopping at mismatches; 3) optimizes base-pairing of the remaining 3′ portion of the miRNA to the 35 bases of the UTR immediately 5′ of each seed match using an RNA folding program such as “RNAfold,” thus extending each seed match to a longer “target site”; 4) assigns a folding free energy G to each such miRNA:target site interaction (ignoring initiation free energy) (for example, using a program such as RNAeval); 5) assigns a Z-score to each UTR, defined as:

$$Z = {\sum }_{k = 1}^{n} {e}^{- {G}_{k} / T} ,$$

where n is the number of seed matches in the UTR, Gₖ is the free energy of the miRNA:target site interaction (kcal/mol) for the k^{th} target site evaluated in the previous step, and T is a parameter described below (UTRs that have no seed match are assigned a Z-score of 1.0); and 6) sorts the UTRs in this organism by Z-score, and assigns a rank Rᵢ to each. Optionally, this method may be repeated for each set of UTRs from each organism. The method also may be used to predict as targets those genes for which both Zᵢ≧Z_{C} and Rᵢ≦R_{C} for an orthologous UTR sequence in each organism, where Z_{C} and R_{C} are pre-chosen Z-score and rank cutoffs. The program used in this particular example is shown in Appendix B, which is a computer program listing appendix, incorporated herein by reference.

The only free parameters in this protocol are R_{C} and Z_{C}, and the T parameter in the formula relating predicted free energy to Z-score. The value of the T parameter influences the relative weighting of UTRs with fewer high-affinity target sites to those with larger numbers of low-affinity target-sites, and in this sense is analogous to temperature. However, there is no thermodynamic meaning to the T parameter or the Z-scores used in this analysis; they merely provide a convenient means of weighting and summing predicted folding free energies. Suitable values for R_{C}, Z_{C}, and T were assigned by optimization over a range of reasonable values using separate training and test sets of miRNAs.

Details of the method follow. Human and mouse miRNA sequences that satisfy established criteria were downloaded from the Rfam website (http://www.sanger.ac.uk/Software/Rfam), which is a publicly-accessible database of RNA sequences from various organisms. Human miRNAs that lacked annotated mouse orthologs and mouse miRNAs that lacked annotated human orthologs were searched against the mouse and human genomes respectively with BLASTN (a publicly-available search tool for comparing nucleotide sequences against a nucleotide sequence database, available from the National Institutes of Health, see http://www.ncbi.nlm.nih.gov/BLAST/) and MiRscan (a program for comparing the sequences of two hairpin structures, based on their similarity to 50 pairs of experimentally verified C. elegans/C. briggsae miRNA hairpins, available at http://genes.mit.edu/mirscan/).

To identify Fugu homologs, the human miRNAs were searched against the Fugu genome using BLASTN and MiRscan, and the 121 human miRNAs with perfectly homologous miRNAs in mouse and clear homologous miRNAs in Fugu were assigned to rMamm. For sets of human miRNAs in rMamm with identical seed heptamers, a single representative was chosen, yielding 79 human miRNAs (nrMamm). The choice was based on conservation to Fugu and C. elegans miRNAs when possible (i.e., the sequence most broadly conserved was chosen), but was otherwise essentially arbitrary (the miRNA with the lowest mir-# was generally chosen). The subset of 55 miRNAs from nrMamm which had perfect conservation to Fugu was assigned to nrVert. (rMamm, nrMamm, and nrVert are described in more detail below.)

3′ UTR sequences for all human genes, and all mouse, rat and Fugu genes associated with a human ortholog, were retrieved using EnsMart version 15.1 (a generic data warehouse for querying large biological data sets and integration with third-party data and tools, available at http://www.ensembl.org/EnsMart). Annotated 3′ UTR sequences were available for only 45% of rat genes in this set and for none of the Fugu genes. Moreover, 14% of annotated rat 3′ UTR sequences were less than 50 nucleotides in length. Therefore, each annotated 3′ UTR was extended with 2 kb of 3′ flanking sequence. Repetitive elements were masked in these sequences using RepeatMasker (a program that screens DNA sequences for interspersed repeats and low complexity DNA sequences, outputting an annotation of the repeats that are present in the query sequence as well as a modified version of the query sequence in which all the annotated repeats have been masked, available http://repeatmasker.genome.washington.edu/cgi-bin/RM2_req.pl) with repeat libraries for primates, rodents or vertebrates, as appropriate.

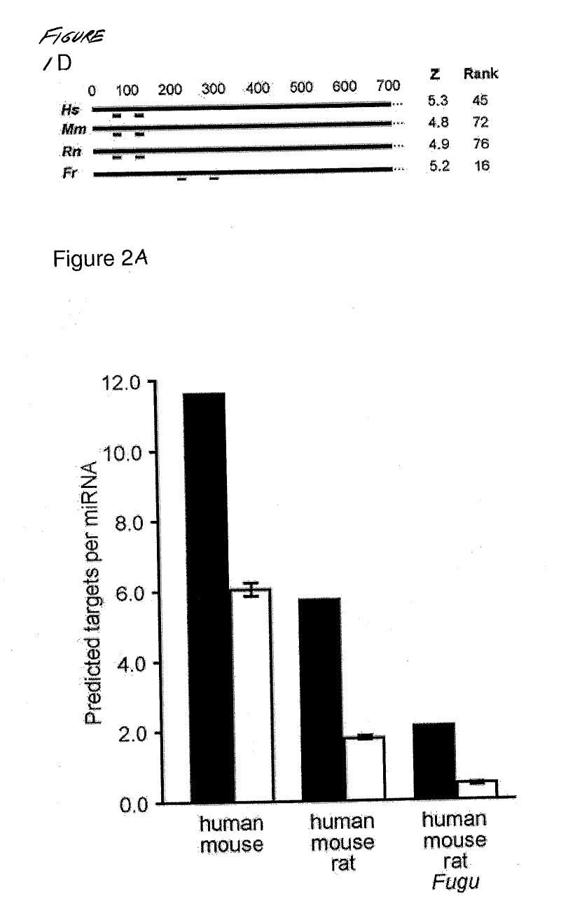

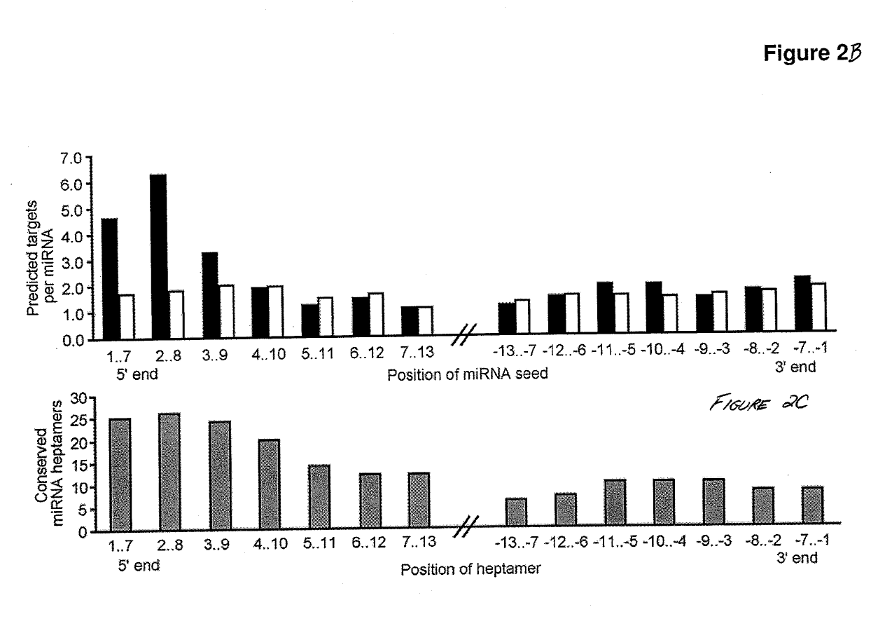

The 3′ UTR sequences were searched for antisense matches to the designated seed region of each miRNA (e.g., bases 2 . . . 8 starting from the 5′ end). The choice of a 7-nucleotide seed was motivated by the observation that shorter seeds gave substantially lower signal:noise ratios, in this example because it is largely based on a three-genome (human, mouse and rat) analysis while longer seeds reduced the number of predicted targets at comparable signal:noise ratios, as shown in Example 8, with the choice of a 6-nucleotide seed. Because changing the size of the seed has a large effect on the noise as well as the signal, these observations were more difficult to interpret in terms of potential mechanistic implications than the “sliding seed” data of FIG. 2B. For seeds located on the 5′ portion of the miRNA, 35 nucleotides flanking the seed match on the 5′ end and 5 nucleotides flanking the seed match on the 3′ end were retrieved (a “mirror” version of this algorithm was used for 3′ seeds in the experiment described in FIG. 2B). Target sites in which the 35-nucleotide flanking region contained masked bases or the seed match occurred less than 20 nucleotides downstream of a previous seed match were discarded. Base-pairing between the miRNA seed and UTR was extended with additional flanking base pairs as far as possible in both directions, allowing G:U pairs but disallowing gaps. The base-pairing pattern of the remaining 3′ end (or in the case of a 3′ seed, the remaining 5′ end) was predicted by running RNAfold on a foldback sequence consisting of an artificial stem-loop (5′-GGGCCCGGGULLLLLLACCCGGGCCC-3′ (SEQ ID NO: 1), where “L” is an anonymous unpaired loop character, and all other bases are paired to a complementary base on the opposite side of the stem) attached to the extended seed match. RNAfold optimization was constrained so that all base pairs found in previous steps were fixed, the structure of the artificial stem was fixed, and bases in the miRNA and UTR were allowed to pair only with bases in the UTR and miRNA, respectively. The stem-loop was removed, and RNAeval was used to estimate the energy of the miRNA:UTR duplex formed by the base pairs determined in the previous steps.

Training sets were constructed with 40 randomly-chosen miRNAs from nrMamm and 27 randomly-chosen miRNAs from nrVert. The remaining microRNAs were assigned to the nrMamm and nrVert reference sets. TargetScan was tested on the training sets with various parameter values: T was varied from 5 to 25 in increments of 5, Z_{C} was varied between 0 and 10 in increments of 0.5, and R_{C} was varied between 50 and 1000 in increments of 50. The parameters T=20, Z_{C}=4.5, R_{C}=200, were found to give an optimal signal:noise of 3.4:1 for the nrMamm training set. When R_{C} was raised to 300 or Z_{C} was lowered to 4 the signal:noise decreased only moderately to ˜3:1. The parameters T=10, Z_{C}=4.5, R_{C}=350, were found to give an optimal signal:noise of 4.6:1 for the nrVert training set used with UTR sets from all four genomes. For both the nrMamm and nrVert sets, the signal:noise ratios obtained using the training sets did not differ significantly from the corresponding signal:noise ratios obtained using the reference sets, and thus results from the two sets were merged.

For each miRNA in nrMamm, randomly-permuted sequences with the same starting base, length, and base composition as the real miRNA were generated until four sequences were found that deviate from the original miRNA by less than 15% in the following properties: (i) E(SM), the 1^{st} order Markov probability of the seed match (ii) E(TM), the 1^{st} order Markov probability of the antisense of the 3′ end of the miRNA (or the 5′ end in the case of a 3′ miRNA seed) (iii) O(SM), the observed count of seed matches in the UTR dataset, and (iv) the predicted folding free energy of a seed:seed match duplex. For a miRNA (or shuffled miRNA, referred to as an miRNA like control sequence) with the initial sequence S₁, S₂, S₃, S₄, S₅, S₆, S₇, S₈, and the seed designated as bases 2 . . . 8, E(SM) was equal to (P_{S}_{₁},_{S}_{₂}·P_{S}_{₂},_{S}_{₃}·P_{S}_{₃},_{S}_{₄}·P_{S}_{₄},_{S}_{₅}·P_{S}_{₅},_{S}_{₆}·P_{S}_{₆},_{S}_{₇}·P_{S}_{₇},_{S}_{₈}) where P_{S}_{ₖ},_{S}_{ₖ₊₁} was the conditional frequency of the nucleotide Sₖ₊₁ given Sₖ at the previous position in the set of inverse complements of the UTRs in the UTR database. E(TM) was the analogous quantity calculated for the remainder of the sequence (i.e., for bases 9, 10, 11, . . . to the end of the miRNA or shuffled miRNA). O(SM) was determined directly from heptamer counts in the UTR dataset. The predicted folding free energy of a seed:seed match duplex was determined using RNAeval. Another program, DiMirShuffle, generated shuffled controls for a given miRNA sequence by shuffling the dinucleotides of the specified miRNA seed (e.g., bases 2 . . . 8 of the miRNA).

### Example 2

The method of Example 1 was applied in this example to two sets of miRNAs (microRNAs of FIG. 6, included within SEQ ID NO: 3 to SEQ ID NO: 468): a nonredundant pan-mammalian set of 79 miRNAs that have homologs in human, mouse and pufferfish and identical sequence in human and mouse, but not necessarily pufferfish, and a nonredundant pan-vertebrate set of 55 miRNAs that have identical sequence in human, mouse and pufferfish. These sets, referred to as nrMamm and nrVert, respectively (FIG. 6), are nonredundant in that when multiple miRNAs had identical seed heptamers, a single representative was chosen. The initial use of miRNAs that were both nonredundant and perfectly conserved among the queried species simplified the analysis of signal to noise.

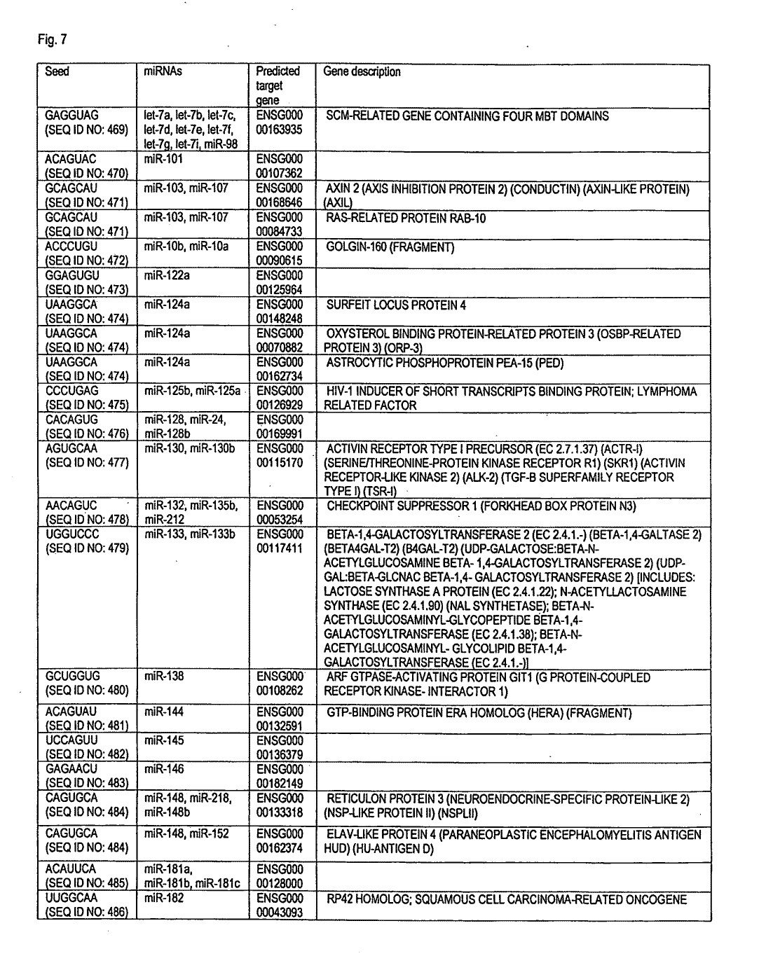

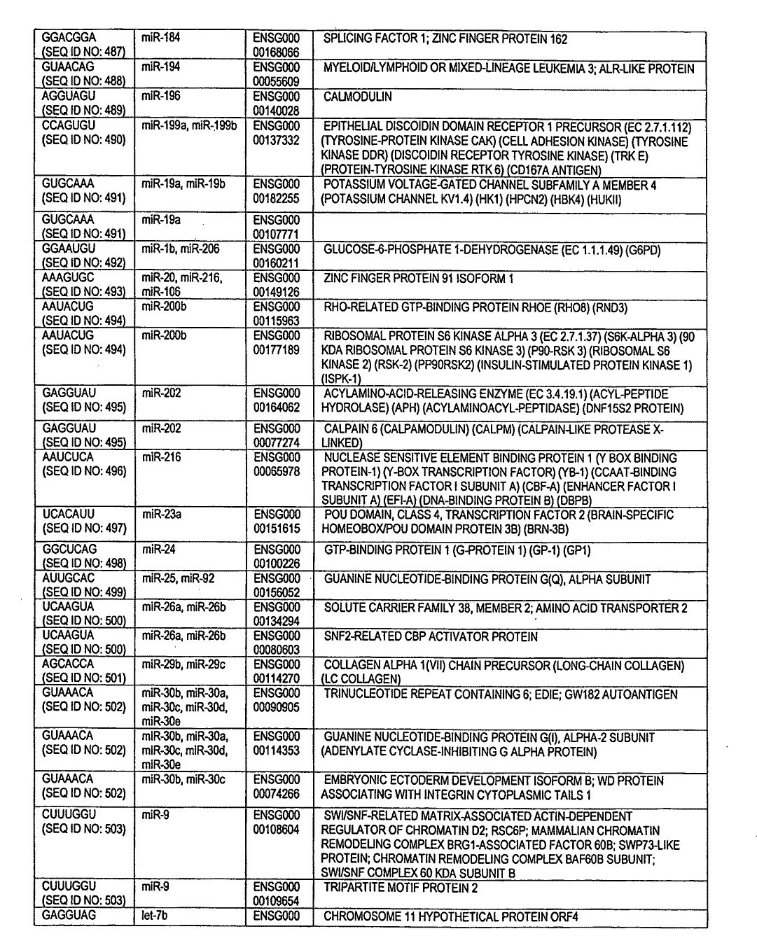

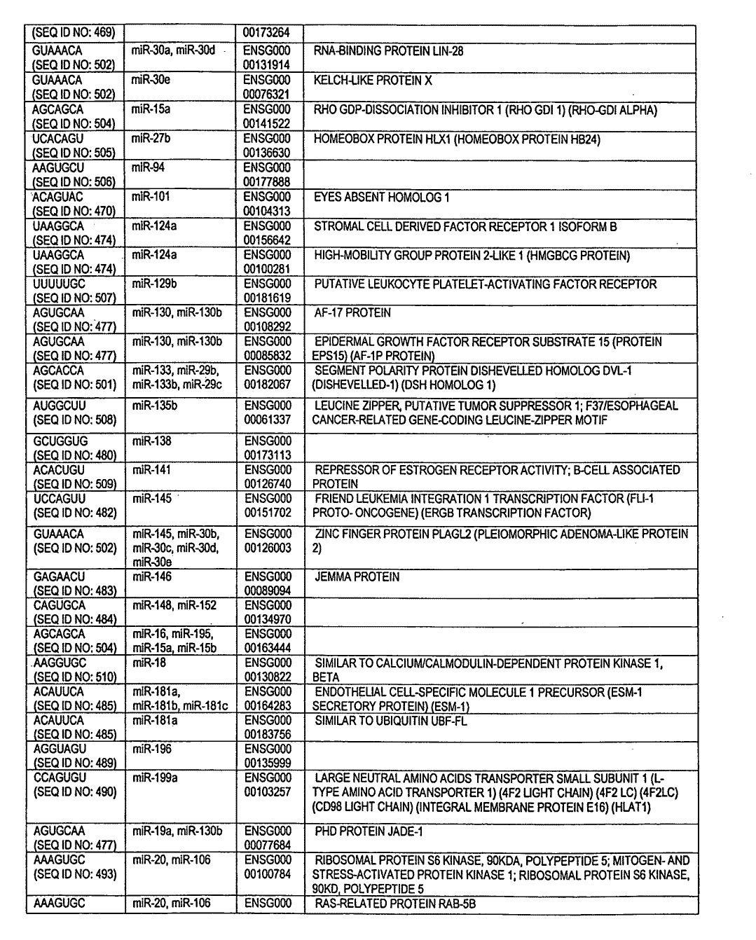

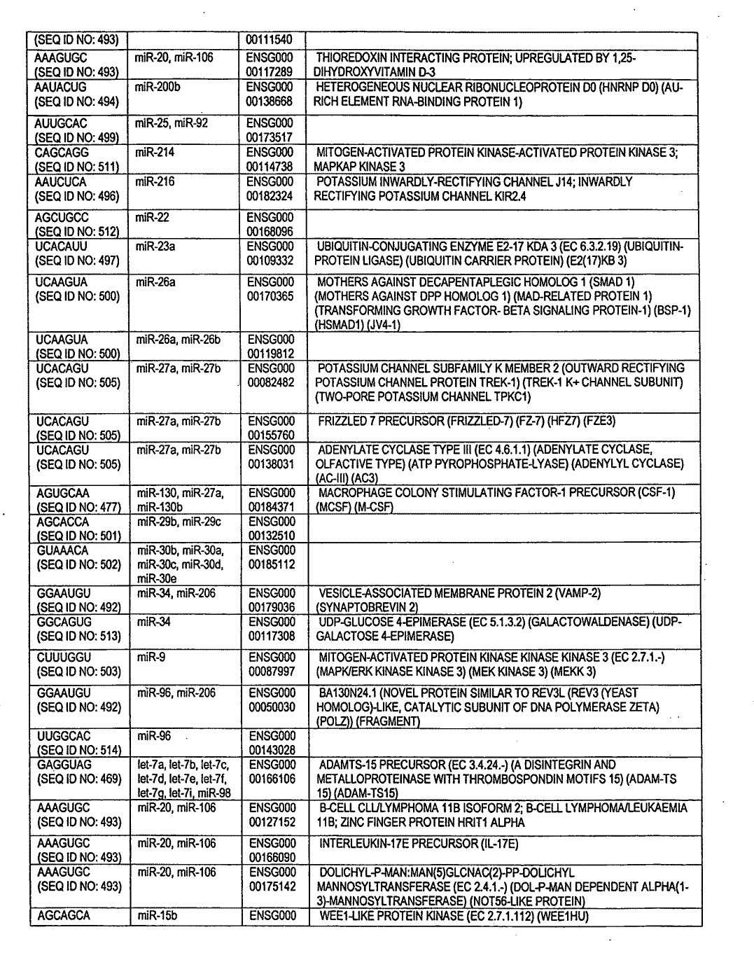

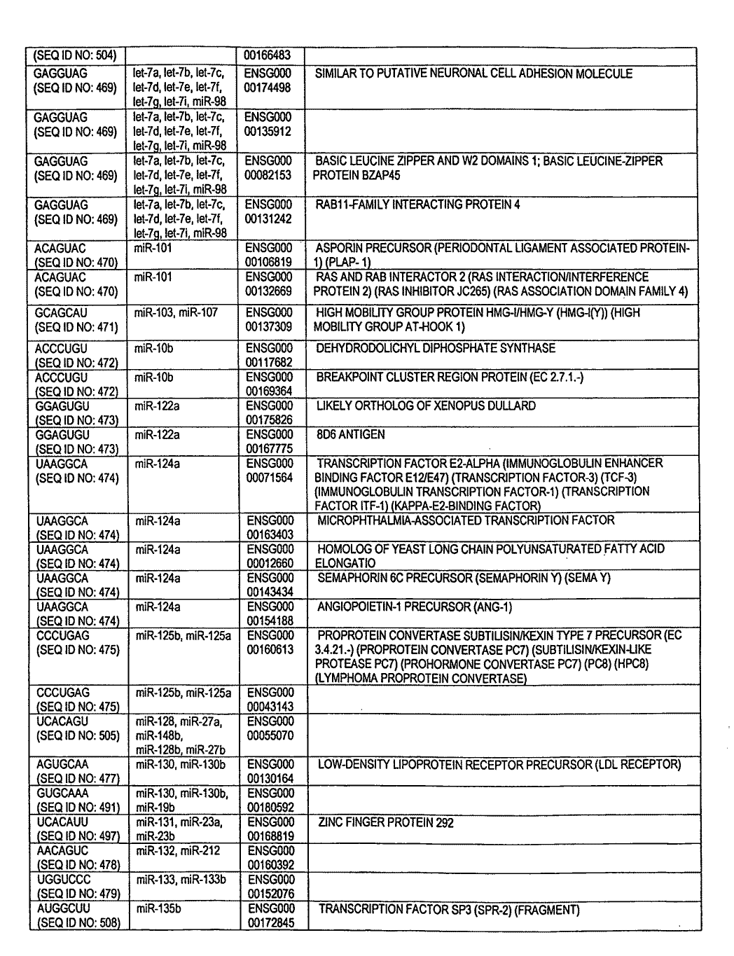

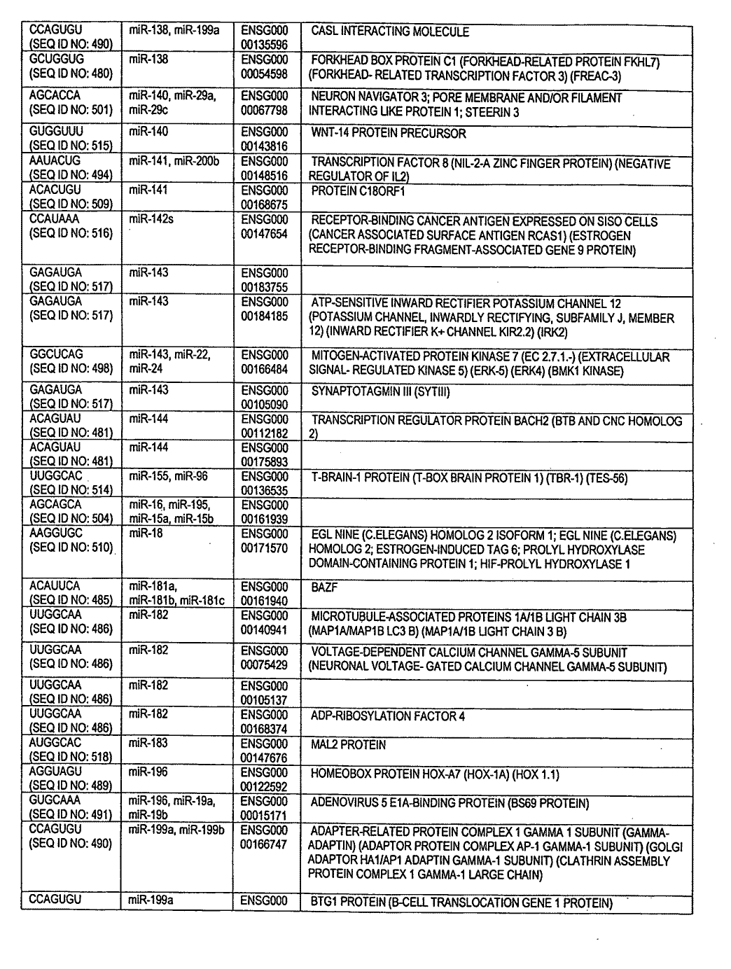

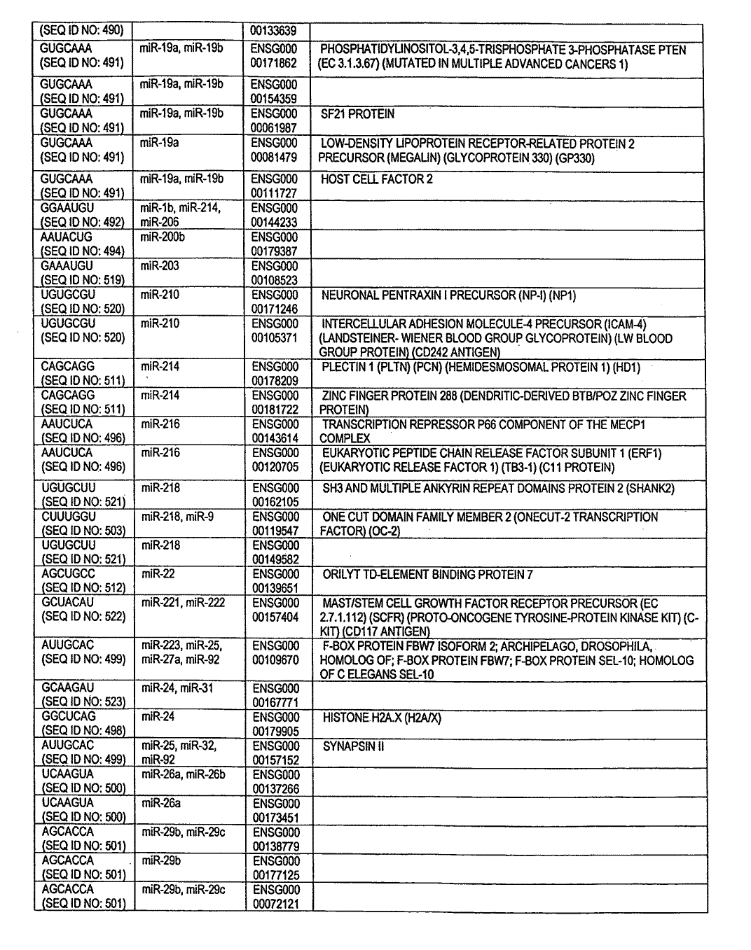

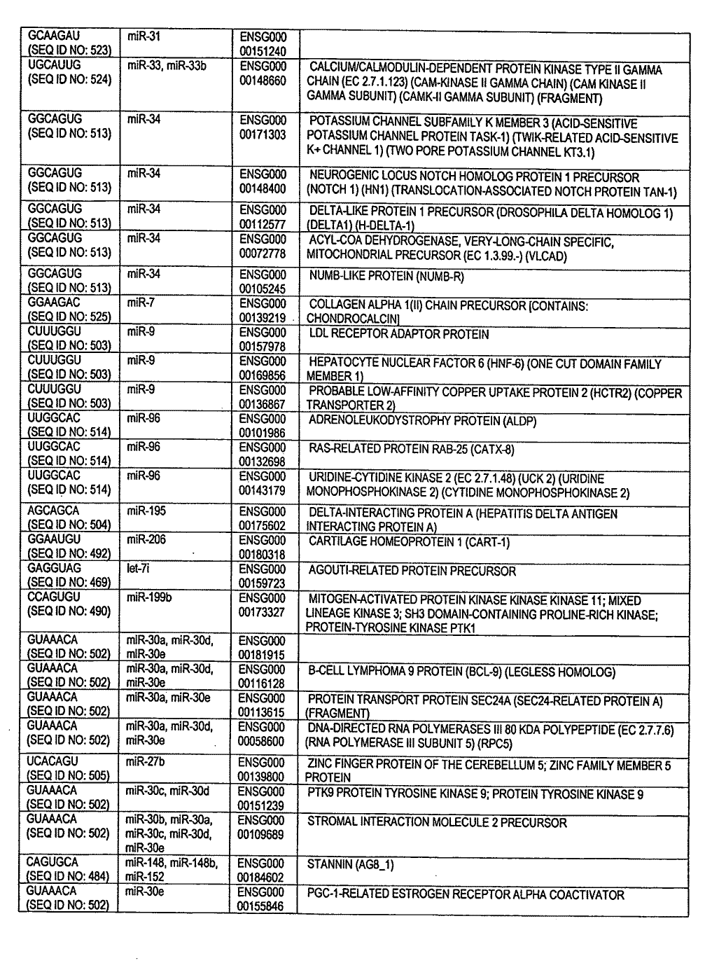

FIG. 7 shows the predicted mammalian targets for miRNAs in rMamm. In this figure, the 442 genes in this set were predicted as targets of rMamm miRNAs by TargetScan in human, mouse, and rat orthologs. MiRNAs with identical seeds that were predicted to target the same gene are shown in a single row of the table. MiRNAs with different seeds that target the same gene are listed on separate lines.

To predict mammalian miRNA targets, the nrMamm set of miRNAs was searched against orthologous human, mouse, and rat 3′ UTRs derived from the Ensembl classification of orthologous genes. Using R_{C}=200, Z_{C}=4.5, and T=20, TargetScan identified an average of 5.7 targets per miRNA (FIG. 2A). This number of predicted targets (the “signal”) was compared to the number of targets predicted for cohorts of shuffled (i.e., randomly permuted) miRNAs (the “noise”). As described below, these shuffled sequences were carefully screened to ensure that the estimates of noise were as accurate as possible, and not artefactually low. An average of only 1.8 targets were identified per shuffled miRNA sequence, for a signal:noise ratio of 3.2:1. This ratio was higher than the roughly 2:1 ratio observed for targets of the nrMamm miRNA set predicted using only the human and mouse UTRs (FIG. 2A), underscoring the importance of evolutionary conservation across multiple genomes in this approach. The signal:noise ratio improved to 4.6:1 when conservation was required additionally in the fourth and most divergent species, Fugu rubripes, using the nrVert set of miRNAs (FIG. 2A).

FIG. 2A illustrates the mean number of predicted targets per miRNA for authentic miRNAs (filled bars), and mean and standard error of number of predicted targets per shuffled sequence for 4 cohorts of randomized miRNAs (open bars). Genomes used for identification of targets are listed below corresponding bars. The nrMamm set of 79 miRNAs was used for human/mouse and human/mouse/rat; the nrVert set of 55 miRNAs was used for human/mouse/rat/Fugu.

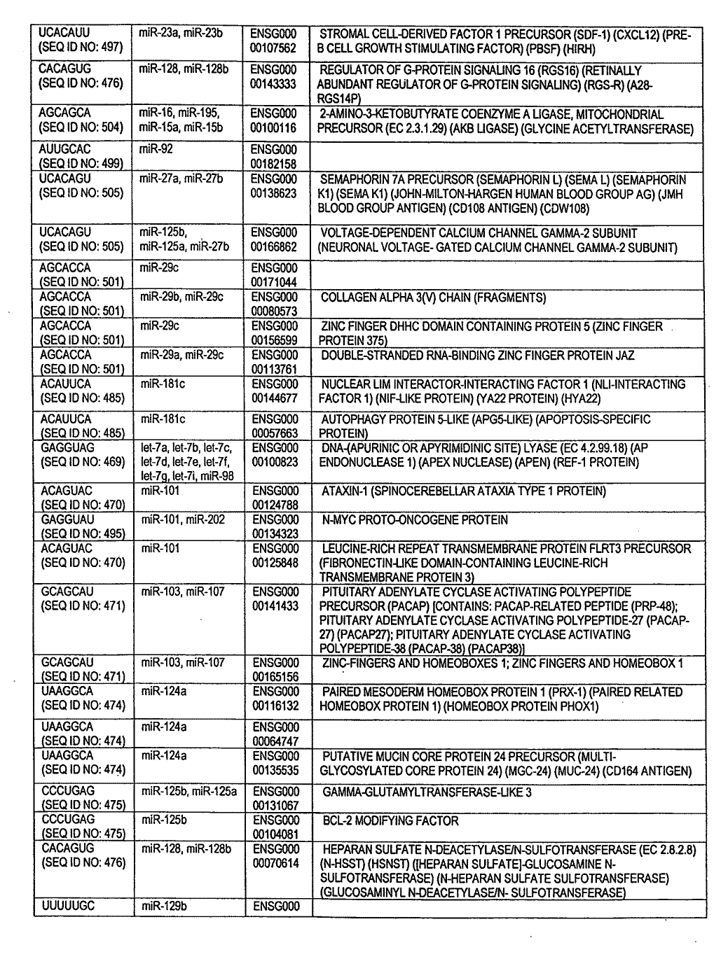

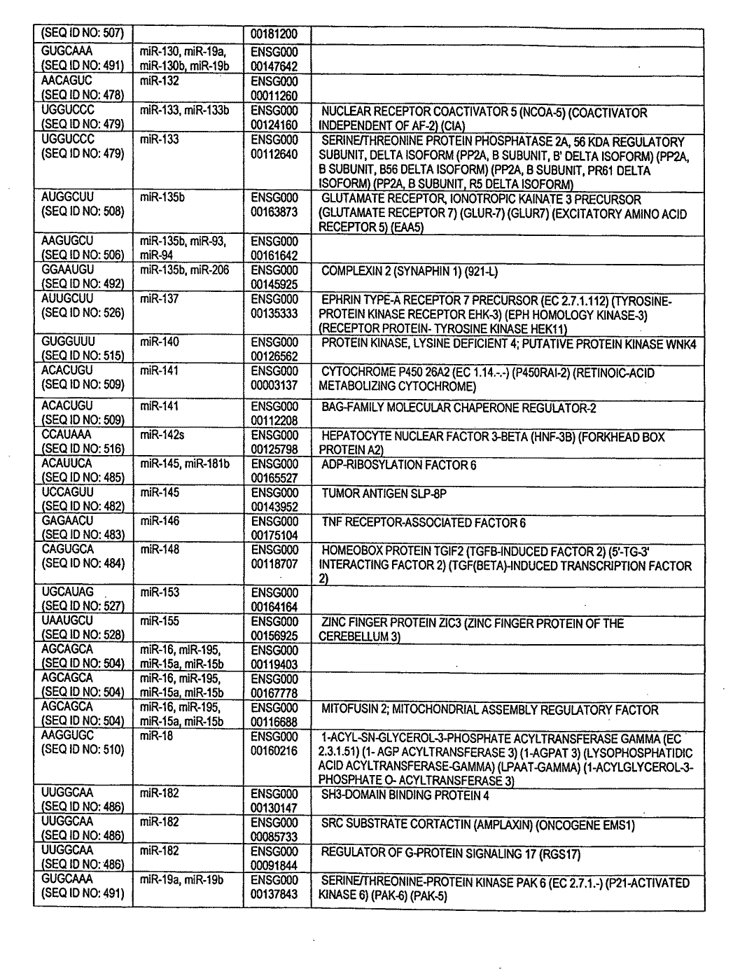

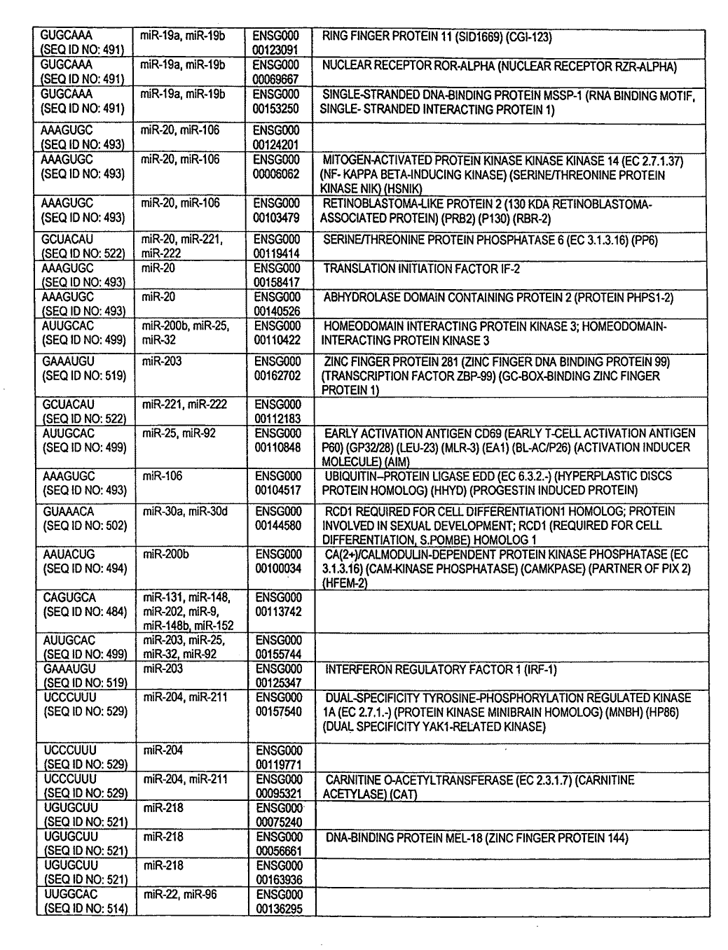

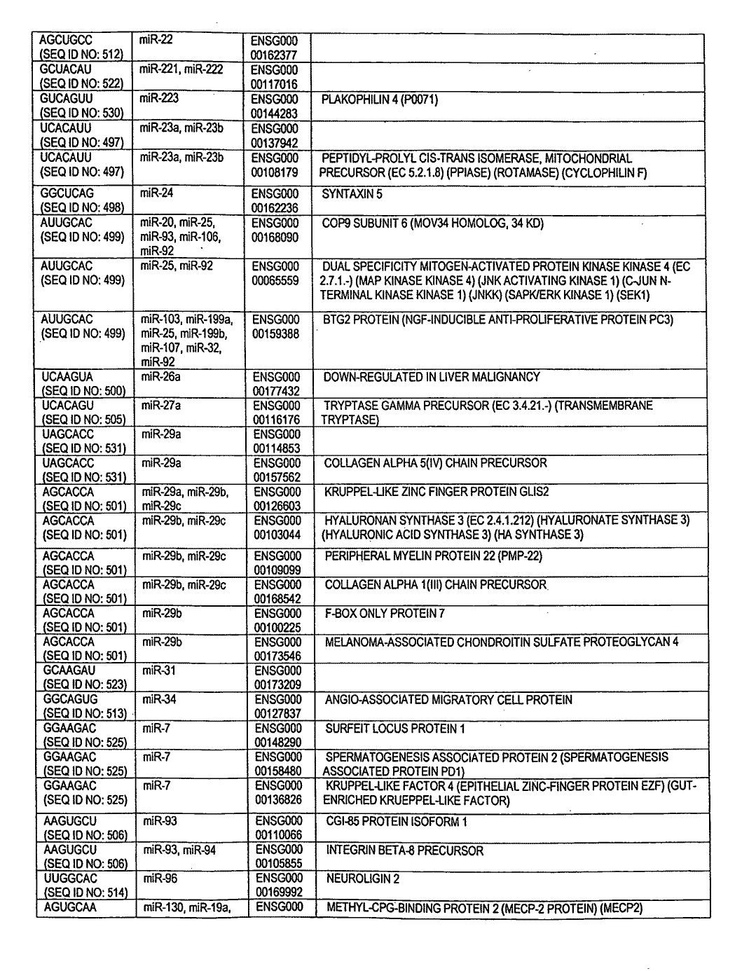

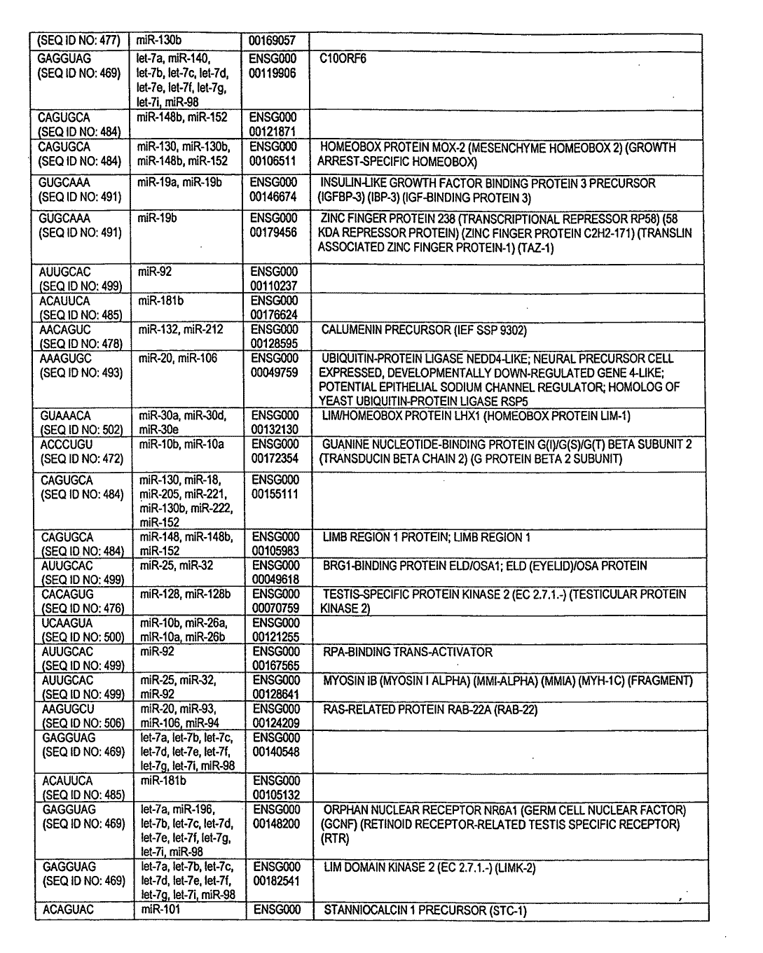

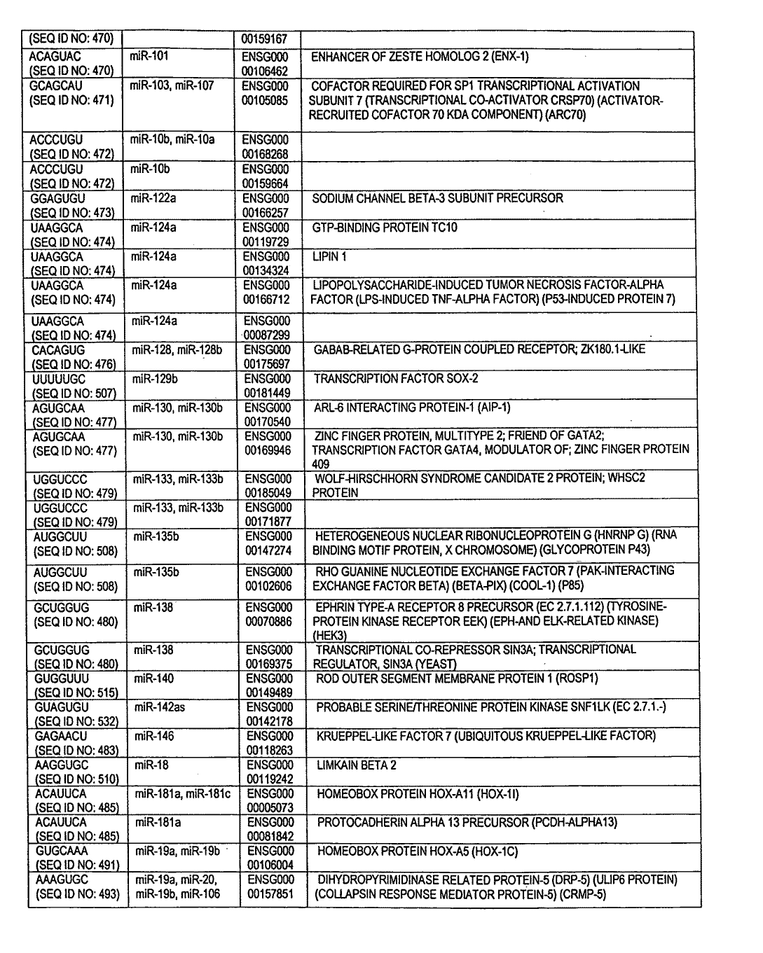

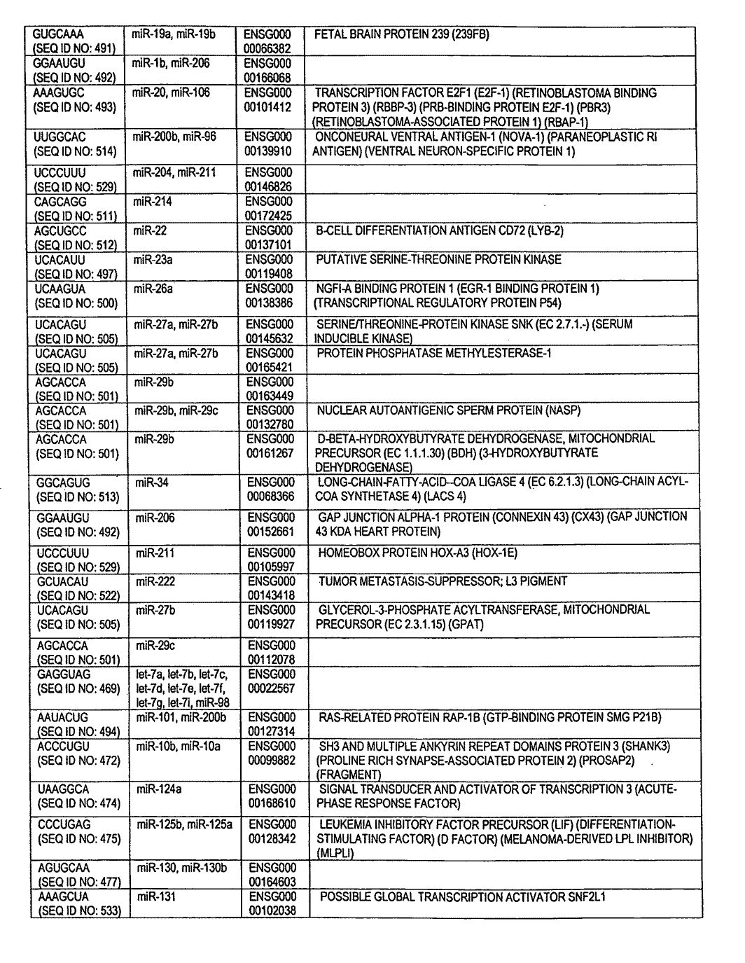

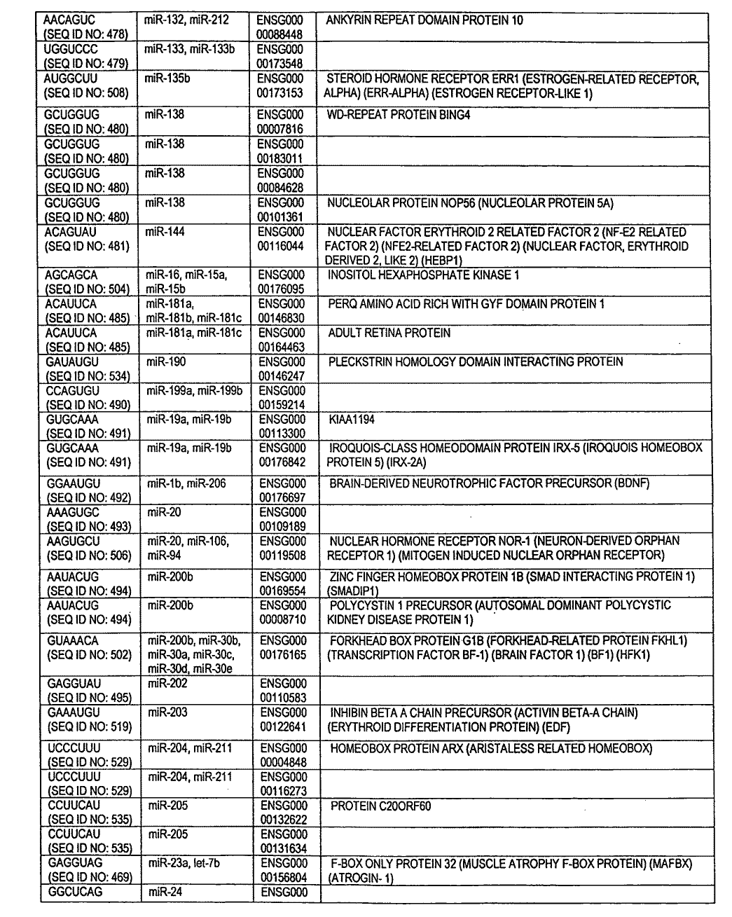

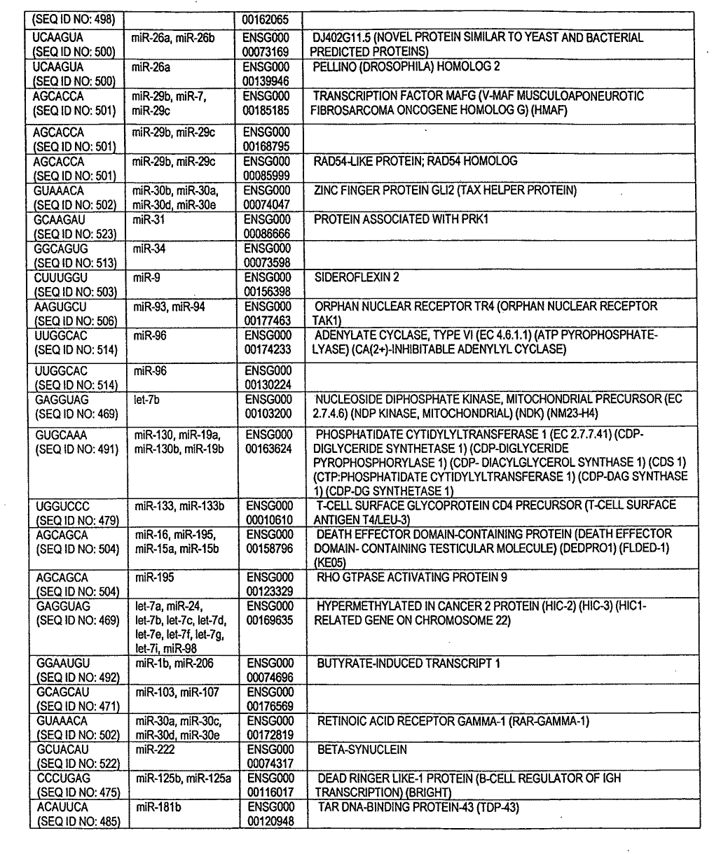

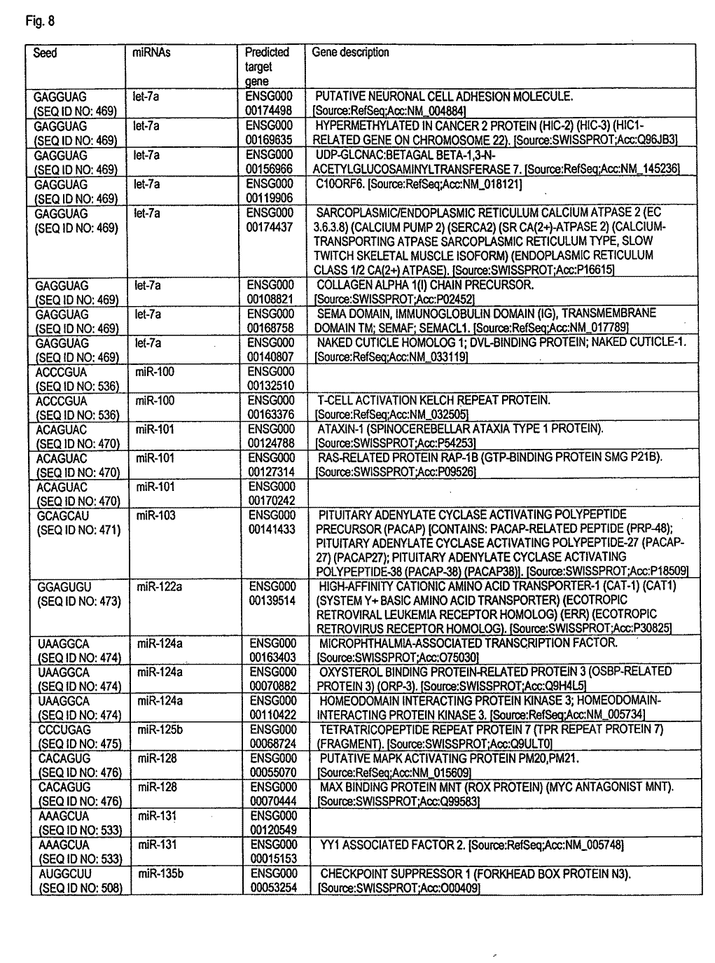

Although the signal:noise ratio improved as more genomes were included, the number of predicted targets per miRNA decreased, even though R_{C} and Z_{C} were relaxed to 350 and 0, respectively, and the value T=10 was used for the four-species analysis (FIG. 2A). Several factors might contribute to this effect, including the increased chance that an orthologous gene will be missing from the annotations of one genome as the number of organisms is increased. For example, the number of ortholog pairs available in human-mouse, 17,166, decreased to 14,539 ortholog sets in human-mouse-rat, and 10,276 ortholog sets in human-mouse-rat-Fugu. In addition, some miRNA:target interactions might not be conserved between mammals and fish. Another factor is that some features used in this method to achieve an acceptable signal:noise ratio might not be strictly required for miRNA regulation. For example, although most known invertebrate miRNA target sites have 7-nucleotide Watson-Crick seed matches (or longer matches), some do not, such as lin-41, a target of the C. elegans let-7 miRNA. Thus, increasing the number of species increases the probability that the orthologous UTR of one or more species harbors functional sites that fail to satisfy the criteria required for TargetScan detection. Nonetheless, in 115 cases involving the UTRs of 107 genes the predicted target sites were sufficiently conserved to be detected by TargetScan in orthologous UTRs from all four vertebrates. Details of these predictions are given in FIG. 8, which shows the predicted vertebrate targets for miRNAs in nrVert. This figure shows the orthologous genes for this set scored highly as targets of nrVert miRNAs in all four organisms studied. MicroRNAs with different seeds that target the same gene are listed on separate lines.

The shuffled control sequences should, in some cases, preserve all relevant compositional features of the authentic miRNAs. For example, when compared to the seeds of shuffled cohorts that had not been screened to control for the expected number of target sites and the expected strength of miRNA:target site interactions, the seeds of vertebrate miRNAs have approximately 1.4 times as many seed matches in vertebrate UTRs. Specifically, the seeds of vertebrate miRNAs each had an average of about 2100 perfect-complement matches in masked vertebrate UTR regions, whereas random heptamers with the same base composition averaged only about 1500 matches. The high number of additional matches seen for the miRNA seed (and also for the antisense of the seed), argues against the biological significance of most of these matches. Instead, these excess matches appear to be the consequence of dinucleotide composition biases shared between vertebrate miRNAs and UTRs, which must be controlled for in order to avoid artificially high estimates of TargetScan signal:noise ratios (particularly in an algorithm that looks for multiple matches). Therefore, it was important to ensure that the shuffled miRNA controls matched the corresponding miRNAs closely in all sequence properties that impact the expected number and quality of TargetScan target sites. The properties considered included: 1) the expected frequency of seed matches in the UTR dataset; 2) the expected frequency of matching to the 3′ end of the miRNA; 3) the observed count of seed matches in the UTR dataset; and 4) the predicted free energy of a seed:seed match duplex. A miRNA shuffling protocol was thus developed to generate randomized control sequences that possess all of these properties. For a given miRNA sequence, this protocol generates a series of random permutations with the same length and base composition as the miRNA, until a shuffled sequence is found that matches the parent miRNA closely in each of the four criteria listed above.

The miRNA shuffling protocol was used to calculate expected frequencies using a first-order Markov model of 3′ UTR composition that accounts for the long-recognized impact of dinucleotide frequency biases on the counts of longer oligonucleotides. As an additional control, another shuffling protocol was developed, which preserved the precise dinucleotide composition of both the seed and the 3′ end of the miRNA, as well as the seed match count and seed:seed match folding free energy. This protocol was less general than the first protocol in that not every oligonucleotide can be randomized while preserving exact dinucleotide composition, e.g., the only heptamer with the same dinucleotide composition as the miR-100 seed, ACCCGUA (SEQ ID NO: 536), is ACCCGUA (SEQ ID NO: 536) itself. Nevertheless, it was possible to generate controls using the second protocol for 47 of the 79 nrMamm miRNAs, and a signal:noise ratio of 3.5 was observed using this control in the three-mammal analysis (data not shown), comparable to the value obtained for MiRshuffled controls. Because of its wider applicability, the first protocol was used in all reported experiments.

In summary, even when the shuffled control sequences were carefully selected to closely match the corresponding miRNAs in all sequence properties expected to influence the number and quality of target sites, these shuffled controls yielded far fewer targets than did the authentic miRNA sequences. This difference results from an increased propensity of vertebrate UTRs to contain multiple conserved regions of complementarity to authentic miRNAs. Thus, it can be concluded that this propensity reflects a functional relationship between the miRNAs and the identified UTRs; that is, to the extent that the signal exceeds the noise, these identified UTRs may be the regulatory targets of the miRNAs.

### Example 3

Correcting for the estimated rate of false positives, the method used in Examples 1 and 2 thus appears to have identified an average of 5.7-1.8=3.9 true targets conserved across mammals per miRNA (FIG. 2A); thus, the actual number of target genes regulated by each miRNA may be substantially higher. This method treats the 5′ and 3′ ends of miRNAs differently, with perfect base-pairing required for the seed at the 5′ end, but no such requirement at the 3′ end. The importance of complementarity to the 5′ portion of invertebrate miRNAs has been suspected since the observation that complementary sites within the lin-14 mRNA have “core elements” of complementarity to the 5′ segment of the lin-4 miRNA is consistent with this concept. It has been corroborated with the observation that the 5′ segments of numerous invertebrate miRNAs are perfectly complementary to 3′ UTR elements that mediate posttranscriptional regulation or are known miRNA targets. Moreover, the 5′ ends of related miRNAs tend to be better conserved than the 3′ ends, further supporting the hypothesis that these segments are most important for mRNA recognition.

To explore this hypothesis, the method was applied in this example to predict targets of the nrVert miRNA set conserved between human, mouse and rat using versions of the algorithm differing in the miRNA heptamer defined as the seed in step 1 (FIG. 2B). This figure shows the mean number of targets per miRNA using the human/mouse/rat UTR set and alternative miRNA seed positions for the nrVert miRNAs (filled bars) and for cohorts of shuffled controls (open bars). Positions of seed heptamer are indicated under bars; positive numbers indicate position relative to 5′ end of miRNA, negative numbers indicate positions relative to 3′ end of miRNA. Note that the signal:noise for the seed at 2 . . . 8 differed slightly from that of the human/mouse/rat analysis in panel A because a different set of miRNAs was used. FIG. 2C shows conserved heptamers among paralogous human miRNAs. For each position, the number of different heptamers that are perfectly conserved across multiple miRNAs in rMamm is shown.

Consistent with residues at the 5′ end of miRNAs being most important for target recognition, the highest signal:noise ratio was observed when the seed was positioned at or near the extreme 5′ end of the miRNA, with signal:noise values of 2.7, 3.4, and 1.6 observed for seeds at segments 1 . . . 7, 2 . . . 8, and 3 . . . 9, respectively, and signal:noise ratios of 1.3 or less at other seed positions. Thus, it may be that the importance of pairing to segment 2 . . . 8 (or 2 . . . 7 as described in Example 7) for target identification in silico reflects its importance for target recognition in vivo, and this segment may thus nucleate pairing between miRNAs and mRNAs.

Those seed positions that had the highest signal:noise ratios in the sliding seed analysis (FIG. 2B) also had the highest degree of heptamer conservation in paralogous human miRNAs (FIG. 2C). This observation strengthens the assertion that the signal seen above noise in this analysis reflected a functional relationship between the miRNAs and the identified UTRs, because otherwise it would be difficult to explain why the most conserved portions of the miRNA and not other miRNA segments have the greatest propensity to match multiple conserved segments in UTRs.

### Example 4

In this example, the set of target genes predicted using conservation of miRNA complementarity across the three mammals was most suitable in size and quality for systematic analysis of gene function. To obtain as large a set of targets as possible, in this example, the set of orthologous mammalian 3′ UTRs was searched using an expanded set of 121 conserved mammalian miRNAs (rMamm, see FIG. 6) that includes miRNAs that were excluded from the nrMamm set because they had redundant seeds, yielding a total of 854 predicted miRNA:UTR pairs conserved across human, mouse and rat (data not shown).

FIG. 6 shows the human miRNAs and shuffled controls used in this study. The inclusion of each miRNA in the three subsets used in this study (rMamm, nrMamm, and nrVert) is indicated by Y (Yes) or N (No). For those miRNAs in nrMamm, the sequences of the four shuffled variants generated by the first shuffling protocol are listed on the next four lines (labeled miR-X_sh0, miR-X_sh1, etc.).

The 19 miRNAs not in rMamm are those for which Fugu homologs could not be identified. When initially expanding the list of mammalian miRNAs, it was found that the set of 19 mammalian miRNAs that were conserved between human and rodents but for which a Fugu homolog was not found gave an unacceptably low signal:noise ratio of 1.2:1, even though the analysis did not extend to the Fugu UTRs. Accordingly, the rMamm set was restricted to those miRNAs with recognized Fugu homologs. The higher signal seen for the more broadly conserved miRNAs can be explained by the idea that miRNAs with larger numbers of targets would be under greater selective constraint, and therefore less likely to change during the course of evolution. Thus more broadly conserved miRNAs would be likely to have more targets and consequently a higher TargetScan signal. This observation again supports the conclusion that TargetScan is detecting authentic targets, since otherwise it would be difficult to explain the observed difference in signal:noise for broadly conserved miRNAs relative to that of less broadly conserved miRNAs.

The 854 miRNA:UTR pairs represented UTRs of just 442 distinct genes because many genes were targeted by multiple miRNAs. In these cases, the miRNAs were usually, but not always, from the same paralogous miRNA family, often with the same seed heptamer. In those cases where the same UTR was targeted by multiple miRNAs from different families (54 genes), the target sites generally did not overlap, consistent with simultaneous binding and regulation of some target genes by combinations of miRNAs. A complete list of the 442 target genes and the corresponding miRNAs is provided (FIG. 7). A representative, abbreviated list also appears as FIG. 4, where genes were chosen on the basis of high biological interest. In FIG. 4, the 442 predicted targets conserved between human, mouse and rat were ranked based on the number of references listed in the publicly accessible RefSeq GenBank flatfiles (11/10/03 download). The top 45 most referenced predicted targets are shown, grouped on the basis of Gene Ontology annotations. The last six digits of the Ensembl ID are also shown (ENSG00000#). MicroRNAs with different seeds that target the same UTR are listed on separate lines. Genes involved in transcription, signal transduction and cell-cell signaling dominate this list, including a number of human disease genes such as the tumor suppressor gene PTEN, and the proto-oncogenes E2F-1, N-MYC, C-KIT, FLI-1, and LIF.

### Example 5

One limitation of the existing sequence databases that complicates the systematic identification of miRNA targets is that UTR annotations are often absent or incomplete. In order to compensate for this limitation, in examples 1, 2, 3, 4, and 8, each annotated 3′ UTR was extended with 2 kb of 3′ flanking sequence. Using extended UTRs substantially increased the number of predicted targets, with signal-to-noise ratios at least as high as they were for unextended UTRs, suggesting that extension of the annotated UTRs allows detection of many additional authentic target genes. Manual inspection of the 15 UTR regions tested in the reporter assays revealed that in all but one of these cases the tested target sites were contained within regions whose status as UTRs was supported by known ESTs and predicted polyadenylation sites, even though some of these regions are not yet annotated as human UTRs. For the single exception, the Notch1 gene, the tested target sites were all located downstream of the annotated 3′ UTR of the human gene, and the end of the annotated Notch1 3′ UTR was supported by a predicted polyadenylation site and alignment of multiple ESTs. However, Notch1 might have additional 3′ UTR isoforms; many human genes—perhaps as many as 50% or more of the genes in the genome—may have alternative polyadenylation sites. In order to investigate the potential expression of the tested Notch1 target sites, which gave a positive result in the assay for miRNA regulation (data not shown), an RT-PCR assay was used with polyA-selected RNA from a pool of human tissues. Consistent with the possibility that these sites lie within an alternative UTR isoform of Notch1, an RT-dependent product of the correct size and sequence was observed (data not shown).

### Example 6

To assess target gene functions, in this example, the frequency of specific gene ontology (GO) molecular function classifications was evaluated among the predicted targets of the nrMamm miRNAs and their shuffled control sequences (FIG. 5). Predicted miRNA targets populate many major GO functional categories, and for each of these categories the number of targets for the real miRNAs exceeded the average for the shuffled cohorts. Therefore, despite the presence of false positives, the data in FIG. 7 may indicate that mammalian miRNAs are involved in regulation of target genes with a wide spectrum of molecular functions.

In this example, the proportion of genes that fell in each of the GO molecular function and GO biological process categories for the predicted targets of miRNAs was also compared, for targets of shuffled control sequences, and for the initial set of orthologous genes (FIGS. 5 and 9). The targets of the shuffled cohorts were enriched relative to the initial set of orthologous genes in certain GO biological process categories such as development (14% versus 8%) and transcription (13% versus 9%) (FIG. 9) and in the molecular function categories such as nucleic acid binding (21% versus 15%), DNA binding (15% versus 10%) and transcriptional regulator activity (10% versus 6%) (FIG. 5). The biases seen for the shuffled cohorts are likely to result primarily from the TargetScan requirement for conserved segments in the 3′ UTRs of predicted targets, and may reflect differences in the occurrence of 3′ UTR regulatory elements in different classes of genes.

Gene ontologies were assigned to human genes from the Ensembl database by cross-referencing Ensembl identifiers with GO identifiers using EnsMart version 15.1 (available at http://www.ensembl.org/EnsMart). The Gene Ontology Consortium database was retrieved from http://www.geneontology.org and function and process ontologies were compiled for all predicted target genes. In addition to the assigned categories, each gene was considered as having all more general (“parent”) categories within the “Molecular Function” and “Biological Process” ontologies. In FIGS. 5 and 9, sets of GO categories were selected that were both broad enough to contain a significant fraction of the predicted targets and specific enough to be meaningful. Because the GO descriptions are not mutually exclusive, the sum of the percentages in these tables is not interpretable. GO categories were also used to produce the categories in FIG. 4. To be included in a category, a gene had to be annotated with at least one out of a set of GO categories. The sets of GO categories used were: Regulation of transcription/DNA binding (GO:0003700, GO:0003713, GO:0003714, GO:0016563, or GO:0045449), Signal transduction/cell-cell signaling (GO:0004871, GO:0004872, GO:0007154, GO:0007165, GO:0007267 or GO:0008083), and Transport (GO:0006810 or GO:0006811).

FIG. 5 is a table showing the molecular function classification of predicted miRNA targets. In this figure, the number and percentage of genes annotated with various Gene Ontology molecular function categories are shown for targets of nrMamm miRNAs, targets of shuffled control miRNAs (mean of four cohorts), and for the initial set of orthologous human-mouse-rat genes. If GO categories have a parent-child relationship, the child is indented. Because one gene can belong to multiple GO categories, the sum of the percentages in each column is not interpretable.

FIG. 10 is a table illustrating the targets of shuffled control sequences. The 558 shuffled sequence:UTR pairs found human, mouse, and rat that were predicted for any of the four cohorts of MiRshuffled variants of nrMamm miRNAs are shown. FIG. 9 illustrates biological function classes of predicted miRNA targets and controls. The number and percentage of UTRs annotated in various Gene Ontology biological process categories are shown for the 400 predicted miRNA-UTR pairs for nrMamm miRNAs; the miRNA-UTR pairs predicted with randomized miRNAs (average of 4 cohorts); and for the total set of orthologous genes conserved between human, mouse, and rat. For cases in which GO categories with a parent-child relationship are shown, the child is indented. Note that the GO categories are not mutually exclusive.

In the GO biological process classifications, the predicted regulatory targets of authentic miRNA genes were enriched in the development category but no more than the targets of shuffled controls, and were substantially more enriched for genes involved in transcription (21% of miRNA targets versus 13% of shuffled targets versus 9% of the initial dataset) and regulation of transcription (21% versus 12% versus 8%) (FIG. 9). In terms of the GO molecular function classifications, targets of authentic miRNAs were enriched in the categories DNA binding (20% versus 15% versus 10%), transcription regulatory activity (14% versus 10% versus 6%), and nucleotide binding (13% versus 8% versus 9%) (FIG. 5).

The differing numbers of predicted targets in the similar-sounding categories “regulation of transcription” (GO biological process classification) and “transcription regulatory activity” (GO molecular function classification) suggests an investigation of the gene content of these two categories. Inspection of the lists of genes showed that all but two of the predicted target genes in the “transcription regulatory activity” category were also included in the larger “regulation of transcription category”, but that the latter category also contained more than two dozen additional target genes, the annotation of which generally supported a role in control of transcription. The GO process category “regulation of transcription” (FIG. 9) therefore appears to provide a more complete listing of known and putative transcription factors.

The proportion of the predicted mammalian miRNA target genes involved in the GO process categories “transcription” and “regulation of transcription” was significantly higher than that seen for either shuffled targets or for the initial gene set (P<0.001). Nonetheless, this bias was much lower in magnitude than that seen in plants: of the 49 targets predicted in a systematic search for complementarity to plant miRNAs, 69% were members of transcription factor gene families. Examples of other types of predicted mammalian targets include translational regulators (e.g., COP9 subunit 6, ERF1), regulators of mRNA stability (e.g., HU-Antigen D), structural proteins (e.g., collagen), and enzymes (e.g., G6PD). The set of predicted miRNA targets conserved across all four vertebrates (FIG. 8) was also somewhat biased toward genes involved in transcription but had annotated functions consistent with the broad array of biological activities seen for the larger mammalian target set. It was concluded that although mammalian miRNAs are sometimes at the center of gene regulatory networks, where they regulate genes, such as transcription factors, that regulate other genes, they are more likely than plant miRNAs to be at the periphery of the regulatory networks, where they regulate genes with a variety of molecular functions.

The predicted mammalian targets also differ from the plant targets with respect to biological function. Nearly all of the transcription factors (TFs) predicted to be plant miRNA targets have known or implied roles in plant development, as do several of the other predicted plant targets. By comparison, only ˜13% of predicted mammalian miRNA targets were involved in development according to the GO biological process categories (FIG. 9). An important caveat to this analysis is that gene annotation and GO categories are still evolving. Nonetheless, this data suggest that mammalian miRNAs are not exclusively, or even primarily, involved in the traditional miRNA role of developmental control.

### Example 7

With the availability of the chicken and dog genome assemblies, together with updated annotations of the human, mouse, and rat genomes, fundamental principles described in previous examples can be used to achieve more sensitive miRNA target predictions are demonstrated in this example. Requiring target-site conservation in all five genomes (human, mouse, rat, dog, and chicken) reduced the noise (estimated number of false-positive predictions) such that the TargetScan score and rank cut-offs could be dramatically relaxed or eliminated. Moreover, the requirement of a 7-nucleotide match to the seed region of the miRNA (nucleotides 2-8) was relaxed to require a 6-nucleotide match to a reduced seed comprising nucleotides 2-7 of the miRNA while still retaining modest specificity. Running the TargetScan program in this way, without cut-offs, amounted to predicting a target simply by virtue of the presence of at least one 6-nucleotide seed match to the miRNA in orthologous UTRs of each of the five genomes. This algorithm is a simplified version of the TargetScan algorithm, described in previous examples, that searches multiple alignments to identify conserved W—C hexamer seed matches to the designated seed region of the miRNA (bases 2 to 7). In a preferred embodiment, known as the TargetScanS algorithm, additional specificity of target prediction is achieved by requiring that these conserved 6-nucleotide seed matches are flanked by either a Watson-Crick (W—C) match to the m8 position of the miRNA or a conserved adenosine in the t1 position of the target, designated as the t1A anchor.

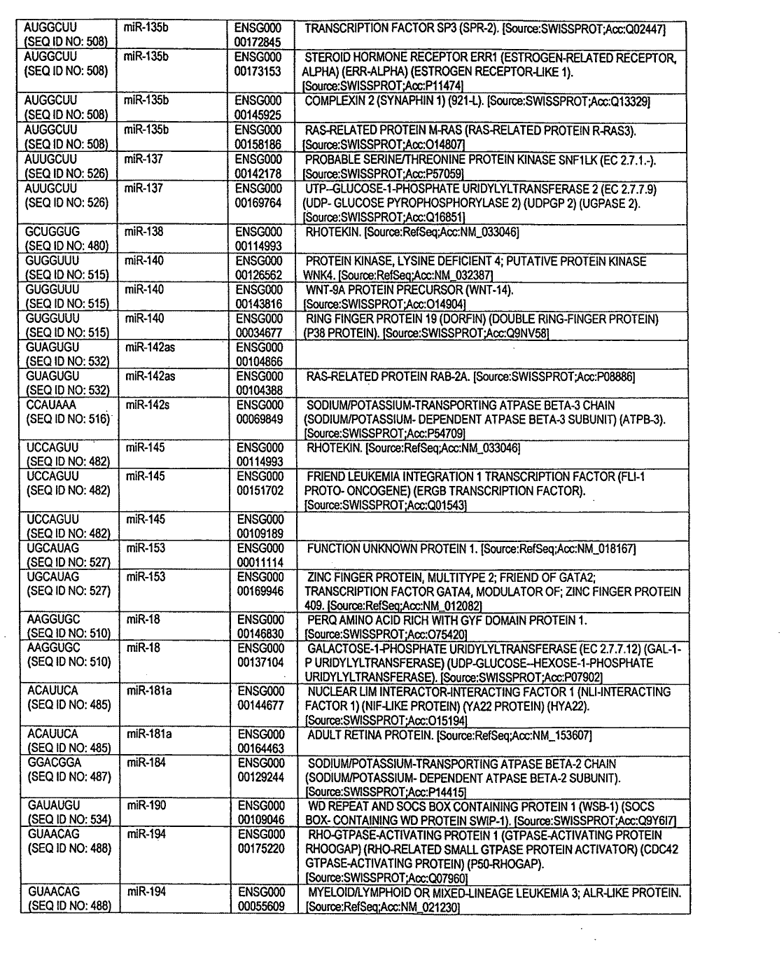

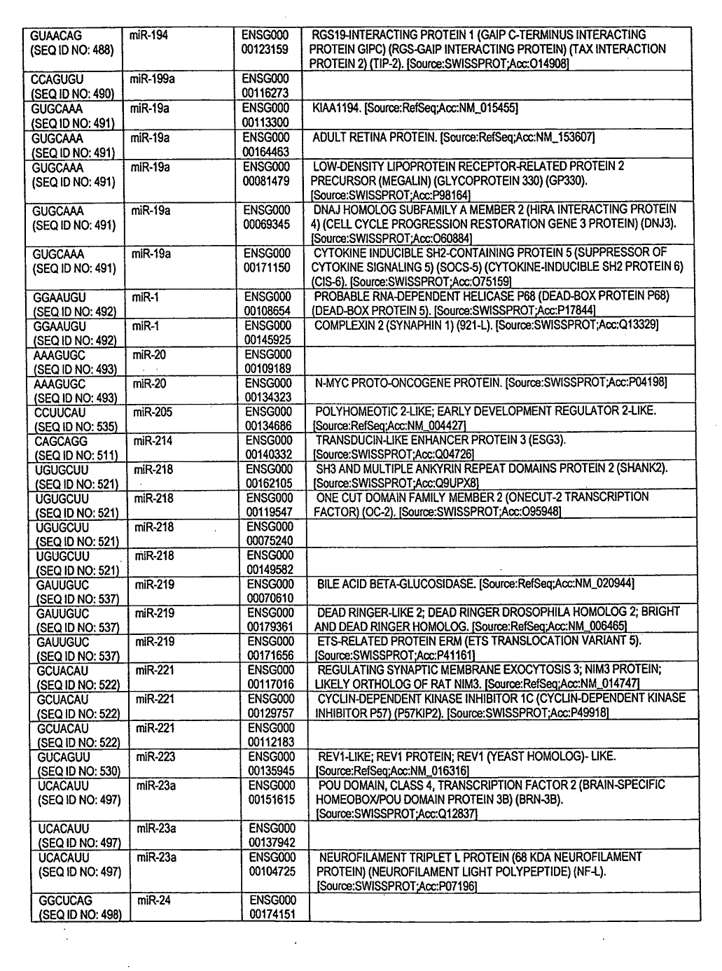

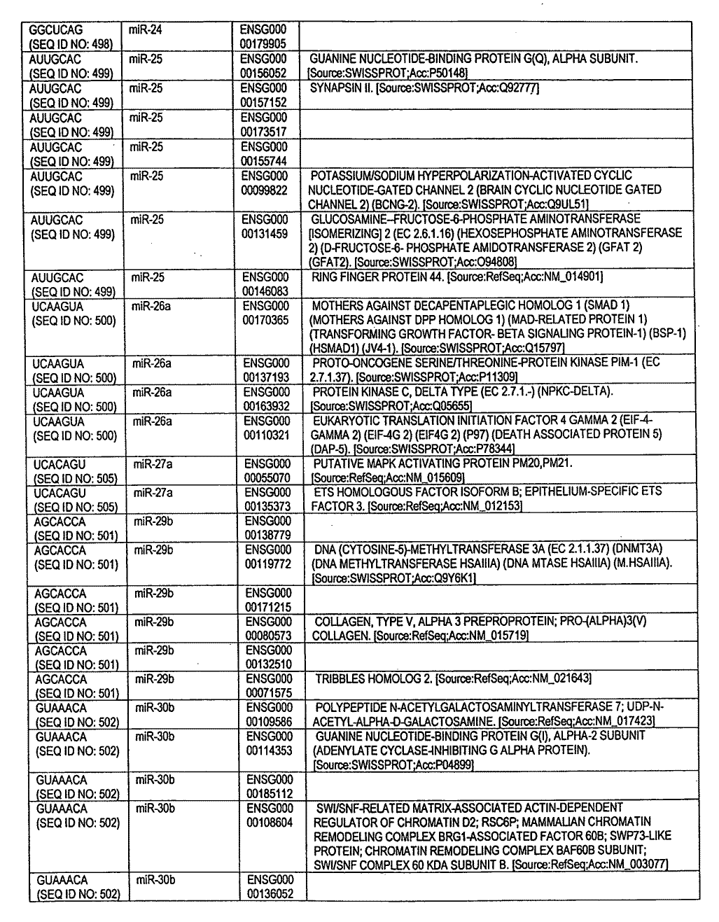

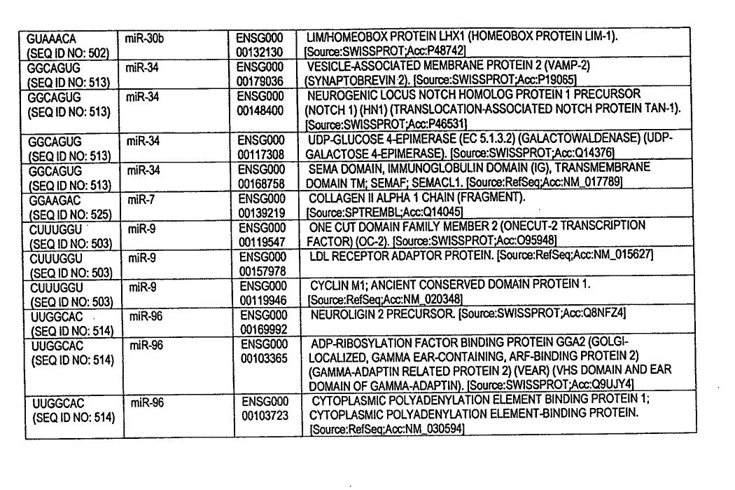

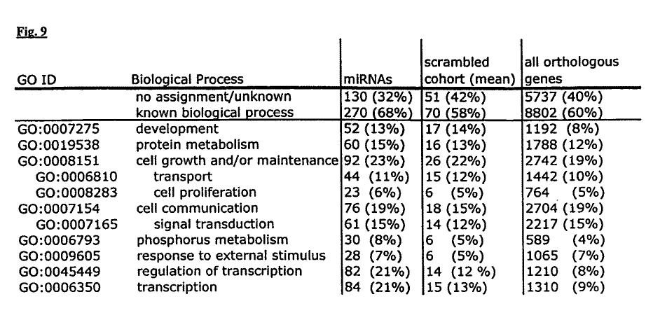

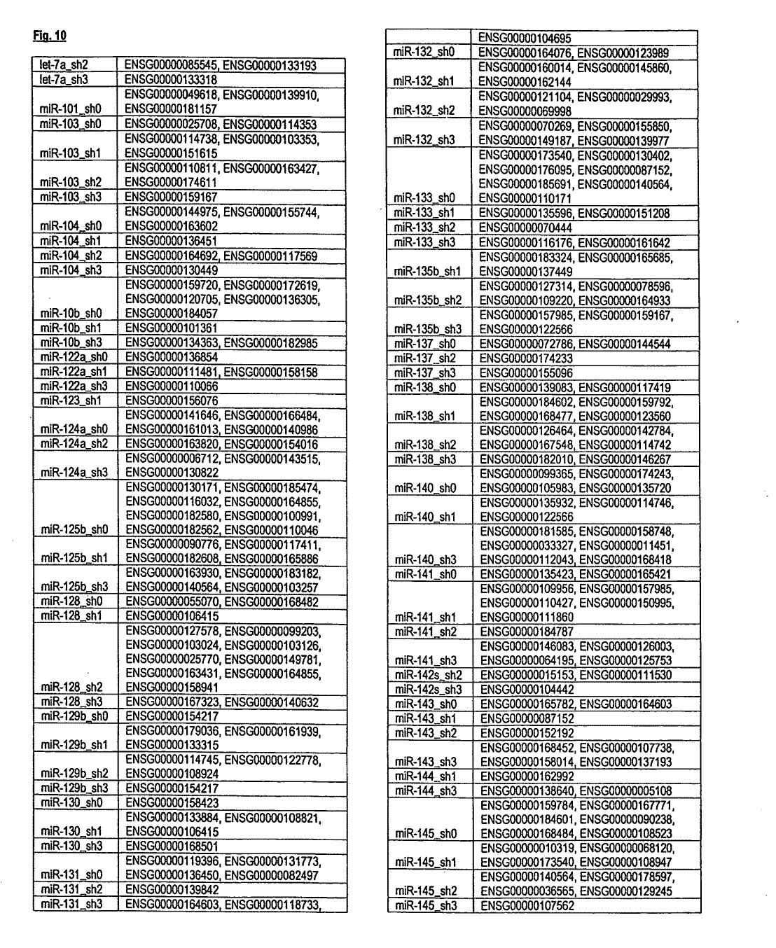

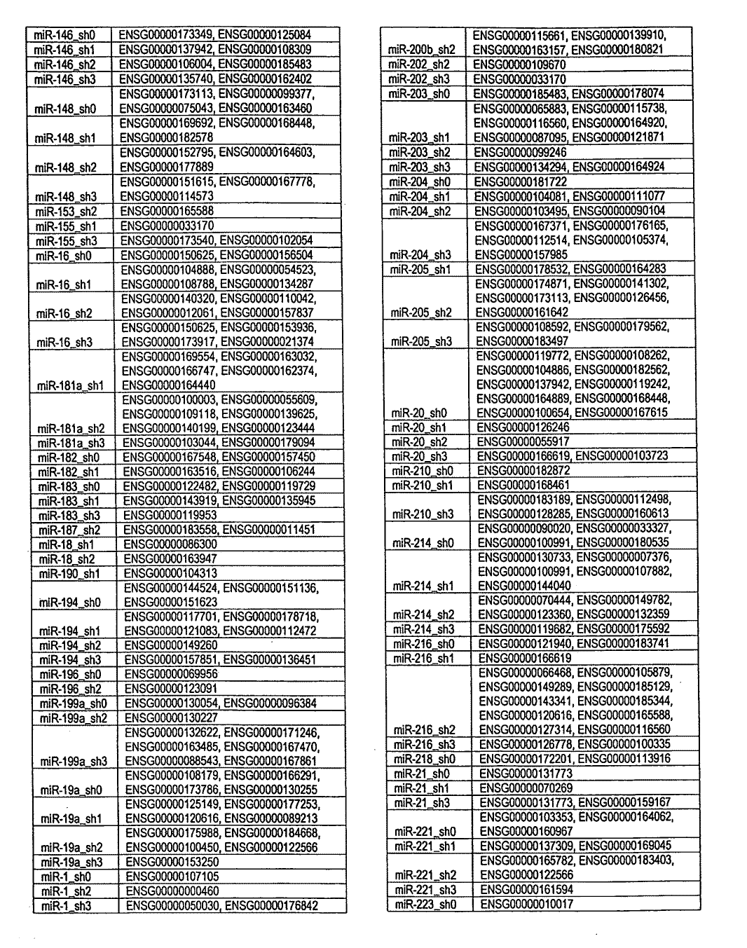

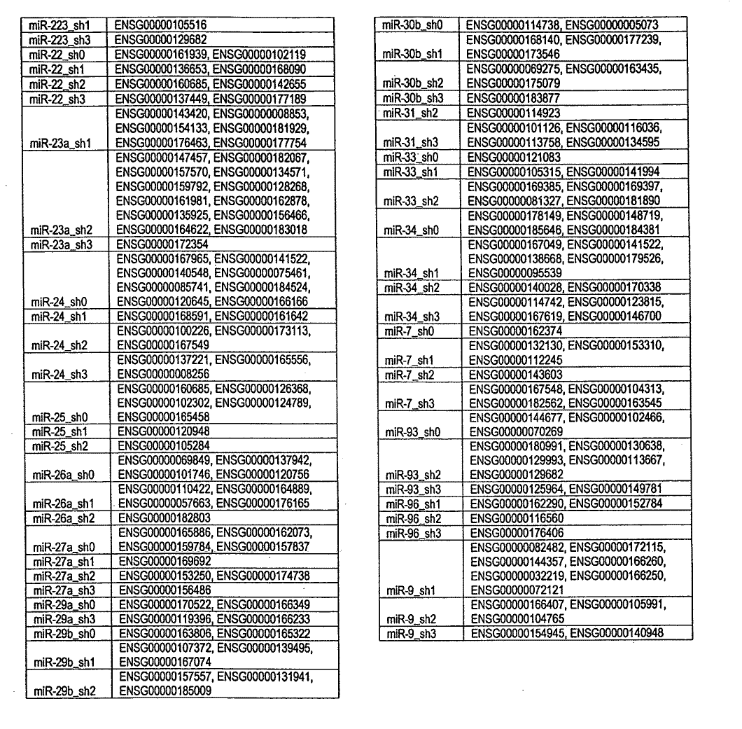

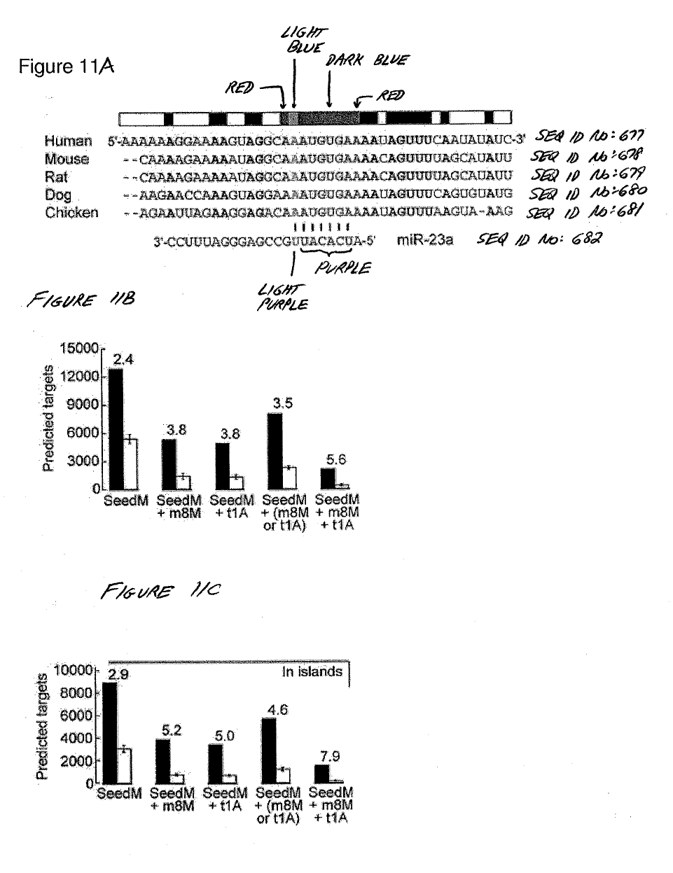

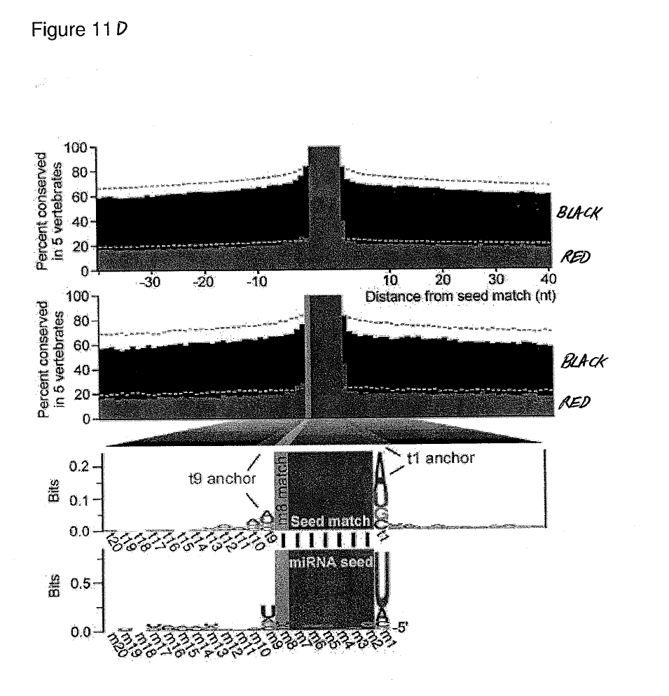

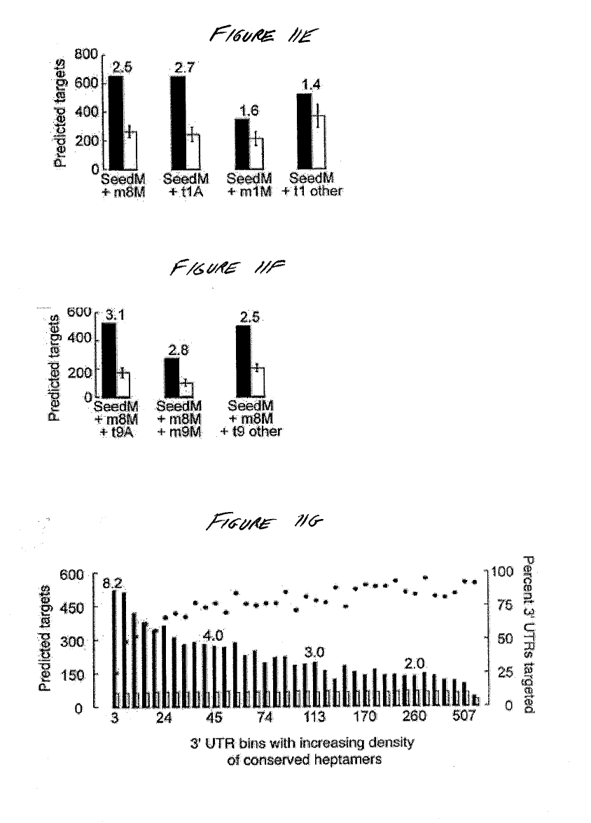

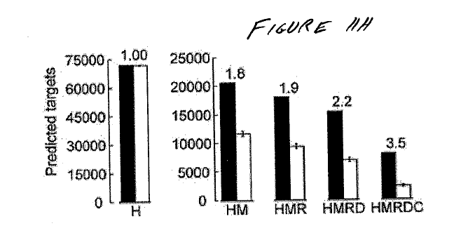

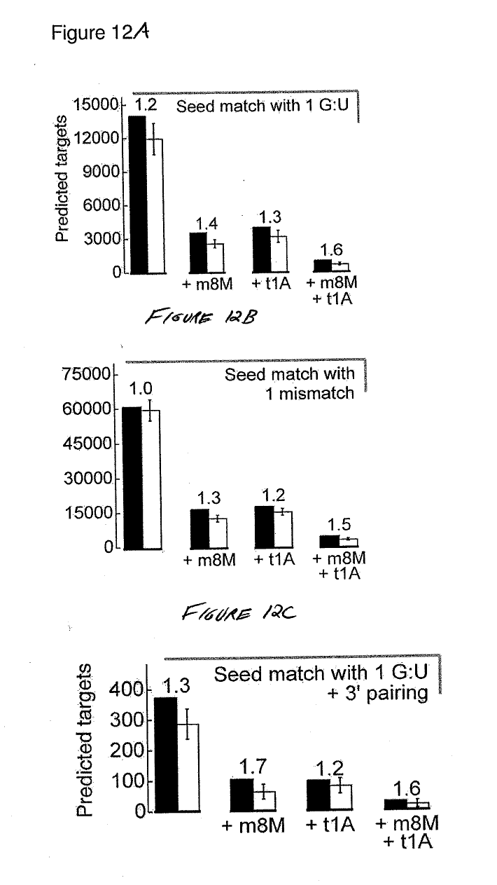

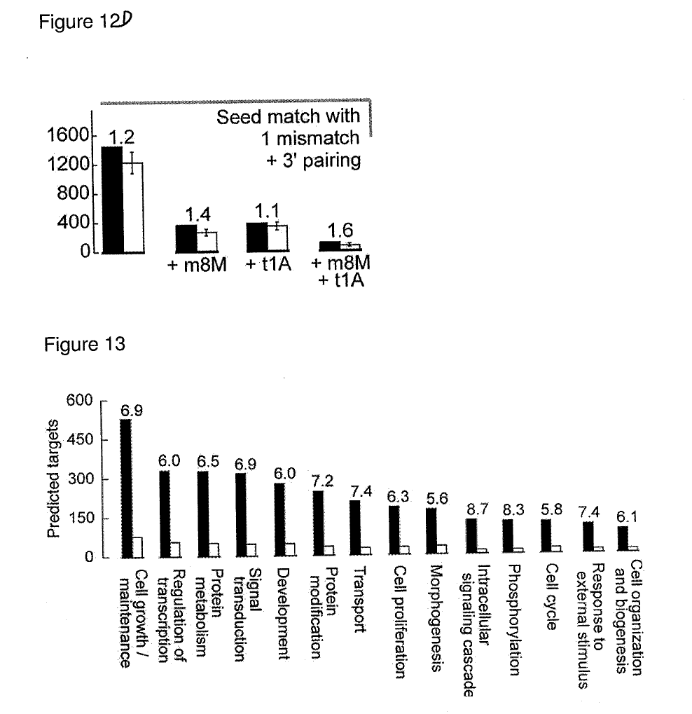

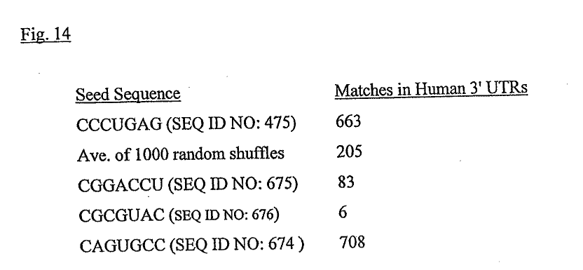

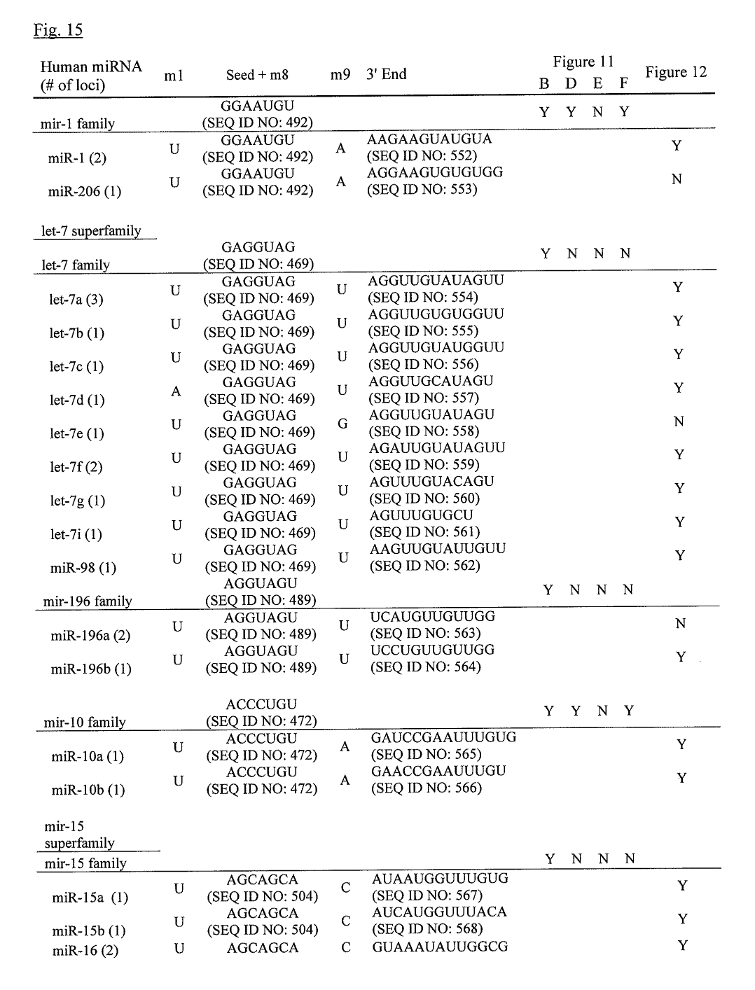

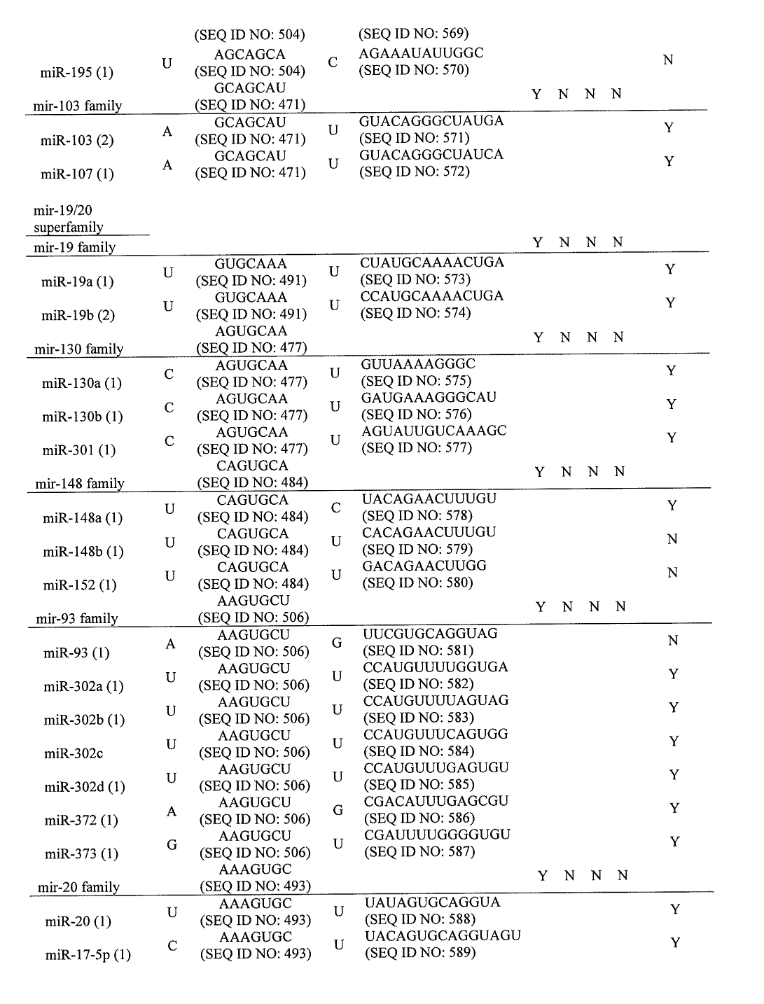

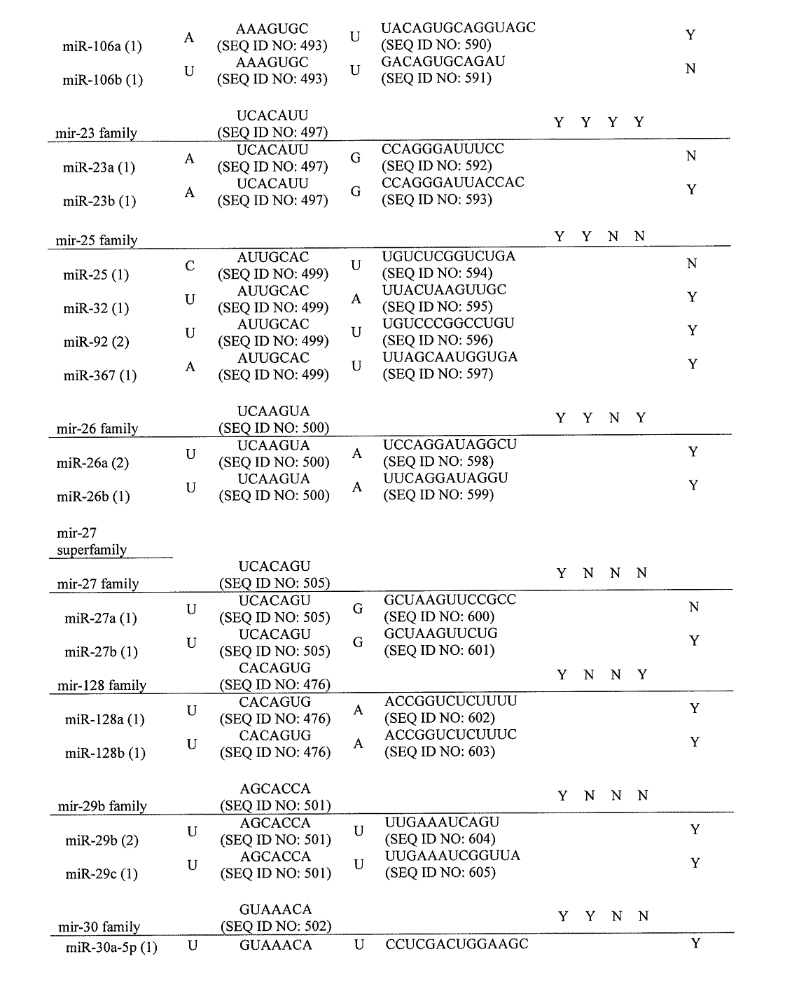

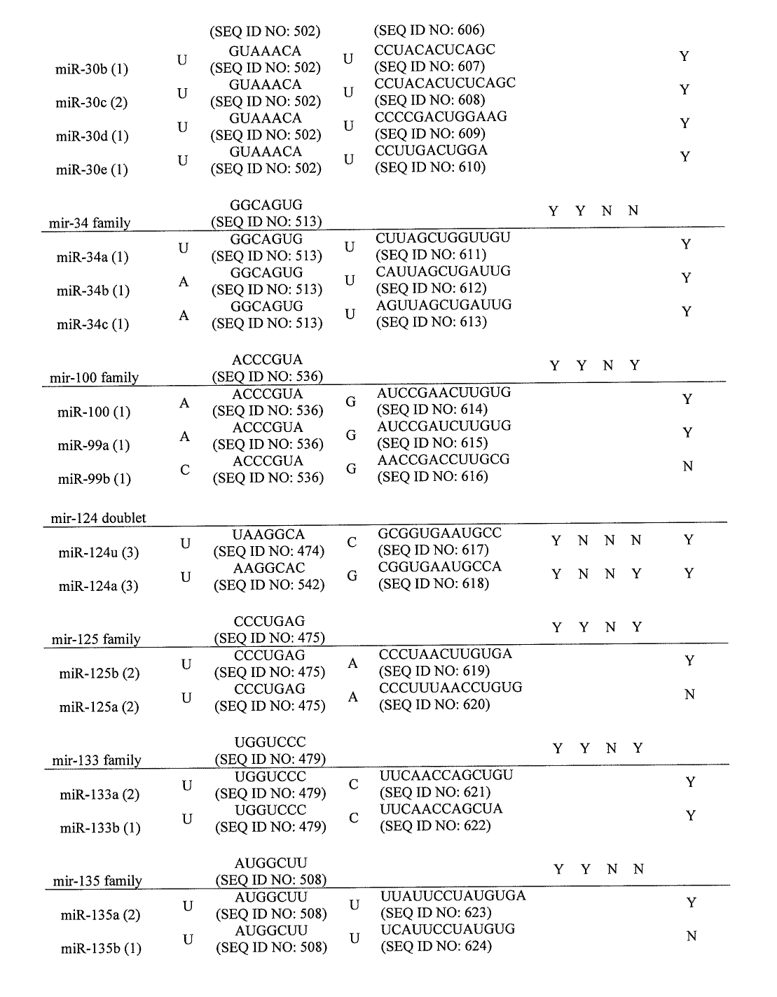

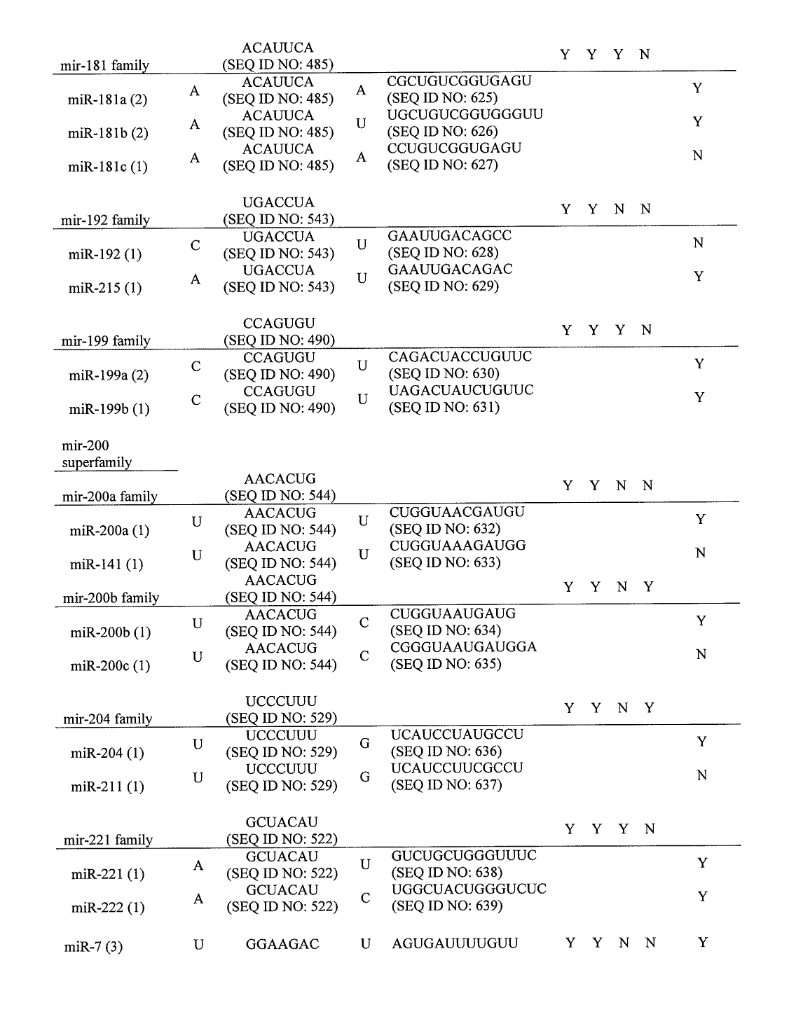

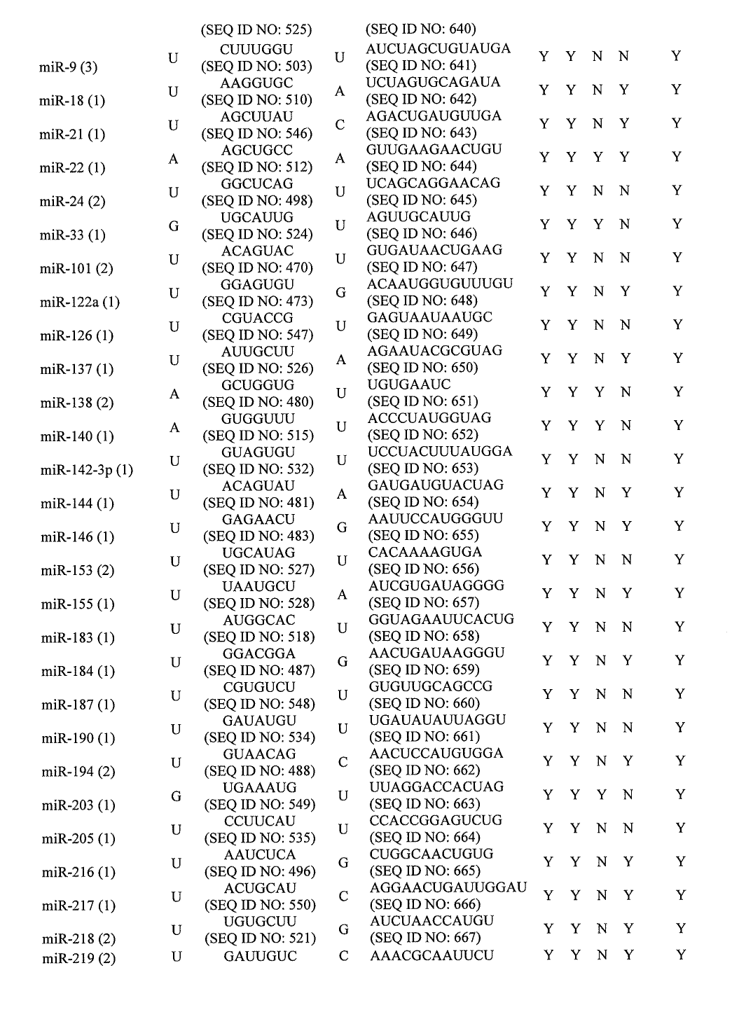

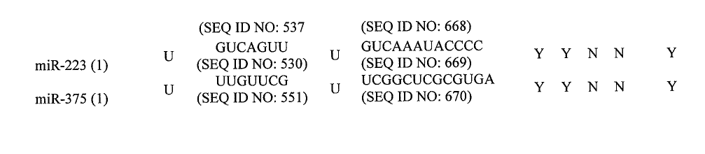

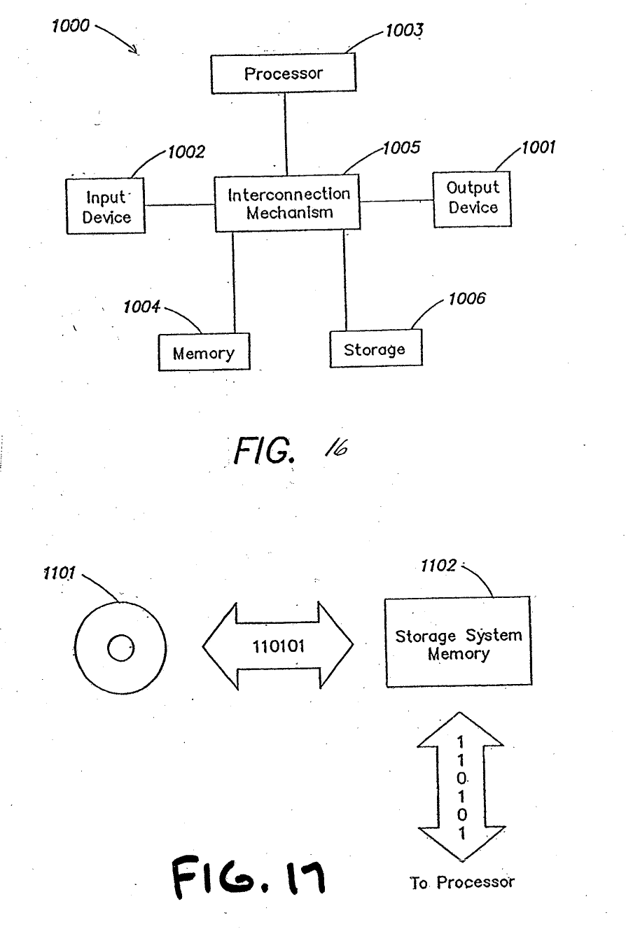

The signal:noise ratio was improved when the seed match was required to occur at corresponding positions in a multiple alignment of the orthologous UTRs. Therefore, the availability of newly sequenced genomes, improved annotations, and whole-genome alignments allowed use of a simplified method. miRNA targets could be predicted by finding perfect Watson-Crick (W-C) seed matches that were conserved in the UTR regions of whole-genome alignments, as exemplified by the miR-23a-HIC seed pairing (FIG. 11A).

FIG. 11A shows the alignment of orthologous segments of the HIC UTR, showing the conserved match to the miR-23a seed. Residues of the seed (purple), seed matches (dark blue), m8 (light purple), m8 matches (light blue), and anchoring A's (red) are indicated.

Starting with the UTRs corresponding to mRNAs annotated in the UC Santa Cruz Genome Browser database, and a set of 62 unique seed matches that represented 148 human miRNA genes and defined the families of known miRNAs conserved in the five genomes (FIG. 15), 14,301 instances of conserved seed matches were identified within the 3′ UTRs, thereby predicting 14,301 unique target sites. Because some UTRs had multiple conserved target sites for the same miRNA seed, this analysis implicated 12,839 unique miRNA-target regulatory relationships FIGS. 11B-11C, left graphs). In these figures, the number of miRNA-target relationships predicted (solid bars), with estimates of the number of false positives (open bars), for searches based on the indicated criteria, are illustrated. In this and subsequent panels, error bars indicate one standard deviation, based on analyses of control cohorts. Standard error on these values was much smaller (not visible if shown as error bars) because each estimate of the number of false positives was calculated using many control sequences. The numbers above each graph indicate the value for signal divided by that of the noise. Also graphed are the subsets of predictions in which seed matches fell within islands of conservation (in islands).

Because many UTRs had conserved target sites for different miRNA seeds, which often could enable combinatorial control of these messages, these 12,839 predictions involved the UTRs 3,227 unique human genes (data not shown).

A set of 117 human miRNAs (FIG. 15) representing 148 human miRNA genes with membership in 62 conserved vertebrate miRNA families was assembled using the Rfam miRNA registry (http://www.sanger.ac.uk/Software/Rfam) and established criteria. MicroRNA families were defined by grouping miRNAs that share a common conserved seed region spanning nucleotides 2 to 7 (although in any analysis involving m8 matches, miR-101 and miR-144 miRNAs, which have the same seed, were regarded as separate families because they differ at m8, bringing to 63 the total number of families for these analyses). One representative from each miRNA family was required to be conserved with no more than one mismatch to the sequence for mouse, rat, dog, and chicken sequences from the UCSC genome browser multiz-8-way whole-genome alignments. In a number of cases, a related miRNA from the chicken genome could not be found in the multiz-8-way alignments, and a chicken miRNA from the miRNA registry that satisfied aforementioned alignment criteria was used in its place. To account for documented 5′ heterogeneity of miR-124, two forms of miR-124 were included separately among the miRNA families: the longer, less frequently observed form contains an additional 5′ U relative to the shorter form and is listed as miR-124u (FIG. 15).

In FIG. 15, known mammalian miRNAs with close orthologs identified in chicken (typically no more than one substitution within the mature miRNA) were included (and are indicated by a Y in the FIG. 12 column). Also listed in FIG. 15 are human miRNAs with the same seed as these highly conserved vertebrate miRNAs. MicroRNAs with the same seed sequence were grouped into families, and families with related seeds were grouped into superfamilies. Members of each family are usually related in origin (an exception is miR-93, which derives from the opposite arm of its precursor than other members of its family). Sequences originally annotated as miRNAs but which instead are likely to be miRNA* sequences were not included. For each figure specified in the columns on the right, the inclusion of the miRNA family or individual miRNA sequence in the analysis is indicated by a Y.

MicroRNA seed sequences corresponding to selected sets of families from the miRNA dataset were used in the analyses in FIGS. 11B to 11H. These sets are listed in FIG. 15 and in FIG. 11 as follows. FIGS. 11B, 11G, and 11H: all 63 miRNAs families described above (62 in the case of Seed or Seed+t1A searches); FIG. 11D: 48 miRNA families representing only those sequences that correspond to seed regions (nucleotides 2 to 7) with no overlapping relationship with a shifted seed sequence of another miRNA family (from the same superfamily) were chosen to ensure the proper register of seed matches and conservation in the surrounding bases; FIG. 11E: 9 miRNA families corresponding to miRNA sequences that have a conserved m1 nucleotide other than U and do not have the same seed sequence as an miRNA with a U at m1; and FIG. 11F: 36 miRNA families corresponding to miRNA sequences that have a conserved m9 nucleotide other than U and do not have the same seed sequence as an miRNA with a U at m9.

To estimate the number of false positives, for each authentic seed match, at least five hexamers of comparable abundance were picked in the UTR dataset. The analysis was repeated with these control sequences, averaging the results for each set of control sequences. The control sequences were generated as follows. Mononucleotide, dinucleotide, hexamer, heptamer, and octamer counts and frequencies were determined for all human 3′ UTR sequences. Sets of control sequences were designed for each seed match sequence and each extended-match variant (SeedM+m8, SeedM+t1A, etc.) so as to preserve the expected frequency of random matching between miRNA seed sequences and complementary 3′ UTR sequences. All hexamers, heptamers, heptamers, and octamers were examined to identify suitable control sequences for each miRNA seed (or augmented seed) that preserve (1) E(SM), the 1st order Markov probability of the seed match, and (2) O(SM), the observed count of seed matches in human UTRs within a total margin of +7.5%. As previously described, for a miRNA seed match heptamer S1, S2, S3, S4, S5, S6, S7, E(SM) was equal to (PS1·PS1, S2·P52, S3·PS3, S4·PS4, S5·PS5, S6·PS6, S7) where PS1 was the frequency of the nucleotide S1 and PSk,Sk+1 was the conditional frequency of the nucleotide Sk+1 given Sk at the previous position, determined by counting dinucleotides in the UTR sequences. Sequences corresponding to known miRNA seeds, as well as sequences known to function in mRNA processing, such as the polyadenylation signals AAUAAA (SEQ ID NO: 671) and AUUAAA (SEQ ID NO: 672) and the consensus RNA binding sequences of the puf protein family, were restricted from use in the control sets. All possible control sequences that met these criteria were assigned to each distinct miRNA seed sequence represented in the dataset. For each miRNA analyzed, an estimate of the false-positive predictions was calculated by averaging the results of each of its control sequences. These averages were then summed to estimate the number of false positives for a set of miRNAs. A few miRNAs were assigned only five control sequences, but most had many more.

To calculate the standard deviation of the number of mRNAs that are predicted to be targets of a single cohort of control sequences, in which a single cohort set consisted of one control sequence per real miRNA, a special procedure was devised that accounted for the varying number of control sequences assigned to each real miRNA in this set. The total number of cohort sets used was defined as N, which was equal to the maximum number of cohorts used for a single real miRNA in the set under consideration. The 1st listed control for each miRNA was assigned to cohort set 1, the 2nd to cohort set 2, . . . , the Nth to cohort set N. When considering cohort set n and a real miRNA with m control sequences in which m<n, the (n mod m)th control sequence was re-chosen to be included in the nth cohort set, thus enabling the construction of N total cohort sets. For each cohort set, the number of predicted target mRNAs was determined, and the standard deviation of the mean was calculated. This single standard deviation value corresponds to the length of the error bars above and below the average noise level in the predicted targets plots (FIGS. 11B, 11E, 11F, 11H, and 12A-12D).

Different approaches have been used to generate control sequences by which to estimate the number of false-positive miRNA target predictions. The approach used in this example is described above and resembles that of earlier examples (e.g. Example 1). The approach differs from the approach of using unfiltered random shuffles. Pitfalls of using unfiltered random shuffles to estimate the false positives can be illustrated with the miR-125 seed heptamer (CCCUGAG, SEQ ID NO: 475, 6-nucleotide seed plus m8). This heptamer has 663 reverse-complement matches in human UTRs, whereas, on average, random shuffles have only 205 hits (FIG. 14). This difference may be readily explained as an artifact of the shuffled sequences containing an oligonucleotide composition that differs from that of the miRNAs. For example, the miRNAs, like the UTRs and vertebrate genomes as a whole, contain few CG dinucleotides. Therefore (since CG is palindromic), random shuffles that create CG dinucleotides have far fewer hits to the UTRs than does the authentic seed heptamer (FIG. 14). To avoid this artifact that would unduly favor the assessment of any algorithm that uses pairing or predicted duplex stability for prediction, controls sequences may be chosen that match the relevant features of the authentic miRNAs, including compositional features. For the miR-125 heptamer, the sequence CAGUGCC (SEQ ID NO. 674) would be a more appropriate control than the typical shuffled derivative. The same principles can be used to generate control sequences that are the same length as the miRNAs. The miR-125 heptamer is GC rich and may be more prone to the vertebrate oligonucleotide-composition artifact of random shuffles than the typical heptamer. On the whole, miRNA heptamer seeds have ˜1.4 times as many hits to vertebrate UTR regions than do their randomly shuffled cohorts.

To summarize, these averages yielded 5,817 target sites corresponding to 5,386 unique false-positive predictions. When considering the 12,839 predictions found when using the real miRNAs, the estimated 5,386 false-positive predictions suggested a signal:noise ratio of 2.4:1 (FIGS. 11B-11C, left graphs). The number of genes targeted above the noise was estimated by removing 5,817 randomly chosen hits from the set of 14,301, leaving 8,484 hits that involved the messages of 2,767 human genes. Thus the five-genome analysis implicated 25% of the set of 10,938 orthologous vertebrate genes as conserved targets of the miRNAs.

The chromosomal coordinates of the 3′ UTRs of all human genes from the “known genes” dataset of the UCSC genome browser annotation database (http://genome.ucsc.edu) were used to define an initial dataset of human 3′ UTR sequences. This set was augmented by taking the union of these regions with analogous regions defining the 3′ UTRs of overlapping human Refseq mRNAs. The corresponding sequence coordinates were retrieved from the UCSC annotation database multiz-8-way multiple alignments, containing aligned orthologous sequences from recent assemblies of the mouse (mm5, 5/2004), rat (rn3, 6/2003), dog (canFam1, 7/2004), chicken (galGal2, 2/2004), Fugu (fr1, 8/2002), and zebrafish genomes (danRer1, 11/2003). When an annotated 3′ UTR sequence overlapped an open reading frame, the overlap was masked to prevent contamination of the 3′ UTR dataset with protein-coding sequences. Using the known Canonical database from the UCSC genome browser, alternate isoforms of a common gene were identified. The longest 3′ UTR among each set of multiple isoforms was chosen. The resulting dataset contained 17,850 aligned mammalian 3′ UTRs (human/mouse/rat/dog) and 10,938 aligned vertebrate 3′ UTRs (human/mouse/rat/dog/chicken). For a few genes, a longer aligned mammalian 3′ UTR isoform was not conserved in chicken while a shorter 3′ UTR isoform was conserved to chicken. These rare cases resulted in a specific vertebrate 3′ UTR isoform not being included in the mammalian set.

Next the sequence flanking the seed matches were examined for conserved positions that might contribute specificity to miRNA:target interactions (FIG. 11D). In FIG. 11D, the overall conservation and sequence identity flanking conserved seed matches and miRNA seeds is shown. Related seeds arising from 5′-end heterogeneity within a miRNA family were excluded from this analysis (FIG. 15). For each position flanking the conserved seed match, the percentage of seed matches in which that position was conserved in all five vertebrates is shown (top panel), with the height of the black bar indicating conservation of any of the four possibilities, and that of the red indicating conservation of adenosine. The gray dashes indicate the same analysis for conserved matches to control sequences. The second panel shows the same analysis for sites that have both a conserved seed match and a conserved m8 match. The third panel shows the sequence identity immediately flanking the seed matches, with the height of the letters corresponding to the information content, measured using the relative entropy relative to the background base composition of 3′ UTRs. The bottom panel shows the analogous representation of the sequence identity at the first 20 positions of the miRNAs, giving equal weight to each miRNA family used in this analysis (FIG. 15).

The position immediately upstream of the seed match was highly conserved in many cases, and appeared to have a high propensity to be a conserved W—C match to the eighth nucleotide of the miRNA. (These target and miRNA positions are designated t8 and m8, respectively, and “M” is used to designate W—C matches between corresponding target and miRNA positions.) Requiring a conserved match at this position markedly increased specificity, improving the signal: noise to 3.8 (FIG. 11B, SeedM+m8M). However, the sensitivity, calculated as signal above noise, decreased substantially, suggesting that some authentic target sites lacked m8 matches.

High conservation was also observed at the first position downstream of the seed match. This nucleotide was often a conserved A, which could pair to the first nucleotide of a miRNA whose first nucleotide is U, a class which includes the majority of miRNAs (FIG. 11D). However, a conserved A was also observed next to seed matches for miRNAs that did not begin with a U. For miRNAs that begin with A, C, or G (and which do not have any known or predicted paralogs that begin with U), the nucleotide immediately downstream of the conserved seed+m8 matches was twice as frequently a conserved A than any other conserved nucleotide, including the nucleotide that could form a W—C match to the first nucleotide of the miRNA.

The discovery that an A appears to anchor the 3′ terminus of the miRNA complementary site suggested that requiring a 6-nucleotide W—C seed match followed by this “A anchor” would increase the specificity of target prediction. Indeed, searching for this type of 7-nucleotide composite match increased signal:noise to 3.8:1 in the five-genome analysis (FIG. 11B, SeedM+t1A). This improved signal:noise was accompanied by a 51% loss in sensitivity. When focusing on the subset of the set of miRNAs that began with A, C, or G, none of this drop in sensitivity was attributed to the loss of matches that involved conserved W—C pairing to the first nucleotide of the miRNA. For these nine representative miRNAs that did not begin with a U and did not share a common seed sequence with a related microRNA that started with a U (FIG. 15), demanding the W—C seed match followed by the A anchor gave 625 predictions (FIG. 11E, SeedM+t1A), whereas demanding that the seed match be followed by a conserved W—C match to the miRNA gave 348 predictions, barely above the estimate of the false positives (FIG. 11E, SeedM+m1M) with signal:noise not significantly better than when requiring conservation of a non-A mismatch at this position (FIG. 11E, SeedM+t1 other). FIG. 11E illustrates the utility of a t1 A anchor for predicting targets when the miRNA does not begin with a U. For this set of miRNAs, the signal:noise ratio in the basic SeedM analysis (before requiring additional conserved pairing or nucleotides) was 1.8:1, which was lower than that for miRNAs that either begin with U or have paralogs that begin with U.

Thus, not all of the specificity of metazoan miRNA-target recognition can be explained by base-pairing to the message; a component of this specificity may instead lie at the level of mRNA primary sequence. A protein of the silencing complex may recognize this A in a manner that allows simultaneous or sequential interaction between the A and the first nucleotide of the miRNA, thereby explaining the strong bias toward a U at the first nucleotide of miRNAs.

Requiring both the m8 match and the t1 anchor improved specificity, with signal:noise of 5.6:1 in the five-genome analysis (FIG. 11B). However, most of the conserved seed matches had only one of these specificity determinants, such that requiring one or the other yielded 8,012 predicted targets with signal:noise of 3.5:1. Calculating, as before, the number of unique genes predicted above, the noise yielded 2,421 unique human genes as miRNA targets, or 22% of the set of 10,968 orthologous genes.

Thus, the ability to predict thousands of targets with a high degree of confidence that most are authentic incorporated two key features of the miRNA target prediction algorithm used in previous examples: a requirement for perfect W—C seed pairing, and the use of rigorous control cohorts to assess the utility of algorithmic refinements (see above). However, the analysis in this example differed by starting with whole-genome alignments, thereby requiring that the conserved seed matches be at conserved positions within the UTRs, and by focusing only on an 8-nucleotide segment of the UTR centered on the seed match, without consideration of other criteria, such as predicted thermodynamic stability of pairing, pairing outside the immediate vicinity of the seed, or presence of multiple complementary sites per UTR, many of which were considered by other target-prediction algorithms, including those described above. The refined algorithm thus has an emphasis on pairing to a 6-nucleotide miRNA seed. Thus, for example, the algorithm may be used to predict targets that have a conserved 6-nucleotide seed match flanked by either a m8 match or a t1A anchor.

Little conservation was detected beyond the residues immediately flanking the conserved seed matches, even though this analysis was restricted to the miRNA families that are highly conserved in the five genomes, each of which has a member with no more than one substitution separating the human and chicken orthologs. Conservation was slightly elevated at t9, particularly when restricting the analysis to sites with m8 matches (FIG. 11D). As seen for t1, there was again enrichment for an A at t9. This bias could not be explained by the nucleotide composition of the miRNAs, even though there is a marked preference for a U at position 9 of the miRNA (FIG. 11D). When closely examining the conserved matches for miRNAs that do not have a U at position 9, an overabundance of a conserved A forming a mismatch to this nucleotide was found. When predicting targets for these miRNAs, requiring a conserved t9A mismatch provided substantially more specificity gain than did requiring a conserved W—C match or conserved non-A mismatches (FIG. 11F). FIG. 11F illustrates the utility of a t9 A anchor for predicting targets when the miRNA does not have a U at position 9. The set of 36 miRNAs used in thus analysis yields a signal:noise of 2.1:1 in a seed-only analysis.

Beyond this modestly conserved t9 anchor, conservation upstream of the seed match, where the 3′ segment of the miRNA would be expected to pair, was no greater than that downstream of the seed match (FIG. 11D). The same was true when restricting the analysis to sites predicted with greater specificity because they had either m8 matches or t1 anchors (FIG. 11D; additional data not shown). The gradual downward slope in conservation observed when going in either direction from the seed match paralleled that of the background expectation and was a consequence of starting at positions that were confidently aligned in the five genomes (FIG. 11D). The lack of conservation upstream of the t9 anchor suggested that thousands of vertebrate miRNA target interactions are mediated primarily by seed matches, supplemented with either a t1A anchor or an m8 match, but with little, if any, role for pairing to the 3′ portion of the miRNA.

The observation that miRNA target sites are often not conserved beyond an 8-nucleotide site centered on the seed match suggested that the specificity of miRNA target prediction might actually be improved by excluding those seed matches that occur in the context of more extensive conservation. Incorporating the criterion that seed matches must fall in short “islands” of conservation surrounded by the expected background level of divergence substantially increased the signal:noise ratios (FIG. 11B, island row of histograms). In FIG. 11B (in islands), the aligned 3′ UTR sequences within 250 nucleotides 5′ and 3′ of a conserved seed match were examined to determine a local density of conservation. All target sites located in a ˜500-nucleotide region with fewer than a total of 50 conserved heptamers in the upstream and downstream windows were designated as occurring in islands of conservation. For cases where a 3′ UTR boundary occurred within 250 nucleotides, the number of conserved 7-mers per 1000 nucleotides was calculated and those sites with a local density of less than 100 conserved 7-mers per 1000 nucleotides were included in the islands of conservation set.

The somewhat counterintuitive use of excess flanking conservation as a contrary indicator for target prediction improved specificity by reducing the frequency of false positives, thereby increasing the signal:noise ratio. To further explore this phenomenon, we binned the UTRs based on their density of conserved heptamers and then calculated the signal:noise ratio of TargetScanS separately for each bin (FIG. 11G). In FIG. 11G, for each 3′ UTR in the dataset used in the 5-genome analysis, the number of conserved 7-mers was counted and a measure of the density of conservation in that 3′ UTR was calculated by determining the average number of conserved 7-mers per 1000 nucleotides. The 3′ UTRs were sorted by this density measure and then assigned to bins such that each bin contained a sufficient number of 3′ UTRs to give a total of ˜8000 conserved 7-mers per bin.

FIG. 11G shows the increased accuracy of target prediction for UTRs with a lower density of conservation. Of the 10,968 UTRs in this dataset, 4,887 had at least one conserved heptamer. These were ranked by their density of conserved heptamers, then binned such that each bin had enough UTRs to contain 8,000 conserved heptamers. For each bin, predictions for the real miRNA seeds (black) are compared to averages for the control cohorts (open). The value for signal divided by that of the noise is shown above representative bins, with the number of conserved heptamers per kb shown below. Also plotted are the percentage of UTRs in each bin that are predicted to be miRNA targets (circles, right axis).

The bins with a low density of conserved heptamers had high signal:noise values (greater than 8:1), whereas those with high-density heptamer conservation had poor signal:noise values (less than 2:1). In other words, as conservation in the UTRs increases, a smaller fraction of the conservation can be explained by pairing to miRNAs. For this reason, the 30 UTRs with the highest density of conserved heptamers were excluded from the analyses reported in this paper (other than that of FIG. 11G). Although these 30 messages are likely to be miRNA targets, it seemed prudent not to include them because of the high likelihood that they would have fortuitous conserved pairing to many other miRNAs that do not regulate them.

For many examples of metazoan miRNA-target interactions with experimental support, recognition appears to involve multiple complementary sites to the same miRNA. However, a number of examples of regulation have been identified that involve what appears to be only a single complementary site for a particular miRNA. The original analyses primarily predicted targets with more than one match to the same miRNA, although the cutoffs used for the four-genome analysis (human, mouse, rat, pufferfish) did include some predicted targets with single sites. In contrast to the original computer program of example 1, in the program of this example targets are predicted without preference for those that have multiple matches. Requiring a second syntenic match to the same miRNA seed increased the signal:noise ratio to 3.2:1 but reduced by 90% the number of predictions (data not shown). Thus, demanding more than one conserved match excluded most of the apparently authentic miRNA-target pairs identified in this analyses. Of course, the finding that single conserved matches are sufficient to confidently predict miRNA-target pairs in a comparative genomic analysis is completely compatible with the idea that, within the cell, biochemical specificity is augmented by additional determinants, such as mRNA structure, binding of accessory proteins, and/or the presence of nonconserved or imperfect seed matches at additional sites in the message.

FIG. 11H illustrates the analysis with one to five genomes (H, human; M, mouse; R, rat; D, dog; C, chicken) using the set of 10,968 genes aligned in the five genomes. Using the computer program on fewer genomes provided modest gains in sensitivity (FIG. 11H), mostly from removing chicken from the analysis, which allowed identification of miRNA-target interactions that were lost in the five-genome analyses either because they are specific to the mammalian lineages or because they lie in portions of the chicken genome that are missing or misassembled in the database. When extending the four-genome analysis to include genes aligned among the mammals but not to chicken, 13,044 regulatory interactions were predicted above the estimate of the false-positive predictions, an average of over 200 targets for each of the miRNA families represented (data not shown). Calculating as for the five-genome analysis the number of unique genes predicted above the noise yielded 5,300 unique human genes as miRNA targets, or 30% of this set of 17,850 orthologous mammalian genes.

The four-genome mammalian analysis provided a set of predictions suitable for comparing to the results of previous mammalian target-prediction efforts. After accounting for the different starting sets of miRNAs and protein-coding genes, 343 of the 451 predictions in the original three-genome TargetScan analysis remained, and 67% of these overlapped (data not shown). However, there was less overlap with the results of other mammalian target predictions. As described below, the program may miss some targets when demanding perfect seed matching confined to the 3′ UTRs. However, it is believed that the program does not miss a large class of authentic targets. Instead, the small overlap could be due to a large number of false positives generated by certain other prediction methods.

The plant miRNAs appeared to have a strong propensity to target messages of developmental regulators, particularly transcription factors involved in plant development. Although many of the predictions were annotated as controlling transcription or development, most had other functions (FIG. 13), as seen previously for the TargetScan predictions (data not shown; also see above). FIG. 13 illustrates a biological process classification of the vertebrate miRNA targets predicted in the Seed+t1A+m8M Analysis, including plots for categories that are represented by targets and have signal:noise ratios of at least 5.6:1, which was the ratio for the overall analysis (FIG. 11B).

Some miRNAs had a propensity to target genes of a particular category. An interesting example is the miRNAs of the mir-17-18-19-20-92 gene-cluster, which resides in a region of the genome that is amplified in many lymphomas and solid tumors. These miRNAs had a striking propensity to target genes with known or suspected roles in growth control, including both oncogenes and genes that repress growth (data not shown). Among those with roles in growth arrest were numerous genes in the TGF-beta (TGFβ) signaling pathway (including TGF, β receptor II, BMP receptor II, Activin receptor I, Smad2, Smad6, Smad7, and SARA, SARA, P300 CREBBP, P/CAF) SOCS genes (SOCS-1, SOCS-3, SOCS-5, and SOCS-6), Runt-related transcription factors/Core-binding factor (AML1/RUNX1, AML2/RUNX3, CBFP/PEPB2)MAPK signaling (MAPKKK2, MAPKKK3, MAPKK5/ASK1, MAPKKK9, p130, E2F5, PTEN, etc. As a consequence, inhibiting on or more genes, in this miRNA cluster (for example, by using oligonucleotides that are substantially antisense to one or more of these miRNAs) may be used to treat diseases of cell proliferation, including human cancers. The genes may be unregulated, for instance to increase proliferation in vitro cell lines.

These analyses therefore indicate that a substantial fraction of the mammalian genes are subject to miRNA control and that primary sequence determinants supplement pairing in specifying target recognition. Initial analyses indicate that the same is true in invertebrates.

Gene ontologies were assigned to human genes from the UCSC known genes database by cross-referencing with GO identifiers listed in the annotation database of the UCSC genome browser (http://hgdownload.cse.ucsc.edu/). The Gene Ontology Consortium database (Harris et al., 2004) was retrieved from http://www.geneontology.org and biological process ontologies were compiled for all predicted target genes of the miRNAs (five-genome SeedM+m8M+t1A analysis) and the corresponding octamer control sequences. As in previous signal-to-noise calculations, the hits to GO categories were averaged to determine a noise estimate for an octamer control set and GO category.

One purpose of miRNA target predictions is to identify authentic regulatory interactions without relying on conservation. The insights gained by the work in this and previous examples suggest that nonconserved regulatory interactions could be identified by finding those messages with 7-nucleotide matches to the seed regions of coexpressed miRNAs.

### Example 8

This example describes, in greater detail, the method of identifying targets of miRNA sequences used in Example 1.

Initially, a method for identifying and scoring interactions between the microRNA and mRNA that incorporates features that might influence microRNA function was performed as follows.

(1) mRNA sequences were searched for “seed match” sites that were perfectly complementary to bases 2-8 of the microRNA (referred to as the microRNA “seed” sequence).

(2) The initial seed pairing was extended with Watson-Crick or G:U pairs flanking the initial seed match.

(3) Next, an artificial linker hairpin sequence (as an example, the sequence 5′-GGGCCCGGGULLLLLLACCCGGGCCC-3′ (SEQ ID NO: 1), where “L” denotes an unpaired nucleotide and all non-“L” bases are paired to their Watson-Crick counterpart across the loop) was attached to the 5′ end of the microRNA and corresponding 3′ end of the paired mRNA sequence so as to form a hairpin sequence containing the microRNA, linker hairpin sequence, seed match region, and 35 bases 5′ of the seed match.

(4) Basepairing of the microRNA-linker-mRNA duplex was optimized using the RNAfold folding optimization routine of the Vienna RNA package, incorporating basepairing constraints corresponding to previously-determined basepairs between the microRNA and mRNA (basepairs determined above in steps 1 and 2), disallowing basepairing of sequences between bases in the mRNA sequence.

(5) The artificial hairpin sequences added in step 3 were removed, while retaining the optimal basepairing pattern, to give a miRNA:mRNA duplex with predicted basepairing between RNA strands. Note that this basepairing contained minimally the Watson-Crick pairing between the microRNA seed (bases 2-8 as numbered from the 5′ end of the microRNA) and the mRNA seed match sequence.

(6) The predicted folding energy of the duplex was evaluated using RNAeval from the Vienna RNA package. The energies are combined in a composite score “Z” for a given miRNA:mRNA interaction, where G₁ . . . Gₙ denote energies for i=1 . . . n candidate sites for a miRNA, miR-x₁, as follows:

$${Z}_{x} = {\sum }_{k = 1}^{n} {e}^{- {G}_{k} / T} ,$$

where T is a parameter that determines the scoring contributions of multiple sites.

(7) The combinatorial score was determined by determining energies and basepairing structures all microRNA target sites on a given mRNA sequence for all microRNAs, miR-x₁, miR-x₂, . . . , miR-xₘ, used in the search, using the formula:

$${Z}_{total} = {\sum }_{k = 1}^{n} {Z}_{{x}_{k}} .$$

Next, a comparative genomics method for evaluating the validity of the model described above was used, by observing preferential conservation of high-scoring predictions at defined cutoffs on the scores and score ranks as determined by the model described above, as follows.

(1) Cohorts of control sequences were generated that preserved the properties of the real microRNAs which contribute to the probability of identifying high-scoring sites in the dataset of mRNA (or 3′ UTR) sequences. Relevant features were preserved within a specified window (e.g. ±7.5%). This protocol has been termed the miRshuffle algorithm in Example 1. These cohort sequences preserve E(Seed Match occurrence in dataset|dinucleotide composition of 3′ UTR sequences) (E expectation).

The expectation for the occurrence of higher order k-mer sequences is determined from empirical 3′ UTR dinucleotide frequencies using a generative first-order Markov model to model the probability of observing the higher order k-mer sequences. The nucleotide at position 1 was preserved exactly, and the probability of matching the reverse complement of the sequence spanning bases 2 . . . 8, given the dinucleotide frequencies in the dataset, were preserved in the generation of cohorts. If positions 1 . . . 20 in a mature microRNA sequence are denoted by m₁, . . . m₂₀ (numbered 5′ to 3′) and the opposing bases of the target are denoted t₁, . . . , tₙ (numbered 3′ to 5′) then the quantity preserved (±7.5%) in the generation of cohorts was (p=probability):

p(t₂|t₁)p(t₃|t₂)p(t₄|t₃)p(t₅|t₄)p(t₆|t₅)p(t₇|t₆)p(t₈|t₇)

Note that this quantity actually differs from the joint probability of the 7-nucleotide seed match sequence (reverse complement of the seed). That joint probability would not include conditioning on p₁:

p(t₂,t₃,t₄,t₅,t₆,t₇,t₈)=p(t₂)p(t₃|t₂)p(t₄|t₃)p(t₅|t₄)p(t₆|t₅)p(t₇|t₆)p(t₈|t₇),

where p(tᵢ, tⱼ)=p(tᵢ) p(tⱼ|tᵢ)=empirical frequency of dinucleotide corresponding to adjacent bases tᵢ,tⱼ in the dataset.

Additionally, the seed match frequency (or expectation given 7-mer composition) E(Seed Match occurrence in dataset|7-nucleotide composition of 3′UTR sequences) was observed. The probability of observing a 7-nucleotide seed match corresponds to the frequency of the 7-nucleotide seed match motif in the dataset.

Watson-Crick binding energies were predicted for 7-nucleotide seed:seed match duplex in kcal/mol (as determined by RNAeval). Each 7-nucleotide seed sequence was incorporated in a duplex with its perfect Watson-Crick complement and the predicted RNA duplex pairing energy is determined using RNAeval.

(2) Orthologous mRNA sequences were searched using the model described above to determine optimal pairing and scores of sites for the complement of real microRNAs and the corresponding control cohort sequences.

(3) Rank and Z cutoffs, R_{c} and Z_{c}, were chosen so as measure relative levels of conservation of sites corresponding to the real microRNAs and the cohorts of control sequences, at different optimal levels of targeting as determined by the scores Z and the rank of those scores.

Next, perturbation analysis of the 2-8 position of the seed in the microRNA was accomplished by performing the above methods using alternate definitions of the seed, as follows.

(1) An analysis that incorporates perturbations of the model for interactions and comparative genomics validation described above was performed using alternate definitions of the seed region, spanning numerous 7-nucleotide registers of the microRNA. This analysis has been termed the “sliding seed” experiment. Searches identical to those described above are performed where the seed was defined alternately as bases 1 . . . 7, 2 . . . 8, 3 . . . 9, 4 . . . 10, and 5 . . . 11 as measured from the 5′ end of the microRNA as well as the corresponding 7-nucleotide regions spanning bases 1 . . . 7, 2 . . . 8, 3 . . . 9, 4 . . . 10, and 5 . . . 11 as measured from the 3′ end of the microRNA and searched with a “mirror” version of the algorithm described above.

In Example 1, the most optimal levels of conserved targeting (relative to the levels of conserved targeting by cohort sequences) were observed when using bases 2 . . . 8 (as measured from the 5′ end). However, appreciable signal above noise was also observed when using bases 1 . . . 7 (as measured from the 5′ end).

### Example 9

This example describes a modified version of the TargetScan algorithm used to investigate the abundance of targets that have conserved G:U pairs or other mismatches between the miRNA seed and target site.

By requiring perfect seed pairing, the respective programs may miss miRNA-target interactions with wobbles or mismatches that disrupt seed pairing, such as the nematode let-7-lin-41 or vertebrate miR-196-HoxB8 interactions, both of which have been validated in animals. The loss of such interactions from the earlier analysis was tolerated because allowing wobbles or mismatches in the seed pairing would have decreased the signal:noise ratio using rigorous estimates of false positives to essentially 1:1, casting doubt on all such interactions identified with imperfect seed matching.

Revisiting this issue in an analysis including newly sequenced genomes revealed some signal above noise, with moderate improvement in specificity when requiring the t1 A anchor or m8 match, but the quality of these predictions was still far below that observed for perfect seed matches (FIG. 12). FIG. 12 shows signals (black) and estimates of false positives (open) when identifying miRNA targets having a conserved G:U pair (FIGS. 12A and 12C) or mismatch (FIGS. 12B and 12D) disrupting the seed match. The effects on signal and noise when requiring pairing to the 3′ portion of the miRNA (six contiguous pairs allowing one G:U wobble) are also shown (FIGS. 12C and 12D).

The algorithm proceeded by first finding matches to the miRNA seed that were W—C pairings, except at one position at which they have either a conserved G:U wobble or a conserved mismatched to the seed. This initial match was then extended with W—C pairing to m8 and by identifying the presence of a t1A anchor (W—C pairing to m1 was not scored). To identify pairing to the 3′ portion of the miRNA, the miRNA and target site candidate that included the seed match and 15 mRNA bases upstream of this match were co-folded using subroutines imported from the RNAlib C program library of the Vienna RNA package, while incorporating constraints on the pairing of the miRNA seed. This routine was repeated using aligned sequences from each vertebrate genome. Predicted target sites were accepted in the 3′-pairing analysis if in each genome they contained a contiguous helix of at least six basepairs (allowing for a single G:U wobble), even if in the different genomes this helix involved different 3′ residues of the miRNA.

For the analysis of target sites with pairing to the 3′ end of the miRNA, control sequences were generated by simply merging each controlled seed sequence with the remaining ˜13-16 nucleotide region of the real miRNA that follows the seed. For the analysis of miRNA target sites with a single G:U pair disrupting the seed match (FIG. 12), control sequences were screened further so as to contain, on average, a similar number of instances, of the 4-mer UGUA (SEQ ID NO: 673) as the corresponding real miRNAs because this 4-mer is the core conserved element of the PUM2 binding site consensus. This extra measure was performed for the G:U analysis due to the fact that the set of 6-mers corresponding to seed matches disrupted with a single G:U are enriched for the UGU 3-mer relative to 6-mers of comparable abundance in a single genome. The enrichment for UGU may be a consequence of the enrichment for U's and G's obtained when specifying that seed:seed match duplexes is disrupted by a G:U pair.

The let-7-lin-41 and miR-196-HoxB8 interactions both included extensive pairing to the 3′ portion of the miRNA, each involving at least nine contiguous W—C pairs, which might compensate for the imperfect seed pairing and impart specificity. Requiring conserved 3′ pairing with at least six contiguous pairs (allowing one G:U wobble) yielded little if any increased specificity of target prediction (FIG. 12, bottom panels). The existence of a class of conserved sites of this type could explain the observed pattern of sequence conservation of vertebrate miRNAs, which typically extends throughout the miRNA. However, compared to searches requiring perfect seed pairing (FIG. 11B), fewer targets were predicted. Overall, it appears that there are relatively few conserved interactions that lack perfect seed pairing. However, additional parameters need to be examined, and it remains possible that many such interactions exist but most of them have not yet been confidently identified by existing algorithms. If relatively few miRNA interactions lack perfect seed pairing, this could be explained if these types of interactions typically require extensive pairing outside the seed, thereby increasing the total required base-pairing to the message. As a result, such interactions would emerge more rarely and be more difficult to maintain over the course of evolution, perhaps occurring under circumstances in which regulation by a specific member of a multi-miRNA family is required. For example, if C. elegans lin-41 were to be repressed by any of the other three let-7 family members, which have the same seeds, but are expressed earlier than is let-7 RNA, then premature down-regulation of lin-41 might cause larval cells to precociously assume adult cell fates. Perhaps to achieve the proper timing of repression, the lin-41 3′ UTR has imperfect seed pairing to the entire let-7 family, which prevents regulation by the other three family members while the extensive pairing to the unique 3′ region of let-7 RNA enables regulation by let-7.

### Example 10

This example describes the method of identifying miRNA targets with target sites that reside in coding regions (also called open reading frames, or ORFs).

The 5′ UTR and ORF datasets were compiled using genomic coordinates from the human RefSeq mRNA database of the UCSC genome browser to retrieve regions of the multiz-8-way alignments in a manner analogous to the construction of the 3′ UTR datasets of example 7. Isoforms of a common gene were identified after mapping the known Canonical database to RefSeq. As before, the single longest sequence was chosen from each set of isoforms. In addition, all ORF sequences were required to begin with a conserved start codon, and all protein-coding sequence was masked in the 5′ UTR dataset. The resulting dataset of 5′ UTRs contained 6,623 sequences, and the resulting dataset of ORFs contained 11,830 sequences.

In animals, previously known target sites are in 3′ UTRs, whereas in plants they are sometimes in the 3′ UTR but are usually in the ORFs and also have been predicted to reside in 5′ UTRs. The program used in example 7, applied to 5′ UTRs, found little or no signal above noise. Because of their high sequence conservation, ORFs were more difficult to analyze by these methods. Nonetheless, a five-genome ORF analysis requiring conserved seed matches flanked by both an m8 match and a t1A yielded 2,371 predicted targets (data not shown), which was significantly above the 1,300 estimated false positives. Although this analysis provided evidence that many messages have functional miRNA complementary sites in ORFs, the data are consistent with the idea that most functional mRNA-miRNA pairing resides in the 3′ UTRs, and that miRNA pairing explains a substantial fraction of the conservation observed in metazoan 3′ UTRs.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-15. (canceled)

16. A method, comprising:
increasing, in a vertebrate cell, expression of a gene regulated by binding of miRNA to an miRNA binding region of an mRNA corresponding to the gene by exposing the cell to an oligonucleotide comprising a sequence that is substantially antisense to at least a portion of the miRNA binding region of the mRNA.

17-20. (canceled)

21. The method of claim 16, wherein the cell is a mammalian cell.

22. The method of claim 16, wherein the cell is a human cell.

23. The method of claim 16, wherein the cell is part of an in vitro culture.

24. The method of claim 16, wherein the cell is part of a living organism.

25. A method of decreasing expression of a gene in a cell, comprising:
introducing, into a vertebrate cell, an isolated oligonucleotide comprising an miRNA sequence in an effective amount to increase expression of the gene.

26. The method of claim 25, wherein the isolated oligonucleotide has a stem-loop structure.

27. The method of claim 25, wherein the isolated oligonucleotide forms an miRNA duplex.

28-29. (canceled)

30. The method of claim 25, wherein the cell is a mammalian cell.

31. The method of claim 25, wherein the cell is a human cell.

32. The method of claim 25, wherein the cell is part of an in vitro culture.

33. The method of claim 25, wherein the cell is part of a living organism.

34. A method, comprising:
transfecting a vertebrate cell with a sequence encoding an miRNA that, when expressed by the cell, causes the cell to overexpress the miRNA.

35-36. (canceled)

37. The method of claim 34, wherein the cell is a mammalian cell.

38. The method of claim 34, wherein the cell is a human cell.

39. The method of claim 34, wherein the cell is part of an in vitro culture.

40. The method of claim 34, wherein the cell is part of a living organism.

41-63. (canceled)


SEQUENCE LISTING (1227 sequences)

SEQ ID NO: 1 - RNA, 26 nt, Artificial Sequence
  Description of Artificial Sequence Synthetic oligonucleotide
gggcccgggunnnnnnacccgggccc

SEQ ID NO: 2 - DNA, 34 bp, Artificial Sequence
  Description of Artificial Sequence Synthetic oligonucleotide
agctctatacgcgtctcaagcttactgctagcgt

SEQ ID NO: 3 - RNA, 22 nt, Artificial Sequence
  Description of Artificial Sequence Synthetic oligonucleotide
ugagguaguagguuguauaguu

SEQ ID NO: 4 - RNA, 22 nt, Artificial Sequence
  Description of Artificial Sequence Synthetic oligonucleotide
uugaugguagugaugugguaua

SEQ ID NO: 5 - RNA, 22 nt, Artificial Sequence
  Description of Artificial Sequence Synthetic oligonucleotide
ugguuuauggaguauaggguau

SEQ ID NO: 6 - RNA, 22 nt, Artificial Sequence
  Description of Artificial Sequence Synthetic oligonucleotide
uauggguguauauggaguuagu

SEQ ID NO: 7 - RNA, 22 nt, Artificial Sequence
  Description of Artificial Sequence Synthetic oligonucleotide
uuauguugaguagguagguaug

SEQ ID NO: 8 - RNA, 22 nt, Artificial Sequence
  Description of Artificial Sequence Synthetic oligonucleotide
ugagguaguagguugugugguu

SEQ ID NO: 9 - RNA, 22 nt, Artificial Sequence
  Description of Artificial Sequence Synthetic oligonucleotide
ugagguaguagguuguaugguu

SEQ ID NO: 10 - RNA, 21 nt, Artificial Sequence
  Description of Artificial Sequence Synthetic oligonucleotide
agagguaguagguugcauagu

SEQ ID NO: 11 - RNA, 21 nt, Artificial Sequence
  Description of Artificial Sequence Synthetic oligonucleotide
ugagguaggagguuguauagu

SEQ ID NO: 12 - RNA, 22 nt, Artificial Sequence
  Description of Artificial Sequence Synthetic oligonucleotide
ugagguaguagauuguauaguu

SEQ ID NO: 13 - RNA, 22 nt, Artificial Sequence
  Description of Artificial Sequence Synthetic oligonucleotide
ugagguaguaguuuguacaguu

SEQ ID NO: 14 - RNA, 22 nt, Artificial Sequence
  Description of Artificial Sequence Synthetic oligonucleotide
ugagguaguaguuugugcuguu

SEQ ID NO: 15 - RNA, 22 nt, Artificial Sequence
  Description of Artificial Sequence Synthetic oligonucleotide
uggaauguaaagaaguauguau

SEQ ID NO: 16 - RNA, 22 nt, Artificial Sequence
  Description of Artificial Sequence Synthetic oligonucleotide
uguaaauuuaauaggggagaua

SEQ ID NO: 17 - RNA, 22 nt, Artificial Sequence
  Description of Artificial Sequence Synthetic oligonucleotide
uaauuuagggaguauaaggaau

SEQ ID NO: 18 - RNA, 22 nt, Artificial Sequence
  Description of Artificial Sequence Synthetic oligonucleotide
ugaagaguuagauuagaaugua

SEQ ID NO: 19 - RNA, 22 nt, Artificial Sequence
  Description of Artificial Sequence Synthetic oligonucleotide
ugaaguaagaguauuugagaua

SEQ ID NO: 20 - RNA, 21 nt, Artificial Sequence
  Description of Artificial Sequence Synthetic oligonucleotide
uggaagacuagugauuuuguu

SEQ ID NO: 21 - RNA, 21 nt, Artificial Sequence
  Description of Artificial Sequence Synthetic oligonucleotide
ucaauguuugaauauguggug

SEQ ID NO: 22 - RNA, 21 nt, Artificial Sequence
  Description of Artificial Sequence Synthetic oligonucleotide
uauuuugguucaaggagaugu

SEQ ID NO: 23 - RNA, 21 nt, Artificial Sequence
  Description of Artificial Sequence Synthetic oligonucleotide
ugauuucuggauuguaugaag

SEQ ID NO: 24 - RNA, 21 nt, Artificial Sequence
  Description of Artificial Sequence Synthetic oligonucleotide
uugugggaugagauaaucuuu

SEQ ID NO: 25 - RNA, 23 nt, Artificial Sequence
  Description of Artificial Sequence Synthetic oligonucleotide
ucuuugguuaucuagcuguauga

SEQ ID NO: 26 - RNA, 23 nt, Artificial Sequence
  Description of Artificial Sequence Synthetic oligonucleotide
ugcucuuuuuuuagcgauugaga

SEQ ID NO: 27 - RNA, 23 nt, Artificial Sequence
  Description of Artificial Sequence Synthetic oligonucleotide
uucugaauuuuuggcgaguucau

SEQ ID NO: 28 - RNA, 23 nt, Artificial Sequence
  Description of Artificial Sequence Synthetic oligonucleotide
uaacucuguauggguaucuuugu

SEQ ID NO: 29 - RNA, 23 nt, Artificial Sequence
  Description of Artificial Sequence Synthetic oligonucleotide
uugcucuuguuguguacaugaua

SEQ ID NO: 30 - RNA, 23 nt, Artificial Sequence
  Description of Artificial Sequence Synthetic oligonucleotide
uacccuguagauccgaauuugug

SEQ ID NO: 31 - RNA, 22 nt, Artificial Sequence
  Description of Artificial Sequence Synthetic oligonucleotide
uacccuguagaaccgaauuugu

SEQ ID NO: 32 - RNA, 22 nt, Artificial Sequence
  Description of Artificial Sequence Synthetic oligonucleotide
uaugccauuuaccgaaugcuga

SEQ ID NO: 33 - RNA, 22 nt, Artificial Sequence
  Description of Artificial Sequence Synthetic oligonucleotide
uaugcugcgaaauacauuugcc

SEQ ID NO: 34 - RNA, 22 nt, Artificial Sequence
  Description of Artificial Sequence Synthetic oligonucleotide
uaugaucaacugacacguugcu

SEQ ID NO: 35 - RNA, 22 nt, Artificial Sequence
  Description of Artificial Sequence Synthetic oligonucleotide
uaccucacggcugauguuuaaa

SEQ ID NO: 36 - RNA, 22 nt, Artificial Sequence
  Description of Artificial Sequence Synthetic oligonucleotide
uagcagcacauaaugguuugug

SEQ ID NO: 37 - RNA, 22 nt, Artificial Sequence
  Description of Artificial Sequence Synthetic oligonucleotide
uagcagcacaucaugguuuaca

SEQ ID NO: 38 - RNA, 22 nt, Artificial Sequence
  Description of Artificial Sequence Synthetic oligonucleotide
uagcagcacguaaauauuggcg

SEQ ID NO: 39 - RNA, 22 nt, Artificial Sequence
  Description of Artificial Sequence Synthetic oligonucleotide
uagaaauagucacgcaggucgu

SEQ ID NO: 40 - RNA, 22 nt, Artificial Sequence
  Description of Artificial Sequence Synthetic oligonucleotide
uacagagaagcgcuaaucgugu

SEQ ID NO: 41 - RNA, 22 nt, Artificial Sequence
  Description of Artificial Sequence Synthetic oligonucleotide
uagaggaaaucgcuuagccgau

SEQ ID NO: 42 - RNA, 22 nt, Artificial Sequence
  Description of Artificial Sequence Synthetic oligonucleotide
uagaaauacgugacguguccag

SEQ ID NO: 43 - RNA, 20 nt, Artificial Sequence
  Description of Artificial Sequence Synthetic oligonucleotide
acugcagugaaggcacuugu

SEQ ID NO: 44 - RNA, 22 nt, Artificial Sequence
  Description of Artificial Sequence Synthetic oligonucleotide
uaaggugcaucuagugcagaua

SEQ ID NO: 45 - RNA, 22 nt, Artificial Sequence
  Description of Artificial Sequence Synthetic oligonucleotide
ucuucuaugaagaacgaauggg

SEQ ID NO: 46 - RNA, 22 nt, Artificial Sequence
  Description of Artificial Sequence Synthetic oligonucleotide
uuggguaaaaagucucaucgag

SEQ ID NO: 47 - RNA, 22 nt, Artificial Sequence
  Description of Artificial Sequence Synthetic oligonucleotide
ucaggguaaaacgauuggacuu

SEQ ID NO: 48 - RNA, 22 nt, Artificial Sequence
  Description of Artificial Sequence Synthetic oligonucleotide
ugaauaugcagugagugauacc

SEQ ID NO: 49 - RNA, 23 nt, Artificial Sequence
  Description of Artificial Sequence Synthetic oligonucleotide
ugugcaaaucuaugcaaaacuga

SEQ ID NO: 50 - RNA, 23 nt, Artificial Sequence
  Description of Artificial Sequence Synthetic oligonucleotide
ugccuuccugaaaaauauaggaa

SEQ ID NO: 51 - RNA, 23 nt, Artificial Sequence
  Description of Artificial Sequence Synthetic oligonucleotide
uggaaccaaauaugacuucugaa

SEQ ID NO: 52 - RNA, 23 nt, Artificial Sequence
  Description of Artificial Sequence Synthetic oligonucleotide
ugacugagcaugaauuuacaaca

SEQ ID NO: 53 - RNA, 23 nt, Artificial Sequence
  Description of Artificial Sequence Synthetic oligonucleotide
uucaauaaggaccuugauacaag

SEQ ID NO: 54 - RNA, 23 nt, Artificial Sequence
  Description of Artificial Sequence Synthetic oligonucleotide
ugugcaaauccaugcaaaacuga

SEQ ID NO: 55 - RNA, 23 nt, Artificial Sequence
  Description of Artificial Sequence Synthetic oligonucleotide
uaaagugcuuauagugcagguag

SEQ ID NO: 56 - RNA, 23 nt, Artificial Sequence
  Description of Artificial Sequence Synthetic oligonucleotide
uggugcuggaacaugauaauagu

SEQ ID NO: 57 - RNA, 23 nt, Artificial Sequence
  Description of Artificial Sequence Synthetic oligonucleotide
ugaggauguacauauugucaagg

SEQ ID NO: 58 - RNA, 23 nt, Artificial Sequence
  Description of Artificial Sequence Synthetic oligonucleotide
uguaaaugcagugugacaguaug

SEQ ID NO: 59 - RNA, 23 nt, Artificial Sequence
  Description of Artificial Sequence Synthetic oligonucleotide
uaaugaggaucugggaucaugua

SEQ ID NO: 60 - RNA, 22 nt, Artificial Sequence
  Description of Artificial Sequence Synthetic oligonucleotide
uagcuuaucagacugauguuga

SEQ ID NO: 61 - RNA, 22 nt, Artificial Sequence
  Description of Artificial Sequence Synthetic oligonucleotide
uuaguuaugcaggguuaucaca

SEQ ID NO: 62 - RNA, 22 nt, Artificial Sequence
  Description of Artificial Sequence Synthetic oligonucleotide
uauuagugacuucagggacuua

SEQ ID NO: 63 - RNA, 22 nt, Artificial Sequence
  Description of Artificial Sequence Synthetic oligonucleotide
ucuauaaugucaaguaguuggc

SEQ ID NO: 64 - RNA, 22 nt, Artificial Sequence
  Description of Artificial Sequence Synthetic oligonucleotide
uuaguuauuuacauggacaggc

SEQ ID NO: 65 - RNA, 22 nt, Artificial Sequence
  Description of Artificial Sequence Synthetic oligonucleotide
aagcugccaguugaagaacugu

SEQ ID NO: 66 - RNA, 22 nt, Artificial Sequence
  Description of Artificial Sequence Synthetic oligonucleotide
agagguggacuuugaaacucca

SEQ ID NO: 67 - RNA, 22 nt, Artificial Sequence
  Description of Artificial Sequence Synthetic oligonucleotide
aagaugugucaacaccaguugg

SEQ ID NO: 68 - RNA, 22 nt, Artificial Sequence
  Description of Artificial Sequence Synthetic oligonucleotide
aaggggcacucuuuaagaaguc

SEQ ID NO: 69 - RNA, 22 nt, Artificial Sequence
  Description of Artificial Sequence Synthetic oligonucleotide
aacaugaaagccuguguuggca

SEQ ID NO: 70 - RNA, 21 nt, Artificial Sequence
  Description of Artificial Sequence Synthetic oligonucleotide
aucacauugccagggauuucc

SEQ ID NO: 71 - RNA, 21 nt, Artificial Sequence
  Description of Artificial Sequence Synthetic oligonucleotide
aucauucugcaagccucuagg

SEQ ID NO: 72 - RNA, 21 nt, Artificial Sequence
  Description of Artificial Sequence Synthetic oligonucleotide
auccuccaagcugucugaaug

SEQ ID NO: 73 - RNA, 21 nt, Artificial Sequence
  Description of Artificial Sequence Synthetic oligonucleotide
auccuggcuaaaucugaccug

SEQ ID NO: 74 - RNA, 21 nt, Artificial Sequence
  Description of Artificial Sequence Synthetic oligonucleotide
aucucccauuuugagaggcca

SEQ ID NO: 75 - RNA, 23 nt, Artificial Sequence
  Description of Artificial Sequence Synthetic oligonucleotide
aucacauugccagggauuaccac

SEQ ID NO: 76 - RNA, 22 nt, Artificial Sequence
  Description of Artificial Sequence Synthetic oligonucleotide
uggcucaguucagcaggaacag

SEQ ID NO: 77 - RNA, 22 nt, Artificial Sequence
  Description of Artificial Sequence Synthetic oligonucleotide
uggccaggaaggcaaugcauuc

SEQ ID NO: 78 - RNA, 22 nt, Artificial Sequence
  Description of Artificial Sequence Synthetic oligonucleotide
ugagggaaauccucccugagag

SEQ ID NO: 79 - RNA, 22 nt, Artificial Sequence
  Description of Artificial Sequence Synthetic oligonucleotide
ugccaggggcaagaaauugccu

SEQ ID NO: 80 - RNA, 22 nt, Artificial Sequence
  Description of Artificial Sequence Synthetic oligonucleotide
ugcucuggaaagcccaauaggg

SEQ ID NO: 81 - RNA, 22 nt, Artificial Sequence
  Description of Artificial Sequence Synthetic oligonucleotide
cauugcacuugucucggucuga

SEQ ID NO: 82 - RNA, 22 nt, Artificial Sequence
  Description of Artificial Sequence Synthetic oligonucleotide
cccccaauugaucguguugguu

SEQ ID NO: 83 - RNA, 22 nt, Artificial Sequence
  Description of Artificial Sequence Synthetic oligonucleotide
cuugagacccguuggucucauu

SEQ ID NO: 84 - RNA, 22 nt, Artificial Sequence
  Description of Artificial Sequence Synthetic oligonucleotide
cauuggcucguccucuaaguug

SEQ ID NO: 85 - RNA, 22 nt, Artificial Sequence
  Description of Artificial Sequence Synthetic oligonucleotide
ccauugguauucgguucaccug

SEQ ID NO: 86 - RNA, 22 nt, Artificial Sequence
  Description of Artificial Sequence Synthetic oligonucleotide
uucaaguaauccaggauaggcu

SEQ ID NO: 87 - RNA, 22 nt, Artificial Sequence
  Description of Artificial Sequence Synthetic oligonucleotide
uuacuucagaaggguacugaac

SEQ ID NO: 88 - RNA, 22 nt, Artificial Sequence
  Description of Artificial Sequence Synthetic oligonucleotide
uuacugcagguaagcuuaagac

SEQ ID NO: 89 - RNA, 22 nt, Artificial Sequence
  Description of Artificial Sequence Synthetic oligonucleotide
ucaaguuaugggaccugacaau

SEQ ID NO: 90 - RNA, 22 nt, Artificial Sequence
  Description of Artificial Sequence Synthetic oligonucleotide
uaacccucuggaggguaaauua

SEQ ID NO: 91 - RNA, 22 nt, Artificial Sequence
  Description of Artificial Sequence Synthetic oligonucleotide
uucaaguaauucaggauagguu

SEQ ID NO: 92 - RNA, 22 nt, Artificial Sequence
  Description of Artificial Sequence Synthetic oligonucleotide
uucacaguggcuaaguuccgcc

SEQ ID NO: 93 - RNA, 22 nt, Artificial Sequence
  Description of Artificial Sequence Synthetic oligonucleotide
ugacagcaucgcucagccuugu

SEQ ID NO: 94 - RNA, 19 nt, Artificial Sequence
  Description of Artificial Sequence Synthetic oligonucleotide
acugcaugggaccauucgu

SEQ ID NO: 95 - RNA, 22 nt, Artificial Sequence
  Description of Artificial Sequence Synthetic oligonucleotide
uaaaauccugucuggccccgug

SEQ ID NO: 96 - RNA, 22 nt, Artificial Sequence
  Description of Artificial Sequence Synthetic oligonucleotide
uaugaaagccccgguuugccuc

SEQ ID NO: 97 - RNA, 20 nt, Artificial Sequence
  Description of Artificial Sequence Synthetic oligonucleotide
uucacaguggcuaaguucug

SEQ ID NO: 98 - RNA, 22 nt, Artificial Sequence
  Description of Artificial Sequence Synthetic oligonucleotide
aaggagcucacagucuauugag

SEQ ID NO: 99 - RNA, 22 nt, Artificial Sequence
  Description of Artificial Sequence Synthetic oligonucleotide
cuagcaccaucugaaaucgguu

SEQ ID NO: 100 - RNA, 22 nt, Artificial Sequence
  Description of Artificial Sequence Synthetic oligonucleotide
ccucacuacgaauuaagggcuu

SEQ ID NO: 101 - RNA, 22 nt, Artificial Sequence
  Description of Artificial Sequence Synthetic oligonucleotide
cugauagacgaaugcacccuuu

SEQ ID NO: 102 - RNA, 22 nt, Artificial Sequence
  Description of Artificial Sequence Synthetic oligonucleotide
cacuaagucggcaauugucuca

SEQ ID NO: 103 - RNA, 22 nt, Artificial Sequence
  Description of Artificial Sequence Synthetic oligonucleotide
cucuagaucaagacuucgcagu

SEQ ID NO: 104 - RNA, 23 nt, Artificial Sequence
  Description of Artificial Sequence Synthetic oligonucleotide
uagcaccauuugaaaucaguguu

SEQ ID NO: 105 - RNA, 23 nt, Artificial Sequence
  Description of Artificial Sequence Synthetic oligonucleotide
uauuagaaaccugcucuuguaag

SEQ ID NO: 106 - RNA, 23 nt, Artificial Sequence
  Description of Artificial Sequence Synthetic oligonucleotide
uaugcagaacucucaauuaguug

SEQ ID NO: 107 - RNA, 23 nt, Artificial Sequence
  Description of Artificial Sequence Synthetic oligonucleotide
uuacuuuaaggacaggaauuccu

SEQ ID NO: 108 - RNA, 23 nt, Artificial Sequence
  Description of Artificial Sequence Synthetic oligonucleotide
uauguucucccauuggauaaaag

SEQ ID NO: 109 - RNA, 22 nt, Artificial Sequence
  Description of Artificial Sequence Synthetic oligonucleotide
uagcaccauuugaaaucgguua

SEQ ID NO: 110 - RNA, 23 nt, Artificial Sequence
  Description of Artificial Sequence Synthetic oligonucleotide
uguaaacauccucgacuggaagc

SEQ ID NO: 111 - RNA, 21 nt, Artificial Sequence
  Description of Artificial Sequence Synthetic oligonucleotide
uguaaacauccuacacucagc

SEQ ID NO: 112 - RNA, 21 nt, Artificial Sequence
  Description of Artificial Sequence Synthetic oligonucleotide
uugucacacacauccacauag

SEQ ID NO: 113 - RNA, 21 nt, Artificial Sequence
  Description of Artificial Sequence Synthetic oligonucleotide
uccagagcaacaccuauucua

SEQ ID NO: 114 - RNA, 21 nt, Artificial Sequence
  Description of Artificial Sequence Synthetic oligonucleotide
uaagcccauguccauuacaca

SEQ ID NO: 115 - RNA, 21 nt, Artificial Sequence
  Description of Artificial Sequence Synthetic oligonucleotide
ucuguccacacaugaccauaa

SEQ ID NO: 116 - RNA, 23 nt, Artificial Sequence
  Description of Artificial Sequence Synthetic oligonucleotide
uguaaacauccuacacucucagc

SEQ ID NO: 117 - RNA, 22 nt, Artificial Sequence
  Description of Artificial Sequence Synthetic oligonucleotide
uguaaacauccccgacuggaag

SEQ ID NO: 118 - RNA, 19 nt, Artificial Sequence
  Description of Artificial Sequence Synthetic oligonucleotide
uguaaacauccuugacugg

SEQ ID NO: 119 - RNA, 21 nt, Artificial Sequence
  Description of Artificial Sequence Synthetic oligonucleotide
ggcaagaugcuggcauagcug

SEQ ID NO: 120 - RNA, 21 nt, Artificial Sequence
  Description of Artificial Sequence Synthetic oligonucleotide
gggcaacugaguccuuagagg

SEQ ID NO: 121 - RNA, 21 nt, Artificial Sequence
  Description of Artificial Sequence Synthetic oligonucleotide
guugaggcuagucaggcacag

SEQ ID NO: 122 - RNA, 21 nt, Artificial Sequence
  Description of Artificial Sequence Synthetic oligonucleotide
gaaugggcauggauuggccca

SEQ ID NO: 123 - RNA, 21 nt, Artificial Sequence
  Description of Artificial Sequence Synthetic oligonucleotide
gguacaaggcaaggucugguc

SEQ ID NO: 124 - RNA, 21 nt, Artificial Sequence
  Description of Artificial Sequence Synthetic oligonucleotide
uauugcacauuacuaaguugc

SEQ ID NO: 125 - RNA, 19 nt, Artificial Sequence
  Description of Artificial Sequence Synthetic oligonucleotide
gugcauuguaguugcauug

SEQ ID NO: 126 - RNA, 19 nt, Artificial Sequence
  Description of Artificial Sequence Synthetic oligonucleotide
gguuugaucaucuaggguu

SEQ ID NO: 127 - RNA, 19 nt, Artificial Sequence
  Description of Artificial Sequence Synthetic oligonucleotide
gggguucuugauuagcuua

SEQ ID NO: 128 - RNA, 19 nt, Artificial Sequence
  Description of Artificial Sequence Synthetic oligonucleotide
gaucuuggcuaagguguuu

SEQ ID NO: 129 - RNA, 19 nt, Artificial Sequence
  Description of Artificial Sequence Synthetic oligonucleotide
gauuguuguaggucacuug

SEQ ID NO: 130 - RNA, 19 nt, Artificial Sequence
  Description of Artificial Sequence Synthetic oligonucleotide
gugcauugcuguugcauug

SEQ ID NO: 131 - RNA, 22 nt, Artificial Sequence
  Description of Artificial Sequence Synthetic oligonucleotide
uggcagugucuuagcugguugu

SEQ ID NO: 132 - RNA, 22 nt, Artificial Sequence
  Description of Artificial Sequence Synthetic oligonucleotide
uguggcugauucucuauggggu

SEQ ID NO: 133 - RNA, 22 nt, Artificial Sequence
  Description of Artificial Sequence Synthetic oligonucleotide
ugggccuguguuuguguguaac

SEQ ID NO: 134 - RNA, 22 nt, Artificial Sequence
  Description of Artificial Sequence Synthetic oligonucleotide
uuugugugucagugggagucuc

SEQ ID NO: 135 - RNA, 22 nt, Artificial Sequence
  Description of Artificial Sequence Synthetic oligonucleotide
uuccuggagggucugguauguu

SEQ ID NO: 136 - RNA, 22 nt, Artificial Sequence
  Description of Artificial Sequence Synthetic oligonucleotide
uauugcacuugucccggccugu

SEQ ID NO: 137 - RNA, 22 nt, Artificial Sequence
  Description of Artificial Sequence Synthetic oligonucleotide
aaagugcuguucgugcagguag

SEQ ID NO: 138 - RNA, 22 nt, Artificial Sequence
  Description of Artificial Sequence Synthetic oligonucleotide
aacagguugccggagauguguu

SEQ ID NO: 139 - RNA, 22 nt, Artificial Sequence
  Description of Artificial Sequence Synthetic oligonucleotide
aaguguggcguaaagugcuugc

SEQ ID NO: 140 - RNA, 22 nt, Artificial Sequence
  Description of Artificial Sequence Synthetic oligonucleotide
auuuuggagcggucagcuagag

SEQ ID NO: 141 - RNA, 22 nt, Artificial Sequence
  Description of Artificial Sequence Synthetic oligonucleotide
agcacuggcgguuuaauuggga

SEQ ID NO: 142 - RNA, 20 nt, Artificial Sequence
  Description of Artificial Sequence Synthetic oligonucleotide
aaagugcugacagugcagau

SEQ ID NO: 143 - RNA, 22 nt, Artificial Sequence
  Description of Artificial Sequence Synthetic oligonucleotide
uucaacggguauuuauugagca

SEQ ID NO: 144 - RNA, 22 nt, Artificial Sequence
  Description of Artificial Sequence Synthetic oligonucleotide
uuuggcacuagcacauuuuugc

SEQ ID NO: 145 - RNA, 22 nt, Artificial Sequence
  Description of Artificial Sequence Synthetic oligonucleotide
ugaaauccuuggauguucucuc

SEQ ID NO: 146 - RNA, 22 nt, Artificial Sequence
  Description of Artificial Sequence Synthetic oligonucleotide
ugcuucacugguuuaaccaguu

SEQ ID NO: 147 - RNA, 22 nt, Artificial Sequence
  Description of Artificial Sequence Synthetic oligonucleotide
ugaaaguccuucugucauuugc

SEQ ID NO: 148 - RNA, 22 nt, Artificial Sequence
  Description of Artificial Sequence Synthetic oligonucleotide
uuguugcaggcacauccuuuua

SEQ ID NO: 149 - RNA, 22 nt, Artificial Sequence
  Description of Artificial Sequence Synthetic oligonucleotide
ugagguaguaaguuguauuguu

SEQ ID NO: 150 - RNA, 20 nt, Artificial Sequence
  Description of Artificial Sequence Synthetic oligonucleotide
acccguagauccgaucuugu

SEQ ID NO: 151 - RNA, 20 nt, Artificial Sequence
  Description of Artificial Sequence Synthetic oligonucleotide
auuguacgaucccgcuguca

SEQ ID NO: 152 - RNA, 20 nt, Artificial Sequence
  Description of Artificial Sequence Synthetic oligonucleotide
auauuccgugagaccgcucu

SEQ ID NO: 153 - RNA, 20 nt, Artificial Sequence
  Description of Artificial Sequence Synthetic oligonucleotide
auccgaucggcacuuguauc

SEQ ID NO: 154 - RNA, 20 nt, Artificial Sequence
  Description of Artificial Sequence Synthetic oligonucleotide
auuaccgacguacuggcucu

SEQ ID NO: 155 - RNA, 22 nt, Artificial Sequence
  Description of Artificial Sequence Synthetic oligonucleotide
cacccguagaaccgaccuugcg

SEQ ID NO: 156 - RNA, 22 nt, Artificial Sequence
  Description of Artificial Sequence Synthetic oligonucleotide
aacccguagauccgaacuugug

SEQ ID NO: 157 - RNA, 22 nt, Artificial Sequence
  Description of Artificial Sequence Synthetic oligonucleotide
acgcauaacguggguuucaacc

SEQ ID NO: 158 - RNA, 22 nt, Artificial Sequence
  Description of Artificial Sequence Synthetic oligonucleotide
agcgauuacucaacucgucagg

SEQ ID NO: 159 - RNA, 22 nt, Artificial Sequence
  Description of Artificial Sequence Synthetic oligonucleotide
auaagcgcauucccgggauuca

SEQ ID NO: 160 - RNA, 22 nt, Artificial Sequence
  Description of Artificial Sequence Synthetic oligonucleotide
aguacggcuauauggcuacacc

SEQ ID NO: 161 - RNA, 20 nt, Artificial Sequence
  Description of Artificial Sequence Synthetic oligonucleotide
uacaguacugugauaacuga

SEQ ID NO: 162 - RNA, 20 nt, Artificial Sequence
  Description of Artificial Sequence Synthetic oligonucleotide
uuauacacuuaguaagaggc

SEQ ID NO: 163 - RNA, 20 nt, Artificial Sequence
  Description of Artificial Sequence Synthetic oligonucleotide
uuauauagaagcuagacugc

SEQ ID NO: 164 - RNA, 20 nt, Artificial Sequence
  Description of Artificial Sequence Synthetic oligonucleotide
uaugcuauguagccaauaga

SEQ ID NO: 165 - RNA, 20 nt, Artificial Sequence
  Description of Artificial Sequence Synthetic oligonucleotide
uaauauagggugcuaucaac

SEQ ID NO: 166 - RNA, 23 nt, Artificial Sequence
  Description of Artificial Sequence Synthetic oligonucleotide
agcagcauuguacagggcuauga

SEQ ID NO: 167 - RNA, 23 nt, Artificial Sequence
  Description of Artificial Sequence Synthetic oligonucleotide
aucagggcaguauugagaugacc

SEQ ID NO: 168 - RNA, 23 nt, Artificial Sequence
  Description of Artificial Sequence Synthetic oligonucleotide
aagugagaguaggcucuagacuc

SEQ ID NO: 169 - RNA, 23 nt, Artificial Sequence
  Description of Artificial Sequence Synthetic oligonucleotide
agggcucuaauggacaggauauc

SEQ ID NO: 170 - RNA, 23 nt, Artificial Sequence
  Description of Artificial Sequence Synthetic oligonucleotide
auggaaguuuccucggagcaaag

SEQ ID NO: 171 - RNA, 23 nt, Artificial Sequence
  Description of Artificial Sequence Synthetic oligonucleotide
agcaacauuguacagggcuauga

SEQ ID NO: 172 - RNA, 22 nt, Artificial Sequence
  Description of Artificial Sequence Synthetic oligonucleotide
ucaacaucagucugauaagcua

SEQ ID NO: 173 - RNA, 22 nt, Artificial Sequence
  Description of Artificial Sequence Synthetic oligonucleotide
uuucauagaagaaaacccuucg

SEQ ID NO: 174 - RNA, 22 nt, Artificial Sequence
  Description of Artificial Sequence Synthetic oligonucleotide
ucuaaagucaggauacauuacc

SEQ ID NO: 175 - RNA, 22 nt, Artificial Sequence
  Description of Artificial Sequence Synthetic oligonucleotide
uugaaucucacacaguagauca

SEQ ID NO: 176 - RNA, 22 nt, Artificial Sequence
  Description of Artificial Sequence Synthetic oligonucleotide
ugcagaauuacccuuaagacua

SEQ ID NO: 177 - RNA, 20 nt, Artificial Sequence
  Description of Artificial Sequence Synthetic oligonucleotide
ucaaaugcucagacuccugu

SEQ ID NO: 178 - RNA, 24 nt, Artificial Sequence
  Description of Artificial Sequence Synthetic oligonucleotide
aaaagugcuuacagugcagguagc

SEQ ID NO: 179 - RNA, 23 nt, Artificial Sequence
  Description of Artificial Sequence Synthetic oligonucleotide
agcagcauuguacagggcuauca

SEQ ID NO: 180 - RNA, 21 nt, Artificial Sequence
  Description of Artificial Sequence Synthetic oligonucleotide
auaaggauuuuuaggggcauu

SEQ ID NO: 181 - RNA, 21 nt, Artificial Sequence
  Description of Artificial Sequence Synthetic oligonucleotide
auguuaugaggcaaugauuug

SEQ ID NO: 182 - RNA, 21 nt, Artificial Sequence
  Description of Artificial Sequence Synthetic oligonucleotide
auaagggaauuguggauucuu

SEQ ID NO: 183 - RNA, 21 nt, Artificial Sequence
  Description of Artificial Sequence Synthetic oligonucleotide
aguuguguuguuagaucaaag

SEQ ID NO: 184 - RNA, 21 nt, Artificial Sequence
  Description of Artificial Sequence Synthetic oligonucleotide
aguagaugaagaguuuguuuc

SEQ ID NO: 185 - RNA, 23 nt, Artificial Sequence
  Description of Artificial Sequence Synthetic oligonucleotide
uggagugugacaaugguguuugu

SEQ ID NO: 186 - RNA, 23 nt, Artificial Sequence
  Description of Artificial Sequence Synthetic oligonucleotide
ugguacagguugugggaauguuu

SEQ ID NO: 187 - RNA, 23 nt, Artificial Sequence
  Description of Artificial Sequence Synthetic oligonucleotide
uggguaggauuuguucuuggaga

SEQ ID NO: 188 - RNA, 23 nt, Artificial Sequence
  Description of Artificial Sequence Synthetic oligonucleotide
uuguagcuguaagugauuugggg

SEQ ID NO: 189 - RNA, 23 nt, Artificial Sequence
  Description of Artificial Sequence Synthetic oligonucleotide
uuaaguuauuguggggugcagug

SEQ ID NO: 190 - RNA, 21 nt, Artificial Sequence
  Description of Artificial Sequence Synthetic oligonucleotide
cauuauuacuuuugguacgcg

SEQ ID NO: 191 - RNA, 21 nt, Artificial Sequence
  Description of Artificial Sequence Synthetic oligonucleotide
cuaauauuuugcgcgugcuua

SEQ ID NO: 192 - RNA, 21 nt, Artificial Sequence
  Description of Artificial Sequence Synthetic oligonucleotide
cuuauauuuuccgcgauguga

SEQ ID NO: 193 - RNA, 21 nt, Artificial Sequence
  Description of Artificial Sequence Synthetic oligonucleotide
cuauaauuuucguuacgcugg

SEQ ID NO: 194 - RNA, 21 nt, Artificial Sequence
  Description of Artificial Sequence Synthetic oligonucleotide
cauguauuucggauucuuacg

SEQ ID NO: 195 - RNA, 22 nt, Artificial Sequence
  Description of Artificial Sequence Synthetic oligonucleotide
uuaaggcacgcggugaaugcca

SEQ ID NO: 196 - RNA, 22 nt, Artificial Sequence
  Description of Artificial Sequence Synthetic oligonucleotide
ugcagaacggaggcgacaucuu

SEQ ID NO: 197 - RNA, 22 nt, Artificial Sequence
  Description of Artificial Sequence Synthetic oligonucleotide
uuagagagcgucgaaggacucc

SEQ ID NO: 198 - RNA, 22 nt, Artificial Sequence
  Description of Artificial Sequence Synthetic oligonucleotide
uugauggccgauaaccgcagag

SEQ ID NO: 199 - RNA, 22 nt, Artificial Sequence
  Description of Artificial Sequence Synthetic oligonucleotide
ugcaggacgucauccgaaggau

SEQ ID NO: 200 - RNA, 23 nt, Artificial Sequence
  Description of Artificial Sequence Synthetic oligonucleotide
ucccugagacccuuuaaccugug

SEQ ID NO: 201 - RNA, 22 nt, Artificial Sequence
  Description of Artificial Sequence Synthetic oligonucleotide
ucccugagacccuaacuuguga

SEQ ID NO: 202 - RNA, 22 nt, Artificial Sequence
  Description of Artificial Sequence Synthetic oligonucleotide
ucugggccaauaugcauccacu

SEQ ID NO: 203 - RNA, 22 nt, Artificial Sequence
  Description of Artificial Sequence Synthetic oligonucleotide
uccaccugcagacauuguagcu

SEQ ID NO: 204 - RNA, 22 nt, Artificial Sequence
  Description of Artificial Sequence Synthetic oligonucleotide
ucagcccaucugcaguacaguu

SEQ ID NO: 205 - RNA, 22 nt, Artificial Sequence
  Description of Artificial Sequence Synthetic oligonucleotide
uaacccagcucuccuggguaau

SEQ ID NO: 206 - RNA, 21 nt, Artificial Sequence
  Description of Artificial Sequence Synthetic oligonucleotide
ucguaccgugaguaauaaugc

SEQ ID NO: 207 - RNA, 21 nt, Artificial Sequence
  Description of Artificial Sequence Synthetic oligonucleotide
uaucgcgacuuaguacaguga

SEQ ID NO: 208 - RNA, 21 nt, Artificial Sequence
  Description of Artificial Sequence Synthetic oligonucleotide
ucguaucguaagauagugacc

SEQ ID NO: 209 - RNA, 21 nt, Artificial Sequence
  Description of Artificial Sequence Synthetic oligonucleotide
uacgaucgcuaaucaugggua

SEQ ID NO: 210 - RNA, 21 nt, Artificial Sequence
  Description of Artificial Sequence Synthetic oligonucleotide
uccguacggaagacuuaugua

SEQ ID NO: 211 - RNA, 22 nt, Artificial Sequence
  Description of Artificial Sequence Synthetic oligonucleotide
ucggauccgucugagcuuggcu

SEQ ID NO: 212 - RNA, 22 nt, Artificial Sequence
  Description of Artificial Sequence Synthetic oligonucleotide
ucacagugaaccggucucuuuu

SEQ ID NO: 213 - RNA, 22 nt, Artificial Sequence
  Description of Artificial Sequence Synthetic oligonucleotide
ucugcucaaguucgcucaaugu

SEQ ID NO: 214 - RNA, 22 nt, Artificial Sequence
  Description of Artificial Sequence Synthetic oligonucleotide
uaacugaacugcguuuccucug

SEQ ID NO: 215 - RNA, 22 nt, Artificial Sequence
  Description of Artificial Sequence Synthetic oligonucleotide
uggcccugcauuaccuauguau

SEQ ID NO: 216 - RNA, 22 nt, Artificial Sequence
  Description of Artificial Sequence Synthetic oligonucleotide
ugacagaaccuguuucgcucuu

SEQ ID NO: 217 - RNA, 22 nt, Artificial Sequence
  Description of Artificial Sequence Synthetic oligonucleotide
ucacagugaaccggucucuuuc

SEQ ID NO: 218 - RNA, 20 nt, Artificial Sequence
  Description of Artificial Sequence Synthetic oligonucleotide
cuuuuucggucugggcuugc

SEQ ID NO: 219 - RNA, 21 nt, Artificial Sequence
  Description of Artificial Sequence Synthetic oligonucleotide
cuuuuugcggucugggcuugc

SEQ ID NO: 220 - RNA, 21 nt, Artificial Sequence
  Description of Artificial Sequence Synthetic oligonucleotide
cuccuuuguguuuggguccgg

SEQ ID NO: 221 - RNA, 21 nt, Artificial Sequence
  Description of Artificial Sequence Synthetic oligonucleotide
cuuggggguuuccgguucuuc

SEQ ID NO: 222 - RNA, 21 nt, Artificial Sequence
  Description of Artificial Sequence Synthetic oligonucleotide
ccuuuuggcguuuggcuuggc

SEQ ID NO: 223 - RNA, 21 nt, Artificial Sequence
  Description of Artificial Sequence Synthetic oligonucleotide
cuccuuuguucugguugggcg

SEQ ID NO: 224 - RNA, 20 nt, Artificial Sequence
  Description of Artificial Sequence Synthetic oligonucleotide
cagugcaauguuaaaagggc

SEQ ID NO: 225 - RNA, 20 nt, Artificial Sequence
  Description of Artificial Sequence Synthetic oligonucleotide
caugagagguggacuucaaa

SEQ ID NO: 226 - RNA, 20 nt, Artificial Sequence
  Description of Artificial Sequence Synthetic oligonucleotide
cugaaugcagacuuggaaga

SEQ ID NO: 227 - RNA, 20 nt, Artificial Sequence
  Description of Artificial Sequence Synthetic oligonucleotide
cuguuggagggaaacauaac

SEQ ID NO: 228 - RNA, 20 nt, Artificial Sequence
  Description of Artificial Sequence Synthetic oligonucleotide
cagcucaaauugaggaugga

SEQ ID NO: 229 - RNA, 20 nt, Artificial Sequence
  Description of Artificial Sequence Synthetic oligonucleotide
cagugcaaugaugaaagggc

SEQ ID NO: 230 - RNA, 21 nt, Artificial Sequence
  Description of Artificial Sequence Synthetic oligonucleotide
uaaagcuagauaaccgaaagu

SEQ ID NO: 231 - RNA, 21 nt, Artificial Sequence
  Description of Artificial Sequence Synthetic oligonucleotide
uaagauaaacgugaaugcaca

SEQ ID NO: 232 - RNA, 21 nt, Artificial Sequence
  Description of Artificial Sequence Synthetic oligonucleotide
uagaagauccgauguaaaaca

SEQ ID NO: 233 - RNA, 21 nt, Artificial Sequence
  Description of Artificial Sequence Synthetic oligonucleotide
uaugaaacgagccuaaaagua

SEQ ID NO: 234 - RNA, 21 nt, Artificial Sequence
  Description of Artificial Sequence Synthetic oligonucleotide
uaugacaacaaagucgagaua

SEQ ID NO: 235 - RNA, 23 nt, Artificial Sequence
  Description of Artificial Sequence Synthetic oligonucleotide
uaacagucuacagccauggucgc

SEQ ID NO: 236 - RNA, 23 nt, Artificial Sequence
  Description of Artificial Sequence Synthetic oligonucleotide
uagcaucccauagucggagaucc

SEQ ID NO: 237 - RNA, 23 nt, Artificial Sequence
  Description of Artificial Sequence Synthetic oligonucleotide
ucacauccugguacggaagaccu

SEQ ID NO: 238 - RNA, 23 nt, Artificial Sequence
  Description of Artificial Sequence Synthetic oligonucleotide
uaaugccauacugccuaccggag

SEQ ID NO: 239 - RNA, 23 nt, Artificial Sequence
  Description of Artificial Sequence Synthetic oligonucleotide
uacuggcauuccgacaguacagc

SEQ ID NO: 240 - RNA, 22 nt, Artificial Sequence
  Description of Artificial Sequence Synthetic oligonucleotide
uugguccccuucaaccagcugu

SEQ ID NO: 241 - RNA, 22 nt, Artificial Sequence
  Description of Artificial Sequence Synthetic oligonucleotide
ugcccaccucauugcuugucag

SEQ ID NO: 242 - RNA, 22 nt, Artificial Sequence
  Description of Artificial Sequence Synthetic oligonucleotide
ucugguucucaugaagccuccc

SEQ ID NO: 243 - RNA, 22 nt, Artificial Sequence
  Description of Artificial Sequence Synthetic oligonucleotide
ucaagucccccuugccuuuagg

SEQ ID NO: 244 - RNA, 22 nt, Artificial Sequence
  Description of Artificial Sequence Synthetic oligonucleotide
ugcaucucuuuggcccacagcu

SEQ ID NO: 245 - RNA, 21 nt, Artificial Sequence
  Description of Artificial Sequence Synthetic oligonucleotide
uugguccccuucaaccagcua

SEQ ID NO: 246 - RNA, 22 nt, Artificial Sequence
  Description of Artificial Sequence Synthetic oligonucleotide
ugugacugguugaccagagggg

SEQ ID NO: 247 - RNA, 24 nt, Artificial Sequence
  Description of Artificial Sequence Synthetic oligonucleotide
uauggcuuuuuauuccuaugugau

SEQ ID NO: 248 - RNA, 24 nt, Artificial Sequence
  Description of Artificial Sequence Synthetic oligonucleotide
uacucugguuuuuuguguacuaau

SEQ ID NO: 249 - RNA, 24 nt, Artificial Sequence
  Description of Artificial Sequence Synthetic oligonucleotide
uaugcauguugugcuauuuuuauc

SEQ ID NO: 250 - RNA, 24 nt, Artificial Sequence
  Description of Artificial Sequence Synthetic oligonucleotide
uaguucuuggcuauuuauauuugc

SEQ ID NO: 251 - RNA, 24 nt, Artificial Sequence
  Description of Artificial Sequence Synthetic oligonucleotide
uauuuauguuagguuuucugcuac

SEQ ID NO: 252 - RNA, 23 nt, Artificial Sequence
  Description of Artificial Sequence Synthetic oligonucleotide
acuccauuuguuuugaugaugga

SEQ ID NO: 253 - RNA, 22 nt, Artificial Sequence
  Description of Artificial Sequence Synthetic oligonucleotide
uauugcuuaagaauacgcguag

SEQ ID NO: 254 - RNA, 22 nt, Artificial Sequence
  Description of Artificial Sequence Synthetic oligonucleotide
uagaagucuuacgauuaacggu

SEQ ID NO: 255 - RNA, 22 nt, Artificial Sequence
  Description of Artificial Sequence Synthetic oligonucleotide
uacaagugacgaauguuacguu

SEQ ID NO: 256 - RNA, 22 nt, Artificial Sequence
  Description of Artificial Sequence Synthetic oligonucleotide
uagagauuaauacgcguacuug

SEQ ID NO: 257 - RNA, 22 nt, Artificial Sequence
  Description of Artificial Sequence Synthetic oligonucleotide
uuuacuaauagacgugagaucg

SEQ ID NO: 258 - RNA, 17 nt, Artificial Sequence
  Description of Artificial Sequence Synthetic oligonucleotide
agcugguguugugaauc

SEQ ID NO: 259 - RNA, 17 nt, Artificial Sequence
  Description of Artificial Sequence Synthetic oligonucleotide
agccuguguauuuggag

SEQ ID NO: 260 - RNA, 17 nt, Artificial Sequence
  Description of Artificial Sequence Synthetic oligonucleotide
aucagugguuacuuggg

SEQ ID NO: 261 - RNA, 17 nt, Artificial Sequence
  Description of Artificial Sequence Synthetic oligonucleotide
auggagggugauuuccu

SEQ ID NO: 262 - RNA, 17 nt, Artificial Sequence
  Description of Artificial Sequence Synthetic oligonucleotide
auggauuuguagccugg

SEQ ID NO: 263 - RNA, 23 nt, Artificial Sequence
  Description of Artificial Sequence Synthetic oligonucleotide
ucuacagugcacgugucuccagu

SEQ ID NO: 264 - RNA, 21 nt, Artificial Sequence
  Description of Artificial Sequence Synthetic oligonucleotide
agugguuuuacccuaugguag

SEQ ID NO: 265 - RNA, 21 nt, Artificial Sequence
  Description of Artificial Sequence Synthetic oligonucleotide
agcaugugaugguauccuguu

SEQ ID NO: 266 - RNA, 21 nt, Artificial Sequence
  Description of Artificial Sequence Synthetic oligonucleotide
acauuguuggcuggguauacu

SEQ ID NO: 267 - RNA, 21 nt, Artificial Sequence
  Description of Artificial Sequence Synthetic oligonucleotide
agugggcuuuucuugacgaau

SEQ ID NO: 268 - RNA, 21 nt, Artificial Sequence
  Description of Artificial Sequence Synthetic oligonucleotide
aagaguccuuuuucggguuag

SEQ ID NO: 269 - RNA, 21 nt, Artificial Sequence
  Description of Artificial Sequence Synthetic oligonucleotide
aacacugucugguaaagaugg

SEQ ID NO: 270 - RNA, 21 nt, Artificial Sequence
  Description of Artificial Sequence Synthetic oligonucleotide
auggcagaaaugugcucagau

SEQ ID NO: 271 - RNA, 21 nt, Artificial Sequence
  Description of Artificial Sequence Synthetic oligonucleotide
acugauuugcaagugagcaga

SEQ ID NO: 272 - RNA, 21 nt, Artificial Sequence
  Description of Artificial Sequence Synthetic oligonucleotide
agugaggagccaguuaacauu

SEQ ID NO: 273 - RNA, 21 nt, Artificial Sequence
  Description of Artificial Sequence Synthetic oligonucleotide
aggugggaucaagcucauuaa

SEQ ID NO: 274 - RNA, 22 nt, Artificial Sequence
  Description of Artificial Sequence Synthetic oligonucleotide
uguaguguuuccuacuuuaugg

SEQ ID NO: 275 - RNA, 22 nt, Artificial Sequence
  Description of Artificial Sequence Synthetic oligonucleotide
ugugguaucuugacuucuauug

SEQ ID NO: 276 - RNA, 22 nt, Artificial Sequence
  Description of Artificial Sequence Synthetic oligonucleotide
uauagccuucuuguagguguuu

SEQ ID NO: 277 - RNA, 22 nt, Artificial Sequence
  Description of Artificial Sequence Synthetic oligonucleotide
uuguaguacuuguuugcuacug

SEQ ID NO: 278 - RNA, 22 nt, Artificial Sequence
  Description of Artificial Sequence Synthetic oligonucleotide
uacuagcuuuggcuuguuguau

SEQ ID NO: 279 - RNA, 22 nt, Artificial Sequence
  Description of Artificial Sequence Synthetic oligonucleotide
cccauaaaguagaaagcacuac

SEQ ID NO: 280 - RNA, 22 nt, Artificial Sequence
  Description of Artificial Sequence Synthetic oligonucleotide
cagagucauaagccauaaacac

SEQ ID NO: 281 - RNA, 22 nt, Artificial Sequence
  Description of Artificial Sequence Synthetic oligonucleotide
cagaagauaauaaaccaugccc

SEQ ID NO: 282 - RNA, 22 nt, Artificial Sequence
  Description of Artificial Sequence Synthetic oligonucleotide
ccacuaaaagagcagacauacu

SEQ ID NO: 283 - RNA, 22 nt, Artificial Sequence
  Description of Artificial Sequence Synthetic oligonucleotide
cuaccaaaaaaucgaaagccug

SEQ ID NO: 284 - RNA, 22 nt, Artificial Sequence
  Description of Artificial Sequence Synthetic oligonucleotide
ugagaugaagcacuguagcuca

SEQ ID NO: 285 - RNA, 22 nt, Artificial Sequence
  Description of Artificial Sequence Synthetic oligonucleotide
uaauguggagcucacacaguga

SEQ ID NO: 286 - RNA, 22 nt, Artificial Sequence
  Description of Artificial Sequence Synthetic oligonucleotide
ucagaauagauggcucagugca

SEQ ID NO: 287 - RNA, 22 nt, Artificial Sequence
  Description of Artificial Sequence Synthetic oligonucleotide
ucaguggaaggaauaccugacu

SEQ ID NO: 288 - RNA, 22 nt, Artificial Sequence
  Description of Artificial Sequence Synthetic oligonucleotide
uguccccauaagaagugagaug

SEQ ID NO: 289 - RNA, 22 nt, Artificial Sequence
  Description of Artificial Sequence Synthetic oligonucleotide
uacaguauagaugauguacuag

SEQ ID NO: 290 - RNA, 22 nt, Artificial Sequence
  Description of Artificial Sequence Synthetic oligonucleotide
uuauauuauugcgaaagagagc

SEQ ID NO: 291 - RNA, 22 nt, Artificial Sequence
  Description of Artificial Sequence Synthetic oligonucleotide
uuauguauaaggguucaacgaa

SEQ ID NO: 292 - RNA, 22 nt, Artificial Sequence
  Description of Artificial Sequence Synthetic oligonucleotide
uauaaugucgucuaaaggaaug

SEQ ID NO: 293 - RNA, 22 nt, Artificial Sequence
  Description of Artificial Sequence Synthetic oligonucleotide
uuaauauaggcauugugcgaaa

SEQ ID NO: 294 - RNA, 24 nt, Artificial Sequence
  Description of Artificial Sequence Synthetic oligonucleotide
guccaguuuucccaggaaucccuu

SEQ ID NO: 295 - RNA, 24 nt, Artificial Sequence
  Description of Artificial Sequence Synthetic oligonucleotide
gucccccugcaagaguuuuucauc

SEQ ID NO: 296 - RNA, 24 nt, Artificial Sequence
  Description of Artificial Sequence Synthetic oligonucleotide
guuccagcucuugcccuugcaaau

SEQ ID NO: 297 - RNA, 24 nt, Artificial Sequence
  Description of Artificial Sequence Synthetic oligonucleotide
gcaccccuugcuguucaagacuuu

SEQ ID NO: 298 - RNA, 24 nt, Artificial Sequence
  Description of Artificial Sequence Synthetic oligonucleotide
gaggccacuccagcuucaucuuuu

SEQ ID NO: 299 - RNA, 22 nt, Artificial Sequence
  Description of Artificial Sequence Synthetic oligonucleotide
ugagaacugaauuccauggguu

SEQ ID NO: 300 - RNA, 22 nt, Artificial Sequence
  Description of Artificial Sequence Synthetic oligonucleotide
uucuggauggcuuacaaaugag

SEQ ID NO: 301 - RNA, 22 nt, Artificial Sequence
  Description of Artificial Sequence Synthetic oligonucleotide
ugaauggauucaguugcacagu

SEQ ID NO: 302 - RNA, 22 nt, Artificial Sequence
  Description of Artificial Sequence Synthetic oligonucleotide
uggaguuucuaacaguugaagc

SEQ ID NO: 303 - RNA, 22 nt, Artificial Sequence
  Description of Artificial Sequence Synthetic oligonucleotide
uugaggacugagcuuguuaaca

SEQ ID NO: 304 - RNA, 21 nt, Artificial Sequence
  Description of Artificial Sequence Synthetic oligonucleotide
guguguggaaaugcuucugcc

SEQ ID NO: 305 - RNA, 22 nt, Artificial Sequence
  Description of Artificial Sequence Synthetic oligonucleotide
ucagugcacuacagaacuuugu

SEQ ID NO: 306 - RNA, 22 nt, Artificial Sequence
  Description of Artificial Sequence Synthetic oligonucleotide
ugcccugcuugauaucaagaau

SEQ ID NO: 307 - RNA, 22 nt, Artificial Sequence
  Description of Artificial Sequence Synthetic oligonucleotide
ucugugaguaaaaugccacuuc

SEQ ID NO: 308 - RNA, 22 nt, Artificial Sequence
  Description of Artificial Sequence Synthetic oligonucleotide
ugcauuuaccugaaguuaccag

SEQ ID NO: 309 - RNA, 22 nt, Artificial Sequence
  Description of Artificial Sequence Synthetic oligonucleotide
ucagugaacugcuauucugcaa

SEQ ID NO: 310 - RNA, 22 nt, Artificial Sequence
  Description of Artificial Sequence Synthetic oligonucleotide
ucagugcaucacagaacuuugu

SEQ ID NO: 311 - RNA, 22 nt, Artificial Sequence
  Description of Artificial Sequence Synthetic oligonucleotide
ucuggcuccgugucuucacucc

SEQ ID NO: 312 - RNA, 23 nt, Artificial Sequence
  Description of Artificial Sequence Synthetic oligonucleotide
ucucccaacccuuguaccagugu

SEQ ID NO: 313 - RNA, 21 nt, Artificial Sequence
  Description of Artificial Sequence Synthetic oligonucleotide
cuagacugaagcuccuugagg

SEQ ID NO: 314 - RNA, 21 nt, Artificial Sequence
  Description of Artificial Sequence Synthetic oligonucleotide
ucagugcaugacagaacuugg

SEQ ID NO: 315 - RNA, 20 nt, Artificial Sequence
  Description of Artificial Sequence Synthetic oligonucleotide
uugcauagucacaaaaguga

SEQ ID NO: 316 - RNA, 20 nt, Artificial Sequence
  Description of Artificial Sequence Synthetic oligonucleotide
uuaagccuaagaugaacaug

SEQ ID NO: 317 - RNA, 20 nt, Artificial Sequence
  Description of Artificial Sequence Synthetic oligonucleotide
ugaguuguaaagcccaauaa

SEQ ID NO: 318 - RNA, 20 nt, Artificial Sequence
  Description of Artificial Sequence Synthetic oligonucleotide
uccaaugucuaagaauaagg

SEQ ID NO: 319 - RNA, 20 nt, Artificial Sequence
  Description of Artificial Sequence Synthetic oligonucleotide
uuagagugacaacacuuaag

SEQ ID NO: 320 - RNA, 22 nt, Artificial Sequence
  Description of Artificial Sequence Synthetic oligonucleotide
uagguuauccguguugccuucg

SEQ ID NO: 321 - RNA, 22 nt, Artificial Sequence
  Description of Artificial Sequence Synthetic oligonucleotide
uuaaugcuaauugugauagggg

SEQ ID NO: 322 - RNA, 22 nt, Artificial Sequence
  Description of Artificial Sequence Synthetic oligonucleotide
uaguugaauguuuagggucaga

SEQ ID NO: 323 - RNA, 22 nt, Artificial Sequence
  Description of Artificial Sequence Synthetic oligonucleotide
ugagugaaugguucaaguguau

SEQ ID NO: 324 - RNA, 22 nt, Artificial Sequence
  Description of Artificial Sequence Synthetic oligonucleotide
uauuuaggagggaacauguugu

SEQ ID NO: 325 - RNA, 22 nt, Artificial Sequence
  Description of Artificial Sequence Synthetic oligonucleotide
uuguagaguauuggucaaugag

SEQ ID NO: 326 - RNA, 23 nt, Artificial Sequence
  Description of Artificial Sequence Synthetic oligonucleotide
aacauucaacgcugucggugagu

SEQ ID NO: 327 - RNA, 23 nt, Artificial Sequence
  Description of Artificial Sequence Synthetic oligonucleotide
auucugugaacaucggacgucag

SEQ ID NO: 328 - RNA, 23 nt, Artificial Sequence
  Description of Artificial Sequence Synthetic oligonucleotide
aaguguuuccgagaacuaucggc

SEQ ID NO: 329 - RNA, 23 nt, Artificial Sequence
  Description of Artificial Sequence Synthetic oligonucleotide
aaguuucugaucgucagacggca

SEQ ID NO: 330 - RNA, 23 nt, Artificial Sequence
  Description of Artificial Sequence Synthetic oligonucleotide
acugagaaggccgcguuucauau

SEQ ID NO: 331 - RNA, 24 nt, Artificial Sequence
  Description of Artificial Sequence Synthetic oligonucleotide
aacauucauugcugucgguggguu

SEQ ID NO: 332 - RNA, 22 nt, Artificial Sequence
  Description of Artificial Sequence Synthetic oligonucleotide
aacauucaaccugucggugagu

SEQ ID NO: 333 - RNA, 22 nt, Artificial Sequence
  Description of Artificial Sequence Synthetic oligonucleotide
uuuggcaaugguagaacucaca

SEQ ID NO: 334 - RNA, 22 nt, Artificial Sequence
  Description of Artificial Sequence Synthetic oligonucleotide
ucugcaagagcagaauaguucu

SEQ ID NO: 335 - RNA, 22 nt, Artificial Sequence
  Description of Artificial Sequence Synthetic oligonucleotide
uugccaaauuggagaacuguac

SEQ ID NO: 336 - RNA, 22 nt, Artificial Sequence
  Description of Artificial Sequence Synthetic oligonucleotide
ugaauuugagucaugaccagac

SEQ ID NO: 337 - RNA, 22 nt, Artificial Sequence
  Description of Artificial Sequence Synthetic oligonucleotide
uugucaaggauagcccaauuag

SEQ ID NO: 338 - RNA, 23 nt, Artificial Sequence
  Description of Artificial Sequence Synthetic oligonucleotide
uauggcacugguagaauucacug

SEQ ID NO: 339 - RNA, 23 nt, Artificial Sequence
  Description of Artificial Sequence Synthetic oligonucleotide
uaacuauggagcagcugguuuca

SEQ ID NO: 340 - RNA, 23 nt, Artificial Sequence
  Description of Artificial Sequence Synthetic oligonucleotide
uaugcacuuguggugagcaucaa

SEQ ID NO: 341 - RNA, 23 nt, Artificial Sequence
  Description of Artificial Sequence Synthetic oligonucleotide
ucugguuacacaucaguuaaggg

SEQ ID NO: 342 - RNA, 23 nt, Artificial Sequence
  Description of Artificial Sequence Synthetic oligonucleotide
uauacaggccaugacuguuugag

SEQ ID NO: 343 - RNA, 22 nt, Artificial Sequence
  Description of Artificial Sequence Synthetic oligonucleotide
uggacggagaacugauaagggu

SEQ ID NO: 344 - RNA, 22 nt, Artificial Sequence
  Description of Artificial Sequence Synthetic oligonucleotide
ugacgugggacaggagauaaug

SEQ ID NO: 345 - RNA, 22 nt, Artificial Sequence
  Description of Artificial Sequence Synthetic oligonucleotide
uaggaacggaggagcauuagug

SEQ ID NO: 346 - RNA, 22 nt, Artificial Sequence
  Description of Artificial Sequence Synthetic oligonucleotide
uccggagaggaaaguguggaua

SEQ ID NO: 347 - RNA, 22 nt, Artificial Sequence
  Description of Artificial Sequence Synthetic oligonucleotide
uaggaacggagaguaagcugug

SEQ ID NO: 348 - RNA, 18 nt, Artificial Sequence
  Description of Artificial Sequence Synthetic oligonucleotide
uggagagaaaggcaguuc

SEQ ID NO: 349 - RNA, 23 nt, Artificial Sequence
  Description of Artificial Sequence Synthetic oligonucleotide
caaagaauucuccuuuugggcuu

SEQ ID NO: 350 - RNA, 22 nt, Artificial Sequence
  Description of Artificial Sequence Synthetic oligonucleotide
ucgugucuuguguugcagccgg

SEQ ID NO: 351 - RNA, 22 nt, Artificial Sequence
  Description of Artificial Sequence Synthetic oligonucleotide
uccuccguuuugcggguuaggg

SEQ ID NO: 352 - RNA, 22 nt, Artificial Sequence
  Description of Artificial Sequence Synthetic oligonucleotide
uccguguuucggcaucugggug

SEQ ID NO: 353 - RNA, 22 nt, Artificial Sequence
  Description of Artificial Sequence Synthetic oligonucleotide
uccguggcggggauguuuuccu

SEQ ID NO: 354 - RNA, 22 nt, Artificial Sequence
  Description of Artificial Sequence Synthetic oligonucleotide
uccguguugcuugcggcuugga

SEQ ID NO: 355 - RNA, 22 nt, Artificial Sequence
  Description of Artificial Sequence Synthetic oligonucleotide
caucccuugcaugguggagggu

SEQ ID NO: 356 - RNA, 23 nt, Artificial Sequence
  Description of Artificial Sequence Synthetic oligonucleotide
gugccuacugagcugacaucagu

SEQ ID NO: 357 - RNA, 22 nt, Artificial Sequence
  Description of Artificial Sequence Synthetic oligonucleotide
ugauauguuugauauauuaggu

SEQ ID NO: 358 - RNA, 22 nt, Artificial Sequence
  Description of Artificial Sequence Synthetic oligonucleotide
ugugguauuagauuauauugau

SEQ ID NO: 359 - RNA, 22 nt, Artificial Sequence
  Description of Artificial Sequence Synthetic oligonucleotide
uguaguuaguuguaauauugua

SEQ ID NO: 360 - RNA, 22 nt, Artificial Sequence
  Description of Artificial Sequence Synthetic oligonucleotide
ugugaguagauguuauuauuau

SEQ ID NO: 361 - RNA, 22 nt, Artificial Sequence
  Description of Artificial Sequence Synthetic oligonucleotide
uguauaauguuauagguuuagu

SEQ ID NO: 362 - RNA, 22 nt, Artificial Sequence
  Description of Artificial Sequence Synthetic oligonucleotide
caacggaaucccaaaagcagcu

SEQ ID NO: 363 - RNA, 21 nt, Artificial Sequence
  Description of Artificial Sequence Synthetic oligonucleotide
cugaccuaugaauugacagcc

SEQ ID NO: 364 - RNA, 21 nt, Artificial Sequence
  Description of Artificial Sequence Synthetic oligonucleotide
cccuaauagucagcaaggucu

SEQ ID NO: 365 - RNA, 21 nt, Artificial Sequence
  Description of Artificial Sequence Synthetic oligonucleotide
caggcuauccucaaucugaga

SEQ ID NO: 366 - RNA, 21 nt, Artificial Sequence
  Description of Artificial Sequence Synthetic oligonucleotide
cuaccuuacaggggccaauua

SEQ ID NO: 367 - RNA, 21 nt, Artificial Sequence
  Description of Artificial Sequence Synthetic oligonucleotide
ccaugguacccucaauuagag

SEQ ID NO: 368 - RNA, 21 nt, Artificial Sequence
  Description of Artificial Sequence Synthetic oligonucleotide
aacuggccuacaaagucccag

SEQ ID NO: 369 - RNA, 22 nt, Artificial Sequence
  Description of Artificial Sequence Synthetic oligonucleotide
uguaacagcaacuccaugugga

SEQ ID NO: 370 - RNA, 22 nt, Artificial Sequence
  Description of Artificial Sequence Synthetic oligonucleotide
ugguccuuacccagaaggaaua

SEQ ID NO: 371 - RNA, 22 nt, Artificial Sequence
  Description of Artificial Sequence Synthetic oligonucleotide
uuccaugcaguagagaugccaa

SEQ ID NO: 372 - RNA, 22 nt, Artificial Sequence
  Description of Artificial Sequence Synthetic oligonucleotide
ugggacauagaaccaucaugcu

SEQ ID NO: 373 - RNA, 22 nt, Artificial Sequence
  Description of Artificial Sequence Synthetic oligonucleotide
ucuaaagugagcuaauccaggc

SEQ ID NO: 374 - RNA, 21 nt, Artificial Sequence
  Description of Artificial Sequence Synthetic oligonucleotide
uagcagcacagaaauauuggc

SEQ ID NO: 375 - RNA, 22 nt, Artificial Sequence
  Description of Artificial Sequence Synthetic oligonucleotide
uagguaguuucauguuguuggg

SEQ ID NO: 376 - RNA, 22 nt, Artificial Sequence
  Description of Artificial Sequence Synthetic oligonucleotide
uguagauaguuugguuucuggg

SEQ ID NO: 377 - RNA, 22 nt, Artificial Sequence
  Description of Artificial Sequence Synthetic oligonucleotide
uauugguaggggucauuuugug

SEQ ID NO: 378 - RNA, 22 nt, Artificial Sequence
  Description of Artificial Sequence Synthetic oligonucleotide
ugguuauaguuuugauggcugg

SEQ ID NO: 379 - RNA, 22 nt, Artificial Sequence
  Description of Artificial Sequence Synthetic oligonucleotide
uguguaguuuggacagguguuu

SEQ ID NO: 380 - RNA, 22 nt, Artificial Sequence
  Description of Artificial Sequence Synthetic oligonucleotide
uucaccaccuucuccacccagc

SEQ ID NO: 381 - RNA, 19 nt, Artificial Sequence
  Description of Artificial Sequence Synthetic oligonucleotide
gguccagaggggagauagg

SEQ ID NO: 382 - RNA, 23 nt, Artificial Sequence
  Description of Artificial Sequence Synthetic oligonucleotide
cccaguguucagacuaccuguuc

SEQ ID NO: 383 - RNA, 23 nt, Artificial Sequence
  Description of Artificial Sequence Synthetic oligonucleotide
ccucaucuaccauugagccugug

SEQ ID NO: 384 - RNA, 23 nt, Artificial Sequence
  Description of Artificial Sequence Synthetic oligonucleotide
cugucugcuccaguuccagauac

SEQ ID NO: 385 - RNA, 23 nt, Artificial Sequence
  Description of Artificial Sequence Synthetic oligonucleotide
cugucacucugggcauccacuua

SEQ ID NO: 386 - RNA, 23 nt, Artificial Sequence
  Description of Artificial Sequence Synthetic oligonucleotide
ccugggguuuacaaccuauccuc

SEQ ID NO: 387 - RNA, 23 nt, Artificial Sequence
  Description of Artificial Sequence Synthetic oligonucleotide
cccaguguuuagacuaucuguuc

SEQ ID NO: 388 - RNA, 21 nt, Artificial Sequence
  Description of Artificial Sequence Synthetic oligonucleotide
uaacacugucugguaacgaug

SEQ ID NO: 389 - RNA, 23 nt, Artificial Sequence
  Description of Artificial Sequence Synthetic oligonucleotide
uaauacugccugguaaugaugac

SEQ ID NO: 390 - RNA, 23 nt, Artificial Sequence
  Description of Artificial Sequence Synthetic oligonucleotide
uacugagaaugguauccaguacu

SEQ ID NO: 391 - RNA, 23 nt, Artificial Sequence
  Description of Artificial Sequence Synthetic oligonucleotide
uaguggcuaacuauuggacacua

SEQ ID NO: 392 - RNA, 23 nt, Artificial Sequence
  Description of Artificial Sequence Synthetic oligonucleotide
uaugaggacaguguacuuaacuc

SEQ ID NO: 393 - RNA, 23 nt, Artificial Sequence
  Description of Artificial Sequence Synthetic oligonucleotide
uacauggacuauuaguggaucca

SEQ ID NO: 394 - RNA, 21 nt, Artificial Sequence
  Description of Artificial Sequence Synthetic oligonucleotide
uacucaguaaggcauuguucu

SEQ ID NO: 395 - RNA, 22 nt, Artificial Sequence
  Description of Artificial Sequence Synthetic oligonucleotide
agagguauagcgcaugggaaga

SEQ ID NO: 396 - RNA, 22 nt, Artificial Sequence
  Description of Artificial Sequence Synthetic oligonucleotide
agagauauggacguaggggcaa

SEQ ID NO: 397 - RNA, 22 nt, Artificial Sequence
  Description of Artificial Sequence Synthetic oligonucleotide
auaaguagggaacgggcugaga

SEQ ID NO: 398 - RNA, 22 nt, Artificial Sequence
  Description of Artificial Sequence Synthetic oligonucleotide
agggaguaagacaggacgaugu

SEQ ID NO: 399 - RNA, 22 nt, Artificial Sequence
  Description of Artificial Sequence Synthetic oligonucleotide
augaguacgguaggaagggaca

SEQ ID NO: 400 - RNA, 22 nt, Artificial Sequence
  Description of Artificial Sequence Synthetic oligonucleotide
ugaaauguuuaggaccacuaga

SEQ ID NO: 401 - RNA, 22 nt, Artificial Sequence
  Description of Artificial Sequence Synthetic oligonucleotide
uacauuugggacacaaugauga

SEQ ID NO: 402 - RNA, 22 nt, Artificial Sequence
  Description of Artificial Sequence Synthetic oligonucleotide
uaaagucuaguaaaugauggcc

SEQ ID NO: 403 - RNA, 22 nt, Artificial Sequence
  Description of Artificial Sequence Synthetic oligonucleotide
uagaacaacaaucuguguguga

SEQ ID NO: 404 - RNA, 22 nt, Artificial Sequence
  Description of Artificial Sequence Synthetic oligonucleotide
uaauggaaugaugauuagcacc

SEQ ID NO: 405 - RNA, 23 nt, Artificial Sequence
  Description of Artificial Sequence Synthetic oligonucleotide
uucccuuugucauccuaugccug

SEQ ID NO: 406 - RNA, 23 nt, Artificial Sequence
  Description of Artificial Sequence Synthetic oligonucleotide
ucacuuuuguuguccccccuaug

SEQ ID NO: 407 - RNA, 23 nt, Artificial Sequence
  Description of Artificial Sequence Synthetic oligonucleotide
uucuccuugccuguacuugcuca

SEQ ID NO: 408 - RNA, 23 nt, Artificial Sequence
  Description of Artificial Sequence Synthetic oligonucleotide
uuccuucuaggucucuccugacu

SEQ ID NO: 409 - RNA, 23 nt, Artificial Sequence
  Description of Artificial Sequence Synthetic oligonucleotide
uuucuccccccuguacaguuguu

SEQ ID NO: 410 - RNA, 22 nt, Artificial Sequence
  Description of Artificial Sequence Synthetic oligonucleotide
uccuucauuccaccggagucug

SEQ ID NO: 411 - RNA, 22 nt, Artificial Sequence
  Description of Artificial Sequence Synthetic oligonucleotide
uaggaauuccuucggccuuccc

SEQ ID NO: 412 - RNA, 22 nt, Artificial Sequence
  Description of Artificial Sequence Synthetic oligonucleotide
ucuacuuucccacagugcgcug

SEQ ID NO: 413 - RNA, 22 nt, Artificial Sequence
  Description of Artificial Sequence Synthetic oligonucleotide
uugcccccaaucgggcuuucua

SEQ ID NO: 414 - RNA, 22 nt, Artificial Sequence
  Description of Artificial Sequence Synthetic oligonucleotide
uuguuccaucgggccuuccaac

SEQ ID NO: 415 - RNA, 22 nt, Artificial Sequence
  Description of Artificial Sequence Synthetic oligonucleotide
uggaauguaaggaagugugugg

SEQ ID NO: 416 - RNA, 23 nt, Artificial Sequence
  Description of Artificial Sequence Synthetic oligonucleotide
gcuucuccuggcucuccucccuc

SEQ ID NO: 417 - RNA, 22 nt, Artificial Sequence
  Description of Artificial Sequence Synthetic oligonucleotide
auaagacgagcaaaaagcuugu

SEQ ID NO: 418 - RNA, 22 nt, Artificial Sequence
  Description of Artificial Sequence Synthetic oligonucleotide
augcgacaaagaaaugaucaug

SEQ ID NO: 419 - RNA, 22 nt, Artificial Sequence
  Description of Artificial Sequence Synthetic oligonucleotide
acgagcaugguuaaaaaugaac

SEQ ID NO: 420 - RNA, 22 nt, Artificial Sequence
  Description of Artificial Sequence Synthetic oligonucleotide
agcguuaaaacaagaaguugac

SEQ ID NO: 421 - RNA, 22 nt, Artificial Sequence
  Description of Artificial Sequence Synthetic oligonucleotide
agacgacuugaugcuaagaaaa

SEQ ID NO: 422 - RNA, 21 nt, Artificial Sequence
  Description of Artificial Sequence Synthetic oligonucleotide
cugugcgugugacagcggcug

SEQ ID NO: 423 - RNA, 21 nt, Artificial Sequence
  Description of Artificial Sequence Synthetic oligonucleotide
cguaggccugucgggcuugga

SEQ ID NO: 424 - RNA, 21 nt, Artificial Sequence
  Description of Artificial Sequence Synthetic oligonucleotide
cguuagccacguggggggcuu

SEQ ID NO: 425 - RNA, 21 nt, Artificial Sequence
  Description of Artificial Sequence Synthetic oligonucleotide
cguuauccuccggggggugag

SEQ ID NO: 426 - RNA, 21 nt, Artificial Sequence
  Description of Artificial Sequence Synthetic oligonucleotide
cccguggagaugcugguugcg

SEQ ID NO: 427 - RNA, 22 nt, Artificial Sequence
  Description of Artificial Sequence Synthetic oligonucleotide
uucccuuugucauccuucgccu

SEQ ID NO: 428 - RNA, 21 nt, Artificial Sequence
  Description of Artificial Sequence Synthetic oligonucleotide
uaacagucuccagucacggcc

SEQ ID NO: 429 - RNA, 22 nt, Artificial Sequence
  Description of Artificial Sequence Synthetic oligonucleotide
accaucgaccguugauuguacc

SEQ ID NO: 430 - RNA, 22 nt, Artificial Sequence
  Description of Artificial Sequence Synthetic oligonucleotide
accgagaucuccuucgcuagua

SEQ ID NO: 431 - RNA, 22 nt, Artificial Sequence
  Description of Artificial Sequence Synthetic oligonucleotide
aucgugaccacguagccuuuac

SEQ ID NO: 432 - RNA, 22 nt, Artificial Sequence
  Description of Artificial Sequence Synthetic oligonucleotide
aaucgucuaugccagcgucuca

SEQ ID NO: 433 - RNA, 22 nt, Artificial Sequence
  Description of Artificial Sequence Synthetic oligonucleotide
aucgucaccacgaugguauucc

SEQ ID NO: 434 - RNA, 21 nt, Artificial Sequence
  Description of Artificial Sequence Synthetic oligonucleotide
acagcaggcacagacaggcag

SEQ ID NO: 435 - RNA, 21 nt, Artificial Sequence
  Description of Artificial Sequence Synthetic oligonucleotide
aggcagagaccaagaccaggc

SEQ ID NO: 436 - RNA, 21 nt, Artificial Sequence
  Description of Artificial Sequence Synthetic oligonucleotide
acaggcagcaccacagaggag

SEQ ID NO: 437 - RNA, 21 nt, Artificial Sequence
  Description of Artificial Sequence Synthetic oligonucleotide
acaggaagggaagcagcccac

SEQ ID NO: 438 - RNA, 21 nt, Artificial Sequence
  Description of Artificial Sequence Synthetic oligonucleotide
acaggagggaagcccccaaga

SEQ ID NO: 439 - RNA, 21 nt, Artificial Sequence
  Description of Artificial Sequence Synthetic oligonucleotide
augaccuaugaauugacagac

SEQ ID NO: 440 - RNA, 21 nt, Artificial Sequence
  Description of Artificial Sequence Synthetic oligonucleotide
uaaucucagcuggcaacugug

SEQ ID NO: 441 - RNA, 21 nt, Artificial Sequence
  Description of Artificial Sequence Synthetic oligonucleotide
ugcugucaagaaugucuccag

SEQ ID NO: 442 - RNA, 21 nt, Artificial Sequence
  Description of Artificial Sequence Synthetic oligonucleotide
uaaccucaagggugcuuugac

SEQ ID NO: 443 - RNA, 21 nt, Artificial Sequence
  Description of Artificial Sequence Synthetic oligonucleotide
uaccauuugcaggcaugcaug

SEQ ID NO: 444 - RNA, 21 nt, Artificial Sequence
  Description of Artificial Sequence Synthetic oligonucleotide
ucuaaauagcagcccuuggug

SEQ ID NO: 445 - RNA, 24 nt, Artificial Sequence
  Description of Artificial Sequence Synthetic oligonucleotide
uacugcaucaggaacugauuggau

SEQ ID NO: 446 - RNA, 21 nt, Artificial Sequence
  Description of Artificial Sequence Synthetic oligonucleotide
uugugcuugaucuaaccaugu

SEQ ID NO: 447 - RNA, 21 nt, Artificial Sequence
  Description of Artificial Sequence Synthetic oligonucleotide
ucacacuuguagucugugauu

SEQ ID NO: 448 - RNA, 21 nt, Artificial Sequence
  Description of Artificial Sequence Synthetic oligonucleotide
uuuguguguucuccacaagua

SEQ ID NO: 449 - RNA, 21 nt, Artificial Sequence
  Description of Artificial Sequence Synthetic oligonucleotide
ugcuuccucaguguuuauaga

SEQ ID NO: 450 - RNA, 21 nt, Artificial Sequence
  Description of Artificial Sequence Synthetic oligonucleotide
uucauuaaguagcuugugucc

SEQ ID NO: 451 - RNA, 21 nt, Artificial Sequence
  Description of Artificial Sequence Synthetic oligonucleotide
ugauuguccaaacgcaauucu

SEQ ID NO: 452 - RNA, 21 nt, Artificial Sequence
  Description of Artificial Sequence Synthetic oligonucleotide
uuaggcucuacccuaugauaa

SEQ ID NO: 453 - RNA, 21 nt, Artificial Sequence
  Description of Artificial Sequence Synthetic oligonucleotide
uuagcuagaccuaugcuaacu

SEQ ID NO: 454 - RNA, 21 nt, Artificial Sequence
  Description of Artificial Sequence Synthetic oligonucleotide
ugggcuaacuuaccuauaacu

SEQ ID NO: 455 - RNA, 21 nt, Artificial Sequence
  Description of Artificial Sequence Synthetic oligonucleotide
uuuaccccuaguggacauaau

SEQ ID NO: 456 - RNA, 21 nt, Artificial Sequence
  Description of Artificial Sequence Synthetic oligonucleotide
ccacaccguaucugacacuuu

SEQ ID NO: 457 - RNA, 23 nt, Artificial Sequence
  Description of Artificial Sequence Synthetic oligonucleotide
agcuacauugucugcuggguuuc

SEQ ID NO: 458 - RNA, 23 nt, Artificial Sequence
  Description of Artificial Sequence Synthetic oligonucleotide
auaugugggugccuuucuccaug

SEQ ID NO: 459 - RNA, 23 nt, Artificial Sequence
  Description of Artificial Sequence Synthetic oligonucleotide
acauccuaguucugcuuuggggu

SEQ ID NO: 460 - RNA, 23 nt, Artificial Sequence
  Description of Artificial Sequence Synthetic oligonucleotide
auaccucuucaguuggguggcuu

SEQ ID NO: 461 - RNA, 23 nt, Artificial Sequence
  Description of Artificial Sequence Synthetic oligonucleotide
auauguucuugcugguuggccac

SEQ ID NO: 462 - RNA, 24 nt, Artificial Sequence
  Description of Artificial Sequence Synthetic oligonucleotide
agcuacaucuggcuacugggucuc

SEQ ID NO: 463 - RNA, 23 nt, Artificial Sequence
  Description of Artificial Sequence Synthetic oligonucleotide
ugucaguuugucaaauaccccaa

SEQ ID NO: 464 - RNA, 23 nt, Artificial Sequence
  Description of Artificial Sequence Synthetic oligonucleotide
uaacuugugagcauccaaaucuc

SEQ ID NO: 465 - RNA, 23 nt, Artificial Sequence
  Description of Artificial Sequence Synthetic oligonucleotide
ugcucacuguaaucagaaauucc

SEQ ID NO: 466 - RNA, 23 nt, Artificial Sequence
  Description of Artificial Sequence Synthetic oligonucleotide
uacaacuccugauucauuagcag

SEQ ID NO: 467 - RNA, 23 nt, Artificial Sequence
  Description of Artificial Sequence Synthetic oligonucleotide
uauucaacagcuguuccaacuga

SEQ ID NO: 468 - RNA, 23 nt, Artificial Sequence
  Description of Artificial Sequence Synthetic oligonucleotide
caagucacuagugguuccguuua

SEQ ID NO: 469 - RNA, 7 nt, Artificial Sequence
  Description of Artificial Sequence Synthetic oligonucleotide
gagguag

SEQ ID NO: 470 - RNA, 7 nt, Artificial Sequence
  Description of Artificial Sequence Synthetic oligonucleotide
acaguac

SEQ ID NO: 471 - RNA, 7 nt, Artificial Sequence
  Description of Artificial Sequence Synthetic oligonucleotide
gcagcau

SEQ ID NO: 472 - RNA, 7 nt, Artificial Sequence
  Description of Artificial Sequence Synthetic oligonucleotide
acccugu

SEQ ID NO: 473 - RNA, 7 nt, Artificial Sequence
  Description of Artificial Sequence Synthetic oligonucleotide
ggagugu

SEQ ID NO: 474 - RNA, 7 nt, Artificial Sequence
  Description of Artificial Sequence Synthetic oligonucleotide
uaaggca

SEQ ID NO: 475 - RNA, 7 nt, Artificial Sequence
  Description of Artificial Sequence Synthetic oligonucleotide
cccugag

SEQ ID NO: 476 - RNA, 7 nt, Artificial Sequence
  Description of Artificial Sequence Synthetic oligonucleotide
cacagug

SEQ ID NO: 477 - RNA, 7 nt, Artificial Sequence
  Description of Artificial Sequence Synthetic oligonucleotide
agugcaa

SEQ ID NO: 478 - RNA, 7 nt, Artificial Sequence
  Description of Artificial Sequence Synthetic oligonucleotide
aacaguc

SEQ ID NO: 479 - RNA, 7 nt, Artificial Sequence
  Description of Artificial Sequence Synthetic oligonucleotide
ugguccc

SEQ ID NO: 480 - RNA, 7 nt, Artificial Sequence
  Description of Artificial Sequence Synthetic oligonucleotide
gcuggug

SEQ ID NO: 481 - RNA, 7 nt, Artificial Sequence
  Description of Artificial Sequence Synthetic oligonucleotide
acaguau

SEQ ID NO: 482 - RNA, 7 nt, Artificial Sequence
  Description of Artificial Sequence Synthetic oligonucleotide
uccaguu

SEQ ID NO: 483 - RNA, 7 nt, Artificial Sequence
  Description of Artificial Sequence Synthetic oligonucleotide
gagaacu

SEQ ID NO: 484 - RNA, 7 nt, Artificial Sequence
  Description of Artificial Sequence Synthetic oligonucleotide
cagugca

SEQ ID NO: 485 - RNA, 7 nt, Artificial Sequence
  Description of Artificial Sequence Synthetic oligonucleotide
acauuca

SEQ ID NO: 486 - RNA, 7 nt, Artificial Sequence
  Description of Artificial Sequence Synthetic oligonucleotide
uuggcaa

SEQ ID NO: 487 - RNA, 7 nt, Artificial Sequence
  Description of Artificial Sequence Synthetic oligonucleotide
ggacgga

SEQ ID NO: 488 - RNA, 7 nt, Artificial Sequence
  Description of Artificial Sequence Synthetic oligonucleotide
guaacag

SEQ ID NO: 489 - RNA, 7 nt, Artificial Sequence
  Description of Artificial Sequence Synthetic oligonucleotide
agguagu

SEQ ID NO: 490 - RNA, 7 nt, Artificial Sequence
  Description of Artificial Sequence Synthetic oligonucleotide
ccagugu

SEQ ID NO: 491 - RNA, 7 nt, Artificial Sequence
  Description of Artificial Sequence Synthetic oligonucleotide
gugcaaa

SEQ ID NO: 492 - RNA, 7 nt, Artificial Sequence
  Description of Artificial Sequence Synthetic oligonucleotide
ggaaugu

SEQ ID NO: 493 - RNA, 7 nt, Artificial Sequence
  Description of Artificial Sequence Synthetic oligonucleotide
aaagugc

SEQ ID NO: 494 - RNA, 7 nt, Artificial Sequence
  Description of Artificial Sequence Synthetic oligonucleotide
aauacug

SEQ ID NO: 495 - RNA, 7 nt, Artificial Sequence
  Description of Artificial Sequence Synthetic oligonucleotide
gagguau

SEQ ID NO: 496 - RNA, 7 nt, Artificial Sequence
  Description of Artificial Sequence Synthetic oligonucleotide
aaucuca

SEQ ID NO: 497 - RNA, 7 nt, Artificial Sequence
  Description of Artificial Sequence Synthetic oligonucleotide
ucacauu

SEQ ID NO: 498 - RNA, 7 nt, Artificial Sequence
  Description of Artificial Sequence Synthetic oligonucleotide
ggcucag

SEQ ID NO: 499 - RNA, 7 nt, Artificial Sequence
  Description of Artificial Sequence Synthetic oligonucleotide
auugcac

SEQ ID NO: 500 - RNA, 7 nt, Artificial Sequence
  Description of Artificial Sequence Synthetic oligonucleotide
ucaagua

SEQ ID NO: 501 - RNA, 7 nt, Artificial Sequence
  Description of Artificial Sequence Synthetic oligonucleotide
agcacca

SEQ ID NO: 502 - RNA, 7 nt, Artificial Sequence
  Description of Artificial Sequence Synthetic oligonucleotide
guaaaca

SEQ ID NO: 503 - RNA, 7 nt, Artificial Sequence
  Description of Artificial Sequence Synthetic oligonucleotide
cuuuggu

SEQ ID NO: 504 - RNA, 7 nt, Artificial Sequence
  Description of Artificial Sequence Synthetic oligonucleotide
agcagca

SEQ ID NO: 505 - RNA, 7 nt, Artificial Sequence
  Description of Artificial Sequence Synthetic oligonucleotide
ucacagu

SEQ ID NO: 506 - RNA, 7 nt, Artificial Sequence
  Description of Artificial Sequence Synthetic oligonucleotide
aagugcu

SEQ ID NO: 507 - RNA, 7 nt, Artificial Sequence
  Description of Artificial Sequence Synthetic oligonucleotide
uuuuugc

SEQ ID NO: 508 - RNA, 7 nt, Artificial Sequence
  Description of Artificial Sequence Synthetic oligonucleotide
auggcuu

SEQ ID NO: 509 - RNA, 7 nt, Artificial Sequence
  Description of Artificial Sequence Synthetic oligonucleotide
acacugu

SEQ ID NO: 510 - RNA, 7 nt, Artificial Sequence
  Description of Artificial Sequence Synthetic oligonucleotide
aaggugc

SEQ ID NO: 511 - RNA, 7 nt, Artificial Sequence
  Description of Artificial Sequence Synthetic oligonucleotide
cagcagg

SEQ ID NO: 512 - RNA, 7 nt, Artificial Sequence
  Description of Artificial Sequence Synthetic oligonucleotide
agcugcc

SEQ ID NO: 513 - RNA, 7 nt, Artificial Sequence
  Description of Artificial Sequence Synthetic oligonucleotide
ggcagug

SEQ ID NO: 514 - RNA, 7 nt, Artificial Sequence
  Description of Artificial Sequence Synthetic oligonucleotide
uuggcac

SEQ ID NO: 515 - RNA, 7 nt, Artificial Sequence
  Description of Artificial Sequence Synthetic oligonucleotide
gugguuu

SEQ ID NO: 516 - RNA, 7 nt, Artificial Sequence
  Description of Artificial Sequence Synthetic oligonucleotide
ccauaaa

SEQ ID NO: 517 - RNA, 7 nt, Artificial Sequence
  Description of Artificial Sequence Synthetic oligonucleotide
gagauga

SEQ ID NO: 518 - RNA, 7 nt, Artificial Sequence
  Description of Artificial Sequence Synthetic oligonucleotide
auggcac

SEQ ID NO: 519 - RNA, 7 nt, Artificial Sequence
  Description of Artificial Sequence Synthetic oligonucleotide
gaaaugu

SEQ ID NO: 520 - RNA, 7 nt, Artificial Sequence
  Description of Artificial Sequence Synthetic oligonucleotide
ugugcgu

SEQ ID NO: 521 - RNA, 7 nt, Artificial Sequence
  Description of Artificial Sequence Synthetic oligonucleotide
ugugcuu

SEQ ID NO: 522 - RNA, 7 nt, Artificial Sequence
  Description of Artificial Sequence Synthetic oligonucleotide
gcuacau

SEQ ID NO: 523 - RNA, 7 nt, Artificial Sequence
  Description of Artificial Sequence Synthetic oligonucleotide
gcaagau

SEQ ID NO: 524 - RNA, 7 nt, Artificial Sequence
  Description of Artificial Sequence Synthetic oligonucleotide
ugcauug

SEQ ID NO: 525 - RNA, 7 nt, Artificial Sequence
  Description of Artificial Sequence Synthetic oligonucleotide
ggaagac

SEQ ID NO: 526 - RNA, 7 nt, Artificial Sequence
  Description of Artificial Sequence Synthetic oligonucleotide
auugcuu

SEQ ID NO: 527 - RNA, 7 nt, Artificial Sequence
  Description of Artificial Sequence Synthetic oligonucleotide
ugcauag

SEQ ID NO: 528 - RNA, 7 nt, Artificial Sequence
  Description of Artificial Sequence Synthetic oligonucleotide
uaaugcu

SEQ ID NO: 529 - RNA, 7 nt, Artificial Sequence
  Description of Artificial Sequence Synthetic oligonucleotide
ucccuuu

SEQ ID NO: 530 - RNA, 7 nt, Artificial Sequence
  Description of Artificial Sequence Synthetic oligonucleotide
gucaguu

SEQ ID NO: 531 - RNA, 7 nt, Artificial Sequence
  Description of Artificial Sequence Synthetic oligonucleotide
uagcacc

SEQ ID NO: 532 - RNA, 7 nt, Artificial Sequence
  Description of Artificial Sequence Synthetic oligonucleotide
guagugu

SEQ ID NO: 533 - RNA, 7 nt, Artificial Sequence
  Description of Artificial Sequence Synthetic oligonucleotide
aaagcua

SEQ ID NO: 534 - RNA, 7 nt, Artificial Sequence
  Description of Artificial Sequence Synthetic oligonucleotide
gauaugu

SEQ ID NO: 535 - RNA, 7 nt, Artificial Sequence
  Description of Artificial Sequence Synthetic oligonucleotide
ccuucau

SEQ ID NO: 536 - RNA, 7 nt, Artificial Sequence
  Description of Artificial Sequence Synthetic oligonucleotide
acccgua

SEQ ID NO: 537 - RNA, 7 nt, Artificial Sequence
  Description of Artificial Sequence Synthetic oligonucleotide
gauuguc

SEQ ID NO: 538 - RNA, 39 nt, Homo sapiens
ugccucuggaaaacuauugagccuugcauguacuugaag

SEQ ID NO: 539 - RNA, 20 nt, Homo sapiens
gagccuugauaauacuugac

SEQ ID NO: 540 - RNA, 106 nt, Homo sapiens
  modified_base (1)..(6): a, c, g, u, unknown or other
nnnnnnugccucuggaannnnnnnnnnnnnnnnnnguacuugaagnnnnnnnnnnnnnnnnnnnnnnnnnnnnnnnnnnnngagccuugauaauacuugacnnnnn

SEQ ID NO: 541 - RNA, 106 nt, Homo sapiens
  modified_base (1)..(6): a, c, g, u, unknown or other
nnnnnnugccucuggaannnnnnnnnnnnnnnnnnguucguuaagnnnnnnnnnnnnnnnnnnnnnnnnnnnnnnnnnnnngagccuugauaauucguuacnnnnn

SEQ ID NO: 542 - RNA, 7 nt, Artificial Sequence
  Description of Artificial Sequence Synthetic oligonucleotide
aaggcac

SEQ ID NO: 543 - RNA, 7 nt, Artificial Sequence
  Description of Artificial Sequence Synthetic oligonucleotide
ugaccua

SEQ ID NO: 544 - RNA, 7 nt, Artificial Sequence
  Description of Artificial Sequence Synthetic oligonucleotide
aacacug

SEQ ID NO: 545 - RNA, 7 nt, Artificial Sequence
  Description of Artificial Sequence Synthetic oligonucleotide
aacacug

SEQ ID NO: 546 - RNA, 7 nt, Artificial Sequence
  Description of Artificial Sequence Synthetic oligonucleotide
agcuuau

SEQ ID NO: 547 - RNA, 7 nt, Artificial Sequence
  Description of Artificial Sequence Synthetic oligonucleotide
cguaccg

SEQ ID NO: 548 - RNA, 7 nt, Artificial Sequence
  Description of Artificial Sequence Synthetic oligonucleotide
cgugucu

SEQ ID NO: 549 - RNA, 7 nt, Artificial Sequence
  Description of Artificial Sequence Synthetic oligonucleotide
ugaaaug

SEQ ID NO: 550 - RNA, 7 nt, Artificial Sequence
  Description of Artificial Sequence Synthetic oligonucleotide
acugcau

SEQ ID NO: 551 - RNA, 7 nt, Artificial Sequence
  Description of Artificial Sequence Synthetic oligonucleotide
uuguucg

SEQ ID NO: 552 - RNA, 12 nt, Artificial Sequence
  Description of Artificial Sequence Synthetic oligonucleotide
aagaaguaugua

SEQ ID NO: 553 - RNA, 13 nt, Artificial Sequence
  Description of Artificial Sequence Synthetic oligonucleotide
aggaagugugugg

SEQ ID NO: 554 - RNA, 13 nt, Artificial Sequence
  Description of Artificial Sequence Synthetic oligonucleotide
agguuguauaguu

SEQ ID NO: 555 - RNA, 13 nt, Artificial Sequence
  Description of Artificial Sequence Synthetic oligonucleotide
agguugugugguu

SEQ ID NO: 556 - RNA, 13 nt, Artificial Sequence
  Description of Artificial Sequence Synthetic oligonucleotide
agguuguaugguu

SEQ ID NO: 557 - RNA, 12 nt, Artificial Sequence
  Description of Artificial Sequence Synthetic oligonucleotide
agguugcauagu

SEQ ID NO: 558 - RNA, 12 nt, Artificial Sequence
  Description of Artificial Sequence Synthetic oligonucleotide
agguuguauagu

SEQ ID NO: 559 - RNA, 13 nt, Artificial Sequence
  Description of Artificial Sequence Synthetic oligonucleotide
agauuguauaguu

SEQ ID NO: 560 - RNA, 12 nt, Artificial Sequence
  Description of Artificial Sequence Synthetic oligonucleotide
aguuuguacagu

SEQ ID NO: 561 - RNA, 10 nt, Artificial Sequence
  Description of Artificial Sequence Synthetic oligonucleotide
aguuugugcu

SEQ ID NO: 562 - RNA, 13 nt, Artificial Sequence
  Description of Artificial Sequence Synthetic oligonucleotide
aaguuguauuguu

SEQ ID NO: 563 - RNA, 12 nt, Artificial Sequence
  Description of Artificial Sequence Synthetic oligonucleotide
ucauguuguugg

SEQ ID NO: 564 - RNA, 12 nt, Artificial Sequence
  Description of Artificial Sequence Synthetic oligonucleotide
uccuguuguugg

SEQ ID NO: 565 - RNA, 14 nt, Artificial Sequence
  Description of Artificial Sequence Synthetic oligonucleotide
gauccgaauuugug

SEQ ID NO: 566 - RNA, 13 nt, Artificial Sequence
  Description of Artificial Sequence Synthetic oligonucleotide
gaaccgaauuugu

SEQ ID NO: 567 - RNA, 13 nt, Artificial Sequence
  Description of Artificial Sequence Synthetic oligonucleotide
auaaugguuugug

SEQ ID NO: 568 - RNA, 13 nt, Artificial Sequence
  Description of Artificial Sequence Synthetic oligonucleotide
aucaugguuuaca

SEQ ID NO: 569 - RNA, 13 nt, Artificial Sequence
  Description of Artificial Sequence Synthetic oligonucleotide
guaaauauuggcg

SEQ ID NO: 570 - RNA, 12 nt, Artificial Sequence
  Description of Artificial Sequence Synthetic oligonucleotide
agaaauauuggc

SEQ ID NO: 571 - RNA, 14 nt, Artificial Sequence
  Description of Artificial Sequence Synthetic oligonucleotide
guacagggcuauga

SEQ ID NO: 572 - RNA, 14 nt, Artificial Sequence
  Description of Artificial Sequence Synthetic oligonucleotide
guacagggcuauca

SEQ ID NO: 573 - RNA, 14 nt, Artificial Sequence
  Description of Artificial Sequence Synthetic oligonucleotide
cuaugcaaaacuga

SEQ ID NO: 574 - RNA, 14 nt, Artificial Sequence
  Description of Artificial Sequence Synthetic oligonucleotide
ccaugcaaaacuga

SEQ ID NO: 575 - RNA, 11 nt, Artificial Sequence
  Description of Artificial Sequence Synthetic oligonucleotide
guuaaaagggc

SEQ ID NO: 576 - RNA, 13 nt, Artificial Sequence
  Description of Artificial Sequence Synthetic oligonucleotide
gaugaaagggcau

SEQ ID NO: 577 - RNA, 14 nt, Artificial Sequence
  Description of Artificial Sequence Synthetic oligonucleotide
aguauugucaaagc

SEQ ID NO: 578 - RNA, 13 nt, Artificial Sequence
  Description of Artificial Sequence Synthetic oligonucleotide
uacagaacuuugu

SEQ ID NO: 579 - RNA, 13 nt, Artificial Sequence
  Description of Artificial Sequence Synthetic oligonucleotide
cacagaacuuugu

SEQ ID NO: 580 - RNA, 12 nt, Artificial Sequence
  Description of Artificial Sequence Synthetic oligonucleotide
gacagaacuugg

SEQ ID NO: 581 - RNA, 13 nt, Artificial Sequence
  Description of Artificial Sequence Synthetic oligonucleotide
uucgugcagguag

SEQ ID NO: 582 - RNA, 14 nt, Artificial Sequence
  Description of Artificial Sequence Synthetic oligonucleotide
ccauguuuugguga

SEQ ID NO: 583 - RNA, 14 nt, Artificial Sequence
  Description of Artificial Sequence Synthetic oligonucleotide
ccauguuuuaguag

SEQ ID NO: 584 - RNA, 14 nt, Artificial Sequence
  Description of Artificial Sequence Synthetic oligonucleotide
ccauguuucagugg

SEQ ID NO: 585 - RNA, 14 nt, Artificial Sequence
  Description of Artificial Sequence Synthetic oligonucleotide
ccauguuugagugu

SEQ ID NO: 586 - RNA, 14 nt, Artificial Sequence
  Description of Artificial Sequence Synthetic oligonucleotide
cgacauuugagcgu

SEQ ID NO: 587 - RNA, 14 nt, Artificial Sequence
  Description of Artificial Sequence Synthetic oligonucleotide
cgauuuuggggugu

SEQ ID NO: 588 - RNA, 13 nt, Artificial Sequence
  Description of Artificial Sequence Synthetic oligonucleotide
uauagugcaggua

SEQ ID NO: 589 - RNA, 15 nt, Artificial Sequence
  Description of Artificial Sequence Synthetic oligonucleotide
uacagugcagguagu

SEQ ID NO: 590 - RNA, 15 nt, Artificial Sequence
  Description of Artificial Sequence Synthetic oligonucleotide
uacagugcagguagc

SEQ ID NO: 591 - RNA, 12 nt, Artificial Sequence
  Description of Artificial Sequence Synthetic oligonucleotide
gacagugcagau

SEQ ID NO: 592 - RNA, 12 nt, Artificial Sequence
  Description of Artificial Sequence Synthetic oligonucleotide
ccagggauuucc

SEQ ID NO: 593 - RNA, 14 nt, Artificial Sequence
  Description of Artificial Sequence Synthetic oligonucleotide
ccagggauuaccac

SEQ ID NO: 594 - RNA, 13 nt, Artificial Sequence
  Description of Artificial Sequence Synthetic oligonucleotide
ugucucggucuga

SEQ ID NO: 595 - RNA, 12 nt, Artificial Sequence
  Description of Artificial Sequence Synthetic oligonucleotide
uuacuaaguugc

SEQ ID NO: 596 - RNA, 13 nt, Artificial Sequence
  Description of Artificial Sequence Synthetic oligonucleotide
ugucccggccugu

SEQ ID NO: 597 - RNA, 13 nt, Artificial Sequence
  Description of Artificial Sequence Synthetic oligonucleotide
uuagcaaugguga

SEQ ID NO: 598 - RNA, 13 nt, Artificial Sequence
  Description of Artificial Sequence Synthetic oligonucleotide
uccaggauaggcu

SEQ ID NO: 599 - RNA, 12 nt, Artificial Sequence
  Description of Artificial Sequence Synthetic oligonucleotide
uucaggauaggu

SEQ ID NO: 600 - RNA, 13 nt, Artificial Sequence
  Description of Artificial Sequence Synthetic oligonucleotide
gcuaaguuccgcc

SEQ ID NO: 601 - RNA, 11 nt, Artificial Sequence
  Description of Artificial Sequence Synthetic oligonucleotide
gcuaaguucug

SEQ ID NO: 602 - RNA, 13 nt, Artificial Sequence
  Description of Artificial Sequence Synthetic oligonucleotide
accggucucuuuu

SEQ ID NO: 603 - RNA, 13 nt, Artificial Sequence
  Description of Artificial Sequence Synthetic oligonucleotide
accggucucuuuc

SEQ ID NO: 604 - RNA, 11 nt, Artificial Sequence
  Description of Artificial Sequence Synthetic oligonucleotide
uugaaaucagu

SEQ ID NO: 605 - RNA, 13 nt, Artificial Sequence
  Description of Artificial Sequence Synthetic oligonucleotide
uugaaaucgguua

SEQ ID NO: 606 - RNA, 14 nt, Artificial Sequence
  Description of Artificial Sequence Synthetic oligonucleotide
ccucgacuggaagc

SEQ ID NO: 607 - RNA, 12 nt, Artificial Sequence
  Description of Artificial Sequence Synthetic oligonucleotide
ccuacacucagc

SEQ ID NO: 608 - RNA, 14 nt, Artificial Sequence
  Description of Artificial Sequence Synthetic oligonucleotide
ccuacacucucagc

SEQ ID NO: 609 - RNA, 13 nt, Artificial Sequence
  Description of Artificial Sequence Synthetic oligonucleotide
ccccgacuggaag

SEQ ID NO: 610 - RNA, 11 nt, Artificial Sequence
  Description of Artificial Sequence Synthetic oligonucleotide
ccuugacugga

SEQ ID NO: 611 - RNA, 13 nt, Artificial Sequence
  Description of Artificial Sequence Synthetic oligonucleotide
cuuagcugguugu

SEQ ID NO: 612 - RNA, 13 nt, Artificial Sequence
  Description of Artificial Sequence Synthetic oligonucleotide
cauuagcugauug

SEQ ID NO: 613 - RNA, 13 nt, Artificial Sequence
  Description of Artificial Sequence Synthetic oligonucleotide
aguuagcugauug

SEQ ID NO: 614 - RNA, 13 nt, Artificial Sequence
  Description of Artificial Sequence Synthetic oligonucleotide
auccgaacuugug

SEQ ID NO: 615 - RNA, 13 nt, Artificial Sequence
  Description of Artificial Sequence Synthetic oligonucleotide
auccgaucuugug

SEQ ID NO: 616 - RNA, 13 nt, Artificial Sequence
  Description of Artificial Sequence Synthetic oligonucleotide
aaccgaccuugcg

SEQ ID NO: 617 - RNA, 12 nt, Artificial Sequence
  Description of Artificial Sequence Synthetic oligonucleotide
gcggugaaugcc

SEQ ID NO: 618 - RNA, 12 nt, Artificial Sequence
  Description of Artificial Sequence Synthetic oligonucleotide
cggugaaugcca

SEQ ID NO: 619 - RNA, 13 nt, Artificial Sequence
  Description of Artificial Sequence Synthetic oligonucleotide
cccuaacuuguga

SEQ ID NO: 620 - RNA, 14 nt, Artificial Sequence
  Description of Artificial Sequence Synthetic oligonucleotide
cccuuuaaccugug

SEQ ID NO: 621 - RNA, 13 nt, Artificial Sequence
  Description of Artificial Sequence Synthetic oligonucleotide
uucaaccagcugu

SEQ ID NO: 622 - RNA, 12 nt, Artificial Sequence
  Description of Artificial Sequence Synthetic oligonucleotide
uucaaccagcua

SEQ ID NO: 623 - RNA, 14 nt, Artificial Sequence
  Description of Artificial Sequence Synthetic oligonucleotide
uuauuccuauguga

SEQ ID NO: 624 - RNA, 13 nt, Artificial Sequence
  Description of Artificial Sequence Synthetic oligonucleotide
ucauuccuaugug

SEQ ID NO: 625 - RNA, 14 nt, Artificial Sequence
  Description of Artificial Sequence Synthetic oligonucleotide
cgcugucggugagu

SEQ ID NO: 626 - RNA, 15 nt, Artificial Sequence
  Description of Artificial Sequence Synthetic oligonucleotide
ugcugucgguggguu

SEQ ID NO: 627 - RNA, 13 nt, Artificial Sequence
  Description of Artificial Sequence Synthetic oligonucleotide
ccugucggugagu

SEQ ID NO: 628 - RNA, 12 nt, Artificial Sequence
  Description of Artificial Sequence Synthetic oligonucleotide
gaauugacagcc

SEQ ID NO: 629 - RNA, 12 nt, Artificial Sequence
  Description of Artificial Sequence Synthetic oligonucleotide
gaauugacagac

SEQ ID NO: 630 - RNA, 14 nt, Artificial Sequence
  Description of Artificial Sequence Synthetic oligonucleotide
cagacuaccuguuc

SEQ ID NO: 631 - RNA, 14 nt, Artificial Sequence
  Description of Artificial Sequence Synthetic oligonucleotide
uagacuaucuguuc

SEQ ID NO: 632 - RNA, 13 nt, Artificial Sequence
  Description of Artificial Sequence Synthetic oligonucleotide
cugguaacgaugu

SEQ ID NO: 633 - RNA, 13 nt, Artificial Sequence
  Description of Artificial Sequence Synthetic oligonucleotide
cugguaaagaugg

SEQ ID NO: 634 - RNA, 12 nt, Artificial Sequence
  Description of Artificial Sequence Synthetic oligonucleotide
cugguaaugaug

SEQ ID NO: 635 - RNA, 14 nt, Artificial Sequence
  Description of Artificial Sequence Synthetic oligonucleotide
cggguaaugaugga

SEQ ID NO: 636 - RNA, 13 nt, Artificial Sequence
  Description of Artificial Sequence Synthetic oligonucleotide
ucauccuaugccu

SEQ ID NO: 637 - RNA, 13 nt, Artificial Sequence
  Description of Artificial Sequence Synthetic oligonucleotide
ucauccuucgccu

SEQ ID NO: 638 - RNA, 14 nt, Artificial Sequence
  Description of Artificial Sequence Synthetic oligonucleotide
gucugcuggguuuc

SEQ ID NO: 639 - RNA, 15 nt, Artificial Sequence
  Description of Artificial Sequence Synthetic oligonucleotide
uggcuacugggucuc

SEQ ID NO: 640 - RNA, 12 nt, Artificial Sequence
  Description of Artificial Sequence Synthetic oligonucleotide
agugauuuuguu

SEQ ID NO: 641 - RNA, 14 nt, Artificial Sequence
  Description of Artificial Sequence Synthetic oligonucleotide
aucuagcuguauga

SEQ ID NO: 642 - RNA, 13 nt, Artificial Sequence
  Description of Artificial Sequence Synthetic oligonucleotide
ucuagugcagaua

SEQ ID NO: 643 - RNA, 13 nt, Artificial Sequence
  Description of Artificial Sequence Synthetic oligonucleotide
agacugauguuga

SEQ ID NO: 644 - RNA, 13 nt, Artificial Sequence
  Description of Artificial Sequence Synthetic oligonucleotide
guugaagaacugu

SEQ ID NO: 645 - RNA, 13 nt, Artificial Sequence
  Description of Artificial Sequence Synthetic oligonucleotide
ucagcaggaacag

SEQ ID NO: 646 - RNA, 10 nt, Artificial Sequence
  Description of Artificial Sequence Synthetic oligonucleotide
aguugcauug

SEQ ID NO: 647 - RNA, 13 nt, Artificial Sequence
  Description of Artificial Sequence Synthetic oligonucleotide
gugauaacugaag

SEQ ID NO: 648 - RNA, 14 nt, Artificial Sequence
  Description of Artificial Sequence Synthetic oligonucleotide
acaaugguguuugu

SEQ ID NO: 649 - RNA, 12 nt, Artificial Sequence
  Description of Artificial Sequence Synthetic oligonucleotide
gaguaauaaugc

SEQ ID NO: 650 - RNA, 13 nt, Artificial Sequence
  Description of Artificial Sequence Synthetic oligonucleotide
agaauacgcguag

SEQ ID NO: 651 - RNA, 8 nt, Artificial Sequence
  Description of Artificial Sequence Synthetic oligonucleotide
ugugaauc

SEQ ID NO: 652 - RNA, 12 nt, Artificial Sequence
  Description of Artificial Sequence Synthetic oligonucleotide
acccuaugguag

SEQ ID NO: 653 - RNA, 14 nt, Artificial Sequence
  Description of Artificial Sequence Synthetic oligonucleotide
uccuacuuuaugga

SEQ ID NO: 654 - RNA, 13 nt, Artificial Sequence
  Description of Artificial Sequence Synthetic oligonucleotide
gaugauguacuag

SEQ ID NO: 655 - RNA, 13 nt, Artificial Sequence
  Description of Artificial Sequence Synthetic oligonucleotide
aauuccauggguu

SEQ ID NO: 656 - RNA, 11 nt, Artificial Sequence
  Description of Artificial Sequence Synthetic oligonucleotide
cacaaaaguga

SEQ ID NO: 657 - RNA, 13 nt, Artificial Sequence
  Description of Artificial Sequence Synthetic oligonucleotide
aucgugauagggg

SEQ ID NO: 658 - RNA, 14 nt, Artificial Sequence
  Description of Artificial Sequence Synthetic oligonucleotide
gguagaauucacug

SEQ ID NO: 659 - RNA, 13 nt, Artificial Sequence
  Description of Artificial Sequence Synthetic oligonucleotide
aacugauaagggu

SEQ ID NO: 660 - RNA, 12 nt, Artificial Sequence
  Description of Artificial Sequence Synthetic oligonucleotide
guguugcagccg

SEQ ID NO: 661 - RNA, 13 nt, Artificial Sequence
  Description of Artificial Sequence Synthetic oligonucleotide
ugauauauuaggu

SEQ ID NO: 662 - RNA, 13 nt, Artificial Sequence
  Description of Artificial Sequence Synthetic oligonucleotide
aacuccaugugga

SEQ ID NO: 663 - RNA, 13 nt, Artificial Sequence
  Description of Artificial Sequence Synthetic oligonucleotide
uuaggaccacuag

SEQ ID NO: 664 - RNA, 13 nt, Artificial Sequence
  Description of Artificial Sequence Synthetic oligonucleotide
ccaccggagucug

SEQ ID NO: 665 - RNA, 12 nt, Artificial Sequence
  Description of Artificial Sequence Synthetic oligonucleotide
cuggcaacugug

SEQ ID NO: 666 - RNA, 15 nt, Artificial Sequence
  Description of Artificial Sequence Synthetic oligonucleotide
aggaacugauuggau

SEQ ID NO: 667 - RNA, 12 nt, Artificial Sequence
  Description of Artificial Sequence Synthetic oligonucleotide
aucuaaccaugu

SEQ ID NO: 668 - RNA, 12 nt, Artificial Sequence
  Description of Artificial Sequence Synthetic oligonucleotide
aaacgcaauucu

SEQ ID NO: 669 - RNA, 12 nt, Artificial Sequence
  Description of Artificial Sequence Synthetic oligonucleotide
gucaaauacccc

SEQ ID NO: 670 - RNA, 13 nt, Artificial Sequence
  Description of Artificial Sequence Synthetic oligonucleotide
ucggcucgcguga

SEQ ID NO: 671 - RNA, 6 nt, Artificial Sequence
  Description of Artificial Sequence Synthetic oligonucleotide
aauaaa

SEQ ID NO: 672 - RNA, 6 nt, Artificial Sequence
  Description of Artificial Sequence Synthetic oligonucleotide
auuaaa

SEQ ID NO: 673 - RNA, 4 nt, Artificial Sequence
  Description of Artificial Sequence Synthetic oligonucleotide
ugua

SEQ ID NO: 674 - RNA, 7 nt, Artificial Sequence
  Description of Artificial Sequence Synthetic oligonucleotide
cagugcc

SEQ ID NO: 675 - RNA, 7 nt, Artificial Sequence
  Description of Artificial Sequence Synthetic oligonucleotide
cggaccu

SEQ ID NO: 676 - RNA, 7 nt, Artificial Sequence
  Description of Artificial Sequence Synthetic oligonucleotide
cgcguac

SEQ ID NO: 677 - RNA, 44 nt, Homo sapiens
aaaaaaggaaaaguaggcaaaugugaaaauaguuucaauauauc

SEQ ID NO: 678 - RNA, 42 nt, Mus sp.
caaaagaaaaauaggcaaaugugaaaacaguuuuagcauauu

SEQ ID NO: 679 - RNA, 42 nt, Rattus sp.; identical to SEQ ID NO: 678

SEQ ID NO: 680 - RNA, 42 nt, Canis familiaris
aagaaccaaaguaggaaaaugugaaaauaguuucaguguaug

SEQ ID NO: 681 - RNA, 41 nt, Gallus gallus
agaauuagaaggagacaaaugugaaaauaguuuaaguaaag

SEQ ID NO: 682 - RNA, 22 nt, Artificial Sequence
  Description of Artificial Sequence Synthetic oligonucleotide
aucacauugccgagggauuucc

SEQ ID NO: 683 - RNA, 7 nt, Artificial Sequence
  Description of Artificial Sequence Synthetic oligonucleotide
cuaccuc

SEQ ID NO: 684 - RNA, 7 nt, Artificial Sequence
  Description of Artificial Sequence Synthetic oligonucleotide
guacugu

SEQ ID NO: 685 - RNA, 7 nt, Artificial Sequence
  Description of Artificial Sequence Synthetic oligonucleotide
augcugc

SEQ ID NO: 686 - RNA, 7 nt, Artificial Sequence
  Description of Artificial Sequence Synthetic oligonucleotide
acagggu

SEQ ID NO: 687 - RNA, 7 nt, Artificial Sequence
  Description of Artificial Sequence Synthetic oligonucleotide
acacucc

SEQ ID NO: 688 - RNA, 7 nt, Artificial Sequence
  Description of Artificial Sequence Synthetic oligonucleotide
cucaggg

SEQ ID NO: 689 - RNA, 7 nt, Artificial Sequence
  Description of Artificial Sequence Synthetic oligonucleotide
cacugug

SEQ ID NO: 690 - RNA, 7 nt, Artificial Sequence
  Description of Artificial Sequence Synthetic oligonucleotide
uugcacu

SEQ ID NO: 691 - RNA, 7 nt, Artificial Sequence
  Description of Artificial Sequence Synthetic oligonucleotide
gacuguu

SEQ ID NO: 692 - RNA, 7 nt, Artificial Sequence
  Description of Artificial Sequence Synthetic oligonucleotide
gggacca

SEQ ID NO: 693 - RNA, 7 nt, Artificial Sequence
  Description of Artificial Sequence Synthetic oligonucleotide
caccagc

SEQ ID NO: 694 - RNA, 7 nt, Artificial Sequence
  Description of Artificial Sequence Synthetic oligonucleotide
auacugu

SEQ ID NO: 695 - RNA, 7 nt, Artificial Sequence
  Description of Artificial Sequence Synthetic oligonucleotide
aacugga

SEQ ID NO: 696 - RNA, 7 nt, Artificial Sequence
  Description of Artificial Sequence Synthetic oligonucleotide
aguucuc

SEQ ID NO: 697 - RNA, 7 nt, Artificial Sequence
  Description of Artificial Sequence Synthetic oligonucleotide
ugcacug

SEQ ID NO: 698 - RNA, 7 nt, Artificial Sequence
  Description of Artificial Sequence Synthetic oligonucleotide
ugaaugu

SEQ ID NO: 699 - RNA, 7 nt, Artificial Sequence
  Description of Artificial Sequence Synthetic oligonucleotide
uugccaa

SEQ ID NO: 700 - RNA, 7 nt, Artificial Sequence
  Description of Artificial Sequence Synthetic oligonucleotide
uccgucc

SEQ ID NO: 701 - RNA, 7 nt, Artificial Sequence
  Description of Artificial Sequence Synthetic oligonucleotide
cuguuac

SEQ ID NO: 702 - RNA, 7 nt, Artificial Sequence
  Description of Artificial Sequence Synthetic oligonucleotide
acuaccu

SEQ ID NO: 703 - RNA, 7 nt, Artificial Sequence
  Description of Artificial Sequence Synthetic oligonucleotide
acacugg

SEQ ID NO: 704 - RNA, 7 nt, Artificial Sequence
  Description of Artificial Sequence Synthetic oligonucleotide
uuugcac

SEQ ID NO: 705 - RNA, 7 nt, Artificial Sequence
  Description of Artificial Sequence Synthetic oligonucleotide
acauucc

SEQ ID NO: 706 - RNA, 7 nt, Artificial Sequence
  Description of Artificial Sequence Synthetic oligonucleotide
gcacuuu

SEQ ID NO: 707 - RNA, 7 nt, Artificial Sequence
  Description of Artificial Sequence Synthetic oligonucleotide
caguauu

SEQ ID NO: 708 - RNA, 7 nt, Artificial Sequence
  Description of Artificial Sequence Synthetic oligonucleotide
auaccuc

SEQ ID NO: 709 - RNA, 7 nt, Artificial Sequence
  Description of Artificial Sequence Synthetic oligonucleotide
ugagauu

SEQ ID NO: 710 - RNA, 7 nt, Artificial Sequence
  Description of Artificial Sequence Synthetic oligonucleotide
aauguga

SEQ ID NO: 711 - RNA, 7 nt, Artificial Sequence
  Description of Artificial Sequence Synthetic oligonucleotide
cugagcc

SEQ ID NO: 712 - RNA, 7 nt, Artificial Sequence
  Description of Artificial Sequence Synthetic oligonucleotide
gugcaau

SEQ ID NO: 713 - RNA, 7 nt, Artificial Sequence
  Description of Artificial Sequence Synthetic oligonucleotide
uacuuga

SEQ ID NO: 714 - RNA, 7 nt, Artificial Sequence
  Description of Artificial Sequence Synthetic oligonucleotide
uggugcu

SEQ ID NO: 715 - RNA, 7 nt, Artificial Sequence
  Description of Artificial Sequence Synthetic oligonucleotide
uguuuac

SEQ ID NO: 716 - RNA, 7 nt, Artificial Sequence
  Description of Artificial Sequence Synthetic oligonucleotide
accaaag

SEQ ID NO: 717 - RNA, 7 nt, Artificial Sequence
  Description of Artificial Sequence Synthetic oligonucleotide
ugcugcu

SEQ ID NO: 718 - RNA, 7 nt, Artificial Sequence
  Description of Artificial Sequence Synthetic oligonucleotide
acuguga

SEQ ID NO: 719 - RNA, 7 nt, Artificial Sequence
  Description of Artificial Sequence Synthetic oligonucleotide
agcacuu

SEQ ID NO: 720 - RNA, 7 nt, Artificial Sequence
  Description of Artificial Sequence Synthetic oligonucleotide
gcaaaaa

SEQ ID NO: 721 - RNA, 7 nt, Artificial Sequence
  Description of Artificial Sequence Synthetic oligonucleotide
aagccau

SEQ ID NO: 722 - RNA, 7 nt, Artificial Sequence
  Description of Artificial Sequence Synthetic oligonucleotide
acagugu

SEQ ID NO: 723 - RNA, 7 nt, Artificial Sequence
  Description of Artificial Sequence Synthetic oligonucleotide
gcaccuu

SEQ ID NO: 724 - RNA, 7 nt, Artificial Sequence
  Description of Artificial Sequence Synthetic oligonucleotide
ccugcug

SEQ ID NO: 725 - RNA, 7 nt, Artificial Sequence
  Description of Artificial Sequence Synthetic oligonucleotide
ggcagcu

SEQ ID NO: 726 - RNA, 7 nt, Artificial Sequence
  Description of Artificial Sequence Synthetic oligonucleotide
cacugcc

SEQ ID NO: 727 - RNA, 7 nt, Artificial Sequence
  Description of Artificial Sequence Synthetic oligonucleotide
gugccaa

SEQ ID NO: 728 - RNA, 7 nt, Artificial Sequence
  Description of Artificial Sequence Synthetic oligonucleotide
aaaccac

SEQ ID NO: 729 - RNA, 7 nt, Artificial Sequence
  Description of Artificial Sequence Synthetic oligonucleotide
uuuaugg

SEQ ID NO: 730 - RNA, 7 nt, Artificial Sequence
  Description of Artificial Sequence Synthetic oligonucleotide
ucaucuc

SEQ ID NO: 731 - RNA, 7 nt, Artificial Sequence
  Description of Artificial Sequence Synthetic oligonucleotide
gugccau

SEQ ID NO: 732 - RNA, 7 nt, Artificial Sequence
  Description of Artificial Sequence Synthetic oligonucleotide
acauuuc

SEQ ID NO: 733 - RNA, 7 nt, Artificial Sequence
  Description of Artificial Sequence Synthetic oligonucleotide
acgcaca

SEQ ID NO: 734 - RNA, 7 nt, Artificial Sequence
  Description of Artificial Sequence Synthetic oligonucleotide
aagcaca

SEQ ID NO: 735 - RNA, 7 nt, Artificial Sequence
  Description of Artificial Sequence Synthetic oligonucleotide
auguagc

SEQ ID NO: 736 - RNA, 7 nt, Artificial Sequence
  Description of Artificial Sequence Synthetic oligonucleotide
aucuugc

SEQ ID NO: 737 - RNA, 7 nt, Artificial Sequence
  Description of Artificial Sequence Synthetic oligonucleotide
caaugca

SEQ ID NO: 738 - RNA, 7 nt, Artificial Sequence
  Description of Artificial Sequence Synthetic oligonucleotide
gucuucc

SEQ ID NO: 739 - RNA, 7 nt, Artificial Sequence
  Description of Artificial Sequence Synthetic oligonucleotide
aagcaau

SEQ ID NO: 740 - RNA, 7 nt, Artificial Sequence
  Description of Artificial Sequence Synthetic oligonucleotide
cuaugca

SEQ ID NO: 741 - RNA, 7 nt, Artificial Sequence
  Description of Artificial Sequence Synthetic oligonucleotide
agcauua

SEQ ID NO: 742 - RNA, 7 nt, Artificial Sequence
  Description of Artificial Sequence Synthetic oligonucleotide
aaaggga

SEQ ID NO: 743 - RNA, 7 nt, Artificial Sequence
  Description of Artificial Sequence Synthetic oligonucleotide
aacugac

SEQ ID NO: 744 - RNA, 7 nt, Artificial Sequence
  Description of Artificial Sequence Synthetic oligonucleotide
ggugcua

SEQ ID NO: 745 - RNA, 7 nt, Artificial Sequence
  Description of Artificial Sequence Synthetic oligonucleotide
acacuac

SEQ ID NO: 746 - RNA, 7 nt, Artificial Sequence
  Description of Artificial Sequence Synthetic oligonucleotide
uagcuuu

SEQ ID NO: 747 - RNA, 7 nt, Artificial Sequence
  Description of Artificial Sequence Synthetic oligonucleotide
acauauc

SEQ ID NO: 748 - RNA, 7 nt, Artificial Sequence
  Description of Artificial Sequence Synthetic oligonucleotide
augaagg

SEQ ID NO: 749 - RNA, 7 nt, Artificial Sequence
  Description of Artificial Sequence Synthetic oligonucleotide
uacgggu

SEQ ID NO: 750 - RNA, 7 nt, Artificial Sequence
  Description of Artificial Sequence Synthetic oligonucleotide
gacaauc

SEQ ID NO: 751 - RNA, 7 nt, Artificial Sequence
  Description of Artificial Sequence Synthetic oligonucleotide
gugccuu

SEQ ID NO: 752 - RNA, 7 nt, Artificial Sequence
  Description of Artificial Sequence Synthetic oligonucleotide
uagguca

SEQ ID NO: 753 - RNA, 7 nt, Artificial Sequence
  Description of Artificial Sequence Synthetic oligonucleotide
caguguu

SEQ ID NO: 754 - RNA, 7 nt, Artificial Sequence
  Description of Artificial Sequence Synthetic oligonucleotide
auaagcu

SEQ ID NO: 755 - RNA, 7 nt, Artificial Sequence
  Description of Artificial Sequence Synthetic oligonucleotide
cgguacg

SEQ ID NO: 756 - RNA, 7 nt, Artificial Sequence
  Description of Artificial Sequence Synthetic oligonucleotide
agacacg

SEQ ID NO: 757 - RNA, 7 nt, Artificial Sequence
  Description of Artificial Sequence Synthetic oligonucleotide
cauuuca

SEQ ID NO: 758 - RNA, 7 nt, Artificial Sequence
  Description of Artificial Sequence Synthetic oligonucleotide
augcagu

SEQ ID NO: 759 - RNA, 7 nt, Artificial Sequence
  Description of Artificial Sequence Synthetic oligonucleotide
cgaacaa

SEQ ID NO: 760 - RNA, 7 nt, Artificial Sequence
  Description of Artificial Sequence Synthetic oligonucleotide
agguccg

SEQ ID NO: 761 - RNA, 7 nt, Artificial Sequence
  Description of Artificial Sequence Synthetic oligonucleotide
guacgcg

SEQ ID NO: 762 - RNA, 22 nt, Artificial Sequence
  Description of Artificial Sequence Synthetic oligonucleotide
aacuauacaaccuacuaccuca

SEQ ID NO: 763 - RNA, 22 nt, Artificial Sequence
  Description of Artificial Sequence Synthetic oligonucleotide
uauaccacaucacuaccaucaa

SEQ ID NO: 764 - RNA, 22 nt, Artificial Sequence
  Description of Artificial Sequence Synthetic oligonucleotide
auacccuauacuccauaaacca

SEQ ID NO: 765 - RNA, 22 nt, Artificial Sequence
  Description of Artificial Sequence Synthetic oligonucleotide
acuaacuccauauacacccaua

SEQ ID NO: 766 - RNA, 22 nt, Artificial Sequence
  Description of Artificial Sequence Synthetic oligonucleotide
cauaccuaccuacucaacauaa

SEQ ID NO: 767 - RNA, 22 nt, Artificial Sequence
  Description of Artificial Sequence Synthetic oligonucleotide
aaccacacaaccuacuaccuca

SEQ ID NO: 768 - RNA, 22 nt, Artificial Sequence
  Description of Artificial Sequence Synthetic oligonucleotide
aaccauacaaccuacuaccuca

SEQ ID NO: 769 - RNA, 21 nt, Artificial Sequence
  Description of Artificial Sequence Synthetic oligonucleotide
acuaugcaaccuacuaccucu

SEQ ID NO: 770 - RNA, 21 nt, Artificial Sequence
  Description of Artificial Sequence Synthetic oligonucleotide
acuauacaaccuccuaccuca

SEQ ID NO: 771 - RNA, 22 nt, Artificial Sequence
  Description of Artificial Sequence Synthetic oligonucleotide
aacuauacaaucuacuaccuca

SEQ ID NO: 772 - RNA, 22 nt, Artificial Sequence
  Description of Artificial Sequence Synthetic oligonucleotide
aacuguacaaacuacuaccuca

SEQ ID NO: 773 - RNA, 22 nt, Artificial Sequence
  Description of Artificial Sequence Synthetic oligonucleotide
aacagcacaaacuacuaccuca

SEQ ID NO: 774 - RNA, 22 nt, Artificial Sequence
  Description of Artificial Sequence Synthetic oligonucleotide
auacauacuucuuuacauucca

SEQ ID NO: 775 - RNA, 22 nt, Artificial Sequence
  Description of Artificial Sequence Synthetic oligonucleotide
uaucuccccuauuaaauuuaca

SEQ ID NO: 776 - RNA, 22 nt, Artificial Sequence
  Description of Artificial Sequence Synthetic oligonucleotide
auuccuuauacucccuaaauua

SEQ ID NO: 777 - RNA, 22 nt, Artificial Sequence
  Description of Artificial Sequence Synthetic oligonucleotide
uacauucuaaucuaacucuuca

SEQ ID NO: 778 - RNA, 22 nt, Artificial Sequence
  Description of Artificial Sequence Synthetic oligonucleotide
uaucucaaauacucuuacuuca

SEQ ID NO: 779 - RNA, 21 nt, Artificial Sequence
  Description of Artificial Sequence Synthetic oligonucleotide
aacaaaaucacuagucuucca

SEQ ID NO: 780 - RNA, 21 nt, Artificial Sequence
  Description of Artificial Sequence Synthetic oligonucleotide
caccacauauucaaacauuga

SEQ ID NO: 781 - RNA, 21 nt, Artificial Sequence
  Description of Artificial Sequence Synthetic oligonucleotide
acaucuccuugaaccaaaaua

SEQ ID NO: 782 - RNA, 21 nt, Artificial Sequence
  Description of Artificial Sequence Synthetic oligonucleotide
cuucauacaauccagaaauca

SEQ ID NO: 783 - RNA, 21 nt, Artificial Sequence
  Description of Artificial Sequence Synthetic oligonucleotide
aaagauuaucucaucccacaa

SEQ ID NO: 784 - RNA, 23 nt, Artificial Sequence
  Description of Artificial Sequence Synthetic oligonucleotide
ucauacagcuagauaaccaaaga

SEQ ID NO: 785 - RNA, 23 nt, Artificial Sequence
  Description of Artificial Sequence Synthetic oligonucleotide
ucucaaucgcuaaaaaaagagca

SEQ ID NO: 786 - RNA, 23 nt, Artificial Sequence
  Description of Artificial Sequence Synthetic oligonucleotide
augaacucgccaaaaauucagaa

SEQ ID NO: 787 - RNA, 23 nt, Artificial Sequence
  Description of Artificial Sequence Synthetic oligonucleotide
acaaagauacccauacagaguua

SEQ ID NO: 788 - RNA, 23 nt, Artificial Sequence
  Description of Artificial Sequence Synthetic oligonucleotide
uaucauguacacaacaagagcaa

SEQ ID NO: 789 - RNA, 23 nt, Artificial Sequence
  Description of Artificial Sequence Synthetic oligonucleotide
cacaaauucggaucuacagggua

SEQ ID NO: 790 - RNA, 22 nt, Artificial Sequence
  Description of Artificial Sequence Synthetic oligonucleotide
acaaauucgguucuacagggua

SEQ ID NO: 791 - RNA, 22 nt, Artificial Sequence
  Description of Artificial Sequence Synthetic oligonucleotide
ucagcauucgguaaauggcaua

SEQ ID NO: 792 - RNA, 22 nt, Artificial Sequence
  Description of Artificial Sequence Synthetic oligonucleotide
ggcaaauguauuucgcagcaua

SEQ ID NO: 793 - RNA, 22 nt, Artificial Sequence
  Description of Artificial Sequence Synthetic oligonucleotide
agcaacgugucaguugaucaua

SEQ ID NO: 794 - RNA, 22 nt, Artificial Sequence
  Description of Artificial Sequence Synthetic oligonucleotide
uuuaaacaucagccgugaggua

SEQ ID NO: 795 - RNA, 22 nt, Artificial Sequence
  Description of Artificial Sequence Synthetic oligonucleotide
cacaaaccauuaugugcugcua

SEQ ID NO: 796 - RNA, 22 nt, Artificial Sequence
  Description of Artificial Sequence Synthetic oligonucleotide
uguaaaccaugaugugcugcua

SEQ ID NO: 797 - RNA, 22 nt, Artificial Sequence
  Description of Artificial Sequence Synthetic oligonucleotide
cgccaauauuuacgugcugcua

SEQ ID NO: 798 - RNA, 22 nt, Artificial Sequence
  Description of Artificial Sequence Synthetic oligonucleotide
acgaccugcgugacuauuucua

SEQ ID NO: 799 - RNA, 22 nt, Artificial Sequence
  Description of Artificial Sequence Synthetic oligonucleotide
acacgauuagcgcuucucugua

SEQ ID NO: 800 - RNA, 22 nt, Artificial Sequence
  Description of Artificial Sequence Synthetic oligonucleotide
aucggcuaagcgauuuccucua

SEQ ID NO: 801 - RNA, 22 nt, Artificial Sequence
  Description of Artificial Sequence Synthetic oligonucleotide
cuggacacgucacguauuucua

SEQ ID NO: 802 - RNA, 20 nt, Artificial Sequence
  Description of Artificial Sequence Synthetic oligonucleotide
acaagugccuucacugcagu

SEQ ID NO: 803 - RNA, 22 nt, Artificial Sequence
  Description of Artificial Sequence Synthetic oligonucleotide
uaucugcacuagaugcaccuua

SEQ ID NO: 804 - RNA, 22 nt, Artificial Sequence
  Description of Artificial Sequence Synthetic oligonucleotide
cccauucguucuucauagaaga

SEQ ID NO: 805 - RNA, 22 nt, Artificial Sequence
  Description of Artificial Sequence Synthetic oligonucleotide
cucgaugagacuuuuuacccaa

SEQ ID NO: 806 - RNA, 22 nt, Artificial Sequence
  Description of Artificial Sequence Synthetic oligonucleotide
aaguccaaucguuuuacccuga

SEQ ID NO: 807 - RNA, 22 nt, Artificial Sequence
  Description of Artificial Sequence Synthetic oligonucleotide
gguaucacucacugcauauuca

SEQ ID NO: 808 - RNA, 23 nt, Artificial Sequence
  Description of Artificial Sequence Synthetic oligonucleotide
ucaguuuugcauagauuugcaca

SEQ ID NO: 809 - RNA, 23 nt, Artificial Sequence
  Description of Artificial Sequence Synthetic oligonucleotide
uuccuauauuuuucaggaaggca

SEQ ID NO: 810 - RNA, 23 nt, Artificial Sequence
  Description of Artificial Sequence Synthetic oligonucleotide
uucagaagucauauuugguucca

SEQ ID NO: 811 - RNA, 23 nt, Artificial Sequence
  Description of Artificial Sequence Synthetic oligonucleotide
uguuguaaauucaugcucaguca

SEQ ID NO: 812 - RNA, 23 nt, Artificial Sequence
  Description of Artificial Sequence Synthetic oligonucleotide
cuuguaucaagguccuuauugaa

SEQ ID NO: 813 - RNA, 23 nt, Artificial Sequence
  Description of Artificial Sequence Synthetic oligonucleotide
ucaguuuugcauggauuugcaca

SEQ ID NO: 814 - RNA, 23 nt, Artificial Sequence
  Description of Artificial Sequence Synthetic oligonucleotide
cuaccugcacuauaagcacuuua

SEQ ID NO: 815 - RNA, 23 nt, Artificial Sequence
  Description of Artificial Sequence Synthetic oligonucleotide
acuauuaucauguuccagcacca

SEQ ID NO: 816 - RNA, 23 nt, Artificial Sequence
  Description of Artificial Sequence Synthetic oligonucleotide
ccuugacaauauguacauccuca

SEQ ID NO: 817 - RNA, 23 nt, Artificial Sequence
  Description of Artificial Sequence Synthetic oligonucleotide
cauacugucacacugcauuuaca

SEQ ID NO: 818 - RNA, 23 nt, Artificial Sequence
  Description of Artificial Sequence Synthetic oligonucleotide
uacaugaucccagauccucauua

SEQ ID NO: 819 - RNA, 22 nt, Artificial Sequence
  Description of Artificial Sequence Synthetic oligonucleotide
ucaacaucagucugauaagcua

SEQ ID NO: 820 - RNA, 22 nt, Artificial Sequence
  Description of Artificial Sequence Synthetic oligonucleotide
ugugauaacccugcauaacuaa

SEQ ID NO: 821 - RNA, 22 nt, Artificial Sequence
  Description of Artificial Sequence Synthetic oligonucleotide
uaagucccugaagucacuaaua

SEQ ID NO: 822 - RNA, 22 nt, Artificial Sequence
  Description of Artificial Sequence Synthetic oligonucleotide
gccaacuacuugacauuauaga

SEQ ID NO: 823 - RNA, 22 nt, Artificial Sequence
  Description of Artificial Sequence Synthetic oligonucleotide
gccuguccauguaaauaacuaa

SEQ ID NO: 824 - RNA, 22 nt, Artificial Sequence
  Description of Artificial Sequence Synthetic oligonucleotide
acaguucuucaacuggcagcuu

SEQ ID NO: 825 - RNA, 22 nt, Artificial Sequence
  Description of Artificial Sequence Synthetic oligonucleotide
uggaguuucaaaguccaccucu

SEQ ID NO: 826 - RNA, 22 nt, Artificial Sequence
  Description of Artificial Sequence Synthetic oligonucleotide
ccaacugguguugacacaucuu

SEQ ID NO: 827 - RNA, 22 nt, Artificial Sequence
  Description of Artificial Sequence Synthetic oligonucleotide
gacuucuuaaagagugccccuu

SEQ ID NO: 828 - RNA, 22 nt, Artificial Sequence
  Description of Artificial Sequence Synthetic oligonucleotide
ugccaacacaggcuuucauguu

SEQ ID NO: 829 - RNA, 21 nt, Artificial Sequence
  Description of Artificial Sequence Synthetic oligonucleotide
ggaaaucccuggcaaugugau

SEQ ID NO: 830 - RNA, 21 nt, Artificial Sequence
  Description of Artificial Sequence Synthetic oligonucleotide
ccuagaggcuugcagaaugau

SEQ ID NO: 831 - RNA, 21 nt, Artificial Sequence
  Description of Artificial Sequence Synthetic oligonucleotide
cauucagacagcuuggaggau

SEQ ID NO: 832 - RNA, 21 nt, Artificial Sequence
  Description of Artificial Sequence Synthetic oligonucleotide
caggucagauuuagccaggau

SEQ ID NO: 833 - RNA, 21 nt, Artificial Sequence
  Description of Artificial Sequence Synthetic oligonucleotide
uggccucucaaaaugggagau

SEQ ID NO: 834 - RNA, 23 nt, Artificial Sequence
  Description of Artificial Sequence Synthetic oligonucleotide
gugguaaucccuggcaaugugau

SEQ ID NO: 835 - RNA, 22 nt, Artificial Sequence
  Description of Artificial Sequence Synthetic oligonucleotide
cuguuccugcugaacugagcca

SEQ ID NO: 836 - RNA, 22 nt, Artificial Sequence
  Description of Artificial Sequence Synthetic oligonucleotide
gaaugcauugccuuccuggcca

SEQ ID NO: 837 - RNA, 22 nt, Artificial Sequence
  Description of Artificial Sequence Synthetic oligonucleotide
cucucagggaggauuucccuca

SEQ ID NO: 838 - RNA, 22 nt, Artificial Sequence
  Description of Artificial Sequence Synthetic oligonucleotide
aggcaauuucuugccccuggca

SEQ ID NO: 839 - RNA, 22 nt, Artificial Sequence
  Description of Artificial Sequence Synthetic oligonucleotide
cccuauugggcuuuccagagca

SEQ ID NO: 840 - RNA, 22 nt, Artificial Sequence
  Description of Artificial Sequence Synthetic oligonucleotide
ucagaccgagacaagugcaaug

SEQ ID NO: 841 - RNA, 22 nt, Artificial Sequence
  Description of Artificial Sequence Synthetic oligonucleotide
aaccaacacgaucaauuggggg

SEQ ID NO: 842 - RNA, 22 nt, Artificial Sequence
  Description of Artificial Sequence Synthetic oligonucleotide
aaugagaccaacgggucucaag

SEQ ID NO: 843 - RNA, 22 nt, Artificial Sequence
  Description of Artificial Sequence Synthetic oligonucleotide
caacuuagaggacgagccaaug

SEQ ID NO: 844 - RNA, 22 nt, Artificial Sequence
  Description of Artificial Sequence Synthetic oligonucleotide
caggugaaccgaauaccaaugg

SEQ ID NO: 845 - RNA, 22 nt, Artificial Sequence
  Description of Artificial Sequence Synthetic oligonucleotide
agccuauccuggauuacuugaa

SEQ ID NO: 846 - RNA, 22 nt, Artificial Sequence
  Description of Artificial Sequence Synthetic oligonucleotide
guucaguacccuucugaaguaa

SEQ ID NO: 847 - RNA, 22 nt, Artificial Sequence
  Description of Artificial Sequence Synthetic oligonucleotide
gucuuaagcuuaccugcaguaa

SEQ ID NO: 848 - RNA, 22 nt, Artificial Sequence
  Description of Artificial Sequence Synthetic oligonucleotide
auugucaggucccauaacuuga

SEQ ID NO: 849 - RNA, 22 nt, Artificial Sequence
  Description of Artificial Sequence Synthetic oligonucleotide
uaauuuacccuccagaggguua

SEQ ID NO: 850 - RNA, 22 nt, Artificial Sequence
  Description of Artificial Sequence Synthetic oligonucleotide
aaccuauccugaauuacuugaa

SEQ ID NO: 851 - RNA, 22 nt, Artificial Sequence
  Description of Artificial Sequence Synthetic oligonucleotide
ggcggaacuuagccacugugaa

SEQ ID NO: 852 - RNA, 22 nt, Artificial Sequence
  Description of Artificial Sequence Synthetic oligonucleotide
acaaggcugagcgaugcuguca

SEQ ID NO: 853 - RNA, 19 nt, Artificial Sequence
  Description of Artificial Sequence Synthetic oligonucleotide
acgaauggucccaugcagu

SEQ ID NO: 854 - RNA, 22 nt, Artificial Sequence
  Description of Artificial Sequence Synthetic oligonucleotide
cacggggccagacaggauuuua

SEQ ID NO: 855 - RNA, 22 nt, Artificial Sequence
  Description of Artificial Sequence Synthetic oligonucleotide
gaggcaaaccggggcuuucaua

SEQ ID NO: 856 - RNA, 20 nt, Artificial Sequence
  Description of Artificial Sequence Synthetic oligonucleotide
cagaacuuagccacugugaa

SEQ ID NO: 857 - RNA, 22 nt, Artificial Sequence
  Description of Artificial Sequence Synthetic oligonucleotide
cucaauagacugugagcuccuu

SEQ ID NO: 858 - RNA, 22 nt, Artificial Sequence
  Description of Artificial Sequence Synthetic oligonucleotide
aaccgauuucagauggugcuag

SEQ ID NO: 859 - RNA, 22 nt, Artificial Sequence
  Description of Artificial Sequence Synthetic oligonucleotide
aagcccuuaauucguagugagg

SEQ ID NO: 860 - RNA, 22 nt, Artificial Sequence
  Description of Artificial Sequence Synthetic oligonucleotide
aaagggugcauucgucuaucag

SEQ ID NO: 861 - RNA, 22 nt, Artificial Sequence
  Description of Artificial Sequence Synthetic oligonucleotide
ugagacaauugccgacuuagug

SEQ ID NO: 862 - RNA, 22 nt, Artificial Sequence
  Description of Artificial Sequence Synthetic oligonucleotide
acugcgaagucuugaucuagag

SEQ ID NO: 863 - RNA, 23 nt, Artificial Sequence
  Description of Artificial Sequence Synthetic oligonucleotide
aacacugauuucaaauggugcua

SEQ ID NO: 864 - RNA, 23 nt, Artificial Sequence
  Description of Artificial Sequence Synthetic oligonucleotide
cuuacaagagcagguuucuaaua

SEQ ID NO: 865 - RNA, 23 nt, Artificial Sequence
  Description of Artificial Sequence Synthetic oligonucleotide
caacuaauugagaguucugcaua

SEQ ID NO: 866 - RNA, 23 nt, Artificial Sequence
  Description of Artificial Sequence Synthetic oligonucleotide
aggaauuccuguccuuaaaguaa

SEQ ID NO: 867 - RNA, 23 nt, Artificial Sequence
  Description of Artificial Sequence Synthetic oligonucleotide
cuuuuauccaaugggagaacaua

SEQ ID NO: 868 - RNA, 22 nt, Artificial Sequence
  Description of Artificial Sequence Synthetic oligonucleotide
uaaccgauuucaaauggugcua

SEQ ID NO: 869 - RNA, 23 nt, Artificial Sequence
  Description of Artificial Sequence Synthetic oligonucleotide
gcuuccagucgaggauguuuaca

SEQ ID NO: 870 - RNA, 21 nt, Artificial Sequence
  Description of Artificial Sequence Synthetic oligonucleotide
gcugaguguaggauguuuaca

SEQ ID NO: 871 - RNA, 21 nt, Artificial Sequence
  Description of Artificial Sequence Synthetic oligonucleotide
cuauguggaugugugugacaa

SEQ ID NO: 872 - RNA, 21 nt, Artificial Sequence
  Description of Artificial Sequence Synthetic oligonucleotide
uagaauagguguugcucugga

SEQ ID NO: 873 - RNA, 21 nt, Artificial Sequence
  Description of Artificial Sequence Synthetic oligonucleotide
uguguaauggacaugggcuua

SEQ ID NO: 874 - RNA, 21 nt, Artificial Sequence
  Description of Artificial Sequence Synthetic oligonucleotide
uuauggucauguguggacaga

SEQ ID NO: 875 - RNA, 23 nt, Artificial Sequence
  Description of Artificial Sequence Synthetic oligonucleotide
gcugagaguguaggauguuuaca

SEQ ID NO: 876 - RNA, 22 nt, Artificial Sequence
  Description of Artificial Sequence Synthetic oligonucleotide
cuuccagucggggauguuuaca

SEQ ID NO: 877 - RNA, 19 nt, Artificial Sequence
  Description of Artificial Sequence Synthetic oligonucleotide
ccagucaaggauguuuaca

SEQ ID NO: 878 - RNA, 21 nt, Artificial Sequence
  Description of Artificial Sequence Synthetic oligonucleotide
cagcuaugccagcaucuugcc

SEQ ID NO: 879 - RNA, 21 nt, Artificial Sequence
  Description of Artificial Sequence Synthetic oligonucleotide
ccucuaaggacucaguugccc

SEQ ID NO: 880 - RNA, 21 nt, Artificial Sequence
  Description of Artificial Sequence Synthetic oligonucleotide
cugugccugacuagccucaac

SEQ ID NO: 881 - RNA, 21 nt, Artificial Sequence
  Description of Artificial Sequence Synthetic oligonucleotide
ugggccaauccaugcccauuc

SEQ ID NO: 882 - RNA, 21 nt, Artificial Sequence
  Description of Artificial Sequence Synthetic oligonucleotide
gaccagaccuugccuuguacc

SEQ ID NO: 883 - RNA, 21 nt, Artificial Sequence
  Description of Artificial Sequence Synthetic oligonucleotide
gcaacuuaguaaugugcaaua

SEQ ID NO: 884 - RNA, 19 nt, Artificial Sequence
  Description of Artificial Sequence Synthetic oligonucleotide
caaugcaacuacaaugcac

SEQ ID NO: 885 - RNA, 19 nt, Artificial Sequence
  Description of Artificial Sequence Synthetic oligonucleotide
aacccuagaugaucaaacc

SEQ ID NO: 886 - RNA, 19 nt, Artificial Sequence
  Description of Artificial Sequence Synthetic oligonucleotide
uaagcuaaucaagaacccc

SEQ ID NO: 887 - RNA, 19 nt, Artificial Sequence
  Description of Artificial Sequence Synthetic oligonucleotide
aaacaccuuagccaagauc

SEQ ID NO: 888 - RNA, 19 nt, Artificial Sequence
  Description of Artificial Sequence Synthetic oligonucleotide
caagugaccuacaacaauc

SEQ ID NO: 889 - RNA, 19 nt, Artificial Sequence
  Description of Artificial Sequence Synthetic oligonucleotide
caaugcaacagcaaugcac

SEQ ID NO: 890 - RNA, 22 nt, Artificial Sequence
  Description of Artificial Sequence Synthetic oligonucleotide
acaaccagcuaagacacugcca

SEQ ID NO: 891 - RNA, 22 nt, Artificial Sequence
  Description of Artificial Sequence Synthetic oligonucleotide
accccauagagaaucagccaca

SEQ ID NO: 892 - RNA, 22 nt, Artificial Sequence
  Description of Artificial Sequence Synthetic oligonucleotide
guuacacacaaacacaggccca

SEQ ID NO: 893 - RNA, 22 nt, Artificial Sequence
  Description of Artificial Sequence Synthetic oligonucleotide
gagacucccacugacacacaaa

SEQ ID NO: 894 - RNA, 22 nt, Artificial Sequence
  Description of Artificial Sequence Synthetic oligonucleotide
aacauaccagacccuccaggaa

SEQ ID NO: 895 - RNA, 22 nt, Artificial Sequence
  Description of Artificial Sequence Synthetic oligonucleotide
acaggccgggacaagugcaaua

SEQ ID NO: 896 - RNA, 22 nt, Artificial Sequence
  Description of Artificial Sequence Synthetic oligonucleotide
cuaccugcacgaacagcacuuu

SEQ ID NO: 897 - RNA, 22 nt, Artificial Sequence
  Description of Artificial Sequence Synthetic oligonucleotide
aacacaucuccggcaaccuguu

SEQ ID NO: 898 - RNA, 22 nt, Artificial Sequence
  Description of Artificial Sequence Synthetic oligonucleotide
gcaagcacuuuacgccacacuu

SEQ ID NO: 899 - RNA, 22 nt, Artificial Sequence
  Description of Artificial Sequence Synthetic oligonucleotide
cucuagcugaccgcuccaaaau

SEQ ID NO: 900 - RNA, 22 nt, Artificial Sequence
  Description of Artificial Sequence Synthetic oligonucleotide
ucccaauuaaaccgccagugcu

SEQ ID NO: 901 - RNA, 20 nt, Artificial Sequence
  Description of Artificial Sequence Synthetic oligonucleotide
aucugcacugucagcacuuu

SEQ ID NO: 902 - RNA, 22 nt, Artificial Sequence
  Description of Artificial Sequence Synthetic oligonucleotide
ugcucaauaaauacccguugaa

SEQ ID NO: 903 - RNA, 22 nt, Artificial Sequence
  Description of Artificial Sequence Synthetic oligonucleotide
gcaaaaaugugcuagugccaaa

SEQ ID NO: 904 - RNA, 22 nt, Artificial Sequence
  Description of Artificial Sequence Synthetic oligonucleotide
gagagaacauccaaggauuuca

SEQ ID NO: 905 - RNA, 22 nt, Artificial Sequence
  Description of Artificial Sequence Synthetic oligonucleotide
aacugguuaaaccagugaagca

SEQ ID NO: 906 - RNA, 22 nt, Artificial Sequence
  Description of Artificial Sequence Synthetic oligonucleotide
gcaaaugacagaaggacuuuca

SEQ ID NO: 907 - RNA, 22 nt, Artificial Sequence
  Description of Artificial Sequence Synthetic oligonucleotide
uaaaaggaugugccugcaacaa

SEQ ID NO: 908 - RNA, 22 nt, Artificial Sequence
  Description of Artificial Sequence Synthetic oligonucleotide
aacaauacaacuuacuaccuca

SEQ ID NO: 909 - RNA, 20 nt, Artificial Sequence
  Description of Artificial Sequence Synthetic oligonucleotide
acaagaucggaucuacgggu

SEQ ID NO: 910 - RNA, 20 nt, Artificial Sequence
  Description of Artificial Sequence Synthetic oligonucleotide
ugacagcgggaucguacaau

SEQ ID NO: 911 - RNA, 20 nt, Artificial Sequence
  Description of Artificial Sequence Synthetic oligonucleotide
agagcggucucacggaauau

SEQ ID NO: 912 - RNA, 20 nt, Artificial Sequence
  Description of Artificial Sequence Synthetic oligonucleotide
gauacaagugccgaucggau

SEQ ID NO: 913 - RNA, 20 nt, Artificial Sequence
  Description of Artificial Sequence Synthetic oligonucleotide
agagccaguacgucgguaau

SEQ ID NO: 914 - RNA, 22 nt, Artificial Sequence
  Description of Artificial Sequence Synthetic oligonucleotide
cgcaaggucgguucuacgggug

SEQ ID NO: 915 - RNA, 22 nt, Artificial Sequence
  Description of Artificial Sequence Synthetic oligonucleotide
cacaaguucggaucuacggguu

SEQ ID NO: 916 - RNA, 22 nt, Artificial Sequence
  Description of Artificial Sequence Synthetic oligonucleotide
gguugaaacccacguuaugcgu

SEQ ID NO: 917 - RNA, 22 nt, Artificial Sequence
  Description of Artificial Sequence Synthetic oligonucleotide
ccugacgaguugaguaaucgcu

SEQ ID NO: 918 - RNA, 22 nt, Artificial Sequence
  Description of Artificial Sequence Synthetic oligonucleotide
ugaaucccgggaaugcgcuuau

SEQ ID NO: 919 - RNA, 22 nt, Artificial Sequence
  Description of Artificial Sequence Synthetic oligonucleotide
gguguagccauauagccguacu

SEQ ID NO: 920 - RNA, 20 nt, Artificial Sequence
  Description of Artificial Sequence Synthetic oligonucleotide
ucaguuaucacaguacugua

SEQ ID NO: 921 - RNA, 20 nt, Artificial Sequence
  Description of Artificial Sequence Synthetic oligonucleotide
gccucuuacuaaguguauaa

SEQ ID NO: 922 - RNA, 20 nt, Artificial Sequence
  Description of Artificial Sequence Synthetic oligonucleotide
gcagucuagcuucuauauaa

SEQ ID NO: 923 - RNA, 20 nt, Artificial Sequence
  Description of Artificial Sequence Synthetic oligonucleotide
ucuauuggcuacauagcaua

SEQ ID NO: 924 - RNA, 20 nt, Artificial Sequence
  Description of Artificial Sequence Synthetic oligonucleotide
guugauagcacccuauauua

SEQ ID NO: 925 - RNA, 23 nt, Artificial Sequence
  Description of Artificial Sequence Synthetic oligonucleotide
ucauagcccuguacaaugcugcu

SEQ ID NO: 926 - RNA, 23 nt, Artificial Sequence
  Description of Artificial Sequence Synthetic oligonucleotide
ggucaucucaauacugcccugau

SEQ ID NO: 927 - RNA, 23 nt, Artificial Sequence
  Description of Artificial Sequence Synthetic oligonucleotide
gagucuagagccuacucucacuu

SEQ ID NO: 928 - RNA, 23 nt, Artificial Sequence
  Description of Artificial Sequence Synthetic oligonucleotide
gauauccuguccauuagagcccu

SEQ ID NO: 929 - RNA, 23 nt, Artificial Sequence
  Description of Artificial Sequence Synthetic oligonucleotide
cuuugcuccgaggaaacuuccau

SEQ ID NO: 930 - RNA, 23 nt, Artificial Sequence
  Description of Artificial Sequence Synthetic oligonucleotide
ucauagcccuguacaauguugcu

SEQ ID NO: 931 - RNA, 22 nt, Artificial Sequence
  Description of Artificial Sequence Synthetic oligonucleotide
uagcuuaucagacugauguuga

SEQ ID NO: 932 - RNA, 22 nt, Artificial Sequence
  Description of Artificial Sequence Synthetic oligonucleotide
cgaaggguuuucuucuaugaaa

SEQ ID NO: 933 - RNA, 22 nt, Artificial Sequence
  Description of Artificial Sequence Synthetic oligonucleotide
gguaauguauccugacuuuaga

SEQ ID NO: 934 - RNA, 22 nt, Artificial Sequence
  Description of Artificial Sequence Synthetic oligonucleotide
ugaucuacugugugagauucaa

SEQ ID NO: 935 - RNA, 22 nt, Artificial Sequence
  Description of Artificial Sequence Synthetic oligonucleotide
uagucuuaaggguaauucugca

SEQ ID NO: 936 - RNA, 20 nt, Artificial Sequence
  Description of Artificial Sequence Synthetic oligonucleotide
acaggagucugagcauuuga

SEQ ID NO: 937 - RNA, 24 nt, Artificial Sequence
  Description of Artificial Sequence Synthetic oligonucleotide
gcuaccugcacuguaagcacuuuu

SEQ ID NO: 938 - RNA, 23 nt, Artificial Sequence
  Description of Artificial Sequence Synthetic oligonucleotide
ugauagcccuguacaaugcugcu

SEQ ID NO: 939 - RNA, 21 nt, Artificial Sequence
  Description of Artificial Sequence Synthetic oligonucleotide
aaugccccuaaaaauccuuau

SEQ ID NO: 940 - RNA, 21 nt, Artificial Sequence
  Description of Artificial Sequence Synthetic oligonucleotide
caaaucauugccucauaacau

SEQ ID NO: 941 - RNA, 21 nt, Artificial Sequence
  Description of Artificial Sequence Synthetic oligonucleotide
aagaauccacaauucccuuau

SEQ ID NO: 942 - RNA, 21 nt, Artificial Sequence
  Description of Artificial Sequence Synthetic oligonucleotide
cuuugaucuaacaacacaacu

SEQ ID NO: 943 - RNA, 21 nt, Artificial Sequence
  Description of Artificial Sequence Synthetic oligonucleotide
gaaacaaacucuucaucuacu

SEQ ID NO: 944 - RNA, 23 nt, Artificial Sequence
  Description of Artificial Sequence Synthetic oligonucleotide
acaaacaccauugucacacucca

SEQ ID NO: 945 - RNA, 23 nt, Artificial Sequence
  Description of Artificial Sequence Synthetic oligonucleotide
aaacauucccacaaccuguacca

SEQ ID NO: 946 - RNA, 23 nt, Artificial Sequence
  Description of Artificial Sequence Synthetic oligonucleotide
ucuccaagaacaaauccuaccca

SEQ ID NO: 947 - RNA, 23 nt, Artificial Sequence
  Description of Artificial Sequence Synthetic oligonucleotide
ccccaaaucacuuacagcuacaa

SEQ ID NO: 948 - RNA, 23 nt, Artificial Sequence
  Description of Artificial Sequence Synthetic oligonucleotide
cacugcaccccacaauaacuuaa

SEQ ID NO: 949 - RNA, 21 nt, Artificial Sequence
  Description of Artificial Sequence Synthetic oligonucleotide
cgcguaccaaaaguaauaaug

SEQ ID NO: 950 - RNA, 21 nt, Artificial Sequence
  Description of Artificial Sequence Synthetic oligonucleotide
uaagcacgcgcaaaauauuag

SEQ ID NO: 951 - RNA, 21 nt, Artificial Sequence
  Description of Artificial Sequence Synthetic oligonucleotide
ucacaucgcggaaaauauaag

SEQ ID NO: 952 - RNA, 21 nt, Artificial Sequence
  Description of Artificial Sequence Synthetic oligonucleotide
ccagcguaacgaaaauuauag

SEQ ID NO: 953 - RNA, 21 nt, Artificial Sequence
  Description of Artificial Sequence Synthetic oligonucleotide
cguaagaauccgaaauacaug

SEQ ID NO: 954 - RNA, 22 nt, Artificial Sequence
  Description of Artificial Sequence Synthetic oligonucleotide
uggcauucaccgcgugccuuaa

SEQ ID NO: 955 - RNA, 22 nt, Artificial Sequence
  Description of Artificial Sequence Synthetic oligonucleotide
aagaugucgccuccguucugca

SEQ ID NO: 956 - RNA, 22 nt, Artificial Sequence
  Description of Artificial Sequence Synthetic oligonucleotide
ggaguccuucgacgcucucuaa

SEQ ID NO: 957 - RNA, 22 nt, Artificial Sequence
  Description of Artificial Sequence Synthetic oligonucleotide
cucugcgguuaucggccaucaa

SEQ ID NO: 958 - RNA, 22 nt, Artificial Sequence
  Description of Artificial Sequence Synthetic oligonucleotide
auccuucggaugacguccugca

SEQ ID NO: 959 - RNA, 23 nt, Artificial Sequence
  Description of Artificial Sequence Synthetic oligonucleotide
cacagguuaaagggucucaggga

SEQ ID NO: 960 - RNA, 22 nt, Artificial Sequence
  Description of Artificial Sequence Synthetic oligonucleotide
ucacaaguuagggucucaggga

SEQ ID NO: 961 - RNA, 22 nt, Artificial Sequence
  Description of Artificial Sequence Synthetic oligonucleotide
aguggaugcauauuggcccaga

SEQ ID NO: 962 - RNA, 22 nt, Artificial Sequence
  Description of Artificial Sequence Synthetic oligonucleotide
agcuacaaugucugcaggugga

SEQ ID NO: 963 - RNA, 22 nt, Artificial Sequence
  Description of Artificial Sequence Synthetic oligonucleotide
aacuguacugcagaugggcuga

SEQ ID NO: 964 - RNA, 22 nt, Artificial Sequence
  Description of Artificial Sequence Synthetic oligonucleotide
auuacccaggagagcuggguua

SEQ ID NO: 965 - RNA, 21 nt, Artificial Sequence
  Description of Artificial Sequence Synthetic oligonucleotide
gcauuauuacucacgguacga

SEQ ID NO: 966 - RNA, 21 nt, Artificial Sequence
  Description of Artificial Sequence Synthetic oligonucleotide
ucacuguacuaagucgcgaua

SEQ ID NO: 967 - RNA, 21 nt, Artificial Sequence
  Description of Artificial Sequence Synthetic oligonucleotide
ggucacuaucuuacgauacga

SEQ ID NO: 968 - RNA, 21 nt, Artificial Sequence
  Description of Artificial Sequence Synthetic oligonucleotide
uacccaugauuagcgaucgua

SEQ ID NO: 969 - RNA, 21 nt, Artificial Sequence
  Description of Artificial Sequence Synthetic oligonucleotide
uacauaagucuuccguacgga

SEQ ID NO: 970 - RNA, 22 nt, Artificial Sequence
  Description of Artificial Sequence Synthetic oligonucleotide
agccaagcucagacggauccga

SEQ ID NO: 971 - RNA, 22 nt, Artificial Sequence
  Description of Artificial Sequence Synthetic oligonucleotide
aaaagagaccgguucacuguga

SEQ ID NO: 972 - RNA, 22 nt, Artificial Sequence
  Description of Artificial Sequence Synthetic oligonucleotide
acauugagcgaacuugagcaga

SEQ ID NO: 973 - RNA, 22 nt, Artificial Sequence
  Description of Artificial Sequence Synthetic oligonucleotide
cagaggaaacgcaguucaguua

SEQ ID NO: 974 - RNA, 22 nt, Artificial Sequence
  Description of Artificial Sequence Synthetic oligonucleotide
auacauagguaaugcagggcca

SEQ ID NO: 975 - RNA, 22 nt, Artificial Sequence
  Description of Artificial Sequence Synthetic oligonucleotide
aagagcgaaacagguucuguca

SEQ ID NO: 976 - RNA, 22 nt, Artificial Sequence
  Description of Artificial Sequence Synthetic oligonucleotide
gaaagagaccgguucacuguga

SEQ ID NO: 977 - RNA, 20 nt, Artificial Sequence
  Description of Artificial Sequence Synthetic oligonucleotide
gcaagcccagaccgaaaaag

SEQ ID NO: 978 - RNA, 21 nt, Artificial Sequence
  Description of Artificial Sequence Synthetic oligonucleotide
gcaagcccagaccgcaaaaag

SEQ ID NO: 979 - RNA, 21 nt, Artificial Sequence
  Description of Artificial Sequence Synthetic oligonucleotide
ccggacccaaacacaaaggag

SEQ ID NO: 980 - RNA, 21 nt, Artificial Sequence
  Description of Artificial Sequence Synthetic oligonucleotide
gaagaaccggaaacccccaag

SEQ ID NO: 981 - RNA, 21 nt, Artificial Sequence
  Description of Artificial Sequence Synthetic oligonucleotide
gccaagccaaacgccaaaagg

SEQ ID NO: 982 - RNA, 21 nt, Artificial Sequence
  Description of Artificial Sequence Synthetic oligonucleotide
cgcccaaccagaacaaaggag

SEQ ID NO: 983 - RNA, 20 nt, Artificial Sequence
  Description of Artificial Sequence Synthetic oligonucleotide
gcccuuuuaacauugcacug

SEQ ID NO: 984 - RNA, 20 nt, Artificial Sequence
  Description of Artificial Sequence Synthetic oligonucleotide
uuugaaguccaccucucaug

SEQ ID NO: 985 - RNA, 20 nt, Artificial Sequence
  Description of Artificial Sequence Synthetic oligonucleotide
ucuuccaagucugcauucag

SEQ ID NO: 986 - RNA, 20 nt, Artificial Sequence
  Description of Artificial Sequence Synthetic oligonucleotide
guuauguuucccuccaacag

SEQ ID NO: 987 - RNA, 20 nt, Artificial Sequence
  Description of Artificial Sequence Synthetic oligonucleotide
uccauccucaauuugagcug

SEQ ID NO: 988 - RNA, 20 nt, Artificial Sequence
  Description of Artificial Sequence Synthetic oligonucleotide
gcccuuucaucauugcacug

SEQ ID NO: 989 - RNA, 21 nt, Artificial Sequence
  Description of Artificial Sequence Synthetic oligonucleotide
acuuucgguuaucuagcuuua

SEQ ID NO: 990 - RNA, 21 nt, Artificial Sequence
  Description of Artificial Sequence Synthetic oligonucleotide
ugugcauucacguuuaucuua

SEQ ID NO: 991 - RNA, 21 nt, Artificial Sequence
  Description of Artificial Sequence Synthetic oligonucleotide
uguuuuacaucggaucuucua

SEQ ID NO: 992 - RNA, 21 nt, Artificial Sequence
  Description of Artificial Sequence Synthetic oligonucleotide
uacuuuuaggcucguuucaua

SEQ ID NO: 993 - RNA, 21 nt, Artificial Sequence
  Description of Artificial Sequence Synthetic oligonucleotide
uaucucgacuuuguugucaua

SEQ ID NO: 994 - RNA, 23 nt, Artificial Sequence
  Description of Artificial Sequence Synthetic oligonucleotide
gcgaccauggcuguagacuguua

SEQ ID NO: 995 - RNA, 23 nt, Artificial Sequence
  Description of Artificial Sequence Synthetic oligonucleotide
ggaucuccgacuaugggaugcua

SEQ ID NO: 996 - RNA, 23 nt, Artificial Sequence
  Description of Artificial Sequence Synthetic oligonucleotide
aggucuuccguaccaggauguga

SEQ ID NO: 997 - RNA, 23 nt, Artificial Sequence
  Description of Artificial Sequence Synthetic oligonucleotide
cuccgguaggcaguauggcauua

SEQ ID NO: 998 - RNA, 23 nt, Artificial Sequence
  Description of Artificial Sequence Synthetic oligonucleotide
gcuguacugucggaaugccagua

SEQ ID NO: 999 - RNA, 22 nt, Artificial Sequence
  Description of Artificial Sequence Synthetic oligonucleotide
acagcugguugaaggggaccaa

SEQ ID NO: 1000 - RNA, 22 nt, Artificial Sequence
  Description of Artificial Sequence Synthetic oligonucleotide
cugacaagcaaugaggugggca

SEQ ID NO: 1001 - RNA, 22 nt, Artificial Sequence
  Description of Artificial Sequence Synthetic oligonucleotide
gggaggcuucaugagaaccaga

SEQ ID NO: 1002 - RNA, 22 nt, Artificial Sequence
  Description of Artificial Sequence Synthetic oligonucleotide
ccuaaaggcaagggggacuuga

SEQ ID NO: 1003 - RNA, 22 nt, Artificial Sequence
  Description of Artificial Sequence Synthetic oligonucleotide
agcugugggccaaagagaugca

SEQ ID NO: 1004 - RNA, 21 nt, Artificial Sequence
  Description of Artificial Sequence Synthetic oligonucleotide
uagcugguugaaggggaccaa

SEQ ID NO: 1005 - RNA, 22 nt, Artificial Sequence
  Description of Artificial Sequence Synthetic oligonucleotide
ccccucuggucaaccagucaca

SEQ ID NO: 1006 - RNA, 24 nt, Artificial Sequence
  Description of Artificial Sequence Synthetic oligonucleotide
aucacauaggaauaaaaagccaua

SEQ ID NO: 1007 - RNA, 24 nt, Artificial Sequence
  Description of Artificial Sequence Synthetic oligonucleotide
auuaguacacaaaaaaccagagua

SEQ ID NO: 1008 - RNA, 24 nt, Artificial Sequence
  Description of Artificial Sequence Synthetic oligonucleotide
gauaaaaauagcacaacaugcaua

SEQ ID NO: 1009 - RNA, 24 nt, Artificial Sequence
  Description of Artificial Sequence Synthetic oligonucleotide
gcaaauauaaauagccaagaacua

SEQ ID NO: 1010 - RNA, 24 nt, Artificial Sequence
  Description of Artificial Sequence Synthetic oligonucleotide
guagcagaaaaccuaacauaaaua

SEQ ID NO: 1011 - RNA, 23 nt, Artificial Sequence
  Description of Artificial Sequence Synthetic oligonucleotide
uccaucaucaaaacaaauggagu

SEQ ID NO: 1012 - RNA, 22 nt, Artificial Sequence
  Description of Artificial Sequence Synthetic oligonucleotide
cuacgcguauucuuaagcaaua

SEQ ID NO: 1013 - RNA, 22 nt, Artificial Sequence
  Description of Artificial Sequence Synthetic oligonucleotide
accguuaaucguaagacuucua

SEQ ID NO: 1014 - RNA, 22 nt, Artificial Sequence
  Description of Artificial Sequence Synthetic oligonucleotide
aacguaacauucgucacuugua

SEQ ID NO: 1015 - RNA, 22 nt, Artificial Sequence
  Description of Artificial Sequence Synthetic oligonucleotide
caaguacgcguauuaaucucua

SEQ ID NO: 1016 - RNA, 22 nt, Artificial Sequence
  Description of Artificial Sequence Synthetic oligonucleotide
cgaucucacgucuauuaguaaa

SEQ ID NO: 1017 - RNA, 17 nt, Artificial Sequence
  Description of Artificial Sequence Synthetic oligonucleotide
gauucacaacaccagcu

SEQ ID NO: 1018 - RNA, 17 nt, Artificial Sequence
  Description of Artificial Sequence Synthetic oligonucleotide
cuccaaauacacaggcu

SEQ ID NO: 1019 - RNA, 17 nt, Artificial Sequence
  Description of Artificial Sequence Synthetic oligonucleotide
cccaaguaaccacugau

SEQ ID NO: 1020 - RNA, 17 nt, Artificial Sequence
  Description of Artificial Sequence Synthetic oligonucleotide
aggaaaucacccuccau

SEQ ID NO: 1021 - RNA, 17 nt, Artificial Sequence
  Description of Artificial Sequence Synthetic oligonucleotide
ccaggcuacaaauccau

SEQ ID NO: 1022 - RNA, 23 nt, Artificial Sequence
  Description of Artificial Sequence Synthetic oligonucleotide
acuggagacacgugcacuguaga

SEQ ID NO: 1023 - RNA, 21 nt, Artificial Sequence
  Description of Artificial Sequence Synthetic oligonucleotide
cuaccauaggguaaaaccacu

SEQ ID NO: 1024 - RNA, 21 nt, Artificial Sequence
  Description of Artificial Sequence Synthetic oligonucleotide
aacaggauaccaucacaugcu

SEQ ID NO: 1025 - RNA, 21 nt, Artificial Sequence
  Description of Artificial Sequence Synthetic oligonucleotide
aguauacccagccaacaaugu

SEQ ID NO: 1026 - RNA, 21 nt, Artificial Sequence
  Description of Artificial Sequence Synthetic oligonucleotide
auucgucaagaaaagcccacu

SEQ ID NO: 1027 - RNA, 21 nt, Artificial Sequence
  Description of Artificial Sequence Synthetic oligonucleotide
cuaacccgaaaaaggacucuu

SEQ ID NO: 1028 - RNA, 21 nt, Artificial Sequence
  Description of Artificial Sequence Synthetic oligonucleotide
ccaucuuuaccagacaguguu

SEQ ID NO: 1029 - RNA, 21 nt, Artificial Sequence
  Description of Artificial Sequence Synthetic oligonucleotide
aucugagcacauuucugccau

SEQ ID NO: 1030 - RNA, 21 nt, Artificial Sequence
  Description of Artificial Sequence Synthetic oligonucleotide
ucugcucacuugcaaaucagu

SEQ ID NO: 1031 - RNA, 21 nt, Artificial Sequence
  Description of Artificial Sequence Synthetic oligonucleotide
aauguuaacuggcuccucacu

SEQ ID NO: 1032 - RNA, 21 nt, Artificial Sequence
  Description of Artificial Sequence Synthetic oligonucleotide
uuaaugagcuugaucccaccu

SEQ ID NO: 1033 - RNA, 22 nt, Artificial Sequence
  Description of Artificial Sequence Synthetic oligonucleotide
ccauaaaguaggaaacacuaca

SEQ ID NO: 1034 - RNA, 22 nt, Artificial Sequence
  Description of Artificial Sequence Synthetic oligonucleotide
caauagaagucaagauaccaca

SEQ ID NO: 1035 - RNA, 22 nt, Artificial Sequence
  Description of Artificial Sequence Synthetic oligonucleotide
aaacaccuacaagaaggcuaua

SEQ ID NO: 1036 - RNA, 22 nt, Artificial Sequence
  Description of Artificial Sequence Synthetic oligonucleotide
caguagcaaacaaguacuacaa

SEQ ID NO: 1037 - RNA, 22 nt, Artificial Sequence
  Description of Artificial Sequence Synthetic oligonucleotide
auacaacaagccaaagcuagua

SEQ ID NO: 1038 - RNA, 22 nt, Artificial Sequence
  Description of Artificial Sequence Synthetic oligonucleotide
guagugcuuucuacuuuauggg

SEQ ID NO: 1039 - RNA, 22 nt, Artificial Sequence
  Description of Artificial Sequence Synthetic oligonucleotide
guguuuauggcuuaugacucug

SEQ ID NO: 1040 - RNA, 22 nt, Artificial Sequence
  Description of Artificial Sequence Synthetic oligonucleotide
gggcaugguuuauuaucuucug

SEQ ID NO: 1041 - RNA, 22 nt, Artificial Sequence
  Description of Artificial Sequence Synthetic oligonucleotide
aguaugucugcucuuuuagugg

SEQ ID NO: 1042 - RNA, 22 nt, Artificial Sequence
  Description of Artificial Sequence Synthetic oligonucleotide
caggcuuucgauuuuuugguag

SEQ ID NO: 1043 - RNA, 22 nt, Artificial Sequence
  Description of Artificial Sequence Synthetic oligonucleotide
ugagcuacagugcuucaucuca

SEQ ID NO: 1044 - RNA, 22 nt, Artificial Sequence
  Description of Artificial Sequence Synthetic oligonucleotide
ucacugugugagcuccacauua

SEQ ID NO: 1045 - RNA, 22 nt, Artificial Sequence
  Description of Artificial Sequence Synthetic oligonucleotide
ugcacugagccaucuauucuga

SEQ ID NO: 1046 - RNA, 22 nt, Artificial Sequence
  Description of Artificial Sequence Synthetic oligonucleotide
agucagguauuccuuccacuga

SEQ ID NO: 1047 - RNA, 22 nt, Artificial Sequence
  Description of Artificial Sequence Synthetic oligonucleotide
caucucacuucuuauggggaca

SEQ ID NO: 1048 - RNA, 22 nt, Artificial Sequence
  Description of Artificial Sequence Synthetic oligonucleotide
cuaguacaucaucuauacugua

SEQ ID NO: 1049 - RNA, 22 nt, Artificial Sequence
  Description of Artificial Sequence Synthetic oligonucleotide
gcucucuuucgcaauaauauaa

SEQ ID NO: 1050 - RNA, 22 nt, Artificial Sequence
  Description of Artificial Sequence Synthetic oligonucleotide
uucguugaacccuuauacauaa

SEQ ID NO: 1051 - RNA, 22 nt, Artificial Sequence
  Description of Artificial Sequence Synthetic oligonucleotide
cauuccuuuagacgacauuaua

SEQ ID NO: 1052 - RNA, 22 nt, Artificial Sequence
  Description of Artificial Sequence Synthetic oligonucleotide
uuucgcacaaugccuauauuaa

SEQ ID NO: 1053 - RNA, 24 nt, Artificial Sequence
  Description of Artificial Sequence Synthetic oligonucleotide
aagggauuccugggaaaacuggac

SEQ ID NO: 1054 - RNA, 24 nt, Artificial Sequence
  Description of Artificial Sequence Synthetic oligonucleotide
gaugaaaaacucuugcagggggac

SEQ ID NO: 1055 - RNA, 24 nt, Artificial Sequence
  Description of Artificial Sequence Synthetic oligonucleotide
auuugcaagggcaagagcuggaac

SEQ ID NO: 1056 - RNA, 24 nt, Artificial Sequence
  Description of Artificial Sequence Synthetic oligonucleotide
aaagucuugaacagcaaggggugc

SEQ ID NO: 1057 - RNA, 24 nt, Artificial Sequence
  Description of Artificial Sequence Synthetic oligonucleotide
aaaagaugaagcuggaguggccuc

SEQ ID NO: 1058 - RNA, 22 nt, Artificial Sequence
  Description of Artificial Sequence Synthetic oligonucleotide
aacccauggaauucaguucuca

SEQ ID NO: 1059 - RNA, 22 nt, Artificial Sequence
  Description of Artificial Sequence Synthetic oligonucleotide
cucauuuguaagccauccagaa

SEQ ID NO: 1060 - RNA, 22 nt, Artificial Sequence
  Description of Artificial Sequence Synthetic oligonucleotide
acugugcaacugaauccauuca

SEQ ID NO: 1061 - RNA, 22 nt, Artificial Sequence
  Description of Artificial Sequence Synthetic oligonucleotide
gcuucaacuguuagaaacucca

SEQ ID NO: 1062 - RNA, 22 nt, Artificial Sequence
  Description of Artificial Sequence Synthetic oligonucleotide
uguuaacaagcucaguccucaa

SEQ ID NO: 1063 - RNA, 21 nt, Artificial Sequence
  Description of Artificial Sequence Synthetic oligonucleotide
ggcagaagcauuuccacacac

SEQ ID NO: 1064 - RNA, 22 nt, Artificial Sequence
  Description of Artificial Sequence Synthetic oligonucleotide
acaaaguucuguagugcacuga

SEQ ID NO: 1065 - RNA, 22 nt, Artificial Sequence
  Description of Artificial Sequence Synthetic oligonucleotide
auucuugauaucaagcagggca

SEQ ID NO: 1066 - RNA, 22 nt, Artificial Sequence
  Description of Artificial Sequence Synthetic oligonucleotide
gaaguggcauuuuacucacaga

SEQ ID NO: 1067 - RNA, 22 nt, Artificial Sequence
  Description of Artificial Sequence Synthetic oligonucleotide
cugguaacuucagguaaaugca

SEQ ID NO: 1068 - RNA, 22 nt, Artificial Sequence
  Description of Artificial Sequence Synthetic oligonucleotide
uugcagaauagcaguucacuga

SEQ ID NO: 1069 - RNA, 22 nt, Artificial Sequence
  Description of Artificial Sequence Synthetic oligonucleotide
acaaaguucugugaugcacuga

SEQ ID NO: 1070 - RNA, 22 nt, Artificial Sequence
  Description of Artificial Sequence Synthetic oligonucleotide
ggagugaagacacggagccaga

SEQ ID NO: 1071 - RNA, 23 nt, Artificial Sequence
  Description of Artificial Sequence Synthetic oligonucleotide
acacugguacaaggguugggaga

SEQ ID NO: 1072 - RNA, 21 nt, Artificial Sequence
  Description of Artificial Sequence Synthetic oligonucleotide
ccucaaggagcuucagucuag

SEQ ID NO: 1073 - RNA, 21 nt, Artificial Sequence
  Description of Artificial Sequence Synthetic oligonucleotide
ccaaguucugucaugcacuga

SEQ ID NO: 1074 - RNA, 20 nt, Artificial Sequence
  Description of Artificial Sequence Synthetic oligonucleotide
ucacuuuugugacuaugcaa

SEQ ID NO: 1075 - RNA, 20 nt, Artificial Sequence
  Description of Artificial Sequence Synthetic oligonucleotide
cauguucaucuuaggcuuaa

SEQ ID NO: 1076 - RNA, 20 nt, Artificial Sequence
  Description of Artificial Sequence Synthetic oligonucleotide
uuauugggcuuuacaacuca

SEQ ID NO: 1077 - RNA, 20 nt, Artificial Sequence
  Description of Artificial Sequence Synthetic oligonucleotide
ccuuauucuuagacauugga

SEQ ID NO: 1078 - RNA, 20 nt, Artificial Sequence
  Description of Artificial Sequence Synthetic oligonucleotide
cuuaaguguugucacucuaa

SEQ ID NO: 1079 - RNA, 22 nt, Artificial Sequence
  Description of Artificial Sequence Synthetic oligonucleotide
cgaaggcaacacggauaaccua

SEQ ID NO: 1080 - RNA, 22 nt, Artificial Sequence
  Description of Artificial Sequence Synthetic oligonucleotide
ccccuaucacaauuagcauuaa

SEQ ID NO: 1081 - RNA, 22 nt, Artificial Sequence
  Description of Artificial Sequence Synthetic oligonucleotide
ucugacccuaaacauucaacua

SEQ ID NO: 1082 - RNA, 22 nt, Artificial Sequence
  Description of Artificial Sequence Synthetic oligonucleotide
auacacuugaaccauucacuca

SEQ ID NO: 1083 - RNA, 22 nt, Artificial Sequence
  Description of Artificial Sequence Synthetic oligonucleotide
acaacauguucccuccuaaaua

SEQ ID NO: 1084 - RNA, 22 nt, Artificial Sequence
  Description of Artificial Sequence Synthetic oligonucleotide
cucauugaccaauacucuacaa

SEQ ID NO: 1085 - RNA, 23 nt, Artificial Sequence
  Description of Artificial Sequence Synthetic oligonucleotide
acucaccgacagcguugaauguu

SEQ ID NO: 1086 - RNA, 23 nt, Artificial Sequence
  Description of Artificial Sequence Synthetic oligonucleotide
cugacguccgauguucacagaau

SEQ ID NO: 1087 - RNA, 23 nt, Artificial Sequence
  Description of Artificial Sequence Synthetic oligonucleotide
gccgauaguucucggaaacacuu

SEQ ID NO: 1088 - RNA, 23 nt, Artificial Sequence
  Description of Artificial Sequence Synthetic oligonucleotide
ugccgucugacgaucagaaacuu

SEQ ID NO: 1089 - RNA, 23 nt, Artificial Sequence
  Description of Artificial Sequence Synthetic oligonucleotide
auaugaaacgcggccuucucagu

SEQ ID NO: 1090 - RNA, 24 nt, Artificial Sequence
  Description of Artificial Sequence Synthetic oligonucleotide
aacccaccgacagcaaugaauguu

SEQ ID NO: 1091 - RNA, 22 nt, Artificial Sequence
  Description of Artificial Sequence Synthetic oligonucleotide
acucaccgacagguugaauguu

SEQ ID NO: 1092 - RNA, 22 nt, Artificial Sequence
  Description of Artificial Sequence Synthetic oligonucleotide
ugugaguucuaccauugccaaa

SEQ ID NO: 1093 - RNA, 22 nt, Artificial Sequence
  Description of Artificial Sequence Synthetic oligonucleotide
agaacuauucugcucuugcaga

SEQ ID NO: 1094 - RNA, 22 nt, Artificial Sequence
  Description of Artificial Sequence Synthetic oligonucleotide
guacaguucuccaauuuggcaa

SEQ ID NO: 1095 - RNA, 22 nt, Artificial Sequence
  Description of Artificial Sequence Synthetic oligonucleotide
gucuggucaugacucaaauuca

SEQ ID NO: 1096 - RNA, 22 nt, Artificial Sequence
  Description of Artificial Sequence Synthetic oligonucleotide
cuaauugggcuauccuugacaa

SEQ ID NO: 1097 - RNA, 23 nt, Artificial Sequence
  Description of Artificial Sequence Synthetic oligonucleotide
cagugaauucuaccagugccaua

SEQ ID NO: 1098 - RNA, 23 nt, Artificial Sequence
  Description of Artificial Sequence Synthetic oligonucleotide
ugaaaccagcugcuccauaguua

SEQ ID NO: 1099 - RNA, 23 nt, Artificial Sequence
  Description of Artificial Sequence Synthetic oligonucleotide
uugaugcucaccacaagugcaua

SEQ ID NO: 1100 - RNA, 23 nt, Artificial Sequence
  Description of Artificial Sequence Synthetic oligonucleotide
cccuuaacugauguguaaccaga

SEQ ID NO: 1101 - RNA, 23 nt, Artificial Sequence
  Description of Artificial Sequence Synthetic oligonucleotide
cucaaacagucauggccuguaua

SEQ ID NO: 1102 - RNA, 22 nt, Artificial Sequence
  Description of Artificial Sequence Synthetic oligonucleotide
acccuuaucaguucuccgucca

SEQ ID NO: 1103 - RNA, 22 nt, Artificial Sequence
  Description of Artificial Sequence Synthetic oligonucleotide
cauuaucuccugucccacguca

SEQ ID NO: 1104 - RNA, 22 nt, Artificial Sequence
  Description of Artificial Sequence Synthetic oligonucleotide
cacuaaugcuccuccguuccua

SEQ ID NO: 1105 - RNA, 22 nt, Artificial Sequence
  Description of Artificial Sequence Synthetic oligonucleotide
uauccacacuuuccucuccgga

SEQ ID NO: 1106 - RNA, 22 nt, Artificial Sequence
  Description of Artificial Sequence Synthetic oligonucleotide
cacagcuuacucuccguuccua

SEQ ID NO: 1107 - RNA, 18 nt, Artificial Sequence
  Description of Artificial Sequence Synthetic oligonucleotide
gaacugccuuucucucca

SEQ ID NO: 1108 - RNA, 23 nt, Artificial Sequence
  Description of Artificial Sequence Synthetic oligonucleotide
aagcccaaaaggagaauucuuug

SEQ ID NO: 1109 - RNA, 22 nt, Artificial Sequence
  Description of Artificial Sequence Synthetic oligonucleotide
ccggcugcaacacaagacacga

SEQ ID NO: 1110 - RNA, 22 nt, Artificial Sequence
  Description of Artificial Sequence Synthetic oligonucleotide
cccuaacccgcaaaacggagga

SEQ ID NO: 1111 - RNA, 22 nt, Artificial Sequence
  Description of Artificial Sequence Synthetic oligonucleotide
cacccagaugccgaaacacgga

SEQ ID NO: 1112 - RNA, 22 nt, Artificial Sequence
  Description of Artificial Sequence Synthetic oligonucleotide
aggaaaacauccccgccacgga

SEQ ID NO: 1113 - RNA, 22 nt, Artificial Sequence
  Description of Artificial Sequence Synthetic oligonucleotide
uccaagccgcaagcaacacgga

SEQ ID NO: 1114 - RNA, 22 nt, Artificial Sequence
  Description of Artificial Sequence Synthetic oligonucleotide
acccuccaccaugcaagggaug

SEQ ID NO: 1115 - RNA, 23 nt, Artificial Sequence
  Description of Artificial Sequence Synthetic oligonucleotide
acugaugucagcucaguaggcac

SEQ ID NO: 1116 - RNA, 22 nt, Artificial Sequence
  Description of Artificial Sequence Synthetic oligonucleotide
accuaauauaucaaacauauca

SEQ ID NO: 1117 - RNA, 22 nt, Artificial Sequence
  Description of Artificial Sequence Synthetic oligonucleotide
aucaauauaaucuaauaccaca

SEQ ID NO: 1118 - RNA, 22 nt, Artificial Sequence
  Description of Artificial Sequence Synthetic oligonucleotide
uacaauauuacaacuaacuaca

SEQ ID NO: 1119 - RNA, 22 nt, Artificial Sequence
  Description of Artificial Sequence Synthetic oligonucleotide
auaauaauaacaucuacucaca

SEQ ID NO: 1120 - RNA, 22 nt, Artificial Sequence
  Description of Artificial Sequence Synthetic oligonucleotide
acuaaaccuauaacauuauaca

SEQ ID NO: 1121 - RNA, 22 nt, Artificial Sequence
  Description of Artificial Sequence Synthetic oligonucleotide
agcugcuuuugggauuccguug

SEQ ID NO: 1122 - RNA, 21 nt, Artificial Sequence
  Description of Artificial Sequence Synthetic oligonucleotide
ggcugucaauucauaggucag

SEQ ID NO: 1123 - RNA, 21 nt, Artificial Sequence
  Description of Artificial Sequence Synthetic oligonucleotide
agaccuugcugacuauuaggg

SEQ ID NO: 1124 - RNA, 21 nt, Artificial Sequence
  Description of Artificial Sequence Synthetic oligonucleotide
ucucagauugaggauagccug

SEQ ID NO: 1125 - RNA, 21 nt, Artificial Sequence
  Description of Artificial Sequence Synthetic oligonucleotide
uaauuggccccuguaagguag

SEQ ID NO: 1126 - RNA, 21 nt, Artificial Sequence
  Description of Artificial Sequence Synthetic oligonucleotide
cucuaauugaggguaccaugg

SEQ ID NO: 1127 - RNA, 21 nt, Artificial Sequence
  Description of Artificial Sequence Synthetic oligonucleotide
cugggacuuuguaggccaguu

SEQ ID NO: 1128 - RNA, 22 nt, Artificial Sequence
  Description of Artificial Sequence Synthetic oligonucleotide
uccacauggaguugcuguuaca

SEQ ID NO: 1129 - RNA, 22 nt, Artificial Sequence
  Description of Artificial Sequence Synthetic oligonucleotide
uauuccuucuggguaaggacca

SEQ ID NO: 1130 - RNA, 22 nt, Artificial Sequence
  Description of Artificial Sequence Synthetic oligonucleotide
uuggcaucucuacugcauggaa

SEQ ID NO: 1131 - RNA, 22 nt, Artificial Sequence
  Description of Artificial Sequence Synthetic oligonucleotide
agcaugaugguucuauguccca

SEQ ID NO: 1132 - RNA, 22 nt, Artificial Sequence
  Description of Artificial Sequence Synthetic oligonucleotide
gccuggauuagcucacuuuaga

SEQ ID NO: 1133 - RNA, 21 nt, Artificial Sequence
  Description of Artificial Sequence Synthetic oligonucleotide
gccaauauuucugugcugcua

SEQ ID NO: 1134 - RNA, 22 nt, Artificial Sequence
  Description of Artificial Sequence Synthetic oligonucleotide
cccaacaacaugaaacuaccua

SEQ ID NO: 1135 - RNA, 22 nt, Artificial Sequence
  Description of Artificial Sequence Synthetic oligonucleotide
cccagaaaccaaacuaucuaca

SEQ ID NO: 1136 - RNA, 22 nt, Artificial Sequence
  Description of Artificial Sequence Synthetic oligonucleotide
cacaaaaugaccccuaccaaua

SEQ ID NO: 1137 - RNA, 22 nt, Artificial Sequence
  Description of Artificial Sequence Synthetic oligonucleotide
ccagccaucaaaacuauaacca

SEQ ID NO: 1138 - RNA, 22 nt, Artificial Sequence
  Description of Artificial Sequence Synthetic oligonucleotide
aaacaccuguccaaacuacaca

SEQ ID NO: 1139 - RNA, 22 nt, Artificial Sequence
  Description of Artificial Sequence Synthetic oligonucleotide
gcuggguggagaagguggugaa

SEQ ID NO: 1140 - RNA, 19 nt, Artificial Sequence
  Description of Artificial Sequence Synthetic oligonucleotide
ccuaucuccccucuggacc

SEQ ID NO: 1141 - RNA, 23 nt, Artificial Sequence
  Description of Artificial Sequence Synthetic oligonucleotide
gaacagguagucugaacacuggg

SEQ ID NO: 1142 - RNA, 23 nt, Artificial Sequence
  Description of Artificial Sequence Synthetic oligonucleotide
cacaggcucaaugguagaugagg

SEQ ID NO: 1143 - RNA, 23 nt, Artificial Sequence
  Description of Artificial Sequence Synthetic oligonucleotide
guaucuggaacuggagcagacag

SEQ ID NO: 1144 - RNA, 23 nt, Artificial Sequence
  Description of Artificial Sequence Synthetic oligonucleotide
uaaguggaugcccagagugacag

SEQ ID NO: 1145 - RNA, 23 nt, Artificial Sequence
  Description of Artificial Sequence Synthetic oligonucleotide
gaggauagguuguaaaccccagg

SEQ ID NO: 1146 - RNA, 23 nt, Artificial Sequence
  Description of Artificial Sequence Synthetic oligonucleotide
gaacagauagucuaaacacuggg

SEQ ID NO: 1147 - RNA, 21 nt, Artificial Sequence
  Description of Artificial Sequence Synthetic oligonucleotide
caucguuaccagacaguguua

SEQ ID NO: 1148 - RNA, 23 nt, Artificial Sequence
  Description of Artificial Sequence Synthetic oligonucleotide
gucaucauuaccaggcaguauua

SEQ ID NO: 1149 - RNA, 23 nt, Artificial Sequence
  Description of Artificial Sequence Synthetic oligonucleotide
aguacuggauaccauucucagua

SEQ ID NO: 1150 - RNA, 23 nt, Artificial Sequence
  Description of Artificial Sequence Synthetic oligonucleotide
uaguguccaauaguuagccacua

SEQ ID NO: 1151 - RNA, 23 nt, Artificial Sequence
  Description of Artificial Sequence Synthetic oligonucleotide
gaguuaaguacacuguccucaua

SEQ ID NO: 1152 - RNA, 23 nt, Artificial Sequence
  Description of Artificial Sequence Synthetic oligonucleotide
uggauccacuaauaguccaugua

SEQ ID NO: 1153 - RNA, 21 nt, Artificial Sequence
  Description of Artificial Sequence Synthetic oligonucleotide
agaacaaugccuuacugagua

SEQ ID NO: 1154 - RNA, 22 nt, Artificial Sequence
  Description of Artificial Sequence Synthetic oligonucleotide
ucuucccaugcgcuauaccucu

SEQ ID NO: 1155 - RNA, 22 nt, Artificial Sequence
  Description of Artificial Sequence Synthetic oligonucleotide
uugccccuacguccauaucucu

SEQ ID NO: 1156 - RNA, 22 nt, Artificial Sequence
  Description of Artificial Sequence Synthetic oligonucleotide
ucucagcccguucccuacuuau

SEQ ID NO: 1157 - RNA, 22 nt, Artificial Sequence
  Description of Artificial Sequence Synthetic oligonucleotide
acaucguccugucuuacucccu

SEQ ID NO: 1158 - RNA, 22 nt, Artificial Sequence
  Description of Artificial Sequence Synthetic oligonucleotide
ugucccuuccuaccguacucau

SEQ ID NO: 1159 - RNA, 22 nt, Artificial Sequence
  Description of Artificial Sequence Synthetic oligonucleotide
ucuagugguccuaaacauuuca

SEQ ID NO: 1160 - RNA, 22 nt, Artificial Sequence
  Description of Artificial Sequence Synthetic oligonucleotide
ucaucauugugucccaaaugua

SEQ ID NO: 1161 - RNA, 22 nt, Artificial Sequence
  Description of Artificial Sequence Synthetic oligonucleotide
ggccaucauuuacuagacuuua

SEQ ID NO: 1162 - RNA, 22 nt, Artificial Sequence
  Description of Artificial Sequence Synthetic oligonucleotide
ucacacacagauuguuguucua

SEQ ID NO: 1163 - RNA, 22 nt, Artificial Sequence
  Description of Artificial Sequence Synthetic oligonucleotide
ggugcuaaucaucauuccauua

SEQ ID NO: 1164 - RNA, 23 nt, Artificial Sequence
  Description of Artificial Sequence Synthetic oligonucleotide
caggcauaggaugacaaagggaa

SEQ ID NO: 1165 - RNA, 23 nt, Artificial Sequence
  Description of Artificial Sequence Synthetic oligonucleotide
cauaggggggacaacaaaaguga

SEQ ID NO: 1166 - RNA, 23 nt, Artificial Sequence
  Description of Artificial Sequence Synthetic oligonucleotide
ugagcaaguacaggcaaggagaa

SEQ ID NO: 1167 - RNA, 23 nt, Artificial Sequence
  Description of Artificial Sequence Synthetic oligonucleotide
agucaggagagaccuagaaggaa

SEQ ID NO: 1168 - RNA, 23 nt, Artificial Sequence
  Description of Artificial Sequence Synthetic oligonucleotide
aacaacuguacaggggggagaaa

SEQ ID NO: 1169 - RNA, 22 nt, Artificial Sequence
  Description of Artificial Sequence Synthetic oligonucleotide
cagacuccgguggaaugaagga

SEQ ID NO: 1170 - RNA, 22 nt, Artificial Sequence
  Description of Artificial Sequence Synthetic oligonucleotide
gggaaggccgaaggaauuccua

SEQ ID NO: 1171 - RNA, 22 nt, Artificial Sequence
  Description of Artificial Sequence Synthetic oligonucleotide
cagcgcacugugggaaaguaga

SEQ ID NO: 1172 - RNA, 22 nt, Artificial Sequence
  Description of Artificial Sequence Synthetic oligonucleotide
uagaaagcccgauugggggcaa

SEQ ID NO: 1173 - RNA, 22 nt, Artificial Sequence
  Description of Artificial Sequence Synthetic oligonucleotide
guuggaaggcccgauggaacaa

SEQ ID NO: 1174 - RNA, 22 nt, Artificial Sequence
  Description of Artificial Sequence Synthetic oligonucleotide
ccacacacuuccuuacauucca

SEQ ID NO: 1175 - RNA, 23 nt, Artificial Sequence
  Description of Artificial Sequence Synthetic oligonucleotide
gagggaggagagccaggagaagc

SEQ ID NO: 1176 - RNA, 22 nt, Artificial Sequence
  Description of Artificial Sequence Synthetic oligonucleotide
acaagcuuuuugcucgucuuau

SEQ ID NO: 1177 - RNA, 22 nt, Artificial Sequence
  Description of Artificial Sequence Synthetic oligonucleotide
caugaucauuucuuugucgcau

SEQ ID NO: 1178 - RNA, 22 nt, Artificial Sequence
  Description of Artificial Sequence Synthetic oligonucleotide
guucauuuuuaaccaugcucgu

SEQ ID NO: 1179 - RNA, 22 nt, Artificial Sequence
  Description of Artificial Sequence Synthetic oligonucleotide
gucaacuucuuguuuuaacgcu

SEQ ID NO: 1180 - RNA, 22 nt, Artificial Sequence
  Description of Artificial Sequence Synthetic oligonucleotide
uuuucuuagcaucaagucgucu

SEQ ID NO: 1181 - RNA, 21 nt, Artificial Sequence
  Description of Artificial Sequence Synthetic oligonucleotide
cagccgcugucacacgcacag

SEQ ID NO: 1182 - RNA, 21 nt, Artificial Sequence
  Description of Artificial Sequence Synthetic oligonucleotide
uccaagcccgacaggccuacg

SEQ ID NO: 1183 - RNA, 21 nt, Artificial Sequence
  Description of Artificial Sequence Synthetic oligonucleotide
aagccccccacguggcuaacg

SEQ ID NO: 1184 - RNA, 21 nt, Artificial Sequence
  Description of Artificial Sequence Synthetic oligonucleotide
cucaccccccggaggauaacg

SEQ ID NO: 1185 - RNA, 21 nt, Artificial Sequence
  Description of Artificial Sequence Synthetic oligonucleotide
cgcaaccagcaucuccacggg

SEQ ID NO: 1186 - RNA, 22 nt, Artificial Sequence
  Description of Artificial Sequence Synthetic oligonucleotide
aggcgaaggaugacaaagggaa

SEQ ID NO: 1187 - RNA, 21 nt, Artificial Sequence
  Description of Artificial Sequence Synthetic oligonucleotide
ggccgugacuggagacuguua

SEQ ID NO: 1188 - RNA, 22 nt, Artificial Sequence
  Description of Artificial Sequence Synthetic oligonucleotide
gguacaaucaacggucgauggu

SEQ ID NO: 1189 - RNA, 22 nt, Artificial Sequence
  Description of Artificial Sequence Synthetic oligonucleotide
uacuagcgaaggagaucucggu

SEQ ID NO: 1190 - RNA, 22 nt, Artificial Sequence
  Description of Artificial Sequence Synthetic oligonucleotide
guaaaggcuacguggucacgau

SEQ ID NO: 1191 - RNA, 22 nt, Artificial Sequence
  Description of Artificial Sequence Synthetic oligonucleotide
ugagacgcuggcauagacgauu

SEQ ID NO: 1192 - RNA, 22 nt, Artificial Sequence
  Description of Artificial Sequence Synthetic oligonucleotide
ggaauaccaucguggugacgau

SEQ ID NO: 1193 - RNA, 21 nt, Artificial Sequence
  Description of Artificial Sequence Synthetic oligonucleotide
cugccugucugugccugcugu

SEQ ID NO: 1194 - RNA, 21 nt, Artificial Sequence
  Description of Artificial Sequence Synthetic oligonucleotide
gccuggucuuggucucugccu

SEQ ID NO: 1195 - RNA, 21 nt, Artificial Sequence
  Description of Artificial Sequence Synthetic oligonucleotide
cuccucuguggugcugccugu

SEQ ID NO: 1196 - RNA, 21 nt, Artificial Sequence
  Description of Artificial Sequence Synthetic oligonucleotide
gugggcugcuucccuuccugu

SEQ ID NO: 1197 - RNA, 21 nt, Artificial Sequence
  Description of Artificial Sequence Synthetic oligonucleotide
ucuugggggcuucccuccugu

SEQ ID NO: 1198 - RNA, 21 nt, Artificial Sequence
  Description of Artificial Sequence Synthetic oligonucleotide
gucugucaauucauaggucau

SEQ ID NO: 1199 - RNA, 21 nt, Artificial Sequence
  Description of Artificial Sequence Synthetic oligonucleotide
cacaguugccagcugagauua

SEQ ID NO: 1200 - RNA, 21 nt, Artificial Sequence
  Description of Artificial Sequence Synthetic oligonucleotide
cuggagacauucuugacagca

SEQ ID NO: 1201 - RNA, 21 nt, Artificial Sequence
  Description of Artificial Sequence Synthetic oligonucleotide
gucaaagcacccuugagguua

SEQ ID NO: 1202 - RNA, 21 nt, Artificial Sequence
  Description of Artificial Sequence Synthetic oligonucleotide
caugcaugccugcaaauggua

SEQ ID NO: 1203 - RNA, 21 nt, Artificial Sequence
  Description of Artificial Sequence Synthetic oligonucleotide
caccaagggcugcuauuuaga

SEQ ID NO: 1204 - RNA, 24 nt, Artificial Sequence
  Description of Artificial Sequence Synthetic oligonucleotide
auccaaucaguuccugaugcagua

SEQ ID NO: 1205 - RNA, 21 nt, Artificial Sequence
  Description of Artificial Sequence Synthetic oligonucleotide
acaugguuagaucaagcacaa

SEQ ID NO: 1206 - RNA, 21 nt, Artificial Sequence
  Description of Artificial Sequence Synthetic oligonucleotide
aaucacagacuacaaguguga

SEQ ID NO: 1207 - RNA, 21 nt, Artificial Sequence
  Description of Artificial Sequence Synthetic oligonucleotide
uacuuguggagaacacacaaa

SEQ ID NO: 1208 - RNA, 21 nt, Artificial Sequence
  Description of Artificial Sequence Synthetic oligonucleotide
ucuauaaacacugaggaagca

SEQ ID NO: 1209 - RNA, 21 nt, Artificial Sequence
  Description of Artificial Sequence Synthetic oligonucleotide
ggacacaagcuacuuaaugaa

SEQ ID NO: 1210 - RNA, 21 nt, Artificial Sequence
  Description of Artificial Sequence Synthetic oligonucleotide
agaauugcguuuggacaauca

SEQ ID NO: 1211 - RNA, 21 nt, Artificial Sequence
  Description of Artificial Sequence Synthetic oligonucleotide
uuaucauaggguagagccuaa

SEQ ID NO: 1212 - RNA, 21 nt, Artificial Sequence
  Description of Artificial Sequence Synthetic oligonucleotide
aguuagcauaggucuagcuaa

SEQ ID NO: 1213 - RNA, 21 nt, Artificial Sequence
  Description of Artificial Sequence Synthetic oligonucleotide
aguuauagguaaguuagccca

SEQ ID NO: 1214 - RNA, 21 nt, Artificial Sequence
  Description of Artificial Sequence Synthetic oligonucleotide
auuauguccacuagggguaaa

SEQ ID NO: 1215 - RNA, 21 nt, Artificial Sequence
  Description of Artificial Sequence Synthetic oligonucleotide
aaagugucagauacggugugg

SEQ ID NO: 1216 - RNA, 23 nt, Artificial Sequence
  Description of Artificial Sequence Synthetic oligonucleotide
gaaacccagcagacaauguagcu

SEQ ID NO: 1217 - RNA, 23 nt, Artificial Sequence
  Description of Artificial Sequence Synthetic oligonucleotide
cauggagaaaggcacccacauau

SEQ ID NO: 1218 - RNA, 23 nt, Artificial Sequence
  Description of Artificial Sequence Synthetic oligonucleotide
accccaaagcagaacuaggaugu

SEQ ID NO: 1219 - RNA, 23 nt, Artificial Sequence
  Description of Artificial Sequence Synthetic oligonucleotide
aagccacccaacugaagagguau

SEQ ID NO: 1220 - RNA, 23 nt, Artificial Sequence
  Description of Artificial Sequence Synthetic oligonucleotide
guggccaaccagcaagaacauau

SEQ ID NO: 1221 - RNA, 24 nt, Artificial Sequence
  Description of Artificial Sequence Synthetic oligonucleotide
gagacccaguagccagauguagcu

SEQ ID NO: 1222 - RNA, 23 nt, Artificial Sequence
  Description of Artificial Sequence Synthetic oligonucleotide
uugggguauuugacaaacugaca

SEQ ID NO: 1223 - RNA, 23 nt, Artificial Sequence
  Description of Artificial Sequence Synthetic oligonucleotide
gagauuuggaugcucacaaguua

SEQ ID NO: 1224 - RNA, 23 nt, Artificial Sequence
  Description of Artificial Sequence Synthetic oligonucleotide
ggaauuucugauuacagugagca

SEQ ID NO: 1225 - RNA, 23 nt, Artificial Sequence
  Description of Artificial Sequence Synthetic oligonucleotide
cugcuaaugaaucaggaguugua

SEQ ID NO: 1226 - RNA, 23 nt, Artificial Sequence
  Description of Artificial Sequence Synthetic oligonucleotide
ucaguuggaacagcuguugaaua

SEQ ID NO: 1227 - RNA, 23 nt, Artificial Sequence
  Description of Artificial Sequence Synthetic oligonucleotide
uaaacggaaccacuagugacuug